SEQUENCE LISTING (39 sequences)

SEQ ID NO: 1 - skipped

SEQ ID NO: 2 - protein, 32 aa, Artificial Sequence
  Description of Artificial Sequence: Synthetic polypeptide
MRVKEKYQHLWRWGWKWGTMLLGILMICSATE

SEQ ID NO: 3 - protein, 4 aa, Artificial Sequence
  Description of Artificial Sequence: Synthetic peptide
CAAL

SEQ ID NO: 4 - protein, 6 aa, Artificial Sequence
  Description of Artificial Sequence: Synthetic peptide
KKKKKK

SEQ ID NO: 5 - protein, 5 aa, Artificial Sequence
  Description of Artificial Sequence: Synthetic peptide
RRRRR

SEQ ID NO: 6 - protein, 21 aa, Artificial Sequence
  Description of Artificial Sequence: Synthetic peptide
MRLLLALLGVLLSVPGPPVLS

SEQ ID NO: 7 - protein, 12 aa, Artificial Sequence
  Description of Artificial Sequence: Synthetic peptide
CSIMNLMCGSTC

SEQ ID NO: 8 - protein, 45 aa, Artificial Sequence
  Description of Artificial Sequence: Synthetic polypeptide
GHKSEEKREKMKRTLLKDWKTRLSYFLQNSSTPGKPKTGKKSKQQ

SEQ ID NO: 9 - protein, 18 aa, Artificial Sequence
  Description of Artificial Sequence: Synthetic peptide
RSTLKLTTLQCQYSTVMD

SEQ ID NO: 10 - protein, 18 aa, Artificial Sequence
  Description of Artificial Sequence: Synthetic peptide
RQGLHNMEDVYELIENSH

SEQ ID NO: 11 - protein, 30 aa, Artificial Sequence
  Description of Artificial Sequence: Synthetic polypeptide
MDCRKMARFSYSVIWIMAISKVFELGLVAG

SEQ ID NO: 12 - protein, 16 aa, Artificial Sequence
  Description of Artificial Sequence: Synthetic peptide
MPAWGALFLLWATAEA

SEQ ID NO: 13 - protein, 4 aa, Artificial Sequence
  Description of Artificial Sequence: Synthetic peptide
RDYR

SEQ ID NO: 14 - protein, 55 aa, Artificial Sequence
  Description of Artificial Sequence: Synthetic polypeptide
KMALRVALNNKQSGQITVKTSSSDHLSLAIAGLVPIALSIYQKFKPGVSPSYSIY

SEQ ID NO: 15 - protein, 21 aa, Artificial Sequence
  Description of Artificial Sequence: Synthetic peptide
MGSKIVQVFLMLALFATSALA

SEQ ID NO: 16 - protein, 21 aa, Artificial Sequence
  Description of Artificial Sequence: Synthetic peptide
MNSKAMQALIFLGFLATSCLA

SEQ ID NO: 17 - protein, 12 aa, Artificial Sequence
  Description of Artificial Sequence: Synthetic peptide
MGAAASIQTTVN

SEQ ID NO: 18 - protein, 11 aa, Artificial Sequence
  Description of Artificial Sequence: Synthetic peptide
FALLGTHGASG

SEQ ID NO: 19 - protein, 7 aa, Artificial Sequence
  Description of Artificial Sequence: Synthetic peptide
RRRTFLK

SEQ ID NO: 20 - protein, 10 aa, Artificial Sequence
  Description of Artificial Sequence: Synthetic peptide
MGGKWSKSSV

SEQ ID NO: 21 - protein, 6 aa, Artificial Sequence
  Description of Artificial Sequence: Synthetic peptide
DDPERE

SEQ ID NO: 22 - protein, 16 aa, Artificial Sequence
  Description of Artificial Sequence: Synthetic peptide
EEANTGENNSLLHPMS

SEQ ID NO: 23 - protein, 6 aa, Artificial Sequence
  Description of Artificial Sequence: Synthetic peptide
SRRGLV

SEQ ID NO: 24 - protein, 6 aa, Artificial Sequence
  Description of Artificial Sequence: Synthetic peptide
SRRRFL

SEQ ID NO: 25 - protein, 6 aa, Artificial Sequence
  Description of Artificial Sequence: Synthetic peptide
SRRQFI

SEQ ID NO: 26 - protein, 6 aa, Artificial Sequence
  Description of Artificial Sequence: Synthetic peptide
QRRDFL

SEQ ID NO: 27 - protein, 52 aa, Artificial Sequence
  Description of Artificial Sequence: Synthetic polypeptide
MNKIYSIKYSAATGGLIAVSELAKKVICKTNRKISAALLSLAVISYTNIIYA

SEQ ID NO: 28 - protein, 40 aa, Artificial Sequence
  Description of Artificial Sequence: Synthetic polypeptide
MNPNQKIITIGSICMVIGIVSLMLQIGNIISIWVSHSIQT

SEQ ID NO: 29 - protein, 16 aa, Artificial Sequence
  Description of Artificial Sequence: Synthetic peptide
LRCLACSCFRTPVWPR

SEQ ID NO: 30 - protein, 10 aa, Artificial Sequence
  Description of Artificial Sequence: Synthetic peptide
MGCGCSSHPE

SEQ ID NO: 31 - protein, 9 aa, Artificial Sequence
  Description of Artificial Sequence: Synthetic peptide
MPFVNKQFN

SEQ ID NO: 32 - protein, 28 aa, Artificial Sequence
  Description of Artificial Sequence: Synthetic peptide
DEQNAKNAAQDRNSNKSSKGFFSKLGCC

SEQ ID NO: 33 - protein, 10 aa, Artificial Sequence
  Description of Artificial Sequence: Synthetic peptide
MLCCMRRTKQ

SEQ ID NO: 34 - protein, 14 aa, Artificial Sequence
  Description of Artificial Sequence: Synthetic peptide
VTNGSTYILVPLSH

SEQ ID NO: 35 - protein, 7 aa, Artificial Sequence
  Description of Artificial Sequence: Synthetic peptide
AETENFV

SEQ ID NO: 36 - protein, 18 aa, Artificial Sequence
  Description of Artificial Sequence: Synthetic peptide
RARHRRNVDRVSIGSYRT

SEQ ID NO: 37 - protein, 4 aa, Artificial Sequence
  Description of Artificial Sequence: Synthetic peptide
YEDQ

SEQ ID NO: 38 - protein, 19 aa, Artificial Sequence
  Description of Artificial Sequence: Synthetic peptide
LLVTSLLLCELPHPAFLIP

SEQ ID NO: 39 - DNA, 11 bp, Artificial Sequence
  Description of Artificial Sequence: Synthetic oligonucleotide
  Description of Combined DNA/RNA Molecule: Synthetic oligonucleotide
aaaaaaaaaaa

(19) United States
(12) Patent Application Publication
(10) Pub. No.: US 2023/0226213 A1
(43) Pub. Date: Jul. 20, 2023

(54) METHODS AND COMPOSITIONS FOR MODULATING CELLS AND CELLULAR MEMBRANES

(71) Applicant: FLAGSHIP PIONEERING INNOVATIONS VI, LLC, Cambridge, MA (US)

(72) Inventors: Douglas Ewen CAMERON, Auburndale, MA (US); Bradley Joseph KUBICK, Watertown, MA (US)

(21) Appl. No.: 18/010,857

(22) PCT Filed: Jun. 18, 2021
(86) PCT No.: PCT/US2021/038087

§ 371 Date: Dec. 17, 2022

Related U.S. Application Data
(60) Provisional application No. 63/040,860, filed on Jun. 18, 2020.

(51) Int. Cl. A61K 47/69; C12N 15/113; C07K 14/435; C07K 14/705; C12N 5/00; C12N 5/078

(52) U.S. Cl. CPC A61K 47/6901; C12N 15/113; C07K 14/43595; C07K 14/705; C12N 5/0006; C12N 5/0634

(57) ABSTRACT

Compositions comprising donor cells, acceptor cells, membrane-enclosed bodies and methods are described herein.

### BACKGROUND

A number of therapeutic strategies comprise modifying a subject's cells, either ex vivo (followed by returning said cells to the subject) or in vivo. However, genetic methods of modifying cells have drawbacks such as off-target genetic modification of other genomic locations or non-target cell types. There is a need for alternative methods of modifying cells or addition of compounds to the target cell.

### SUMMARY

The present disclosure relates to the transfer of a membrane-associated agent and/or a cargo molecule from a first membrane (e.g., of a donor cell or membrane containing body) to a second membrane (e.g., of a target cell, e.g., an acceptor cell), to the donor cells and acceptor cells themselves, as well as systems and compositions comprising the same, and methods of making and using the same. Without wishing to be bound by theory, it is thought that natural processes in which a portion of the cell membrane of a cell is transferred to another cell, can be adapted as a method of modifying a cell (e.g., to express an exogenous protein, to alter expression of one or more genes, or reprogram/undifferentiate the cell).

In some aspects, the disclosure provides a donor cell comprising an membrane-associated agent, the agent comprising a membrane-associated moiety, and one or both of an extracellular moiety or an intracellular moiety, wherein the membrane-associated agent is configured to be transferred to an acceptor cell. In some embodiments, the donor cell comprises a cargo molecule configured to be transferred to an acceptor cell. In some embodiments, at least one of the membrane-associated moiety, extracellular moiety, intracellular moiety, or the cargo molecule is exogenous to the donor cell.

In another aspect, the disclosure provides a donor cell comprising: a membrane-associated agent comprising a membrane-associated moiety, and an extracellular moiety, an intracellular moiety, a cargo molecule, or a combination thereof. In some embodiments, at least one of the membrane-associated moiety, extracellular moiety, intracellular moiety, or cargo molecule is present at a different level in the donor cell than a source cell (e.g., from which the donor cell was derived), e.g., is differentially expressed. In some embodiments, the membrane-associated agent is transferred to an acceptor cell.

In another aspect, the disclosure provides an acceptor cell comprising: a membrane-associated agent, the agent comprising: a membrane-associated moiety, and one or both of an extracellular moiety or an intracellular moiety. In some embodiments, the acceptor cell does not comprise a nucleic acid encoding the membrane-associated agent (e.g., wherein the acceptor cells is not genetically modified to express the membrane-associated agent). In some embodiments, the acceptor cell comprises a cargo molecule, e.g., received from a donor cell. In some embodiments, at least one of the membrane-associated moiety, extracellular moiety, intracellular moiety, or cargo molecule is exogenous to the acceptor cell. In some embodiments, the acceptor cell comprises, e.g., received, the membrane-associated agent from a donor cell.

In another aspect, the disclosure provides an acceptor cell comprising: a membrane-associated agent comprising: a membrane-associated moiety, and one or both of an extracellular moiety or an intracellular moiety. In some embodiments, the acceptor cell does not substantially express, e.g., does not express, a nucleic acid encoding the membrane-associated agent. In some embodiments, the acceptor cell comprises a cargo molecule, e.g., received from a donor cell. In some embodiments, the acceptor cell received the membrane-associated agent from the donor cell.

In another aspect, the disclosure provides a composition, e.g., a preparation, comprising a plurality of donor or acceptor cells described herein.

In another aspect, the disclosure provides a system, e.g., a reaction mixture, comprising: a donor cell described herein, and an acceptor cell, wherein the donor cell and acceptor cell are provided under conditions suitable for transfer of the membrane-associated agent and/or cargo molecule from the donor cell to the acceptor cell. In some embodiments, the acceptor cell does not comprise a nucleic acid encoding the membrane-associated agent and/or the cargo molecule, or differentially expresses the membrane-associated agent and/or cargo molecule, e.g., relative to an endogenously-expressed membrane-associated agent and/or cargo molecule, if any, or relative to the acceptor cell prior to transfer.

In another aspect, the disclosure provides a pharmaceutical composition comprising a donor cell (e.g., plurality of donor cells) described herein, an acceptor cell (e.g., plurality of acceptor cells), or a combination thereof. In some embodiments, the pharmaceutical composition comprises one or more pharmaceutically acceptable excipients or carriers.

In another aspect, the disclosure provides a method of modifying an acceptor cell, comprising: contacting the acceptor cell with a donor cell or system described herein, under conditions suitable for transfer of the membrane-associated agent and/or cargo molecule to the acceptor cell, thereby modifying the acceptor cell. In some embodiments, the acceptor cell does not comprise a nucleic acid encoding the membrane-associated agent and/or cargo molecule. In some embodiments, after the transfer the acceptor cell comprises an increased amount of the membrane-associated agent and/or cargo molecule,

In another aspect, the disclosure provides a method of making a modified cell, comprising: providing an unmodified cell, and contacting the unmodified cell with a donor cell or system described herein, under conditions suitable for transfer of the membrane-associated agent and/or cargo molecule to the unmodified cell, thereby making a modified cell. In some embodiments, neither the unmodified cell or modified cell comprise a nucleic acid encoding the membrane-associated agent. In some embodiments after the transfer the modified cell comprises an increased amount of the membrane-associated agent and/or cargo molecule than the unmodified cell. In some embodiments, neither the unmodified cell or modified cell comprise a nucleic acid encoding the membrane-associated agent and after the transfer the modified cell comprises an increased amount of the membrane-associated agent and/or cargo molecule than the unmodified cell.

In another aspect, the disclosure provides a method of delivering a cargo molecule to a cell, comprising: providing a donor cell or the system described herein, wherein the donor cell comprises comprising the cargo molecule; providing an acceptor cell that does not comprise a nucleic acid encoding the membrane-associated agent and/or cargo molecule or does not substantially express (e.g., does not express) a nucleic acid encoding the membrane-associated agent and/or cargo molecule; and contacting the acceptor cell with the donor cell or system under conditions suitable for transfer of the membrane-associated agent to the acceptor cell, thereby delivering the cargo molecule to the cell.

In another aspect, the disclosure provides a method of modulating, e.g., enhancing or decreasing, a biological function in a subject, a target tissue, or a cell, comprising administering to the subject, or contacting the target tissue or the cell with, a donor cell, acceptor cell, system, or a pharmaceutical composition described herein, thereby modulating the biological function in the subject.

Any of the aspects herein, e.g., the donor cells, acceptor cells, membrane-associated bodies, membrane-enclosed bodies, compositions or preparations thereof, and methods above, can be combined with one or more of the embodiments herein, e.g., one or of the embodiments described herein.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jun. 18, 2020. When one gene or protein references a plurality of sequence accession numbers, all of the sequence variants are encompassed. In addition, the materials, methods, and examples are illustrative only and not intended to be limiting.

### BRIEF DESCRIPTION OF THE DRAWINGS

The following detailed description of the invention will be better understood when read in conjunction with the appended drawings. For the purpose of illustrating the invention, there are shown in the drawings described herein certain embodiments, which are presently exemplified. It should be understood, however, that the invention is not limited to the precise arrangement and instrumentalities of the embodiments shown in the drawings.

The patent or application file contains at least one drawing executed in color. Copies of this patent or patent application publication with color drawing(s) will be provided by the Office upon request and payment of the necessary fee.

FIG. 1 shows an exemplary donor cell, acceptor cell, membrane-associated agent, and vector encoding the same. The exemplary membrane-associated agent comprises a HLA-G-Lumio-MCP fusion protein comprising an MS2 Coat Protein (MCP) fused to the cytoplasmic C-terminus of HLA-G with a Lumio tag added to the linker. The vector also encodes an mRNA with a MS2 stem loops in the 3′UTR. The fusion protein is capable of binding the mRNA via MS2-MCP interaction, enabling the transfer of the entire complex to acceptor cells.

FIG. 2 shows a post-transfer acceptor cell comprising a membrane-associated agent associated with the inner leaflet of the cell membrane, the agent comprising an engineered protein comprising a Lck tyrosine kinase myristoylation or palmitoylation sequence as a membrane-associated moiety and an EGFP domain as an intracellular moiety.

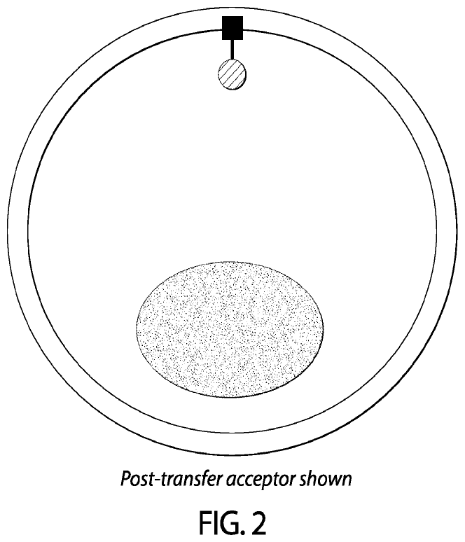

FIG. 3 shows a post-transfer acceptor cell comprising a membrane-associated agent associated with the inner leaflet of the cell membrane, the agent comprising a fusion protein comprising a Lck tyrosine kinase myristoylation or palmitoylation sequence as a membrane-associated moiety, and a Lumio tag and β-lactamase domain as an intracellular moiety. Activity of the β-lactamase enzyme can be assessed in acceptor cells via application of substrates that exhibit cytoplasmic fluorescence after cleavage.

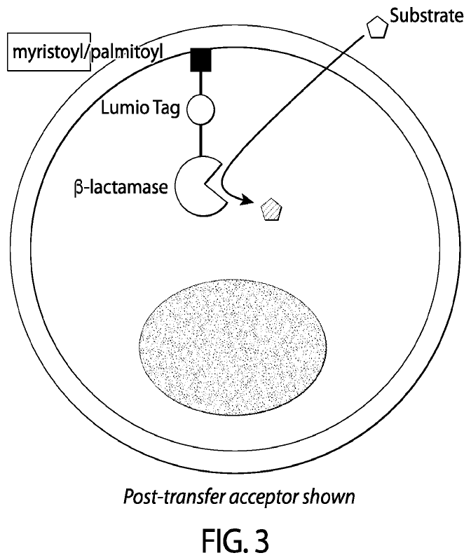

FIG. 4 shows a post-transfer acceptor cell comprising a membrane-associated agent comprising a membrane-associated moiety and extracellular moiety comprising a transmembrane domain and extracellular domain from E-selectin, and a Lumio tag and β-lactamase domain as an intracellular moiety. Activity of the β-lactamase enzyme can be assessed in acceptor cells via application of substrates that exhibit cytoplasmic fluorescence after cleavage.

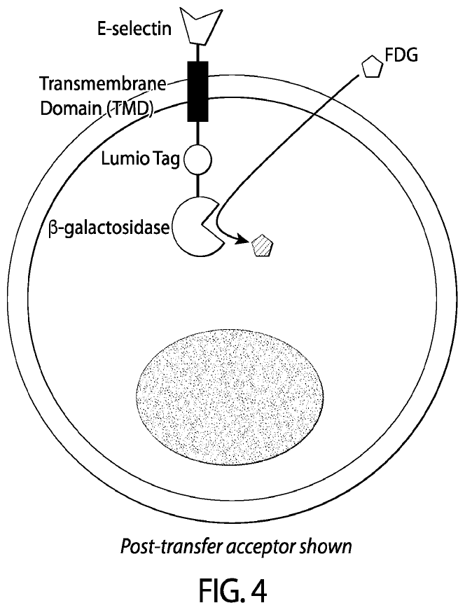

FIG. 5 shows a post-transfer acceptor cell comprising a membrane-associated agent comprising a membrane-associated moiety and extracellular moiety comprising a transmembrane domain and extracellular domain from Notch receptor and an intracellular moiety comprising a T-secretase cleavable Cre recombinase. This exemplary post-transfer acceptor cell comprises a reporter gene (EGFP) in the genomic DNA comprising a premature stop codon flanked by loxP sites.

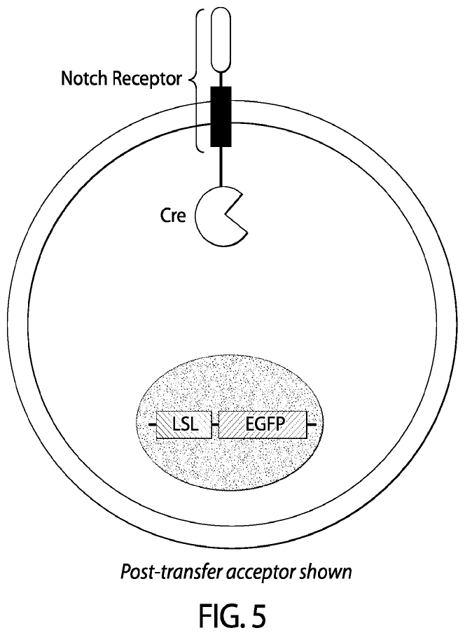

FIG. 6 shows a post-transfer acceptor cell comprising a membrane-associated agent comprising a membrane-associated moiety comprising a thrombomodulin transmembrane domain, an HLA-G extracellular moiety, and an intracellular moiety comprising a RHBDL2-releasable Cre recombinase and a Lumio tag. This exemplary post-transfer acceptor cell comprises a reporter gene (EGFP) in the genomic DNA comprising a premature stop codon flanked by loxP sites.

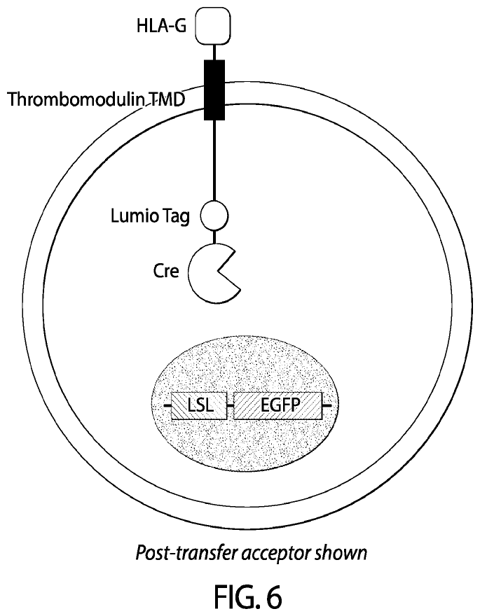

FIG. 7 shows a post-transfer acceptor cell comprising a membrane-associated agent associated with the inner leaflet of the cell membrane, the agent comprising a Lck tyrosine kinase myristoylation or palmitoylation sequence as a membrane-associated moiety, and an intracellular moiety comprising a Lumio tag, and a Cre recombinase and TEV cleavage tag situated such that cleavage by TEV frees the Cre recombinase. In this exemplary acceptor cell, an exogenous nucleic acid supplies a gene encoding TEV protease and a reporter gene (EGFP) comprising a premature stop codon flanked by loxP sites.

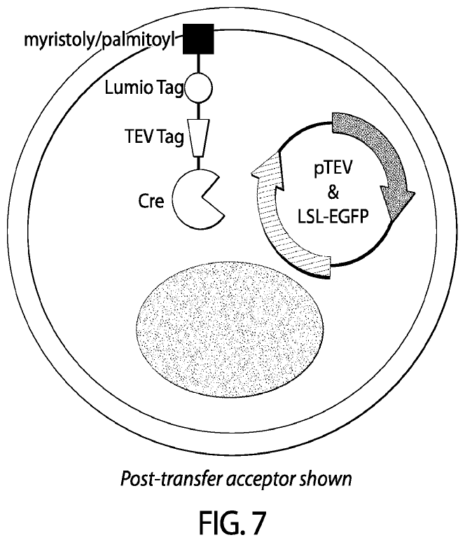

FIG. 8 shows a post-transfer acceptor cell comprising a membrane-associated agent comprising a membrane-associated moiety comprising a thrombomodulin transmembrane domain, an extracellular moiety comprising a streptavidin domain bound to a biotinylated antibody, and a Lumio tag and β-lactamase domain as an intracellular moiety. Without wishing to be bound by theory, a membrane-associated agent comprising a streptavidin domain may easily be conjugated to a biotinylated moiety, such as a variety of biotinylated antibodies or portions thereof. Activity of the β-lactamase enzyme can be assessed in acceptor cells via application of substrates that exhibit cytoplasmic fluorescence after cleavage.

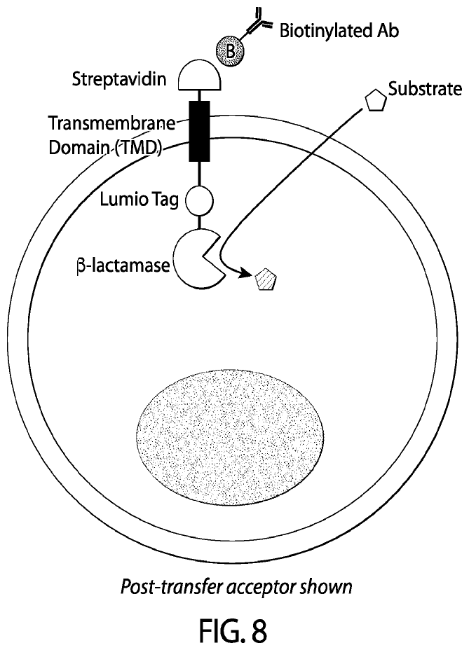

FIG. 9 shows a diagram of an exemplary membrane-associated agent, delineating different moieties and portions thereof, as well as exemplary functions or components of said moieties/portions.

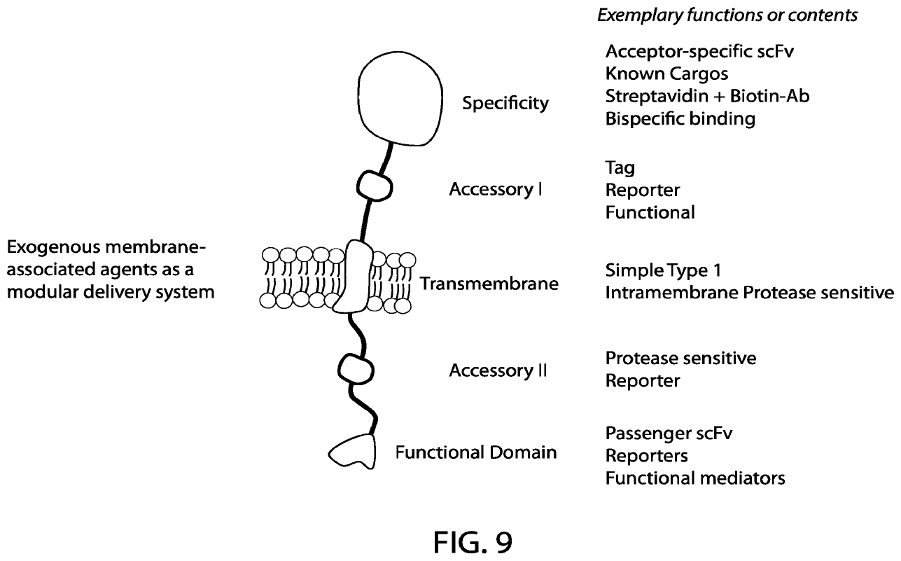

FIG. 10 shows a diagram of an exemplary donor cell comprising one or more modifications that modify which membrane-associated agents are configured for transfer to a target cell (e.g., acceptor cell).

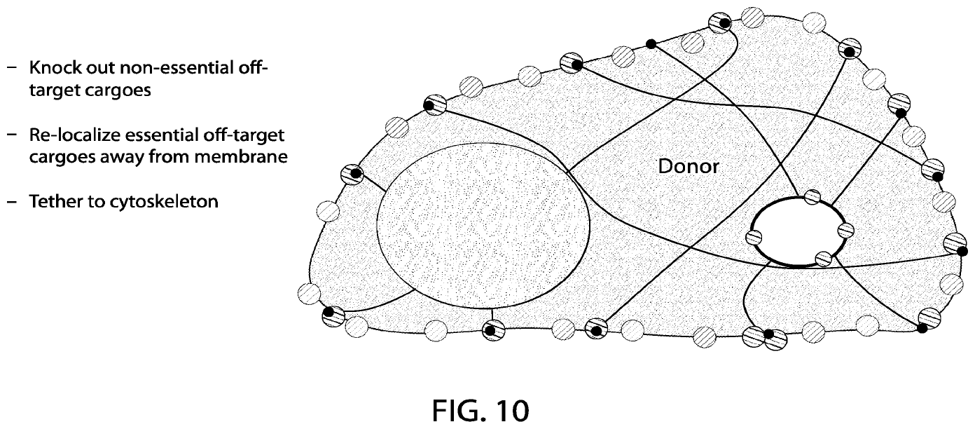

FIG. 11 shows a graph of transfer-positive exemplary acceptor Jurkat cells over increasing co-culture cell density.

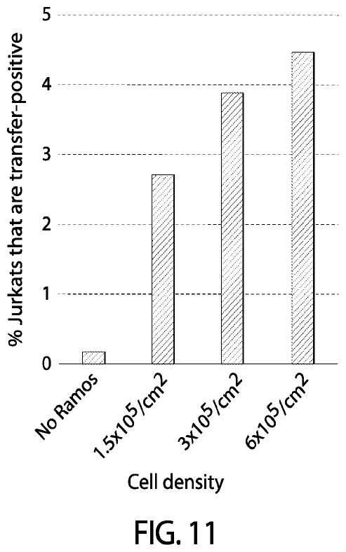

FIG. 12 shows flow cytometry data and gating strategies analyzing exemplary donor K562 cells and exemplary acceptor THP-1 cells, stimulated and unstimulated.

FIG. 13 shows flow cytometry data, applying the gating strategies from FIG. 12, analyzing the membrane content of exemplary acceptor THP-1 cells co-cultured with exemplary donor K562 cells.

FIG. 14 shows quantification of transfer incidence to exemplary acceptor THP-1 cells (stimulated or unstimulated) from exemplary donor K562 cells.

FIG. 15 shows flow cytometry data analyzing the membrane content of exemplary acceptor THP-1 cells co-cultured in the presence of exemplary donor K562 cells and PBMC cells.

FIG. 16 shows flow cytometry data analyzing the membrane content of subsets of PBMCs co-cultured with exemplary donor K562 cells.

FIG. 17 shows quantification of transfer incidence to subsets of PBMCs (stimulated or unstimulated) from exemplary donor JEG-3 cells or exemplary donor K562 cells.

FIG. 18 shows flow cytometry data analyzing the membrane content of exemplary acceptor THP-1 cells (stimulated or unstimulated) and showing transfer from exemplary donor JEG-3 cells, exemplary donor K562 cells, and both.

FIG. 19 shows flow cytometry data analyzing transfer of a membrane-associated agent (HLAG-lumio-MCP) from exemplary donor K562 cells to exemplary acceptor Jurkat cell.

FIG. 20 shows flow cytometry analyzing transfer of a membrane-associated agent from exemplary biotin labeled donor K562 cells to exemplary acceptor Jurkat cells and exemplary acceptor THP-1 cells.

FIG. 21 shows a chart depicting an exemplary application of the methods described herein: transfer of a cargo molecule (mRNA) from donor cells to a target endothelial cell using a membrane-associated agent.

FIG. 22 is a series of graphs showing siRNA-Cy3 transfer from J76 cells to Ramos cells.

FIG. 23 is a diagram showing an experimental design for co-culture of K562 donor cells expressing HLA-G with THP and Jurkat acceptor cells.

FIG. 24 is a series of graphs showing identification of K562 donor cells expressing HLA-G, THP-1 acceptor cells (stimulated with PMA or unstimulated), and Jurkat acceptor cells (stimulated with PMA and PHA-L, or unstimulated). Shown for each acceptor cell type are the results of culture alone, or co-culture with K562-HLA-G cells at a ratio of 1 donor: 5 acceptors, or at a ratio of 5 donors: 1 acceptor.

FIG. 25 is a series of graphs showing transfer of HLA-G and cell membrane from K562 donor cells to THP-1 acceptor cells.

FIG. 26 is a series of graphs showing transfer of HLA-G and cell membrane from K562 donor cells to Jurkat acceptor cells.

FIG. 27 is a series of graphs showing either the percent HLA-G-positive or the population MFI of HLA-G of the THP-1 acceptor cells and Jurkat acceptor cells (as indicated). Each graph shows inactivated or activated populations of acceptor cells.

FIG. 28 is a series of graphs showing detection of membrane transfer after co-culture of K562 cells with unstimulated PBMCs.

FIG. 29 is a series of graphs showing transfer of GFP protein from J76 donor cells to Ramos acceptor cells in co-culture.

### DETAILED DESCRIPTION

The invention describes donor cells, acceptor cells, membrane-enclosed bodies, membrane-containing substrates, membrane-associated agents, and related methods including methods of modifying acceptor cells, methods of delivering an membrane-associated agent and optionally one or more cargo molecules to an acceptor cell, methods of treating a subject, methods of transferring an membrane-associated agent from a donor cell to an acceptor cell, and methods of making said cells and membrane-enclosed bodies. Without wishing to be bound by theory, it is thought that a number of natural and artificially induced processes facilitate the transfer of membrane-associated agents from a first cell (e.g., a donor cell) to a second cell (e.g., an acceptor cell). Employing such processes to modify a feature of the second cell has a number of advantages over other cellular modification techniques. For example, such a method does not require genetic modification of the second cell (e.g., acceptor cell). A donor cell, acceptor cell, or membrane-enclosed body comprising a membrane-associated agent or cargo molecule administered to a subject may be able to travel to a target cell, e.g., target tissue, e.g., target organ, in a subject more effectively than the agent or cargo molecule alone (e.g., delivering a higher amount of, a more specifically targeted amount of, or delivering with improved pharmacokinetics (e.g., a higher or lower half-life) the membrane-associated agent or cargo molecule).

Cells, Membrane-Enclosed Bodies, and Membrane-Containing Substrates

The present disclosure is directed, in part, to cells (e.g., donor cells or acceptor cells), membrane-enclosed bodies, and membrane-containing substrates comprising a membrane-associated agent and optionally one or more cargo molecules. In some embodiments, a donor cell, membrane-containing substrate, or membrane-enclosed body described herein can be used to deliver a membrane-associated agent and optionally one or more cargo molecules to target cell, e.g., an acceptor cell. In some embodiments, a cell (e.g., a source cell, donor cell, or acceptor cell) is a purified cell, e.g., isolated from a culture, sample (e.g., apheresis sample), tissue, organ, or subject. In some embodiments, a cell (e.g., an acceptor cell) is part of a sample (e.g., apheresis sample), tissue, organ, or subject.

In some embodiments, the cell is a donor cell comprising an membrane-associated agent configured to be transferred to a target cell, e.g., an acceptor cell, and optionally comprising one or more cargo molecules also configured to be transferred to said target cell, e.g., an acceptor cell.

As used herein, “configured to be transferred” refers to a status of an agent that is associated with a membrane of a first cell (e.g., donor cell), first membrane-containing substrate, or first membrane-enclosed body and capable of being transferred to a membrane of a second cell (e.g., acceptor cell), second membrane-containing substrate, or second membrane-enclosed body by a membrane transfer process upon contacting the first cell (e.g., donor cell), first membrane-containing substrate, or first membrane-enclosed body with the second cell (e.g., acceptor cell), second membrane-containing substrate, or second membrane-enclosed body.

In some embodiments, the membrane-enclosed body lacks one or more features of a cell and comprises a membrane-associated agent configured to be transferred to a target cell, e.g., an acceptor cell, and optionally comprising one or more cargo molecules also configured to be transferred to said target cell, e.g., an acceptor cell. In some embodiments, the membrane-enclosed body is derived from a source cell.

In some embodiments, the membrane-containing substrate is a solid polymeric substrate (e.g., a scaffold or bead) comprising (e.g., on its surface, e.g., coated with) a plurality of lipids (e.g., a lipid layer, e.g., a lipid bilayer). In some embodiments, the membrane-containing substrate comprises a membrane-associated agent configured to be transferred to a target cell, e.g., an acceptor cell, and optionally comprising one or more cargo molecules also configured to be transferred to said target cell, e.g., an acceptor cell. In general, where a donor cell or membrane-enclosed body is described herein, so too a membrane-containing substrate could be utilized. As used herein, a “source cell” refers to a cell from which a donor cell, acceptor cell, or membrane-enclosed body is derived, e.g., obtained. In some embodiments, derived includes obtaining a membrane-enclosed body from a source cell and adding a membrane-associated agent to the membrane-enclosed body. In some embodiments, derived includes adding a membrane-associated agent or a nucleic acid encoding the same, and optionally adding one or more cargo molecules or nucleic acids encoding the same, to a source cell, e.g., to provide a donor cell comprising a membrane-associated agent and optionally one or more cargo molecules. In some embodiments, derived includes a modification, e.g., a genetic, epigenetic, transient expression modification (e.g., a knockdown), or other modification to a source cell (e.g., in addition to adding a membrane-associated agent or a nucleic acid encoding the same, and optionally adding one or more cargo molecules or nucleic acids encoding the same). In some embodiments, derived includes a modification, e.g., a genetic, epigenetic, transient expression modification (e.g., a knockdown), or other modification to a source cell without the addition of exogenous membrane-associated agent, one or more cargo molecules, or nucleic acids encoding the same, e.g., to provide an acceptor cell. In some embodiments, deriving a donor cell, acceptor cell, or membrane-enclosed body from a source cell consumes or destroys the source cell. One of skill in the art will understand that when comparing a donor cell, acceptor cell, or membrane-enclosed body to the source cell from which it was derived, the comparison is made between the donor cell, acceptor cell, or membrane-enclosed body and other source cells of the same kind as the one(s) used to derive the donor cell, acceptor cell, or membrane-enclosed body.

In some embodiments, the cell is an acceptor cell comprising a membrane-associated agent and optionally comprising one or more cargo molecules, wherein the acceptor cell does not comprise a nucleic acid encoding the membrane-associated agent or cargo molecule(s). In some embodiments, the acceptor cell received said membrane-associated agent and optionally one or more cargo molecules from a donor cell or membrane-enclosed body.

In some embodiments, the cell is an acceptor cell capable of receiving a membrane-associated agent and optionally one or more cargo molecules from a donor cell or membrane-enclosed body. In some embodiments, said acceptor cell does not comprise a membrane-associated agent or cargo molecule (e.g., yet). In some embodiments, the acceptor cell comprises one or more modifications increasing the efficacy and/or likelihood of receiving a membrane-associated agent or cargo molecule from a donor cell or membrane-enclosed body.

A cell (e.g., donor cell or acceptor cell) may be a naturally occurring cell, e.g., a mammalian cell, e.g., a human cell. In some embodiments, a donor cell is a naturally occurring cell modified to comprise a membrane-associated agent and optionally one or more cargo molecules. In some embodiments, an acceptor cell is a naturally occurring cell modified to comprise one or more modifications increasing the efficacy and/or likelihood of receiving a membrane-associated agent or cargo molecule from a donor cell or membrane-enclosed body.

In some embodiments, a donor cell, acceptor cell, or membrane-enclosed body is derived from a source cell. In some embodiments, deriving said cell or membrane-enclosed body from a source cell comprising modifying the source cell, e.g., to contain or express a membrane-associated agent and optionally one or more cargo molecules, or to comprise one or more modifications increasing the efficacy and/or likelihood of receiving a membrane-associated agent or cargo molecule from a donor cell or membrane-enclosed body.

In some embodiments, DNA in the donor cell, acceptor cell, or membrane-enclosed body or DNA in the source cell from which the aforementioned is derived is edited to correct a genetic mutation using a gene editing technology, e.g. a guide RNA and CRISPR-Cas9/Cpf1, or using a different targeted endonuclease (e.g., Zinc-finger nucleases, transcription-activator-like nucleases (TALENs)). In some embodiments, the genetic mutation is linked to a disease in a subject (e.g., from whom the source cell was taken or to whom the donor cell, acceptor cell, or membrane-enclosed body will be returned). Examples of edits to DNA include small insertions/deletions, large deletions, gene corrections with template DNA, or large insertions of DNA. In some embodiments, gene editing is accomplished with non-homologous end joining (NHEJ) or homology directed repair (HDR). In some embodiments, the edit is a knockout. In some embodiments, the edit is a knock-in. In some embodiments, both alleles of DNA are edited. In some embodiments, a single allele is edited. In some embodiments, multiple edits are made. In some embodiments, the donor cell or membrane-enclosed body (or source cell from which the donor cell or membrane-enclosed body is derived) is derived from a subject, or is genetically matched to the subject, or is immunologically compatible with the subject (e.g. having similar MEW).

In some embodiments, the cell (e.g., source cell, donor cell, or acceptor cell) is a stem cell, red blood cell, white blood cell, neutrophil, eosinophil, basophil, lymphocyte, platelet, nerve cell, neuroglial cell, muscle cell (e.g., skeletal, cardiac, or smooth muscle cell), cartilage cell, bone cell (e.g., osteoclast, osteoblast, or osteocyte), lining cell, skin cell, endothelial cell, epithelial cell, fat cell, or sex cell (e.g., spermatozoa or ova).

In some embodiments, the cell (e.g., source cell, donor cell, or acceptor cell) is a primary cell or an immortalized cell, e.g., a cell line (e.g., a human cell line). In some embodiments, the cell (e.g., source cell, donor cell, or acceptor cell) is chosen from an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial glial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell). In some embodiments, the cell (e.g., source cell, donor cell, or acceptor cell) is other than a 293 cell, HEK cell, human endothelial cell, or a human epithelial cell, monocyte, macrophage, dendritic cell, or stem cell. In some embodiments, the cell (e.g., source cell, donor cell, or acceptor cell) is a white blood cell or a stem cell. In some embodiments, the cell (e.g., source cell, donor cell, or acceptor cell) is selected from a neutrophil, a lymphocyte (e.g., a T cell, a B cell, a natural killer cell), a macrophage, a granulocyte, a mesenchymal stem cell, a bone marrow stem cell, an induced pluripotent stem cell, an embryonic stem cell, or a myeloblast.

In some embodiments, the cell (e.g., donor cell or acceptor cell) or membrane-enclosed body is a synthetic cell. In some embodiments, the cell (e.g., donor cell or acceptor cell) or membrane-enclosed body is a recombinant cell, e.g., a cell comprising a non-naturally occurring nucleic acid sequence.

In some embodiments the cell (e.g., source cell, donor cell, or acceptor cell) or membrane-enclosed body is not an immune effector cell or not derived from an immune effector cell. In some embodiments the cell (e.g., source cell, donor cell, or acceptor cell) or membrane-enclosed body is an immune effector cell or is derived from an immune effector cell. In some embodiments, an immune effector cell refers to a cell that is involved in an immune response, e.g., in the promotion of an immune effector response. Examples of immune effector cells include, but are not limited to, T cells, e.g., CD4+ and CD8+ T cells, alpha/beta T cells and gamma/delta T cells, B cells, natural killer (NK) cells, natural killer T (NKT) cells, and mast cells.

In some embodiments, a population of donor cells is contacted with a population of acceptor cells at a particular ratio of donor cells to acceptor cells. In some embodiments, the ratio of donor cells to acceptor cells is about 1:10,000, 1:7,500, 1:5,000, 1:2,500, 1:1000, 1:750, 1:500, 1:250, 1:100, 1:90, 1:80, 1:70, 1:60, 1:50, 1:40, 1:30, 1:20, 1:10, 1:5, 1:4, 1:3, 1:2, or 1:1. In embodiments, the ratio of donor cells to acceptor cells is about 1:5. In some embodiments, the ratio of acceptor cells to donor cells is about 1:10,000, 1:7,500, 1:5,000, 1:2,500, 1:1000, 1:750, 1:500, 1:250, 1:100, 1:90, 1:80, 1:70, 1:60, 1:50, 1:40, 1:30, 1:20, 1:10, 1:5, 1:4, 1:3, 1:2, or 1:1. In embodiments, the ratio of acceptor cells to donor cells is about 1:5. In some embodiments, the donor cells are K562 cells. In some embodiments, the acceptor cell is a peripheral blood mononuclear cell (PBMC), e.g., a lymphocyte (e.g., a T cell, B cell, or NK cell). In some embodiments, the acceptor cell is a monocyte. In some embodiments, the acceptor cell is a T cell.

In some embodiments, a donor cell transfers a membrane-associated agent or cargo molecule to an acceptor cell, e.g., as described herein. In some embodiments, a donor cell transfers a portion of its cell membrane to an acceptor cell, e.g., as described herein. In some embodiments, a donor cell transfers one or more cell membrane constituents (e.g., lipids, proteins, polysaccharides, or other molecules associated with and/or attached to the cell membrane) to an acceptor cell, e.g., as described herein.

In some embodiments, an acceptor cell transfers a membrane-associated agent or cargo molecule to a donor cell, e.g., as described herein. In some embodiments, an acceptor cell transfers a portion of its cell membrane to a donor cell, e.g., as described herein. In some embodiments, an acceptor cell transfers one or more cell membrane constituents (e.g., lipids, proteins, polysaccharides, or other molecules associated with and/or attached to the cell membrane) to a donor cell, e.g., as described herein.

In some embodiments, the membrane-associated agent or cargo molecule is present, per cell (e.g., donor cell or acceptor cell) or membrane-enclosed body, at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by quantitative flow cytometry. In some embodiments, the membrane-associated agent or cargo molecule is present at a copy number of at least 1,000 copies, e.g., as measured by quantitative flow cytometry. In some embodiments, at least 10%, 20%, 30%, 40%, 50%, 60%, 70%, 80%, 90%, 95%, 96%, 97%, 98%, or 99% of the membrane-associated agent or cargo molecule comprised by the cell (e.g., donor cell or acceptor cell) or membrane-enclosed body is disposed in the cell membrane. In embodiments, the cell (e.g., donor cell or acceptor cell) or membrane-enclosed body also comprises cargo molecule internally, e.g., in the cytoplasm or an organelle.

In some embodiments, the cell (e.g., donor cell or acceptor cell) or membrane-enclosed body comprises a therapeutic agent (e.g., a therapeutic membrane protein payload agent) at a copy number per cell or membrane-enclosed body of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by quantitative flow cytometry. In some embodiments, the cell (e.g., donor cell or acceptor cell) or membrane-enclosed body comprises a protein therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by quantitative flow cytometry. In some embodiments, the cell (e.g., donor cell or acceptor cell) or membrane-enclosed body comprises a nucleic acid therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) or membrane-enclosed body comprises a DNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) or membrane-enclosed body comprises an RNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) or membrane-enclosed body comprises an siRNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) or membrane-enclosed body comprises a therapeutic agent that is exogenous relative to the source cell at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) or membrane-enclosed body comprises a protein therapeutic agent that is exogenous relative to the source cell at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) or membrane-enclosed body comprises a nucleic acid (e.g., DNA or RNA) therapeutic agent that is exogenous relative to the source cell at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the ratio of the copy number of the membrane-associated agent to the copy number of the therapeutic agent is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000. In some embodiments, the ratio of the copy number of the membrane-associated agent to the copy number of the cargo molecule is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000.

In some embodiments, the cell (e.g., donor cell) or membrane-enclosed body delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a therapeutic agent (e.g., a therapeutic membrane protein payload agent). In some embodiments, the cell (e.g., donor cell) or membrane-enclosed body delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a protein therapeutic agent. In some embodiments, the cell (e.g., donor cell) or membrane-enclosed body delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a nucleic acid therapeutic agent. In some embodiments, the cell (e.g., donor cell) or membrane-enclosed body delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of an RNA therapeutic agent (e.g., an siRNA). In some embodiments, the cell (e.g., donor cell) or membrane-enclosed body delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a DNA therapeutic agent.

In some embodiments, the cell (e.g., donor cell) or membrane-enclosed body delivers to a target cell (e.g., acceptor cell) at least 10%, 20%, 30%, 40%, 50%, 60%, 70%, 80%, 90%, 95%, 96%, 97%, 98%, or 99% of a membrane-associated agent or cargo molecule (e.g., a therapeutic agent, e.g., a therapeutic agent that is endogenous or exogenous relative to the source cell) comprised by the cell (e.g., donor cell) or membrane-enclosed body delivers. In some embodiments, the cells (e.g., donor cell) or membrane-enclosed bodies that interact with the target cell(s) (e.g., acceptor cell) deliver to the target cell an average of at least 10%, 20%, 30%, 40%, 50%, 60%, 70%, 80%, 90%, 95%, 96%, 97%, 98%, or 99% of the membrane-associated agent or cargo molecule comprised by the cells (e.g., donor cell) or membrane-enclosed bodies that interact with the target cell(s). In some embodiments, the donor cell or membrane-enclosed body composition delivers to a target tissue (e.g., comprising a plurality of target cells, e.g., acceptor cells) at least 10%, 20%, 30%, 40%, 50%, 60%, 70%, 80%, 90%, 95%, 96%, 97%, 98%, or 99% of the membrane-associated agents or cargo molecules comprised by the donor cell or membrane-enclosed body composition.

In some embodiments, a provided cell (e.g., donor cell or acceptor cell) or membrane-enclosed body, and/or compositions or preparations thereof, comprise 0.00000001 mg exogenous membrane-associated agent or cargo molecule to 1 mg exogenous membrane-associated agent or cargo molecule per mg of total protein in the cell or membrane-enclosed body, e.g., 0.00000001-0.0000001, 0.0000001-0.000001, 0.000001-0.00001, 0.00001-0.0001, 0.0001-0.001, 0.001-0.01, 0.01-0.1, or 0.1-1 mg exogenous membrane-associated agent or cargo molecule per mg of total protein in the cell or membrane-enclosed body. In some embodiments, a provided cell (e.g., donor cell or acceptor cell) or membrane-enclosed body, and/or compositions or preparations thereof, comprises 0.00000001 mg exogenous membrane-associated agent or cargo molecule to 5 mg exogenous membrane-associated agent or cargo molecule per mg of lipid in the cell or membrane-enclosed body, e.g., 0.00000001-0.0000001, 0.0000001-0.000001, 0.000001-0.00001, 0.00001-0.0001, 0.0001-0.001, 0.001-0.01, 0.01-0.1, 0.1-1, or 1-5 mg exogenous membrane-associated agent or cargo molecule per mg of lipid in the cell or membrane-enclosed body.

In some embodiments, provided cells (e.g., donor cells or acceptor cells), membrane-enclosed bodies, and/or compositions or preparations thereof, meet a pharmaceutical or good manufacturing practices (GMP) standard. In some embodiments, provided cells (e.g., donor cells or acceptor cells), membrane-enclosed bodies, and/or compositions or preparations thereof, were made according to good manufacturing practices (GMP). In some embodiments, provided cells (e.g., donor cells or acceptor cells), membrane-enclosed bodies, and/or compositions or preparations thereof, are characterized by a pathogen level below a predetermined reference value, e.g., are substantially free of pathogens. In some embodiments, provided cells (e.g., donor cells or acceptor cells), membrane-enclosed bodies, and/or compositions or preparations thereof, have a contaminant (e.g., nuclear component such as nuclear DNA) level below a predetermined reference value, e.g., are substantially free of one or more specified contaminants. In some embodiments, provided cells (e.g., donor cells or acceptor cells), membrane-enclosed bodies, and/or compositions or preparations thereof, are characterized by low immunogenicity, e.g., as described herein.

In some embodiments, a donor cell comprises a membrane-associated agent and optionally a cargo molecule configured for transfer to an acceptor cell. In some embodiments, the donor cell comprises a T cell and the acceptor cell comprises an NK cell. In some embodiments, the donor cell comprises a dendritic cell and the acceptor cell comprises a T cell. In some embodiments, the donor cell comprises a dendritic cell and the acceptor cell comprises a NK cell. In some embodiments, the donor cell comprises a T cell and the acceptor cell comprises a T cell. In some embodiments, the membrane-associated agent and/or cargo molecule comprises a T cell Receptor (TCR) or a chimeric antigen receptor (CAR).

In some embodiments, a donor cell comprises a membrane-associated agent and optionally a cargo molecule configured for transfer to an acceptor cell. In some embodiments, the donor cell is a cancer cell and the acceptor cell is a T cell. In some embodiments, the membrane-associated agent and/or cargo molecule comprises a cancer driving agent (e.g., the expression product of an oncogene), a tissue specific marker, or a tumor marker. In some embodiments, the donor cell is a cancer cell and the acceptor cell is a circulating cell. In some embodiments, the donor cell is a cancer cell and the acceptor cell is a circulating cell.

In some embodiments, a donor cell comprises a membrane-associated agent and optionally a cargo molecule configured for transfer to an acceptor cell. In some embodiments, the donor cell is a dendritic cell and the acceptor cell is a cell that has been transplanted into a subject (e.g., as part of a tissue or organ transplant). In some embodiments, the membrane-associated agent and/or cargo molecule comprises a MHC protein, e.g., MHC class I.

In some embodiments, a donor cell comprises a membrane-associated agent and optionally a cargo molecule configured for transfer to an acceptor cell. In some embodiments, the donor cell is a progenitor cell and the acceptor cell is a differentiated cell. In some embodiments, the membrane-associated agent and/or cargo molecule comprises a reprogramming factor.

In some embodiments, a donor cell or membrane-enclosed body comprises a receptor and an acceptor cell comprises a ligand of said receptor. In some embodiments, a donor cell or membrane-enclosed body comprises a ligand and an acceptor cell comprises a receptor for said ligand. In some embodiments, a membrane-associated agent comprises the receptor, the ligand, or a functional portion thereof, e.g., as part of an extracellular moiety (e.g., a targeting domain) or membrane-associated moiety. In some embodiments, a targeting domain (e.g., not operably associated with a membrane-associated agent or cargo molecule) comprises the receptor, the ligand, or a functional portion thereof. In some embodiments, a donor cell or membrane-enclosed body comprises an interleukin and an acceptor cell comprises a receptor of said interleukin. In some embodiments, a donor cell or membrane-enclosed body comprises a receptor and an acceptor cell comprises an interleukin that can be bound by said receptor.

In some embodiments, a receptor, interleukin, ligand, or pair thereof for use in the compositions or methods of the disclosure is selected from Tables 1-6.

TABLE 1

| Receptor (T cell) | Ligands (APC/tumor) |
|---|---|
| PD1 | PD-L1, PD-L2 |
| CD28 | CD80, CD86 |
| CTLA-4 | CD80, CD86 |
| ICOS | B7RP1 |
| TMIGD2 | B7-H7 (HHLA2) |
| 4-1BB | 4-1BBL |
| BTLA | HVEM |
| CD160 | HVEM |
| LIGHT | HVEM |
| LAG3 | MHC class I/II |
| OX40 | OX40L |
| CD27 | CD70 |
| CD40L | CD40 |
| GITR | GITRL |
| DNAM-1 | CD155 |
| TIGIT | CD155 |
| CD96 | CD155 |
| 2B4 | CD48 |
| TIM-3 | Galaectin-9 |
| Adenosine A2a receptor | Adenosine |
| | IDO |
| | TDO |
| CEACAM1 | CEACAM1 |
| SIRP alpha | CD47 |
| DC-SIGN | BTN2A1 |
| CD200R | CD200 |
| DR3 | TL1A |
| Tryptophan | IDO1/TDO |
| CD40L | CD40 |

TABLE 2

| Receptor (NK) | Ligand (target) |
|---|---|
| Trail | TRAILR1, TRAILR2 |
| CD16 | Fc of IgG |
| NKp30a, b | b7-H6, BAT3, BAG6 |
| NKG2C | HLA-E |
| NKG2D | MICA/B, ULBP1-6 |
| 2B4 | CD48 |
| DNAM-1 | CD112, CD155 |
| NKG2A | HLA-E |
| CD137 | antibodies |
| OX40 | antibodies |
| CD27 | CD70 |
| KIR2DL1 | MHC receptors |
| KIR2DL2 | |
| KIR2DL3 | |
| KIR2DL4 | |
| KIR2DL5A | |
| KIR2DL5B | |
| KIR2DS1 | |
| KIR2DS2 | |
| KIR2DS3 | |
| KIR2DS4 | |
| KIR2DS5 | |
| KIR3DL1 | |
| KIR3DL2 | |
| KIR3DL3 | |
| KIR3DS1 | |
| FcR gamma | antibody Fc |
| CD3 zeta | |
| DAP12 | |
| NKp44 | PCNA |
| NKp46 | |
| NKp80 | AICL |
| CD94 | HLA-E |
| DAP10 | |
| CRTAM | Necl-2 |
| TIGIT | CD112, CD155, CD113 |
| CD96 | CD155, CD111 |
| NTB-A (SLAMF6) | NTB-A (SLAMF6) |
| CRACC (SLAMF7) | CRACC (SLAMF7) |
| CD27 | CD27L |
| CD100 | CD72 |
| CD160 | HLA-C |
| CEACAM-1 | CEACAM-1, CEACAM-5 |
| ILT2/LILRB1 | HLA Class I |
| KLRG1 | E-, N-, R- Cadherins |
| LAIR1 | Collagen |
| CD161/NKR-P1A | OCIL/CLEC2d |
| Siglec-3 | Sialic Acid |
| Siglec-7 | |
| Siglec-9 | |

TABLE 3

| Interleukin | Target Receptors |
|---|---|
| IL-1 | CD121a/IL1R1, CD121b/IL1R2 |
| IL-2 | CD25/IL2RA, CD122/IL2RB, CD132/IL2RG |
| IL-3 | CD123/IL3RA, CD131/IL3RB |
| IL-4 | CD124/IL4R, CD132/IL2RG |
| IL-5 | CD125/IL5RA, CD131/IL3RB |
| IL-6 | CD126/IL6RA, CD130/IR6RB |
| IL-7 | CD127/IL7RA, CD132/IL2RG |
| IL-8 | CXCR1/IL8RA, CXCR2/IL8RB/CD128 |
| IL-9 | CD129/IL9R |
| IL-10 | CD210/IL10RA, CDW210B/IL10RB |
| IL-11 | IL11RA |
| IL-12 | CD212/IL12RB1, IR12RB2 |
| IL-13 | IL13R |
| IL-14 | |
| IL-15 | IL15RA |
| IL-16 | CD4 |
| IL-17 | CDw217/IL17RA, IL17RB |
| IL-18 | CDw218a/IL18R1 |
| IL-19 | IL20R |
| IL-20 | IL20R |
| IL-21 | IL21R |
| IL-22 | IL22R |
| IL-23 | IL23R |
| IL-24 | IL20R |
| IL-25 | LY6E |
| IL-26 | IL20R1 |
| IL-27 | IL27RA |
| IL-28 | IL28R |
| IL-29 | |
| IL-30 | |
| IL-31 | IL31RA |
| IL-32 | |
| IL-33 | |
| IL-35 | |
| IL-36 | |

TABLE 4

| | | Exemplary Cell | |
|---|---|---|---|
| CXC | Corresponding | Types Associated | Exemplary |
| Chemokine | CXCR | with Receptor | Associated |
| Receptors | Ligands | and Ligand | Processes |
| CXCR1 | CXCL6, CXCL8, | Neutrophils | Neutrophil |
| | | | chemotaxis |
| CXCR2 | CXCL1, CXCL7 | Neutrophils | Neutrophil |
| | | | chemotaxis |
| CXCR3 | CXCL9, CXCL10, | T cells & B/NK | Activated |
| | CXCL11 | cells, epithelial | state homing |
| | | cells | |
| CXCR4 | CXCL12, | hematopoietic | Lymphocyte |
| | Ubiquitin, | cells, vascular | chemotaxis, |
| | MIF | endothelial cells, | BM homing, |
| | | Neurons, | B cell CD20 |
| | | endometrium | expression, |
| | | | HIV entry, |
| | | | embryo |
| | | | implantation, |
| | | | Neuronal |
| | | | guidance |
| CXCR5 | CXCL13 | B cells | Migration B |
| | | | cells, |
| | | | migration of |
| | | | T cells to |
| | | | lymph node B |
| | | | cell zones |
| CXCR6 | CXCL16 | T cells (HIV | HIV entry, |
| | | target) | homing to |
| | | | liver |
| CXCR7 | CXCL12 | T cells & memory | T cell |
| | | B cells | chemotaxis to |
| | | | CXCL12 |

TABLE 5

| | | Exemplary Cell | |
|---|---|---|---|
| | | Types Associated | Exemplary |
| CC Chemokine | Corresponding | with Receptor and | Associated |
| Receptors | CCR Ligands | Ligand | Processes |
| CCR1 | CCL4, CCL5, CCL6, | Peripheral blood | Effector immune |
| | CCL14, CCL15, | lymphocytes/mono- | recruitment to |
| | CCL16, CCL23 | cytes | inflammation |
| CCR2 | CCL2, CCL8, CCL16 | Monocytes, activated | Monocyte |
| | | memory T/B cells, | chemotaxis |
| | | basophils, perioneal | |
| | | Macs | |
| CCR3 | CCL11, CCL26, | Eosinophils, | Allergy |
| | CCL7, CCL13, CCL15, | basophils, Th1, Th2, | |
| | CCL24, CCL5, | airway epithelial cells | |
| | CCL28, CCL18 | | |
| CCR4 | CCL3, CCL5, | Th2 cells, dendritic | Activation trafficking |
| | CCL17, CCL22 | cells | |
| CCR5 | CCL3, CCL4, CCL5, | peripheral blood- | HIV coreceptor, |
| | CCL8, CCL11, | derived dendritic | trafficking |
| | CCL13, CCL14, | cells, | |
| | CCL16 | CD34+ hematopoietic | |
| | | progenitor cells and | |
| | | certain | |
| | | activated/memory Th1 | |
| | | lymphocytes | |
| CCR6 | CCL20 | unactivated memory | naïve trafficking |
| | | T cells, dendritic | |
| | | cells, Th17 | |
| CCR7 | CCL19, CCL21 | B cells, T cells, | trafficking across |
| | | dendritic cells, | high endothelial |
| | | metastic tumor cells | venules, sub-tissue |
| | | | secondary lymph |
| | | | tissue homing |
| CCR8 | CCL1, CCL16 | Th2 cells | Thymic homing |
| CCR9 | CCL25 | Thymic T cells, | Thymic/gut homing |
| | | intestinal T cells | |
| CCR10 | CCL27, CCL28 | Tregs | Skin inflammation, |
| | | | Mucosal Treg |
| | | | recruitment |
| CCR11 | CCL19, CCL21, | Tissue stromal cells | Putative scavenger of |
| | CCL25 | | CCL19, Dendritic |
| | | | cell mobilization to |
| | | | lymph nodes |

TABLE 6

| | | Exemplary Cell | |
|---|---|---|---|
| | | Types Associated | Exemplary |
| Formylpeptide | | with Receptor and | Associated |
| receptors (FPRs) | Ligands | Ligand | Processes |
| FPR1 | bacterial/mitochon- | Multiple tissues, | PARP detection, |
| | drial N-formyl peptides, | phagocytes, | homing, and response |
| | HIV envelope, | leukocytes | |
| | Annexin 1 | | |
| FPR2 | Various PARPs | neutrophils, | PARP detection, |
| | | eosinophils, | homing, and response |
| | | monocytes, | |
| | | macrophages, T cells, | |
| | | synovial fibroblasts, | |
| | | and intestinal and | |
| | | airway epithelium | |
| FPR3 | F2L | Monocytes, DCs | Scavenger/decoy, |
| | | | recycled, vesicle- |
| | | | localized |
| C Chemokine | | | |
| Receptor | | | |
| XCR1 | XCL1, XCL2 | Splenic DCs, thymic | Cross-presentation of |
| | | DCs, CD8 T cells | oral antigens, T cell |
| | | | repertoire selection |
| CX3C Chemokine | | | |
| receptor | | | |
| CX3CR1 | CX3CL1 | Leukocytes | migration and |
| | | (lymphocytes and | synapse pruning by |
| | | monocytes), | microglia, monocyte |
| | | microglia | survival |

In some embodiments, a receptor is a G-protein coupled receptor (GPCR), e.g., a Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors), or Class F (or 6) (Frizzled/Smoothened) GPCR. In some embodiments, the GPCR is associated with visual sense, gustatory sense, olfactory sense, neural-behavior and mood, immune regulation, neural-autonomic functions, cell density sensing, homeostasis, tumor biology, or endocrine receptors. In some embodiments, the GPCR is a member of or associated with the 5-hydroxytryptamine receptors, acetylcholine receptors (muscarinic), adenosine receptors, adhesion class GPCRs, adrenoceptors, angiotensin receptors, apelin receptor, bile acid receptor, bombesin receptors, bradykinin receptors, calcitonin receptors, calcium-sensing receptor, cannabinoid receptors, chemerin receptors, chemokine receptors, cholecystokinin receptors, class frizzled GPCRs, complement peptide receptors, corticotropin-releasing factor receptors, dopamine receptors, endothelin receptors, G protein-coupled estrogen receptor, formylpeptide receptors, free fatty acid receptors, GABAB receptors, galanin receptors, ghrelin receptor, glucagon receptor family, glycoprotein hormone receptors, gonadotrophin-releasing hormone receptors, GPR18, GPR55, GPR119, histamine receptors, hydroxycarboxylic acid receptors, kisspeptin receptor, leukotriene receptors, lysophospholipid (LPA) receptors, lysophospholipid (S1P) receptors, melanin-concentrating hormone receptors, melanocortin receptors, melatonin receptors, metabotropic glutamate receptors, motilin receptor, neuromedin U receptors, neuropeptide FF/neuropeptide AF receptors, neuropeptide S receptor, neuropeptide W/neuropeptide B receptors, neuropeptide Y receptors, neurotensin receptors, opioid receptors, orexin receptors, oxoglutarate receptor, P2Y receptors, parathyroid hormone receptors, platelet-activating factor receptor, prokineticin receptors, prolactin-releasing peptide receptor, prostanoid receptors, proteinase-activated receptors, QRFP receptor, relaxin family peptide receptors, somatostatin receptors, succinate receptor, tachykinin receptors, thyrotropin-releasing hormone receptors, trace amine receptor, urotensin receptor, vasopressin and oxytocin receptors, VIP or PACAP receptors.

In some embodiments, a receptor is a receptor tyrosine kinase (RTK), e.g., of RTK class I (EGF receptor family) (ErbB family), RTK class II (Insulin receptor family), RTK class III (PDGF receptor family), RTK class IV (VEGF receptors family), RTK class V (FGF receptor family), RTK class VI (CCK receptor family), RTK class VII (NGF receptor family), RTK class VIII (HGF receptor family), RTK class IX (Eph receptor family), RTK class X (AXL receptor family), RTK class XI (TIE receptor family), RTK class XII (RYK receptor family), RTK class XIII (DDR receptor family), RTK class XIV (RET receptor family), RTK class XV (ROS receptor family), RTK class XVI (LTK receptor family), RTK class XVII (ROR receptor family), RTK class XVIII (MuSK receptor family), RTK class XIX (LMR receptor), or RTK class XX (Undetermined).

In some embodiments, a receptor is a scavenger receptor, e.g., a Class A, B, C, D, E, F, G, H, I, or J scavenger receptor. In some embodiments, the scavenger receptor targets LDLs, apoptotic cells, PAMPs, leukocyte ligands/adhesion molecules, or chemokines. In some embodiments, the scavenger receptor is chosen from MSR1, an alternatively spliced form of SR-A1, MARCO, SCARA3, COLEC12, SCARA5, CD36, SCARB1, SCARB2, CD68, OLR1, Dectin 1, SCARF1, SCARF2, MEGF10, CXCL16, STAB1, STAB2, CD163, CD163L1, SRCRB4D, SSC5D, CD14, CD205, CD206, CD207, CD209\DC-SIGN, RAGE (membrane form), or RAGE (soluble form).

### Donor Cells

In some embodiments, a donor cell comprises a membrane-associated agent configured to be transferred to a target cell, e.g., an acceptor cell, and optionally one or more cargo molecules also configured to be transferred to the target cell (e.g., the acceptor cell).

As used herein, the term “donor cell” refers to a cell (e.g., a purified cell) comprising an membrane-associated agent configured to be transferred to a target cell, e.g., an acceptor cell. In some embodiments, a donor cell comprises one or more nucleic acids encoding the membrane-associated agent. In some embodiments, the donor cell comprises one or more modifications (e.g., in addition to the membrane-associated agent) relative to a source cell (e.g., from which the donor cell was derived), e.g., that enhance the donor cell's capability to transfer a membrane-associated agent to a target cell (e.g., an acceptor cell).

In some embodiments, a donor cell or the source cell used to make a donor cell is a cancer cell, a T cell, a B cell, or a cell derived from any thereof.

In some embodiments, a donor cell or source cell used to make a donor cell is a cell from a cell line (e.g., an immortalized cell line), e.g., K562, Ramos, HUVEC, 293T, RAW264.7, BT-474, SK-BR-3, MDA-MB-231, or BT-20 cell (e.g., as available from ATCC).

In some embodiments, a donor cell or membrane-enclosed body is capable of transferring a membrane-associated agent and optionally one or more cargo molecules to at least two, at least three, at least four, at least five, or more (e.g., any or all) types of acceptor cells. In some embodiments, a donor cell or membrane-enclosed body is capable of transferring the membrane-associated agent and optionally one or more cargo molecules to a specific acceptor cell type (e.g., and not to other acceptor cell types). In some embodiments, a donor cell or membrane-enclosed body is capable of transferring the membrane-associated agent and optionally one or more cargo molecules to a specific acceptor cell type and not to at least one, two, three, four, five, or more (e.g., all other) types of cells.

In some embodiments, donor cells or membrane-enclosed bodies, and/or compositions or preparations thereof, are characterized by a half-life in a subject, e.g., in a mouse, that is within 1%, 2%, 3%, 4%, 5%, 10%, 20%, 30%, 40%, 50%, 60%, 70%, 80%, 90%, 100% of the half-life of a reference cell, e.g., the source cell. In some embodiments, donor cells or membrane-enclosed bodies, and/or compositions or preparations thereof, are characterized by a half-life in a subject, e.g., in a mouse, that is at least 1 hour, 2 hours, 3 hours, 4 hours, 5 hours, 6 hours, 12 hours, or 24 hours, e.g., in a human subject or in a mouse. In some embodiments, donor cells or membrane-enclosed bodies, and/or compositions or preparations thereof, are capable of delivering (e.g., deliver) a exogenous membrane-associated agent or cargo molecule (e.g., a therapeutic agent) that is characterized by a half-life in a subject that is longer than the half-life of the donor cell, e.g., by at least 10%, 20%, 50%, 2-fold, 5-fold, or 10-fold. For instance, the donor cell may deliver the therapeutic agent to the target cell (e.g., acceptor cell), and the therapeutic agent may be present after the donor cell is no longer present or detectable.

In some embodiments, a characteristic of a provided cell (e.g., donor cell or acceptor cell) or membrane-enclosed body, and/or of a composition or preparations thereof, is described by comparison to a reference cell. In embodiments, the reference cell is the source cell from which the donor cell, acceptor cell, or membrane-enclosed body was derived. In embodiments, the reference cell is a Huvec, K562, THP-1, Jurkat, KHYG-1, Ramos, PBMC, and isolated PBMC subset cell. In some embodiments, a characteristic of a population of donor cells, acceptor cells, or membrane-enclosed bodies, and/or of a composition or preparation thereof, is described by comparison to a population of reference cells, e.g., a population of source cells, or a population of Huvec, K562, THP-1, Jurkat, KHYG-1, Ramos, PBMC, and isolated PBMC subset cell.

Donor Cells Generated from Culture Cells

Compositions of donor cells or membrane-enclosed bodies may be generated from source cells in culture, for example cultured mammalian cells, e.g., cultured human cells. The cells may be progenitor cells or non-progenitor (e.g., differentiated) cells. The cells may be primary cells or cell lines (e.g., a mammalian, e.g., human, cell line described herein). In embodiments, the cultured cells are progenitor cells, e.g., bone marrow stromal cells, marrow derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, or hepatoblasts.

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial glial cell, a bone marrow stromal cell, a pancreatic progenitor cell, an endothelial progenitor cell, a blast cell), an immortalized cell (e.g., HeLa, HEK293), or a Huvec, K562, THP-1, Jurkat, KHYG-1, Ramos, PBMC, or isolated PBMC subset cell.

The cultured cells may be from epithelial, connective, muscular, or nervous tissue or cells, and combinations thereof. Donor cells or membrane-enclosed bodies can be generated from cultured cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In embodiments, the cells are from a highly mitotic tissue (e.g., a highly mitotic healthy tissue, such as epithelium, embryonic tissue, bone marrow, intestinal crypts). In embodiments, the tissue sample is a highly metabolic tissue (e.g., skeletal tissue, neural tissue, cardiomyocytes).

In some embodiments a donor cell or membrane-enclosed body (or a source cell used to derive the same) is a suspension cell. In some embodiments a donor cell or membrane-enclosed body (or a source cell used to derive the same) is an adherent cell.

In some embodiments, a donor cell or membrane-enclosed body (or a source cell used to derive the same) are from a young donor, e.g., a donor 25 years, 20 years, 18 years, 16 years, 12 years, 10 years, 8 years of age, 5 years of age, 1 year of age, or less. In some embodiments, a donor cell or membrane-enclosed body (or a source cell used to derive the same) are from fetal tissue.

In some embodiments, the donor cells or membrane-enclosed bodies are derived from cells from a subject and administered to the same subject or a subject with a similar genetic signature (e.g., WIC-matched).

In certain embodiments, a donor cell or membrane-enclosed body (or a source cell used to derive the same) has telomeres of average size greater than 3000, 4000, 5000, 6000, 7000, 8000, 9000, or 10000 nucleotides in length (e.g., between 4,000-10,000 nucleotides in length, between 6,000-10,000 nucleotides in length).

Donor cells or membrane-enclosed bodies may be generated from cells generally cultured according to methods known in the art. In some embodiments, the cells may be cultured in 2 or more “phases”, e.g., a growth phase, wherein the cells are cultured under conditions to multiply and increase biomass of the culture, and a “production” phase, wherein the cells are cultured under conditions to alter cell phenotype (e.g., to maximize mitochondrial phenotype, to increase number or diameter of mitochondria, to increase oxidative phosphorylation status). There may also be an “expression” phase, wherein the cells are cultured under conditions to maximize expression of exogenous membrane-associated agent, cargo molecules, or other agents exogenous relative to the source cell, on the cell membrane and to restrict transfer in other phases.

In some embodiments, donor cells or membrane-enclosed bodies are generated from cells synchronized, e.g., during a growth phase or the production phase. For example, cells may be synchronized at G1 phase by elimination of serum from the culture medium (e.g., for about 12-24 hours) or by the use in the culture media of DNA synthesis inhibitors such as thymidine, aminopterin, hydroxyurea and cytosine arabinoside. Additional methods for mammalian cell cycle synchronization are known and disclosed, e.g., in Rosner et al. 2013. Nature Protocols 8:602-626 (specifically Table 1 in Rosner).

In some embodiments, the cells can be evaluated and optionally enriched for a desirable phenotype or genotype for use as a source for donor cell or membrane-enclosed body composition as described herein. For example, cells can be evaluated and optionally enriched, e.g., before culturing, during culturing (e.g., during a growth phase or a production phase) or after culturing but before donor cell or membrane-enclosed body production, for example, for one or more of: membrane potential (e.g., a membrane potential of −5 to −200 mV; cardiolipin content (e.g., between 1-20% of total lipid); cholesterol, phosphatidylethanolamine (PE), diglyceride (DAG), phosphatidic acid (PA), or fatty acid (FA) content; genetic quality >80%, >85%, >90%; exogenous membrane-associated agent expression or content; or cargo molecule expression or content.

In some embodiments, donor cells or membrane-enclosed bodies are generated from a cell clone identified, chosen, or selected based on a desirable phenotype or genotype for use as a source for a donor cell or membrane-enclosed body composition described herein. For example, a cell clone is identified, chosen, or selected based on low mitochondrial mutation load, long telomere length, differentiation state, or a particular genetic signature (e.g., a genetic signature to match a recipient).

A donor cell or membrane-enclosed body composition described herein may be comprised of donor cells or membrane-enclosed bodies from one cellular or tissue source, or from a combination of sources. For example, a donor cell or membrane-enclosed body composition may comprise donor cells or membrane-enclosed bodies from xenogeneic sources (e.g., animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise donor cells or membrane-enclosed bodies in different metabolic states, e.g. coupled or uncoupled, as described elsewhere herein.

In some embodiments, donor cells or membrane-enclosed bodies are generated from source cells expressing a membrane-associated agent (and optionally one or more cargo molecules), e.g., a membrane-associated agent described herein. In some embodiments, the membrane-associated agent is disposed in a membrane of the source cell, e.g., a lipid bilayer membrane, e.g., a cell surface membrane, or a subcellular membrane (e.g., lysosomal membrane). In some embodiments, donor cells or membrane-enclosed bodies are generated from source cells with a membrane-associated agent disposed in a cell surface membrane.

In some embodiments, donor cells or membrane-enclosed bodies are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, donor cells or membrane-enclosed bodies are generated by inducing cell enucleation. Enucleation may be performed using assays such as genetic, chemical (e.g., using Actinomycin D, see Bayona-Bafaluy et al., “A chemical enucleation method for the transfer of mitochondrial DNA to p° cells” Nucleic Acids Res. 2003 Aug. 15; 31(16): e98), mechanical methods (e.g., squeezing or aspiration, see Lee et al., “A comparative study on the efficiency of two enucleation methods in pig somatic cell nuclear transfer: effects of the squeezing and the aspiration methods.” Anim. Biotechnol. 2008; 19(2):71-9), or combinations thereof. Enucleation refers not only to a complete removal of the nucleus but also the displacement of the nucleus from its typical location such that the cell contains the nucleus but it is non-functional.

In embodiments, making donor cells or membrane-enclosed bodies comprises producing cell ghosts, giant plasma membrane vesicle, or apoptotic bodies. In embodiments, a donor cell or membrane-enclosed body composition comprises one or more of cell ghosts, giant plasma membrane vesicle, and apoptotic bodies.

In some embodiments, donor cells or membrane-enclosed bodies are generated by inducing cell fragmentation. In some embodiments, cell fragmentation can be performed using the following methods, including, but not limited to, chemical methods, mechanical methods (e.g., centrifugation (e.g., ultracentrifugation, or density centrifugation), freeze-thaw, or sonication), or combinations thereof.

In some embodiments, a membrane-containing substrate is generated by applying lipids to a substrate. The substrate may be a solid, polymeric substance. In some embodiments, the substrate is a flat surface. In some embodiments, the substrate is a spherical bead. In some embodiments, the substrate is porous. In some embodiments, the substrate is glass. In some embodiments, the lipids used to generate a membrane-containing substrate form a lipid bilayer on and/or in the substrate. In some embodiments, the lipids used to generate a membrane-containing substrate are derived from a source cell, e.g., from a cell ghost, giant plasma membrane vesicle, or apoptotic body, or by inducing cell fragmentation.

For avoidance of doubt, it is understood that in many cases the source cell actually used to make the donor cell or membrane-enclosed body will not be available for testing after the donor cell or membrane-enclosed body is made. Thus, a comparison between a source cell and a donor cell or membrane-enclosed body does not need to assay the source cell that was actually modified (e.g., enucleated) to make the donor cell or membrane-enclosed body. Rather, cells otherwise similar to the source cell, e.g., from the same culture, the same genotype same tissue type, or any combination thereof, can be assayed instead.

Modifications to Cells Prior to Donor Cell or Membrane-Enclosed Body Generation

In some aspects, a modification is made to a cell, such as modification of a subject, tissue or cell, prior to donor cell or membrane-enclosed body generation. Such modifications can be effective to, e.g., improve transfer of the membrane-associated agent (and optionally one or more cargo molecules), targeting of a target cell (e.g., acceptor cell), exogenous membrane-associated agent expression or activity, structure or function of the cargo molecule, or structure or function of the target cell.

In some embodiments, a modification is made to a source cell or donor cell or membrane-enclosed body derived therefrom to modulate which membrane-associated agents (e.g., membrane proteins (e.g., endogenous membrane proteins), receptors, ligands, or cell surface markers) are configured for transfer to a target cell (e.g., acceptor cell). Such a modification can decrease the level of one or more (e.g., all) membrane-associated agents that are configured to transfer. Without wishing to be bound by theory, such modification may be desirable to control which membrane-associated agents a donor cell or membrane-enclosed body is capable of transferring to a target cell (e.g., acceptor cell). Such modifications include, but are not limited to, decreasing (e.g., eliminating) expression of a membrane-associated agent (e.g., transiently or stably); altering localization of a membrane-associated agent (e.g., away from the cell membrane); and tethering a membrane-associated agent to an agent not configured to be transferred (e.g., linking a membrane-associated agent to a component of the cytoskeleton or an organelle).

Physical Modifications

In some embodiments, a cell is physically modified prior to generating the donor cell or membrane-enclosed body. For example, as described elsewhere herein, a membrane-associated agent, one or more cargo molecules, and/or one or more targeting domains may be linked to the surface of the cell.

In some embodiments, a cell is treated with a chemical agent prior to generating the donor cell or membrane-enclosed body. For example, the cell may be treated with a chemical agent, such that the chemical agent non-covalently or covalently attaches a membrane-associated agent, one or more cargo molecules, and/or one or more targeting domains to the surface of the cell.

In some embodiments, the cell is physically modified prior to generating the donor cell or membrane-enclosed body with one or more covalent or non-covalent attachment sites for synthetic or endogenous small molecules or lipids on the cell surface that enhance targeting of the donor cell or membrane-enclosed body to an organ, tissues, or cell-type.

In embodiments, a donor cell, acceptor cell, or membrane-enclosed body comprises increased or decreased levels of an endogenous molecule. For instance, the donor cell, acceptor cell, or membrane-enclosed body may comprise an endogenous molecule that also naturally occurs in the naturally occurring source cell but at a higher or lower level than in the donor cell, acceptor cell, or membrane-enclosed body. In some embodiments, the polypeptide is expressed from an exogenous nucleic acid in the source cell, donor cell, acceptor cell, or membrane-enclosed body. In some embodiments, the polypeptide is isolated from a source and loaded into or conjugated to a source cell, donor cell, acceptor cell, or membrane-enclosed body.

In some embodiments, a cell is treated with a chemical agent, e.g., small molecule, prior to generating the donor cell, acceptor cell, or membrane-enclosed body to increase the expression or activity of an endogenous agent, e.g., targeting domain, in the cell (e.g., in some embodiments, endogenous relative to the source cell, and in some embodiments, endogenous relative to the target cell). In some embodiments, a small molecule may increase expression or activity of a transcriptional activator of the endogenous agent (e.g., targeting domain). In some embodiments, a small molecule may decrease expression or activity of a transcriptional repressor of the endogenous agent (e.g., targeting domain). In some embodiments, a small molecule is an epigenetic modifier that increases expression of the endogenous agent (e.g., targeting domain).

In some embodiments, a source cell is physically modified with, e.g., CRISPR activators, prior to generating a donor cell or membrane-enclosed body to add or increase the concentration of exogenous membrane-associated agent, cargo molecule, or targeting domain.

In some embodiments, the cell is physically modified to increase or decrease the quantity, or enhance the structure or function of organelles, e.g., mitochondria, Golgi apparatus, endoplasmic reticulum, intracellular vesicles (such as lysosomes, autophagosomes).

In some embodiments, the cell is physically modified to increase or decrease the presence of an endogenous agent on the cell membrane. In some embodiments, the physical modification increases the level of an endogenous agent, e.g., targeting domain, on the cell membrane. In some embodiments, the physical modification decreases the level of other cell membrane components (e.g., cell membrane components that are not the membrane-associated agent, targeting domain, or cargo molecule) in the cell membrane. Without wishing to be bound by theory, it is thought that transfer of a membrane-associated agent and optionally one or more cargo molecules can be improved by decreasing the level of unnecessary or interfering endogenous agents on the cell membrane and/or increasing the level of endogenous agents that promote transfer (e.g., targeting domains) on the cell membrane.

In some embodiments, the cell is physically modified to attach (e.g., covalently or non-covalently) the cell to a surface, scaffold, or solid matrix, e.g., of an apparatus or device.

In some embodiments, the cell (e.g., donor cell or acceptor cell) is treated with an activating agent that promotes or stimulates transfer of the membrane-associated agent and/or cargo molecule from the donor cell to a target cell, e.g., acceptor cell. In some embodiments, the activating agent is a PKC activator, e.g., phorbol 12-myristate-13-acetate (PMA). In some embodiments, the activating agent is DMSO. In some embodiments, the activating agent is DMSO and PMA. In some embodiments, the activating agent is leucoagglutinin (PHA-L). In some embodiments, the activating agent is PMA and PHA-L.

Genetic Modifications

In some embodiments, a cell is genetically modified prior to generating the donor cell or membrane-enclosed body to increase the expression of an endogenous agent (e.g., targeting domain) in the cell (e.g., endogenous relative to the source cell or endogenous relative to the target cell). In some embodiments, a genetic modification may increase expression or activity of a transcriptional activator of the endogenous agent (e.g., targeting domain). In some embodiments, a genetic modification may decrease expression or activity of a transcriptional repressor of the endogenous agent (e.g., targeting domain). In some embodiments the activator or repressor is a nuclease-inactive Cas9 (dCas9) linked to a transcriptional activator or repressor that is targeted to the endogenous agent or nucleic acid encoding the same by a guide RNA. In some embodiments, a genetic modification epigenetically modifies an endogenous agent-encoding gene to increase its expression. In some embodiments the epigenetic activator a nuclease-inactive Cas9 (dCas9) linked to an epigenetic modifier that is targeted to the endogenous agent by a guide RNA.

In some embodiments, a cell is genetically modified prior to generating the donor cell or membrane-enclosed body to increase the expression of a membrane-associated agent or cargo molecule in the cell, e.g., via delivery of a transgene. In some embodiments, a nucleic acid, e.g., DNA, mRNA or siRNA, is transferred to the cell prior to generating the donor cell or membrane-enclosed body, e.g., to increase or decrease the expression of a cell surface molecule (protein, glycan, lipid or low molecular weight molecule) used for organ, tissue, or cell targeting, e.g., a targeting domain. In some embodiments, the nucleic acid targets a repressor of a membrane-associated agent, targeting domain, or cargo molecule, e.g., an shRNA, siRNA construct. In some embodiments, the nucleic acid encodes an inhibitor of a membrane-associated agent, targeting domain, or cargo molecule repressor.

In some embodiments, the method comprises introducing a nucleic acid that is exogenous relative to the source cell into the source cell, wherein the nucleic acid encodes one or more of a membrane-associated agent, targeting domain, or cargo molecule. The exogenous nucleic acid may be, e.g., DNA or RNA. In some embodiments the exogenous nucleic acid may be e.g., a DNA, a gDNA, a cDNA, an RNA, a pre-mRNA, an mRNA, an miRNA, an siRNA, etc. In some embodiments, the exogenous DNA may be linear DNA, circular DNA, or an artificial chromosome. In some embodiments the DNA is maintained episomally. In some embodiments the DNA is integrated into the genome. The exogenous RNA may be chemically modified RNA, e.g., may comprise one or more backbone modification, sugar modifications, noncanonical bases, or caps. Backbone modifications include, e.g., phosphorothioate, N3′ phosphoramidite, boranophosphate, phosphonoacetate, thio-PACE, morpholino phosphoramidites, or PNA. Sugar modifications include, e.g., 2′-O-Me, 2′F, 2′F-ANA, LNA, UNA, and 2′-O-MOE. Noncanonical bases include, e.g., 5-bromo-U, and 5-iodo-U, 2,6-diaminopurine, C-5 propynyl pyrimidine, difluorotoluene, difluorobenzene, dichlorobenzene, 2-thiouridine, pseudouridine, and dihydrouridine. Caps include, e.g., ARCA. Additional modifications are discussed, e.g., in Deleavey et al., “Designing Chemically Modified Oligonucleotides for Targeted Gene Silencing” Chemistry & Biology Volume 19, Issue 8, 24 Aug. 2012, Pages 937-954, which is herein incorporated by reference in its entirety.

In some embodiments, a cell is treated with a chemical agent, e.g. a small molecule, prior to generating a donor cell or membrane-enclosed body to increase the expression, stability, or activity of a membrane-associated agent, cargo molecule, or targeting domain that is exogenous relative to the source cell. In some embodiments, a small molecule may increase expression or activity of a transcriptional activator of the membrane-associated agent, cargo molecule, or targeting domain. In some embodiments, a small molecule may decrease expression or activity of a transcriptional repressor of the membrane-associated agent, cargo molecule, or targeting domain. In some embodiments, a small molecule is an epigenetic modifier that increases expression of the membrane-associated agent, cargo molecule, or targeting domain.

In some embodiments, the nucleic acid encodes a modified exogenous membrane-associated agent, cargo molecule, or targeting domain. For example, a membrane-associated agent that has regulatable transfer activity, e.g., specific cell-type, tissue-type or local microenvironment activity. Such regulatable transfer activity may include activation and/or initiation of transfer activity by low pH, high pH, heat, infrared light, extracellular enzyme activity (eukaryotic or prokaryotic), or exposure of a small molecule, a protein, or a lipid. For example, a modified exogenous membrane-associated agent with regulatable transfer activity may only be configured for transfer from a donor cell to an acceptor cell in the presence of a particular small molecule or class of small molecules and not be configured for transfer in the absence of the small molecule. In some embodiments, the small molecule, protein, or lipid is displayed on a target cell.

In some embodiments, a cell (e.g., a source cell) is genetically modified prior to generating the donor cell or membrane-enclosed body to alter (i.e., upregulate or downregulate) the expression of signaling pathways (e.g., membrane metabolism, e.g., TOR pathway, e.g., ALG-2 signaling). In some embodiments, a cell (e.g., source cell) is genetically modified prior to generating the donor cell or membrane-enclosed body to alter (e.g., upregulate or downregulate) the expression of a gene or genes of interest. In some embodiments, a cell (e.g., a source cell) is genetically modified prior to generating the donor cell or membrane-enclosed body to alter (e.g., upregulate or downregulate) the expression of a nucleic acid (e.g. miRNA, siRNA, or mRNA) or nucleic acids of interest. In some embodiments, nucleic acids, e.g., DNA, mRNA or siRNA, are transferred to the cell (e.g., source cell) prior to generating the donor cell or membrane-enclosed body, e.g., to increase or decrease the expression of signaling pathways, genes, or nucleic acids. In some embodiments, the nucleic acid targets a repressor of a signaling pathway, gene, or nucleic acid, or represses a signaling pathway, gene, or nucleic acid. In some embodiments, the nucleic acid encodes a transcription factor that upregulates or downregulates a signaling pathway, gene, or nucleic acid. In some embodiments the activator or repressor is a nuclease-inactive cas9 (dCas9) linked to a transcriptional activator or repressor that is targeted to the signaling pathway, gene, or nucleic acid by a guide RNA. In some embodiments, a genetic modification epigenetically modifies an endogenous signaling pathway, gene, or nucleic acid to its expression. In some embodiments the epigenetic activator a nuclease-inactive cas9 (dCas9) linked to a epigenetic modifier that is targeted to the signaling pathway, gene, or nucleic acid by a guide RNA. In some embodiments, a cell's DNA is edited prior to generating the donor cell or membrane-enclosed body to alter (e.g., upregulate or downregulate) the expression of signaling pathways (e.g. membrane metabolism, e.g., TOR pathway, e.g., ALG-2 signaling), gene, or nucleic acid. In some embodiments, the DNA is edited using a guide RNA and CRISPR-Cas9/Cpf1 or other gene editing technology.

A cell (e.g., source cell) may be genetically modified using recombinant methods. A nucleic acid sequence coding for a desired gene can be obtained using recombinant methods, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter and incorporating the construct into an expression vector. The vectors can be suitable for replication and integration in eukaryotes. Typical cloning vectors contain transcription and translation terminators, initiation sequences, and promoters useful for expression of the desired nucleic acid sequence.

In some embodiments, a cell may be genetically modified with one or more expression regions, e.g., a gene. In some embodiments, the cell may be genetically modified with an exogenous gene (e.g., capable of expressing an exogenous gene product such as an RNA or a polypeptide product) and/or an exogenous regulatory nucleic acid. In some embodiments, the cell may be genetically modified with an exogenous sequence encoding a gene product that is endogenous to a source cell or target cell and/or an exogenous regulatory nucleic acid capable of modulating expression of an endogenous gene. In some embodiments, the cell may be genetically modified with an exogenous gene and/or a regulatory nucleic acid that modulates expression of an exogenous gene. In some embodiments, the cell may be genetically modified with an exogenous gene and/or a regulatory nucleic acid that modulates expression of an endogenous gene. It will be understood by one of skill in the art that the cell described herein may be genetically modified to express a variety of exogenous genes that encode proteins or regulatory molecules, which may, e.g., act on a gene product of the endogenous or exogenous genome of a source cell or target cell. In some embodiments, such genes confer characteristics to the donor cell or membrane-enclosed body, e.g., modulate transfer with a target cell (e.g., acceptor cell). In some embodiments, the cell may be genetically modified to express an endogenous gene and/or regulatory nucleic acid. In some embodiments, the endogenous gene or regulatory nucleic acid modulates the expression of other endogenous genes. In some embodiments, the cell may be genetically modified to express an endogenous gene and/or regulatory nucleic acid which is expressed differently (e.g., inducibly, tissue-specifically, constitutively, or at a higher or lower level) than a version of the endogenous gene and/or regulatory nucleic acid on other chromosomes.

The promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to, a tissue-specific promoter, metallothionine promoter, a glucocorticoid promoter, a progesterone promoter, and a tetracycline promoter. In some embodiments, expression of an endogenous agent, exogenous membrane-associated agent, targeting domain, or cargo molecule is upregulated before donor cells or membrane-enclosed bodies are generated, e.g., 3, 6, 9, 12, 24, 26, 48, 60, or 72 hours before donor cells or membrane-enclosed bodies are generated.

The expression vector to be introduced into the source cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transfected cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, beta-lactamase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, a cell may be genetically modified to alter expression of one or more proteins. Expression of the one or more proteins may be modified for a specific time, e.g., development or differentiation state of the source. In some embodiments, donor cells or membrane-enclosed bodies are generated from a source of cells genetically modified to alter expression of one or more proteins, e.g., exogenous membrane-associated agents, cargo molecules, or other proteins that affect targeting of an acceptor cell or transfer of the membrane-associated agent or cargo molecule. Expression of the one or more proteins may be restricted to a specific location(s) or widespread throughout the source.

In some embodiments, the expression of an endogenous agent, e.g., targeting domain, is modified. In some embodiments, donor cells or membrane-enclosed bodies are generated from source cells with modified expression of an endogenous agent (e.g., targeting domain), e.g., an increase or a decrease in expression of an endogenous agent by at least 10%, 15%, 20%, 30%, 40%, 50%, 60%, 75%, 80%, 90% or more.

In some embodiments, cells may be engineered to express a cytosolic enzyme (e.g., proteases, phosphatases, kinases, demethylases, methyltransferases, acetylases) that targets a membrane-associated agent, cargo molecule, or targeting domain. In some embodiments, the cytosolic enzyme affects one or more exogenous membrane-associated agents, cargo molecules, or targeting domains by altering post-translational modifications. Post-translational protein modifications of proteins may affect responsiveness to nutrient availability and redox conditions, and protein-protein interactions. In some embodiments, a donor cell or membrane-enclosed body comprises exogenous membrane-associated agents, cargo molecules, or targeting domains with altered post-translational modifications, e.g., an increase or a decrease in post-translational modifications by at least 10%, 15%, 20%, 30%, 40%, 50%, 60%, 75%, 80%, 90% or more.

Methods of introducing a modification into a cell include physical, biological and chemical methods. See, for example, Geng. & Lu, Microfluidic electroporation for cellular analysis and delivery. Lab on a Chip. 13(19):3803-21. 2013; Sharei, A. et al. A vector-free microfluidic platform for intracellular delivery. PNAS vol. 110 no. 6. 2013; Yin, H. et al., Non-viral vectors for gene-based therapy. Nature Reviews Genetics. 15: 541-555. 2014. Suitable methods for modifying a cell for use in generating the donor cells, acceptor cells, or membrane-enclosed bodies described herein include, for example, diffusion, osmosis, osmotic pulsing, osmotic shock, hypotonic lysis, hypotonic dialysis, ionophoresis, electroporation, sonication, microinjection, calcium precipitation, membrane intercalation, lipid mediated transfection, detergent treatment, viral infection, receptor mediated endocytosis, use of protein transduction domains, particle firing, membrane fusion, freeze-thawing, mechanical disruption, and filtration.

Confirming the presence of a genetic modification includes a variety of assays. Such assays include, for example, molecular biological assays, such as Southern and Northern blotting, RT-PCR and PCR; biochemical assays, such as detecting the presence or absence of a particular peptide, e.g., by immunological means (ELISAs and Western blots) or by assays described herein.

Modifications to Donor Cells or Membrane-Enclosed Bodies

In some aspects, a modification is made to the donor cell or membrane-enclosed body. Such modifications can be effective to, e.g., improve targeting, function, or structure.

In some embodiments, the donor cell or membrane-enclosed body is treated with a membrane-associated agent, targeting domain, or cargo molecule that may non-covalently or covalently link to the surface of the membrane. In some embodiments, the donor cell or membrane-enclosed body is treated with a membrane-associated agent, targeting domain, or cargo molecule, e.g., a protein or a lipid, that may non-covalently or covalently link or embed itself in the membrane

In some embodiments, a ligand is conjugated to the surface of the donor cell or membrane-enclosed body via a functional chemical group (carboxylic acids, aldehydes, amines, sulfhydryls and hydroxyls) that is present on the surface of the donor cell or membrane-enclosed body.

Such reactive groups include without limitation maleimide groups. As an example, a donor cell or membrane-enclosed body may be synthesized to include maleimide conjugated phospholipids such as without limitation DSPE-MaL-PEG2000.

In some embodiments, a small molecule or lipid, synthetic or native, may be covalently or non-covalently linked to the surface of the donor cell or membrane-enclosed body. In some embodiments, a membrane lipid in the donor cell or membrane-enclosed body may be modified to promote, induce, or enhance targeting of an acceptor cell or transfer (e.g., of a membrane-associated agent or cargo molecule) to an acceptor cell.

In some embodiments, the donor cell or membrane-enclosed body is modified by loading with modified proteins (e.g., that enable novel functionality, alter post-translational modifications, bind to the mitochondrial membrane and/or mitochondrial membrane proteins, form a cleavable protein with a heterologous function, form a protein destined for proteolytic degradation, assay the agent's location and levels, or deliver the agent as a carrier). In some embodiments, a donor cell or membrane-enclosed body is loaded with one or more modified proteins.

In some embodiments, a protein exogenous relative to the source cell is non-covalently bound to the donor cell or membrane-enclosed body. The protein may include a cleavable domain for release. In some embodiments, the invention includes a donor cell or membrane-enclosed body comprising an exogenous protein with a cleavable domain.

In some embodiments, the donor cell, acceptor cell, or membrane-enclosed body is modified with a protein destined for proteolytic degradation. A variety of proteases recognize specific protein amino acid sequences and target the proteins for degradation. These protein degrading enzymes can be used to specifically degrade proteins having a proteolytic degradation sequence. In some embodiments, a donor cell, acceptor cell, or membrane-enclosed body comprises modulated levels of one or more protein degrading enzymes, e.g., an increase or a decrease in protein degrading enzymes by at least 10%, 15%, 20%, 30%, 40%, 50%, 60%, 75%, 80%, 90% or more.

As described herein, additives that are not exogenous membrane-associated agents may be added to the donor cell or membrane-enclosed body to modify their structure and/or properties. For example, either cholesterol or sphingomyelin may be added to the membrane to help stabilize the structure and to prevent the leakage of, e.g., cargo molecules. Further, membranes can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review).

In some embodiments, the donor cell or membrane-enclosed body comprises one or more targeting domains on the exterior surface to target a specific cell or tissue type (e.g., cardiomyocytes). These targeting domains include without limitation receptors, ligands, antibodies, and the like. These targeting domains bind their partner on the target cells' (e.g., acceptor cells') surface. In embodiments, the targeting domain is specific for a target cell moiety, e.g., a cell surface marker on a target cell (e.g., acceptor cell) described herein, e.g., an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell).

In some embodiments, the targeting domain binds a cell surface marker on a target cell (e.g., acceptor cell). In embodiments, the cell surface marker comprises a protein, glycoprotein, receptor, cell surface ligand, class I transmembrane protein, class II transmembrane protein, or class III transmembrane protein.

In some embodiments, the targeting domain is comprised by a polypeptide that is a separate polypeptide from the membrane-associated agent. Such a separate polypeptide may be exogenous to the donor cell (e.g., added to the donor cell by genetic engineering) or endogenous to the donor cell. In some embodiments, the polypeptide comprising a targeting domain comprises a transmembrane domain and an extracellular targeting domain (e.g., corresponding to transmembrane moieties and extracellular moieties described herein). In embodiments, the extracellular targeting domain comprises an scFv, DARPin, nanobody, receptor ligand, or antigen. In some embodiments, the extracellular targeting domain comprises an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), or camelid VHH domains), an antigen-binding fibronectin type III (Fn3) scaffold such as a fibronectin polypeptide minibody, a ligand, a cytokine, a chemokine, or a T cell receptor (TCR). In some embodiments, the targeting domain comprises a chimeric receptor (e.g., a chimeric antigen receptor (CAR)).

In some embodiments, a targeting domain binds to multiple cell surface markers, e.g., two, three, four, five, or six cell surface markers. In some embodiments, the targeting domain binds to a first cell surface marker on a first cell (e.g., a first cell type), and a second cell surface marker on a second cell (e.g., a second cell type). Such a targeting domain may be described as bispecific, in that it provides specific binding to two different cells. In some embodiments, a targeting domain is bispecific, trispecific, or tetraspecific. In some embodiments, a targeting domain that specifically binds to cell surface markers on different cells promotes the interaction of those cells, e.g., and with the cell upon which the targeting domain is disposed.

In some embodiments, the donor cell, membrane-enclosed body, or acceptor cell described herein is functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MM include gadolinium chelates, as well as iron, magnesium, manganese, copper, and chromium.

Another example of introducing functional groups to the donor cell or membrane-enclosed body is during post-preparation, by direct crosslinking donor cell or membrane-enclosed body and ligands with homo- or heterobifunctional crosslinkers. This procedure may use a suitable chemistry and a class of crosslinkers (CDI, EDAC, glutaraldehydes, etc. as discussed herein) or any other crosslinker that couples a ligand to the donor cell, acceptor cell, or membrane-enclosed body surface via chemical modification of the donor cell or membrane-enclosed body surface after preparation. This also includes a process whereby amphiphilic molecules such as fatty acids, lipids or functional stabilizers may be passively adsorbed and adhered to the donor cell or membrane-enclosed body surface, thereby introducing functional end groups for tethering to ligands.

Immunogenicity

In some embodiments of any of the aspects described herein, the donor cell, acceptor cell, or membrane-enclosed body composition is substantially non-immunogenic. Immunogenicity can be quantified, e.g., as described herein.

In some embodiments, the donor cell, acceptor cell, or membrane-enclosed body composition comprises elevated levels of an immunosuppressive agent as compared to a reference cell, e.g., an unmodified cell otherwise similar to the source cell. In some embodiments, the elevated level is at least 5%, 10%, 20%, 30%, 40%, 50%, 60%, 70%, 80%, 90%, 2-fold, 3-fold, 5-fold, 10-fold, 20-fold, 50-fold, or 100-fold. In some embodiments, the donor cell, acceptor cell, or membrane-enclosed body composition comprises an immunosuppressive agent that is absent from the reference cell. In some embodiments, the donor cell, acceptor cell, or membrane-enclosed body composition comprises reduced levels of an immune activating agent as compared to a reference cell, e.g., an unmodified cell otherwise similar to the source cell. In some embodiments, the reduced level is at least 5%, 10%, 20%, 30%, 40%, 50%, 60%, 70%, 80%, 90%, 95%, 98%, or 99% compared to the reference cell. In some embodiments, the immune activating agent is substantially absent from the donor cell, acceptor cell, or membrane-enclosed body.

In some embodiments, the donor cell, acceptor cell, or membrane-enclosed body composition, or the source cell from which the donor cell, acceptor cell, or membrane-enclosed body composition is derived from, has one, two, three, four, five, six, seven, eight, nine, ten, eleven, twelve, or more of the following characteristics:
a) less than 50%, 40%, 30%, 20%, 15%, 10%, or 5% or lesser expression of MHC class I or MHC class II, compared to a reference cell, e.g., an unmodified cell otherwise similar to the source cell, or a HeLa cell;
b) less than 50%, 40%, 30%, 20%, 15%, 10%, or 5% or lesser expression of one or more costimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference cell, e.g., an unmodified cell otherwise similar to the source cell, or a reference cell described herein;
c) expression of surface proteins which suppress macrophage engulfment e.g., CD47, e.g., detectable expression by a method described herein, e.g., more than 1.5-fold, 2-fold, 3-fold, 4-fold, 5-fold, 10-fold, or more expression of the surface protein which suppresses macrophage engulfment, e.g., CD47, compared to a reference cell, e.g., an unmodified cell otherwise similar to the source cell;
d) expression of soluble immunosuppressive cytokines, e.g., IL-10, e.g., detectable expression by a method described herein, e.g., more than 1.5-fold, 2-fold, 3-fold, 4-fold, 5-fold, 10-fold, or more expression of soluble immunosuppressive cytokines, e.g., IL-10, compared to a reference cell, e.g., an unmodified cell otherwise similar to the source cell;
e) expression of soluble immunosuppressive proteins, e.g., PD-L1, e.g., detectable expression by a method described herein, e.g., more than 1.5-fold, 2-fold, 3-fold, 4-fold, 5-fold, 10-fold, or more expression of soluble immunosuppressive proteins, e.g., PD-L1, compared to a reference cell e.g., an unmodified cell otherwise similar to the source cell;
f) less than 50%, 40%, 30%, 20%, 15%, 10%, or 5% or lesser expression of soluble immune stimulating cytokines, e.g., IFN-gamma or TNF-α, compared to a reference cell, e.g., an unmodified cell otherwise similar to the source cell;
g) expression of, e.g., detectable expression by a method described herein, HLA-E or HLA-G, compared to a reference cell, e.g., an unmodified cell otherwise similar to the source cell;
h) surface glycosylation profile, e.g., containing sialic acid, which acts to, e.g., suppress NK cell activation;
i) less than 50%, 40%, 30%, 20%, 15%, 10%, or 5% or lesser expression of TCRa/(3, compared to a reference cell, e.g., an unmodified cell otherwise similar to the source cell;
j) less than 50%, 40%, 30%, 20%, 15%, 10%, or 5% or lesser expression of Minor Histocompatibility Antigen (MHA), compared to a reference cell, e.g., an unmodified cell otherwise similar to the source cell; or
k) has less than 10%, 9%, 8%, 7%, 6%, 5%, 4%, 3%, 2%, 1%, or less, of mitochondrial MHAs, compared to a reference cell e.g., an unmodified cell otherwise similar to the source cell, or has no detectable mitochondrial MHAs.

In some embodiments, the donor cell, acceptor cell, or membrane-enclosed body composition does not substantially elicit an immunogenic response by the immune system, e.g., innate immune system. In some embodiments, an immunogenic response by the innate immune system comprises a response by innate immune cells including, but not limited to NK cells, macrophages, neutrophils, basophils, eosinophils, dendritic cells, mast cells, or gamma/delta T cells. In some embodiments, an immunogenic response by the innate immune system comprises a response by the complement system which includes soluble blood components and membrane bound components.

In some embodiments, the donor cell, acceptor cell, or membrane-enclosed body composition does not substantially elicit an immunogenic response by the immune system, e.g., adaptive immune system. In some embodiments, an immunogenic response by the adaptive immune system comprises an immunogenic response by an adaptive immune cell including, but not limited to a change, e.g., increase, in number or activity of T lymphocytes (e.g., CD4 T cells, CD8 T cells, and or gamma-delta T cells), or B lymphocytes. In some embodiments, an immunogenic response by the adaptive immune system includes increased levels of soluble blood components including, but not limited to a change, e.g., increase, in number or activity of cytokines or antibodies (e.g., IgG, IgM, IgE, IgA, or IgD).

In some embodiments, the donor cell, acceptor cell, or membrane-enclosed body composition is modified to have reduced immunogenicity. Immunogenicity can be quantified, e.g., as described herein. In some embodiments, the donor cell, acceptor cell, or membrane-enclosed body composition has an immunogenicity less than 5%, 10%, 20%, 30%, 40%, or 50% lesser than the immunogenicity of a reference cell, e.g., an unmodified cell otherwise similar to the source cell.

In some embodiments of any of the aspects described herein, the donor cell, acceptor cell, or membrane-enclosed body composition is derived from a source cell, e.g., a mammalian cell, having a modified genome, e.g., modified using a method described herein, to reduce, e.g., lessen, immunogenicity. Immunogenicity can be quantified, e.g., as described herein.

In some embodiments, the donor cell, acceptor cell, or membrane-enclosed body composition is derived from a source cell, e.g., a mammalian cell, wherein the mammalian cell comprises a therapeutic agent.

In some embodiments, the donor cell, acceptor cell, or membrane-enclosed body composition is derived from a source cell, e.g., a mammalian cell, wherein the mammalian cell is a recombinant cell.

In some embodiments, the donor cell, acceptor cell, or membrane-enclosed body is derived from a mammalian cell genetically modified to express viral immunoevasins, e.g., hCMV US2, or US11.

In some embodiments, the surface of the donor cell, acceptor cell, or membrane-enclosed body, or the surface of the mammalian cell the donor cell, acceptor cell, or membrane-enclosed body is derived from, is covalently or non-covalently modified with a polymer, e.g., a biocompatible polymer that reduces immunogenicity and immune-mediated clearance, e.g., PEG.

In some embodiments, the surface of the donor cell, acceptor cell, or membrane-enclosed body, or the surface of the mammalian cell the donor cell, acceptor cell, or membrane-enclosed body is derived from is covalently or non-covalently modified with a sialic acid, e.g., a sialic acid comprising glycopolymers, which contain NK-suppressive glycan epitopes.

In some embodiments, the surface of the donor cell, acceptor cell, or membrane-enclosed body, or the surface of the mammalian cell the donor cell, acceptor cell, or membrane-enclosed body is derived from is enzymatically treated, e.g., with glycosidase enzymes, e.g., α-N-acetylgalactosaminidases, to remove ABO blood groups

In some embodiments, the surface of the donor cell, acceptor cell, or membrane-enclosed body, or the surface of the mammalian cell the donor cell, acceptor cell, or membrane-enclosed body is derived from is enzymatically treated, to give rise to, e.g., induce expression of, ABO blood groups which match the recipient's blood type.

### Membrane-Enclosed Bodies

In some embodiments of the compositions and methods described herein include membrane-enclosed bodies, e.g., naturally derived bilayers of amphipathic lipids comprising a membrane-associated agent, and membrane-containing substrates, e.g., solid polymeric substrate (e.g., a scaffold or bead) comprising (e.g., on its surface, e.g., coated with) a plurality of lipids (e.g., a lipid layer, e.g., a lipid bilayer).

As referred to herein, a “membrane-enclosed body” refers to a closed space surrounded by a lipid bilayer which can comprise a membrane-associated agent and is capable of (e.g., is configured to) transferring the membrane-associated agent from its membrane to a target cell (e.g., acceptor cell), wherein the membrane-enclosed body lacks at least one function of cell. In some embodiments, the at least one function of a cell is chosen from, but not limited to, cell growth or cell division. In some embodiments, a membrane-enclosed body is derived from a source cell, but lacks at least one feature of said source cell. In some embodiments, a membrane-enclosed body lacks one or more of a nucleus, DNA, metabolism, ribosomes, one or more organelles (e.g., mitochondria, endoplasmic reticulum, Golgi apparatus, or lysosomes), or at least one marker (e.g., cell surface protein) of the source cell. In some embodiments, a membrane-enclosed body is or comprises vesicle, apoptotic body, microvesicle, an exosome, an apoptotic body, a microparticle (which may be derived from e.g. platelets), or an ectosome. Exemplary membrane-enclosed bodies are described, e.g., in US2016137716, WO/2017/161010, WO/2016/077639, US20160168572, US20150290343, and US20070298118, each of which is incorporated by reference herein in its entirety.

Membrane-enclosed bodies and membrane-containing substrates may comprise several different types of lipids, e.g., amphipathic lipids, such as phospholipids. Membrane-enclosed bodies and membrane-containing substrates may comprise a lipid bilayer, e.g., as the outermost surface. In some instances, membranes may take the form of an autologous, allogeneic, xenogeneic or engineered cell such as is described in Ahmad et al. 2014 Mirol regulates intercellular mitochondrial transport & enhances mesenchymal stem cell rescue efficacy. EMBO Journal. 33(9):994-1010. In some embodiments, the compositions include engineered membranes such as described in, e.g. in Orive. et al. 2015. Cell encapsulation: technical and clinical advances. Trends in Pharmacology Sciences; 36 (8):537-46; and in Mishra. 2016. Handbook of Encapsulation and Controlled Release. CRC Press. In some embodiments, the compositions include naturally occurring membranes (McBride et al. 2012. A Vesicular Transport Pathway Shuttles Cargo from mitochondria to lysosomes. Current Biology 22:135-141).

In some embodiments, a membrane-enclosed body or membrane-containing substrates described herein includes a naturally derived membrane, e.g., membrane vesicles prepared from cells or tissues. In some embodiments, a membrane-enclosed body or membrane-containing substrates is a vesicle derived from MSCs or astrocytes or comprises membrane from the same.

In some embodiments, a membrane-enclosed body is an exosome.

In some embodiments, the membrane-enclosed body comprises a vesicle that is, for instance, obtainable from a cell, for instance a microvesicle, an exosome, an apoptotic body (from apoptotic cells), a microparticle (which may be derived from e.g. platelets), an ectosome (derivable from, e.g., neutrophiles and monocytes in serum), a prostatosome (obtainable from prostate cancer cells), a cardiosome (derivable from cardiac cells), and the like.

In some embodiments, the membrane-enclosed body comprises an extracellular vesicle, nanovesicle, or exosome. In some embodiments a membrane-enclosed body comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the membrane-enclosed body comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells. In embodiments, the membrane-enclosed body comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide. In embodiments, the membrane-enclosed body comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide. In some embodiments, a membrane-containing substrate comprises lipid and/or membrane from an exosome or vesicle described herein.

Exemplary exosomes and other membrane-enclosed bodies are also described in US 20160354313, which is herein incorporated by reference in its entirety. In embodiments, the membrane-enclosed body comprises a Biocompatible Delivery Module, an exosome (e.g., about 30 nm to about 200 nm in diameter), a microvesicle (e.g., about 100 nm to about 2000 nm in diameter) an apoptotic body (e.g., about 300 nm to about 2000 nm in diameter), a membrane particle, a membrane vesicle, an exosome-like vesicle, an ectosome-like vesicle, an ectosome, or an exovesicle.

In some embodiments, a membrane-enclosed body is a microvesicle. In some embodiments, a membrane-enclosed body is a cell ghost. In some embodiments, a vesicle is a plasma membrane vesicle, e.g. a giant plasma membrane vesicle.

In some aspects, the disclosure provides a donor cell or membrane-enclosed body composition (e.g., a pharmaceutical composition) comprising: (i) one or more of a chondrisome (e.g., as described in international application, PCT/US16/64251), a mitochondrion, an organelle (e.g., Mitochondria, Lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, and networks of organelles), or an enucleated cell, e.g., an enucleated cell comprising any of the foregoing, and (ii) a membrane-associated agent.

In embodiments, the membrane-associated agent is present in a lipid bilayer external to the mitochondrion or chondrisome. In embodiments, the chondrisome has one or more of the properties as described, for example, in international application, PCT/US16/64251, which is herein incorporated by reference in its entirety, including the Examples and the

### SUMMARY OF THE INVENTION

Membrane-enclosed bodies or membrane-containing substrates can be made from several different types of lipids, e.g., amphipathic lipids, such as phospholipids. The membrane-enclosed body may comprise a lipid bilayer as the outermost surface. This bilayer may be comprised of one or more lipids of the same or different type. Examples include without limitation phospholipids such as phosphocholines and phosphoinositols. Specific examples include without limitation DMPC, DOPC, and DSPC.

In some embodiments, provided cells or membrane-enclosed bodies, and/or compositions or preparations thereof, have a density of <1, 1-1.1, 1.05-1.15, 1.1-1.2, 1.15-1.25, 1.2-1.3, 1.25-1.35, or >1.35 g/mL.

### Acceptor Cells

The present disclosure is directed, in part, to acceptor cells capable of receiving a membrane-associated agent and optionally one or more cargo molecules. In addition, the present disclosure is directed, in part, to acceptor cells comprising said exogenous membrane-associated agent and optionally one or more cargo molecules (e.g., having received the membrane-associated agent and optionally one or more cargo molecules from a donor cell or membrane-enclosed body) but not comprising a nucleic acid encoding the membrane-associated agent and optionally one or more cargo molecules.

As used herein, the term “acceptor cell” refers to a cell (e.g., a purified cell) capable of receiving a membrane-associated agent from a donor cell, membrane-associated body or membrane-enclosed body. In some embodiments, an acceptor cell is a cell within a tissue, e.g., within an organ, e.g., within a subject (e.g., a human subject). In some embodiments, an acceptor cell is a purified cell. In some embodiments, an acceptor cell does not comprise the membrane-associated agent (e.g., the acceptor cell has not yet received the membrane-associated agent). In some embodiments, an acceptor cell comprises the membrane-associated agent (e.g., having received the membrane-associated agent from a donor cell). An acceptor cell may comprise one or more modifications (e.g., in addition to the membrane-associated agent) relative to a source cell (e.g., from which the acceptor cell was derived), e.g., that enhance the acceptor cell's capability to receive a membrane-associated agent (e.g., from a donor cell).

The disclosure encompasses acceptor cells modified to comprise a membrane-associated agent and optionally one or more cargo molecules. Said modification may occur ex vivo or in vivo.

In some embodiments, an acceptor cell is modified ex vivo (e.g., using a donor cell or membrane-enclosed body described herein) and then administered to a tissue, organ, or subject. In some embodiments, the acceptor cell interacts with a target cell present in a subject after administration and transfers the membrane-associated agent and optionally one or more cargo molecules to said target cell. In some embodiments, such an acceptor cell also acts as a donor cell for a target cell, and as such the membrane-associated agent and optionally one or more cargo molecules are configured for transfer to said target cell. In other embodiments, the acceptor cell is administered to a tissue, organ, or subject without reference to any further transfer of the membrane-associated agent and optionally one or more cargo molecules. In some embodiments, the acceptor cell does not transfer a membrane-associated agent or optionally one or more cargo molecules to a target cell.

As used herein, a target cell refers to a cell capable of receiving a membrane-associated agent and optionally one or more cargo molecules. In some embodiments, a target cell is an acceptor cell. In some embodiments, a target cell receives a membrane-associated agent or optionally one or more cargo molecules from an acceptor cell. In some embodiments, a target cell, e.g., acceptor cell, is present ex vivo, in vitro, or in vivo.

In some embodiments, an acceptor cell is modified in vivo, e.g., by a donor cell or membrane-enclosed body administered to a tissue, organ, or subject.

In some embodiments, the target cell, e.g., acceptor cell, is an immune cell (e.g., an immune effector cell), e.g., a T cell, B cell, NK cell, a PMN (e.g., a granulocyte), a monocyte, a dendritic cell, or a macrophage, or is derived from the same.

In some embodiments, a target cell, e.g., acceptor cell, is a cell from a cell line (e.g., an immortalized cell line), e.g., NK92, THP1, Jurkat, RAW264.7, BT-474, SK-BR-3, MDA-MB-231, BT-20, or KHYG-1 (e.g., as available from ATCC or AcceGen).

In some embodiments, the target cell, e.g., acceptor cell, is in an organism. In some embodiments, the target cell, e.g., acceptor cell, is a primary cell isolated from an organism. In some embodiments, the targeting domain interacts with a target cell moiety on the target cell (e.g., acceptor cell), e.g., a cell surface feature. In some embodiments, the donor cell or membrane-enclosed body does not comprise said target cell moiety. In some embodiments, the donor cell or membrane-enclosed body comprises a membrane-associated agent or targeting domain which interacts with a binding partner on the target cell (e.g., acceptor cell), thereby allowing the donor cell or membrane-enclosed body to bind to the target cell (e.g., acceptor cell) and/or transfer a membrane-associated agent and optionally one or more cargo molecules to the target cell (e.g., acceptor cell). In some embodiments, the donor cell or membrane-enclosed body does not comprise said binding partner. In some embodiments, the targeting domain is not part of the membrane-associated agent or cargo molecule. In some embodiments, the membrane-associated agent comprises the targeting domain. In some embodiments, the binding partner is or is a portion of a different entity from the target cell moiety. In some embodiments, the binding partner is or is a portion of the target cell moiety.

In some embodiments, the target cell (e.g., acceptor cell) or tissue comprising the same is modified (e.g., by inducing stress or cell division) to increase the rate of transfer prior to, at the same time, or after the delivery of donor cell or membrane-enclosed body. Some nonlimiting examples include, inducing ischemia, treatment with chemotherapy, antibiotic, irradiation, toxin, inflammation, inflammatory molecules, anti-inflammatory molecules, acid injury, basic injury, burn, polyethylene glycol, neurotransmitters, myelotoxic drugs, growth factors, or hormones, tissue resection, starvation, and/or exercise.

In some embodiments, the target cell (e.g., acceptor cell) or tissue comprising the same is treated with a vasodilator (e.g. nitric oxide (NO), carbon monoxide, prostacyclin (PGI2), nitroglycerine, phentolamine) or vasoconstrictors (e.g. angiotensin (AGT), endothelin (EDN), norepinephrine)) to increase the rate of donor cell or membrane-enclosed body transport to the tissue.

In some embodiments, the target cell (e.g., acceptor cell) or tissue comprising the same is treated with a chemical agent, e.g., a chemotherapeutic. In such embodiments, the chemotherapeutic induces damage to the target cell (e.g., acceptor cell) or tissue that enhances transfer of a membrane-associated agent or cargo molecule to the target cells (e.g., acceptor cells) or tissue.

In some embodiments, the target cell (e.g., acceptor cell) or tissue comprising the same is treated with a physical stress, e.g., electrofusion. In such embodiments, the physical stress destabilizes the membranes of the target cell (e.g., acceptor cell) or tissue to enhance transfer of a membrane-associated agent or cargo molecule to the target cells (e.g., acceptor cells) or tissue.

In some embodiments, the target cell (e.g., acceptor cell) or tissue comprising the same is treated with an activating agent that stimulates or promotes the receipt of a membrane-associated agent and/or cargo molecule from a donor cell, membrane-enclosed body, or membrane-containing substrate. In some embodiments, the activating agent is DMSO or PMA, or a combination thereof.

In some embodiments, the target cell (e.g., acceptor cell) is not a naturally occurring antigen-presenting cell or derived from a naturally occurring antigen-presenting cell.

### Target Cell Moiety

In some embodiments, a target cell (e.g., acceptor cell) comprises one or more target cell moieties. As used herein, the term “target cell moiety” is used to refer to a feature of a target cell (e.g., an acceptor cell) which may be used to specifically (relative to at least one other cell in the relevant system) target a donor cell or membrane-enclosed body to the cell. In some embodiment, a target cell moiety may be used to promote transfer of a membrane-associated agent and optionally one or more cargo molecules from a donor cell or membrane-enclosed body to a target cell, e.g., acceptor cell. In some embodiments, a target cell moiety is a surface feature of a target cell. In some embodiments, a target cell moiety is or is a portion of a protein associated with the cell membrane of a target cell. In some embodiments, a target cell moiety is, or is a portion of, a peptide or protein associated with the membrane of a target cell. In some embodiments, a target cell moiety is or is a portion of a lipid associated with the membrane of a target cell. In some embodiments, a target cell moiety is or is a portion of a saccharide associated with the membrane of a target cell. In some embodiments, the target cell moiety is endogenous to the target cell (e.g., acceptor cell). In some embodiments, the target cell moiety is exogenous to the target cell (e.g., acceptor cell), e.g., comprises a compound introduced to the target cell (e.g., acceptor cell), or a tissue, organ, or subject comprising said cell.

A target cell moiety may be used to target a donor cell or membrane-enclosed body to a target cell (e.g., acceptor cell). A target cell moiety may be used to promote transfer of a membrane-associated agent and/or one or more cargo molecules from a donor cell or membrane-enclosed body to a target cell (e.g., acceptor cell). In some embodiments, a donor cell or membrane-enclosed body binds (e.g., via a membrane-associated agent and/or targeting domain) to a target cell moiety on a target cell, e.g., acceptor cell.

In some embodiments, a target cell moiety is, e.g., a protein, disposed in a membrane (e.g., a lipid bilayer), of a target cell (e.g., acceptor cell) disclosed herein. In some embodiments, the membrane can be a cell surface membrane, or a subcellular membrane of an organelle, e.g., a mitochondrion, lysosome, or Golgi apparatus. In some embodiments, a target cell moiety can be endogenously expressed, overexpressed, or exogenously expressed (e.g., by a method described herein). In some embodiments, the target cell moiety can cluster with other target cell moieties at the membrane.

In some embodiments, a target cell moiety comprises CD121a/IL1R1, CD121b/IL1R2, CD25/IL2RA, CD122/IL2RB, CD132/IL2RG, CD123/IL3RA, CD131/IL3RB, CD124/IL4R, CD132/IL2RG, CD125/IL5RA, CD131/IL3RB, CD126/IL6RA, CD130/IR6RB, CD127/IL7RA, CD132/IL2RG, CXCR1/IL8RA, CXCR2/IL8RB/CD128, CD129/IL9R, CD210/IL10RA, CDW210B/IL10RB, IL11RA, CD212/IL12RB1, IR12RB2, IL13R, IL15RA, CD4, CDw217/IL17RA, IL17RB, CDw218a/IL18R1, IL20R, IL21R, IL22R, IL23R, LY6E, IL20R1, IL27RA, IL28R, or IL31RA.

In some embodiments, the presence of a target cell moiety, or a plurality of target cell moieties, in a membrane of a target cell (e.g., acceptor cell), creates an interface that can facilitate the interaction, e.g., binding, between a target cell moiety on a target cell (e.g., an acceptor cell), and a membrane-associated agent or targeting domain on a donor cell or membrane-enclosed body. In some embodiments, the membrane-associated agent or targeting domain on a donor cell or membrane-enclosed body interacts with, e.g., binds to, a target cell moiety on a target cell (e.g., acceptor cell), e.g., on the membrane (e.g., lipid bilayer), of a target cell, to induce transfer of the membrane-associated agent and/or one or more cargo molecules from the donor cell or membrane-enclosed body to the target cell (e.g., acceptor cell) membrane.

A target cell moiety can be introduced in a target cell (e.g., acceptor cell), e.g., by any of the methods discussed below.

In some embodiments, a method of introducing a target cell moiety to a target cell (e.g., acceptor cell) comprises removal, e.g., extraction, of a target cell (e.g., via apheresis or biopsy), from a subject (e.g., a subject described herein), and administration of, e.g., exposure to, a target cell moiety under conditions that allow the target cell moiety to be expressed on a membrane of the target cell (e.g., acceptor cell). In some embodiments, a method comprises contacting the target cell (e.g., acceptor cell) expressing a target cell moiety ex vivo with a donor cell or membrane-enclosed body comprising a membrane-associated agent and/or one or more cargo molecules to induce transfer of the membrane-associated agent and/or one or more cargo molecules to the target cell (e.g., acceptor cell) membrane. In some embodiments, a target cell (e.g., acceptor cell) that has received the membrane-associated agent and/or one or more cargo molecules is returned to a subject (e.g., the subject from which the target cell (e.g., acceptor cell) was removed).

In some embodiments, a target cell (e.g., acceptor cell) expressing a target cell moiety is reintroduced into the subject, e.g., intravenously. In some embodiments, a method comprises administering to the subject a donor cell or membrane-enclosed body comprising a membrane-associated agent and/or a targeting domain to allow interaction, e.g., binding, of the membrane-associated agent and/or targeting domain on the donor cell or membrane-enclosed body with the target cell moiety on the target cell (e.g., acceptor cell), and transfer of the membrane-associated agent and/or one or more cargo molecules from the donor cell or membrane-enclosed body to the target cell (e.g., acceptor cell) membrane.

In some embodiments, the target cells (e.g., acceptor cells) are treated with an epigenetic modifier, e.g., a small molecule epigenetic modifier, to increase or decrease expression of an endogenous cell surface molecule, e.g., a target cell moiety, e.g., a protein, glycan, lipid or low molecular weight molecule. In some embodiments, a target cell (e.g., acceptor cell) is genetically modified to increase the expression of an endogenous cell surface molecule, e.g., a target cell moiety. In some embodiments, a genetic modification may decrease expression of a transcriptional activator of the endogenous cell surface molecule, e.g., a target cell moiety.

In some embodiments, a target cell (e.g., acceptor cell) is genetically modified to express, e.g., overexpress, an exogenous cell surface molecule, e.g., a target cell moiety, where the cell surface molecule is a protein, glycan, lipid or low molecular weight molecule. In some embodiments, a nucleic acid, e.g., DNA, mRNA or siRNA, is transferred to the target cell (e.g., acceptor cell), e.g., to increase or decrease the expression of a cell surface molecule, e.g., a target cell moiety, e.g., a protein, glycan, lipid or low molecular weight molecule. In some embodiments, the nucleic acid targets a repressor of a target cell moiety, e.g., an shRNA, or siRNA construct. In some embodiments, the nucleic acid encodes an inhibitor of a target cell moiety repressor.

Membrane-Associated Agents

The donor cells, acceptor cells, and membrane-enclosed bodies described herein may comprise a membrane-associated agent. A membrane-associated agent comprises, minimally, a membrane-associated moiety and one or more of an extracellular moiety, an intracellular moiety, or a cargo molecule. Using the methods described herein, a donor cell or membrane-enclosed body comprising a membrane-associated agent can transfer said agent and/or one or more cargo molecules to a target cell, e.g., an acceptor cell.

In general, the term “agent”, as used herein, may be used to refer to a compound or entity including, for example, a peptide, a polypeptide, a nucleic acid (e.g., DNA, a chromosome (e.g. a human artificial chromosome), RNA, mRNA, siRNA, miRNA), a saccharide or a polysaccharide, a lipid, a small molecule, or a combination or complex thereof. The term may refer to an entity that is or comprises an organelle, or a fraction, extract, or component thereof.

As used herein, “membrane-associated agent” refers to an agent configured for transfer from a first cell (e.g., a donor cell) or membrane-enclosed body to a second cell (e.g., a target cell, e.g., an acceptor cell). A membrane-associated agent comprises a membrane-associated moiety and one or more one, two, or all of an extracellular moiety, an intracellular moiety, or a cargo molecule. In some embodiments, a membrane-associated agent comprises more than one membrane-associated moiety, extracellular moiety, intracellular moiety, or cargo molecule. In some embodiments, at least one moiety (e.g., membrane-associated moiety, extracellular moiety, intracellular moiety, or cargo molecule) of a membrane-associated agent is exogenous to 1) a donor comprising the membrane-associated agent, 2) the target cell, e.g., acceptor cell, to which the membrane-associated agent was or will be transferred, or 3) both. In some embodiments, an exogenous membrane-associated agent is a fusion protein.

As used herein, the term “exogenous” refers to an agent (e.g., a protein or lipid) that is not naturally found in a relevant system (e.g., a cell, a tissue, an organism, a source cell or a target cell, etc.). In embodiments, the agent is engineered and/or introduced into the relevant system. For example, in some embodiments, a donor cell, acceptor cell, or a membrane-enclosed preparation may be said to contain one or more “exogenous” lipids and/or proteins when the relevant lipids and/or proteins are not naturally found in a source cell from which the donor cell, acceptor cell, or membrane-enclosed preparation is obtained or derived (e.g., the source cell of the donor cell, acceptor cell, or membrane-enclosed. In some embodiments, an exogenous membrane-associated agent is or comprises a variant of an endogenous agent, such as, for example, a protein variant that differs in one or more structural aspects such as amino acid sequence, post-translational modification, etc. from a reference endogenous protein, etc.).

In some embodiments, for a given exogenous membrane-associated agent, at least one of the membrane-associated moiety, extracellular moiety, intracellular moiety, or cargo molecule is exogenous to the donor cell, acceptor cell, membrane-enclosed body, or a source cell from which the aforementioned were derived. In some embodiments, at least one of (e.g., one, two, three, or all of) the membrane-associated moiety, extracellular moiety, intracellular moiety, or cargo molecule is exogenous to the donor cell or a source cell from which the donor cell was derived. In some embodiments, at least one of (e.g., one, two, three, or all of) the membrane-associated moiety, extracellular moiety, intracellular moiety, or cargo molecule is exogenous to the acceptor cell or a source cell from which the acceptor cell was derived. In some embodiments, at least one of (e.g., one, two, three, or all of) the membrane-associated moiety, extracellular moiety, intracellular moiety, or cargo molecule is exogenous to the membrane-enclosed body or a source cell from which the membrane-enclosed body was derived.

In some embodiments, for a given exogenous membrane-associated agent, none of the membrane-associated moiety, extracellular moiety, intracellular moiety, or cargo molecule are exogenous to the donor cell, acceptor cell, membrane-enclosed body, or a source cell from which the aforementioned were derived but the combination of said one or more moieties and/or molecule(s) in a single agent is exogenous to the donor cell, acceptor cell, membrane-enclosed body, or a source cell from which the aforementioned were derived. In some embodiments, each of the membrane-associated moiety, extracellular moiety, intracellular moiety, and/or cargo molecule are endogenous to the donor cell, acceptor cell, membrane-enclosed body, or a source cell from which the aforementioned were derived, but the combination of said one or more moieties and/or molecule(s) in a single agent is exogenous to the donor cell, acceptor cell, membrane-enclosed body, or a source cell from which the aforementioned were derived.

A membrane-associated agent may modulate (e.g., increase or decrease) one or more biological functions in a donor cell, acceptor cell, and/or membrane-enclosed body. In some embodiments, the biological function is chosen from:
(i) modulating, e.g., increasing or decreasing a level or activity of a molecule (e.g., a protein, nucleic acid, or metabolite, drug, or toxin) in an acceptor cell or a plurality of acceptor cells;
(ii) modulating, e.g., increasing or decreasing, enzyme activity in an acceptor cell or a plurality of acceptor cells;
(iii) modulating, e.g., increasing or decreasing, a genetic or an epigenetic event in the acceptor cell or the plurality of acceptor cells;
(iv) modulating, e.g., promoting or inhibiting, acceptor cell differentiation or the differentiation of a plurality of acceptor cells;
(v) modulating acceptor cell reprogramming; or
(vi) modulating, e.g., activating or inhibiting, a signaling pathway in an acceptor cell or a plurality of acceptor cells;
(vii) modulating, e.g., increasing, decreasing, or redistributing, cell adhesion or trafficking;
(viii) introducing a genetic alteration (e.g., a substitution, insertion, or deletion) into an acceptor cell or plurality of acceptor cells, e.g., inserting an exogenous nucleic acid (e.g., encoding a gene) or mutating (e.g., knocking out) an endogenous gene.

A membrane-associated agent may target a donor cell or membrane-enclosed body to a target cell, e.g., acceptor cell. In some embodiments, a membrane-associated agent may specifically target a donor cell or membrane-enclosed body to a target cell (e.g., acceptor cell) type, and not target the donor cell or membrane-enclosed body to one or more (e.g., two, three, four, five, six, or more) non-target cell types. In some embodiments, targeting comprises a membrane-associated agent binding to a target cell moiety, e.g., on the surface of the target cell, e.g., acceptor cell. In some embodiments, the extracellular moiety binds specifically to the target cell moiety (e.g., and not to other moieties or receptors on the surface of non-target cells). In some embodiments, the extracellular moiety binds specifically to a plurality of target cell moieties. Without wishing to be bound by theory, it is thought that increasing the number of target cell moieties the membrane-associated agent, targeting domain, or extracellular moiety bind may increase specificity in a combinatorial manner.

A membrane-associated agent may promote transfer of the membrane-associated agent and/or one or more cargo molecules from a donor cell or membrane-enclosed body to a target cell, e.g., acceptor cell. In some embodiments, a membrane-associated agent may specifically promote transfer from a donor cell or membrane-enclosed body to a target cell (e.g., acceptor cell) type, and not promote transfer from the donor cell or membrane-enclosed body to one or more (e.g., two, three, four, five, six, or more) non-target cell types. In some embodiments, promoting transfer comprises a membrane-associated agent binding to a target cell moiety, e.g., on the surface of the target cell, e.g., acceptor cell. In some embodiments, promoting transfer comprises the extracellular moiety binding specifically to the target cell moiety as described above.

In some embodiments, a membrane-associated agent modulates one or more one or more biological functions in a donor cell, acceptor cell, and/or membrane-enclosed body, and targets a donor cell or membrane-enclosed body to a target cell, e.g., acceptor cell. In some embodiments, a membrane-associated agent modulates one or more one or more biological functions in a donor cell, acceptor cell, and/or membrane-enclosed body, and promotes transfer of the membrane-associated agent and/or one or more cargo molecules from a donor cell or membrane-enclosed body to a target cell, e.g., acceptor cell. In some embodiments, a membrane-associated agent targets a donor cell or membrane-enclosed body to a target cell, e.g., acceptor cell, and promotes transfer of the membrane-associated agent and/or one or more cargo molecules from a donor cell or membrane-enclosed body to a target cell, e.g., acceptor cell. In some embodiments, a membrane-associated agent modulates one or more one or more biological functions in a donor cell, acceptor cell, and/or membrane-enclosed body, targets a donor cell or membrane-enclosed body to a target cell, e.g., acceptor cell, and promotes transfer of the membrane-associated agent and/or one or more cargo molecules from a donor cell or membrane-enclosed body to a target cell, e.g., acceptor cell.

In some embodiments, a membrane-associated agent comprises a membrane-associated moiety and an extracellular moiety (e.g., operably associated or linked to (e.g., tethered to) the membrane-associated moiety). In some embodiments, a membrane-associated agent comprises a membrane-associated moiety and an intracellular moiety (e.g., operably associated or linked to (e.g., tethered to) the membrane-associated moiety). In some embodiments, a membrane-associated agent comprises a membrane-associated moiety and a cargo molecule (e.g., operably associated or linked to (e.g., tethered to) the membrane-associated moiety).

As used herein, “operably associated” or “linked” refers to the state of two entities being connected in a functional way. For example, a promoter and a gene may both be situated on a nucleic acid: if they are operably linked, the promoter can promote expression of the gene (e.g., when the nucleic acid is appropriately situated in a cell, etc.); if they are not operably linked, the promoter cannot promote expression of the gene. In said example, operably linked encompasses the functional alignment of several details a skilled person would understand to be relevant to the functional connection of a promoter and a gene, e.g., the distance between the promoter and the gene, the reading frame of the gene, the position of the transcription start site, etc. As a further example, a membrane-associated moiety may be operably associated or linked with an extracellular moiety; such a status may imply that the moieties are part of a fusion protein wherein both moieties assume their native structures, provide any functions they are capable of, and/or the membrane-associated agent comprising said moieties is functional (e.g., configured for transfer or able to provide the functions of its component parts). As yet a further example, an intracellular moiety may be operably associated or linked with a cargo molecule, wherein the intracellular moiety is non-covalently associated with the cargo molecule and the intracellular moiety and cargo molecule are, e.g., able to assume their native structures, provide any functions they are capable of, and/or the membrane-associated agent comprising said moieties is functional (e.g., configured for transfer or able to provide the functions of its component parts). In some embodiments, operably associated or linked comprises a non-covalent interaction. In some embodiments, operably associated or linked comprises a covalent interaction, e.g., a peptide bond or a linker.

In some embodiments, a membrane-associated agent comprises a membrane-associated moiety, an extracellular moiety, and an intracellular moiety. In some embodiments, the extracellular moiety and intracellular moiety are operably associated or linked to (e.g., tethered to) the membrane-associated moiety.

In some embodiments, a membrane-associated agent comprises a membrane-associated moiety, an extracellular moiety, and one or more cargo molecules. In some embodiments, the extracellular moiety is operably associated or linked to (e.g., tethered to) the membrane-associated moiety, and the one or more cargo molecules are operably associated or linked to (e.g., tethered to) the extracellular moiety. In some embodiments, the extracellular moiety and one or more cargo molecules are operably associated or linked to (e.g., tethered to) the membrane-associated moiety. In some embodiments, the extracellular moiety and one or more cargo molecules are operably associated or linked to (e.g., tethered to) the membrane-associated moiety, and one or more different cargo molecules are operably associated or linked to (e.g., tethered to) the extracellular moiety.

In some embodiments, a membrane-associated agent comprises a membrane-associated moiety, an intracellular moiety, and one or more cargo molecules. In some embodiments, the intracellular moiety is operably associated or linked to (e.g., tethered to) the membrane-associated moiety, and the one or more cargo molecules are operably associated or linked to (e.g., tethered to) the intracellular moiety. In some embodiments, the intracellular moiety and one or more cargo molecules are operably associated or linked to (e.g., tethered to) the membrane-associated moiety. In some embodiments, the intracellular moiety and one or more cargo molecules are operably associated or linked to (e.g., tethered to) the membrane-associated moiety, and one or more different cargo molecules are operably associated or linked to (e.g., tethered to) the intracellular moiety.

In some embodiments, a membrane-associated agent comprises a membrane-associated moiety, an extracellular moiety, an intracellular moiety, and one or more cargo molecules. In some embodiments, the extracellular moiety and intracellular moiety are operably associated or linked to (e.g., tethered to) the membrane-associated moiety, and the one or more cargo molecules are operably associated or linked to (e.g., tethered to) the extracellular moiety. In some embodiments, the extracellular moiety and intracellular moiety are operably associated or linked to (e.g., tethered to) the membrane-associated moiety, and the one or more cargo molecules are operably associated or linked to (e.g., tethered to) the intracellular moiety. In some embodiments, one or more cargo molecules are operably associated or linked (e.g., tethered) with multiple different moieties of the membrane-associated agent. For example, a membrane-associated agent may comprise a membrane-associated moiety, an extracellular moiety, and an intracellular moiety, wherein one or more cargo molecules are operably associated or linked to (e.g., tethered to) a first and a second moiety, or a first, second, and third moiety chosen from the membrane-associated moiety, an extracellular moiety, and an intracellular moiety.

In some embodiments, a membrane-associated agent comprises a peptide, a polypeptide, a nucleic acid (e.g., DNA, RNA, mRNA, siRNA, miRNA), a saccharide or a polysaccharide, a lipid, a small molecule, or a combination or complex thereof. In some embodiments, a membrane-associated agent is or comprises a fusion protein. In some embodiments, the fusion protein comprises the membrane-associated moiety and one or both of an extracellular moiety and intracellular moiety. In some embodiments, the fusion protein comprises a cargo molecule. In some embodiments, a membrane-associated agent comprises a fusion protein (e.g., comprising the membrane-associated moiety and optionally one or both of an extracellular moiety and intracellular moiety) and a cargo molecule, wherein the cargo molecule is operably associated or linked (e.g., tethered) to the fusion protein. In some embodiments, said cargo molecule is not connected to the fusion protein by a peptide bond.

In some embodiments, the membrane-associated agent (e.g., an extracellular moiety, membrane-associated moiety, and/or intracellular moiety) is or comprises a receptor, a ligand, or a functional portion of either thereof. In some embodiments, the receptor or ligand is chosen from Tables 1-6 or a receptor described herein (e.g., a receptor tyrosine kinase).

In some embodiments, the membrane-associated agent (e.g., an extracellular moiety, membrane-associated moiety, and/or intracellular moiety) is or comprises a cancer driver, e.g., a protein or gene product encoded by a cancer driver gene as described in Bailey et al. Cell. 2018 Apr. 5;173(2):371-385, the list of which is hereby incorporated by reference, or a functional portion thereof.

In some embodiments, the membrane-associated agent (e.g., an extracellular moiety, membrane-associated moiety, and/or intracellular moiety) is or comprises a Cluster of Differentiation protein or a functional portion or variant thereof.

In some embodiments, the membrane-associated agent (e.g., an extracellular moiety, membrane-associated moiety, and/or intracellular moiety) is or comprises a membrane protein. In some embodiments, the membrane protein is or comprises an immunoglobulin moiety or entity (e.g., an antibody, an Fab, an scFV, an scFab, a sdAb, a duobody, a minibody, a nanobody, a diabody, a zybody, a camelid antibody, a BiTE, a quadroma, a bsDb, etc). In some embodiments, a membrane protein may include one or more covalently-associated non-peptide moieties such as, for example, one or more carbohydrate moieties, lipid moieties, polyethylene glycol moieties, small molecules, etc., and combinations thereof. In some embodiments, the membrane protein is a bitopic protein (a single-pass membrane protein), and integral monotopic protein, a multipass protein, a multi-subunit protein, a peripheral membrane protein, a fatty acid-anchored protein, a GPI anchored protein, or a chemically conjugated protein.

In some embodiments, the membrane-associated agent comprises a chimeric receptor, e.g., that binds to one or more target cell moieties on an acceptor cell and comprises one or more additional biological functionalities.

In some embodiments, the membrane protein is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB 1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gp1OO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RUL RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A, B, C) CD49f, CD151 CD340, CD200, tkrA, trkB, or trkC.

In some embodiments, the membrane-associated agent comprises a membrane protein or portion thereof comprising a cleavage site, e.g., a protease cleavage site. In some embodiments, the cleavage site is a [symbol] -secretase cleavage site. Exemplary [symbol] -secretase cleavable membrane proteins include, but are not limited to, proteins described in Haapasalo and Kovacs. J Alzheimers Dis. 2011; 25(1):3-28 which are incorporated herein by reference.

In some embodiments, membrane protein or portion thereof comprises a RHBDL2 cleavage site. Exemplary RHBDL2 cleavable membrane proteins include, but are not limited to, proteins described in Johnson et al. Sci Rep. 2017 Aug. 4;7(1):7283 which are incorporated herein by reference.

Alternatively or additionally, in some embodiments, one or more of the following is true:

the membrane protein is or comprises a receptor, such as an antigen receptor, which in some embodiments may be a natural receptor or an engineered receptor, e.g., a CAR;

the membrane protein is or comprises an integrin;

the membrane protein is or comprises a T cell receptor;

the membrane protein is or comprises a toll-like receptor;

the membrane protein is or comprises an interleukin receptor (e.g., IL-1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, or 36);

the membrane protein is or comprises a membrane enzyme;

the membrane protein is or comprises a cell adhesion protein (e.g., cadherin protein, selectin protein, mucin protein, etc.).

In some embodiments, the membrane-associated agent does not comprise a TCR polypeptide, a domain of a TCR polypeptide, or a functional portion of either thereof. In some embodiments, the membrane-associated agent does not comprise a MHC polypeptide, a domain of a MHC polypeptide, or a functional portion of either thereof.

In some embodiments, the membrane-associated agent comprises one or more linkers. In some embodiments, the membrane-associated moiety is connected to another moiety (e.g., an extracellular moiety or intracellular moiety) or cargo molecule by a linker. In some embodiments, the extracellular moiety is connected to a cargo molecule by a linker. In some embodiments, the intracellular moiety is connected to a cargo molecule by a linker. In some embodiments, the portions of an extracellular moiety (e.g., specificity portion and accessory portion) or intracellular moiety (e.g., functional portion and accessory portion) are connected by a linker. A linker comprises a covalent connection or series of connections between two agents, e.g., between two moieties. In some embodiments, a linker comprises a peptide, e.g., and the two agents are connected via peptide bonds. In some embodiments, a linker comprises non-amino acid components. In some embodiments, a linker for use in a membrane-associated agent is flexible linker.

Modifications to Exogenous Membrane-Associated Agents

In some embodiments a membrane-associated agent can be altered to reduce immunoreactivity. For instance, a membrane-associated agent may be decorated with molecules that reduce immune interactions, such as PEG (DOI: 10.1128/JVI.78.2.912-921.2004). Thus, in some embodiments, the membrane-associated agent comprises PEG, e.g., is a PEGylated polypeptide. Amino acid residues in the membrane-associated agent that are targeted by the immune system may be altered to be unrecognized by the immune system (doi: 10.1016/j.viro1.2014.01.027, doi:10.1371/journal.pone.0046667). In some embodiments the protein sequence of the membrane-associated agent is altered to resemble amino acid sequences found in humans (humanized). In some embodiments the protein sequence of the membrane-associated agent is changed to a protein sequence that binds MHC complexes less strongly. In some embodiments, the membrane-associated agent is derived, at least in part, from viruses or organisms that do not infect humans (and which humans have not been vaccinated against), increasing the likelihood that a patient's immune system is naïve to the membrane-associated agent (e.g., there is a negligible humoral or cell-mediated adaptive immune response towards the membrane-associated agent) (doi:10.1006/mthe.2002.0550, doi:10.1371/journal.ppat.1005641, doi:10.1038/gt.2011.209, DOI 10.1182/blood-2014-02-558163). In some embodiments, glycosylation of the membrane-associated agent may be changed to alter immune interactions or reduce immunoreactivity. Without wishing to be bound by theory, in some embodiments, a membrane-associated agent derived from a virus or organism that does not infect humans does not have a natural target cell moiety (e.g., to which it binds) in patients, and thus has high specificity.

In some embodiments, a membrane-associated agent comprises one or more (e.g., one, two, three, or more) cleavage sites recognized by a protease. In some embodiments, the protease is not present in a donor cell, membrane-enclosed body, or a source cell from which either the donor cell or membrane-enclosed body are derived. In some embodiments, the protease is present in an acceptor cell or source cell from which the acceptor cell is derived. Without wishing to be bound by theory, a membrane-associated agent comprising a cleavage site for a protease not present in a donor cell may be safely carried, e.g., without being cleaved, by a donor cell or membrane-enclosed body. Only upon transfer to a cell containing the appropriate protease (e.g., an acceptor cell) would the membrane-associated agent be cleaved. Exemplary cleavage sites include, but are not limited to, a TEV protease cleavage site and a Rhomboid, veinlet-like 2 (RHBDL2) cleavage site.

In some embodiments, the membrane-associated agent comprises a cleavage site in the membrane-associated moiety. In some embodiments, the membrane-associated agent comprises a cleavage site in the extracellular moiety. In some embodiments, the membrane-associated agent comprises a cleavage site in the intracellular moiety. In some embodiments, the membrane-associated agent comprises a protein that is inactive or unable to activate its function until cleaved from the membrane-associated agent. For example, in some embodiments the intracellular moiety comprises a transcription factor and a cleavage site, wherein upon cleavage the transcription factor is free to translocate to the nucleus and alter transcription. As a further example, in some embodiments, the intracellular moiety comprises a zymogen that is transformed into an active enzyme by cleavage.

### Membrane-Associated Moieties

A membrane-associated moiety associates with (e.g., is localized in and/or on) or is capable of associating with a membrane (e.g., a cell membrane). Generally, the membrane-associated moiety localizes a membrane-associated agent to a membrane.

As used herein, “membrane-associated moiety” refers to an agent that associates with (e.g., is localized in and/or on) or is capable of associating with a membrane (e.g., a cell membrane). In some embodiments, a membrane-associated moiety comprises a domain that at least partially (e.g., completely) spans a membrane, e.g., cell membrane. In some embodiments, a membrane-associated moiety is a transmembrane moiety that completely spans a membrane, e.g., cell membrane. In some embodiments a membrane-associated moiety is or comprises a transmembrane protein or the transmembrane domain of a transmembrane protein. In some embodiments, a membrane-associated moiety comprises a lipidation modification sequence, e.g., a N-myristoylation, N-palmitoylation, or S-palmitoylation sequence, or a hydrophobic signal sequence suitable for addition of Glycosylphosphatidylinositol (GPI), e.g., comprises a myristoyl, palmitoyl, or GPI modification. In some embodiments, a membrane-associated moiety is associated with an interior (e.g., cytosolic) portion of a membrane lipid bilayer. In some embodiments a membrane-associated moiety is associated with an exterior portion of a membrane lipid bilayer (e.g., with a cell surface or with a surface of a donor cell, acceptor cell, or a membrane-enclosed preparation as described herein). In some embodiments, a membrane-associated moiety is associated with an exterior portion of a membrane lipid bilayer and is or comprises a cell surface protein. In some embodiments a membrane-associated moiety is a naturally occurring protein. In some embodiments a membrane-associated moiety is an engineered and/or synthetic protein (e.g., a chimeric antigen receptor). In some embodiments a membrane-associated moiety is a therapeutic agent. In some embodiments, a membrane-associated moiety is operably associated or linked (e.g., tethered) to one or more of an intracellular moiety, an extracellular moiety, or a cargo molecule.

In some embodiments, a membrane-associated moiety is or comprises a lipidation modification sequence. A lipidation modification sequence comprises an amino acid sequence recognized by and/or modified by a fatty acid transferase enzyme present in a cell, e.g., a source cell or donor cell. A membrane-associated moiety comprising a lipidation modification sequence may have a lipid anchor attached that associates, e.g., anchors, the membrane-associated moiety to a membrane. In some embodiments, a lipid anchor may be attached to the terminus of a polypeptide (e.g., of a membrane-associated agent, e.g., the membrane-associated moiety). Exemplary lipid anchors include, but are not limited to, myristoyl, palmitoyl, farnesyl, or glycosylphosphatidylinositol (GPI). Exemplary lipidation modification sequences are known to those of skill in the art and include but are not limited to myristolyation or palmitoylation (MYR/PA)-binding sequence (e.g., a MYR/PA sequence from an LCK tyrosine kinase).

In some embodiments, a membrane-associated moiety is or comprises a membrane protein, e.g., a naturally occurring or synthetic membrane protein, or a portion thereof. In some embodiments, a membrane-associated moiety is or comprises a membrane protein described herein or a portion thereof.

In some embodiments, a membrane protein relevant to the present disclosure is an integral membrane protein; in some embodiments, a membrane protein is a peripheral membrane protein. In other embodiments, a membrane protein is temporarily associated with a membrane. In some embodiments, a membrane protein is a protein that is associated with, and/or wholly or partially spans (e.g., a transmembrane protein) a target cell's membrane. In some embodiments, a membrane protein is an integral monotopic protein (i.e., associated with only one side of a membrane). In some embodiments, a membrane protein is or becomes associated with (e.g., is partly or wholly present on) an outer surface of a target cell's membrane. In some embodiments, a membrane protein is or becomes associated with (e.g., is partly or wholly present on) an inner surface of a target cell's membrane.

In some embodiments, a membrane protein relevant to the present disclosure is a therapeutic membrane protein. In some embodiments, a membrane protein relevant to the present disclosure is or comprises a receptor (e.g., a cell surface receptor and/or a transmembrane receptor), a cell surface ligand, a membrane transport protein (e.g., an active or passive transport protein such as, for example, an ion channel protein, a pore-forming protein [e.g., a toxin protein], etc), a membrane enzyme, and/or a cell adhesion protein).

In some embodiments, a membrane protein relevant to the present disclosure comprises a sequence of a naturally-occurring membrane protein. In some embodiments, a membrane protein relevant to the present disclosure is or comprises a variant or modified version of a naturally-occurring membrane protein. In some embodiments, a membrane protein relevant to the present disclosure is or comprises an engineered membrane protein. In some embodiments, a membrane protein relevant to the present disclosure is or comprises a fusion protein.

### Extracellular Moieties

An extracellular moiety is an optional part of a membrane-associated agent positioned on the exterior (e.g., non-lumen or non-cytosolic side) of a membrane (e.g., a cell membrane or the membrane of a membrane-enclosed body). In some embodiments, an extracellular moiety comprises one or more specificity portions, one or more accessory portions, or both.

In some embodiments, an extracellular moiety comprises a specificity portion which may comprise a targeting domain or a transfer promoting domain. As used herein, the term “targeting domain” is an agent (e.g., a polypeptide) which associates or interacts with (e.g., binds) a target cell moiety. In some embodiments, a targeting domain specifically (e.g., under conditions of exposure, e.g., of donor cell contact/proximity to an acceptor cell) associates or interacts with a target cell moiety. In some embodiments, a targeting domain specifically binds to a target cell moiety present on a target cell. In some embodiments, a targeting domain is or comprises a domain of a membrane-associated agent e.g., is covalently linked to a membrane-associated agent, e.g., is part of a membrane-associated agent polypeptide. In some embodiments, a targeting domain is a separate entity from any exogenous membrane-associated agent, e.g., is not covalently linked to a membrane-associated agent, e.g., is not part of a membrane-associated agent polypeptide. In some embodiments, the targeting domain facilitates contact between a donor cell or membrane-enclosed body and a target cell, e.g., acceptor cell, e.g., by binding a target cell moiety on the target cell or being bound by a target cell moiety on the target cell. In some embodiments, an extracellular moiety, e.g., a specificity portion, e.g., a targeting domain, comprises a membrane protein or a portion thereof (e.g., a membrane protein described herein). Exemplary specificity portions (e.g., targeting domains) include, but are not limited to, an antibody or functional fragment thereof (e.g., a Fab, F(ab′)2, Fab′, scFv, or di-scFv), a streptavidin domain (e.g., associated with a biotinylated agent, e.g., a biotinylated antibody), a receptor (e.g., a surface receptor) (e.g., that specifically binds a ligand on the acceptor cell), a ligand (e.g., a ligand that binds a target cell moiety, e.g., receptor, on an acceptor cell), a cell surface protein, a sugar, or a lipid.

In some embodiments, the transfer promoting domain promotes transfer of the membrane-associated agent from a membrane of a first cell (e.g., donor cell) or first membrane-enclosed body to a membrane of a second cell (e.g., acceptor cell) or second membrane-enclosed body. Exemplary specificity portions, e.g., transfer promoting domains, include but are not limited to E-selectin, P-selectin, L-selectin, or a portion of any thereof (e.g., the extracellular and/or transmembrane domains of the selectin); a claudin, a gap junction protein, an annexin, an integrin, a lectin, a tight junction protein, a desmosomal protein, a member of the immunoglobulin superfamily of molecules (e.g., an antibody or functional fragment thereof), e.g., an HLA-G domain or portion thereof; or a cell adhesion molecule involved in the leukocyte adhesion cascade.

In some embodiments, an extracellular moiety comprises a trafficking receptor, e.g., a chemokine receptor, e.g., a CCR protein, a CXCR protein, or a formyl peptide receptor (FPR) protein. In some embodiments, an extracellular moiety comprises an activation or inhibition receptor, e.g., a Notch receptor, interleukin (IL) receptor, or a cluster of differentiation (CD) molecule. In some embodiments, an extracellular moiety comprises a reprogramming receptor. In some embodiments, an extracellular moiety comprises a therapeutic protein.

In some embodiments, an accessory portion provides an ancillary function to the membrane-associated agent, e.g., unrelated to targeting the donor cell to the acceptor cell or with promoting transfer. In some embodiments, an accessory portion comprises one or more of a tag (e.g., a label (e.g., a fluorescent or radio label) or a cleavage site), a reporter agent, or a marker.

### Intracellular Moieties

An intracellular moiety is an optional part of a membrane-associated agent positioned on the interior (e.g., lumen or cytosolic side) of a membrane (e.g., a cell membrane or the membrane of a membrane-enclosed body). In some embodiments, an intracellular moiety comprises one or more functional portions, one or more accessory portions, or both.

In some embodiments, an intracellular moiety comprises a functional portion which modulates a biological function in the acceptor cell (e.g., and optionally does not modulate or modulates to a lesser extent the biological process in the donor cell). Exemplary modulation includes but is not limited to: altering (e.g., decreasing or increasing) expression of a gene, epigenetic modification, increasing or decreasing activity of an intra- or inter-cell signaling pathway, altering the stability (e.g., degradation and/or half-life) of one or more cell component (e.g., signaling molecule or protein), altering secretion of a biological effector, altering cellular metabolism, inducing or inhibiting cellular migration, inducing or inhibiting apoptosis, or altering potency or the cell identity/differentiation of the cell.

In some embodiments, the biological function is chosen from:
(i) modulating, e.g., increasing or decreasing a level or activity of a molecule (e.g., a protein, nucleic acid, or metabolite, drug, or toxin) in an acceptor cell or a plurality of acceptor cells;
(ii) modulating, e.g., increasing or decreasing, enzyme activity in an acceptor cell or a plurality of acceptor cells;
(iii) modulating, e.g., increasing or decreasing, a genetic or an epigenetic event in the acceptor cell or the plurality of acceptor cells;
(iv) modulating, e.g., promoting or inhibiting, acceptor cell differentiation or the differentiation of a plurality of acceptor cells;
(v) modulating acceptor cell reprogramming; or
(vi) modulating, e.g., activating or inhibiting, a signaling pathway in an acceptor cell or a plurality of acceptor cells;
(vii) modulating, e.g., increasing, decreasing, or redistributing, cell adhesion or trafficking;
(viii) introducing a genetic alteration (e.g., a substitution, insertion, or deletion) into an acceptor cell or plurality of acceptor cells, e.g., inserting an exogenous nucleic acid (e.g., encoding a gene) or mutating (e.g., knocking out) an endogenous gene.

In some embodiments, the intracellular moiety, e.g., functional portion, comprises one or more of an antibody or functional fragment thereof (e.g., a Fab, F(ab′)2, Fab′, scFv, or di-scFv), a reporter agent (e.g., a fluorescent tag), a signaling protein, an enzyme (or functional portion thereof), a transcription factor, an epigenetic remodeling agent, a protein binding domain, an RNA-binding protein or domain, a hydrophobic domain, a lipid raft targeting domain, or drug-binding domain. In some embodiments, the intracellular moiety, e.g., functional portion, comprises EGFP, β-galactosidase, β-lactamase, Cre recombinase, a CRISPR/Cas protein (e.g., Cas9), and optionally a guide RNA, or a functional portion or variant of any thereof. In some embodiments, the intracellular moiety, e.g., functional portion, comprises a nucleic acid binding domain, e.g., an RNA binding protein or domain, e.g., an mRNA binding protein or domain.

In some embodiments, the intracellular moiety, e.g., functional portion, comprises an agent that binds to another agent (e.g., comprises a protein that binds to a cargo molecule). In some embodiments, the intracellular moiety, e.g., functional portion, comprises MS2 coat protein (e.g., bound to an mRNA), an scFv (e.g., bound to a protein or an organelle), one part of a protein binding pair (e.g., bound to the other partner of the protein binding pair), streptavidin (e.g., bound to biotin or a biotin-conjugated agent), an organelle-specific integral membrane protein (e.g., bound or associated with an organelle), a CRISPR protein (e.g., a Cas9, Cas12, or MAD7 protein) (e.g., bound or associated with a guide sequence, e.g., gRNA), or a poly-A binding protein (e.g., bound or associated with an mRNA).

In some embodiments, an accessory portion provides an ancillary function to the membrane-associated agent, e.g., unrelated to the functional portion's one or more functions. In some embodiments, an accessory portion comprises one or more of a tag (e.g., a label (e.g., a fluorescent or radio label) or a cleavage site), a reporter agent, or a marker. In some embodiments, the intracellular moiety, e.g., accessory portion, comprises a Lumio tag, a TEV protease cleavage site, or a rhomboid protease cleavage site, e.g., RHBDL2.

In some embodiments, an intracellular moiety comprises a cleavage site. In some embodiments, a target cell (e.g., acceptor cell) comprises a protease that recognizes an intracellular moiety cleavage site, but the donor cell or membrane-enclosed body do not comprise a protease that recognizes the intracellular moiety cleavage site. Without wishing to be bound by theory, it is thought that this will allow a membrane-associated agent comprising a cleavable intracellular moiety to be delivered by a donor cell or membrane-enclosed body without being cleaved, wherein upon arrival in the target cell (e.g. acceptor cell) a protease will recognize the site and cleave the intracellular moiety or a portion thereof. In some embodiments, cleavage of the intracellular moiety activates a function of the intracellular moiety or a portion thereof (e.g., an enzymatic or signaling function). In some embodiments, cleavage of the intracellular moiety causes the intracellular moiety or a portion thereof to dissociate from the membrane-associated agent and/or the membrane, e.g., and enables it to fulfill its function. For example, an intracellular moiety may comprise a cleavable Cre recombinase or a transcription factor, where the Cre recombinase or transcription factor require translocation to the nucleus to function and cleavage enables said translocation.

### Cargo Molecules

In some embodiments, a donor cell or membrane-enclosed body described herein includes a cargo molecule. In some embodiments, a cargo molecule is an agent that may be transferred from a first membrane (e.g., a donor cell or membrane-enclosed body membrane) to a second membrane (e.g., an acceptor cell membrane). Transfer of a cargo molecule may be promoted by a membrane-associated agent. A cargo molecule may be non-covalently associated with the membrane-associated agent, covalently associated via a non-peptide bond, or not associated with the membrane-associated agent (e.g., and separately associated with the membrane, e.g., of the donor cell, membrane-enclosed body, or acceptor cell).

As used herein, “cargo molecule” comprises an agent which may be delivered to a target cell (e.g., acceptor cell), or by an acceptor cell to another target cell (e.g., acceptor cell). The cargo molecule is: non-covalently associated with a component of the membrane-associated agent (e.g., an extracellular moiety, intracellular moiety, or membrane-associated moiety); or covalently associated with said a component of the membrane-associated agent (e.g., an extracellular moiety, intracellular moiety, or membrane-associated moiety) via a non-peptide bond; or not associated with the membrane-associated agent, e.g., the cargo molecule is operably associated or linked (e.g., tethered) to the membrane of a donor cell or membrane-enclosed body (e.g., separately from the membrane-associated agent). In some embodiments a cargo molecule comprises one or more a protein, e.g., an enzyme, a transmembrane protein, a receptor, or an antibody; a nucleic acid, e.g., a circular or linear nucleic acid, e.g., DNA, a chromosome (e.g. a human artificial chromosome), or RNA, e.g., mRNA, siRNA, miRNA, piRNA, or lncRNA; a lipid; or a small molecule (e.g., a signaling molecule (e.g., a second messenger) or a drug molecule). In some embodiments, a cargo is or comprises an organelle.

In some embodiments, the cargo molecule may be or may encode a therapeutic protein. In some embodiments, a donor cell or membrane-enclosed body described herein includes cargo molecules that are or comprise a plurality of therapeutic agents. In some embodiments, a cargo molecule may be a therapeutic agent that is exogenous or endogenous relative to the donor cell, membrane-enclosed body, acceptor cell, or source cell from which the aforementioned were derived.

In some embodiments, a donor cell, membrane-enclosed body, or acceptor cell comprises a plurality of different cargo molecules. In some embodiments, a donor cell, membrane-enclosed body, or acceptor cell comprises 1, 2, 3, 4, 5, 6, 7, 8, 9, 10, or more different cargo molecules (and optionally no more than 20, 15, or 10 different cargo molecules). For example, a donor cell may comprise a first cargo molecule comprising a first therapeutic agent and a second cargo molecule comprising a second different therapeutic agent.

In some embodiments a donor cell or membrane-enclosed body comprises a cargo molecule associated with the donor cell or membrane-enclosed body lipid bilayer. In some embodiments a donor cell or membrane-enclosed body comprises a cargo molecule disposed within the cytosol or lumen of the donor cell or membrane-enclosed body. In some embodiments, a donor cell or membrane-enclosed body comprises a cargo molecule associated with the donor cell or membrane-enclosed body lipid bilayer and a cargo molecule disposed within the cytosol or lumen of the donor cell or membrane-enclosed body.

In some embodiments, a cargo molecule is not expressed naturally in a donor cell or membrane-enclosed body, or source cell from which the donor cell or membrane-enclosed body is derived. In some embodiments, a cargo molecule is expressed naturally in the donor cell or membrane-enclosed body, or source cell from which the donor cell or membrane-enclosed body is derived. In some embodiments, a cargo molecule is a mutant of a wild type nucleic acid or protein expressed naturally in a donor cell or membrane-enclosed body, or source cell from which the donor cell or membrane-enclosed body is derived, or the cargo molecule is a wild type variant of a mutant cargo molecule expressed naturally in a donor cell or membrane-enclosed body, or source cell from which the donor cell or membrane-enclosed body is derived.

In some embodiments, a cargo molecule is loaded into a donor cell or membrane-enclosed body via expression in a source cell from which the donor cell or membrane-enclosed body is derived (e.g. expression from DNA introduced via transfection, transduction, or electroporation). In some embodiments, a cargo molecule is expressed from DNA integrated into the genome of the source cell from which the donor cell or membrane-enclosed body is derived or maintained episosomally in the source cell from which the donor cell or membrane-enclosed body is derived. In some embodiments, expression of a cargo molecule is constitutive in the source cell from which the donor cell or membrane-enclosed body is derived. In some embodiments, expression of a cargo molecule in the source cell from which the donor cell or membrane-enclosed body is derived is induced. In some embodiments, expression of the cargo molecule is induced in the source cell from which the donor cell or membrane-enclosed body is derived immediately prior to generating the donor cell or membrane-enclosed body. In some embodiments, expression of a cargo molecule in the source cell from which the donor cell or membrane-enclosed body is derived is induced at the same time as expression of the membrane-associated agent in the cell from which the donor cell or membrane-enclosed body is derived.

In some embodiments, a cargo molecule is loaded into a donor cell or membrane-enclosed body via electroporation into the donor cell or membrane-enclosed body itself or into a source cell from which the donor cell or membrane-enclosed body is derived. In some embodiments, a cargo molecule is loaded into a donor cell or membrane-enclosed body via transfection into the donor cell or membrane-enclosed body itself or into a source cell from which the donor cell or membrane-enclosed body is derived.

In some embodiments, the cargo molecule may include one or more nucleic acid sequences, one or more polypeptides, a combination of nucleic acid sequences and/or polypeptides, one or more organelles, and any combination thereof. In some embodiments, the cargo molecule may include one or more cellular components. In some embodiments, the cargo molecule includes one or more cytosolic and/or nuclear components.

In some embodiments, the cargo molecule includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In some embodiments, the cargo molecule may include a nucleic acid. For example, the cargo molecule may comprise RNA to enhance expression of an endogenous protein (e.g., in some embodiments, endogenous relative to the source cell, and in some embodiments, endogenous relative to the target cell), or a siRNA or miRNA that inhibits protein expression of an endogenous protein. For example, the endogenous protein may modulate structure or function in the target cells (e.g., acceptor cells). In some embodiments, the cargo molecule may include a nucleic acid encoding an engineered protein that modulates structure or function in the target cells (e.g., acceptor cells). In some embodiments, the cargo molecule is a nucleic acid that targets a transcriptional activator that modulate structure or function in the target cells (e.g., acceptor cells).

In some embodiments, the cargo molecule comprises a self-replicating RNA, e.g., as described herein. In some embodiments, the self-replicating RNA is single stranded RNA and/or linear RNA. In some embodiments, the self-replicating RNA encodes one or more proteins, e.g., a protein described herein, e.g., a membrane protein or a secreted protein. In some embodiments, the self-replicating RNA comprises a partial or complete genome from arterivirus or alphavirus, or a variant thereof.

In some embodiments, the cargo molecule can comprise an RNA that can be delivered into a target cell (e.g., an acceptor cell), and RNA is replicated inside the target cell (e.g., acceptor cell). Replication of the self-replicating RNA can involve RNA replication machinery that is exogenous to the target cell (e.g., acceptor cell), and/or RNA replication machinery that is endogenous to the target cell (e.g., acceptor cell).

In some embodiments, the self-replicating RNA comprises a viral genome, or a self-replicating portion or analog thereof. In some embodiments, the self-replicating RNA is from a positive-sense single-stranded RNA virus. In some embodiments, the self-replicating RNA comprises a partial or complete arterivirus genome, or a variant thereof. In some embodiments, the arterivirus comprises Equine arteritis virus (EAV), Porcine respiratory and reproductive syndrome virus (PRRSV), Lactate dehydrogenase elevating virus (LDV), and Simian hemorrhagic fever virus (SHFV). In some embodiments, the self-replicating RNA comprises a partial or complete alphavirus genome, or a variant thereof. In some embodiments, the alphavirus belongs to the VEEV/EEEV group (e.g., Venezuelan equine encephalitis virus), the SF group, or the SIN group.

In some embodiments, the donor cell or membrane-enclosed body that comprises the self-replicating RNA further comprises: (i) one or more proteins that promote replication of the RNA, or (ii) a nucleic acid encoding one or more proteins that promote replication of the RNA, e.g., as part of the self-replicating RNA or in a separate nucleic acid molecule.

In some embodiments, the self-replicating RNA lacks at least one functional gene encoding one or more viral structural protein relative to the corresponding wild-type genome. For instance, in some embodiments the self-replicating RNA fully lacks one or more genes for viral structural proteins or comprises a non-functional mutant gene for a viral structural protein. In some embodiments, the self-replicating RNA does not comprise any genes for viral structural proteins.

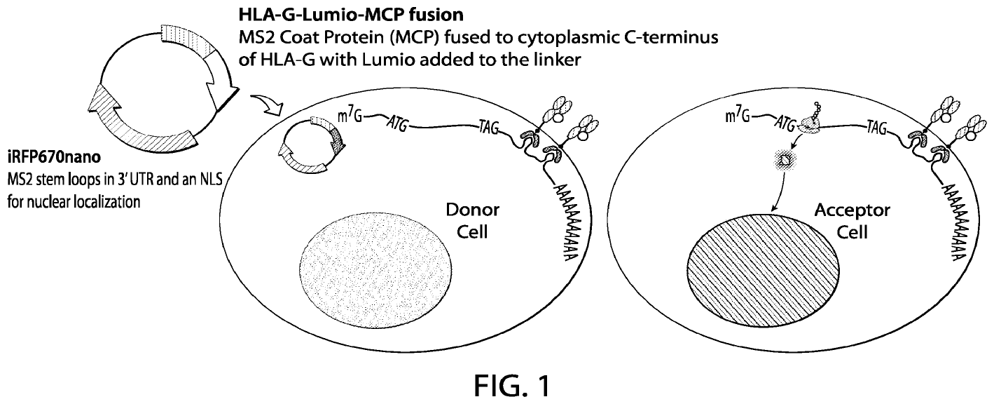

In some embodiments, the self-replicating RNA comprises a viral capsid enhancer, e.g., as described in International Application WO2018/106615, which is hereby incorporated by reference in its entirety. In some embodiments, the viral capsid enhancer is an RNA structure that increases translation of a coding sequence in cis, e.g., by allowing eIF2alpha independent translation of the coding sequence. In some embodiments, a host cell has impaired translation, e.g., due to PKR-mediated phosphorylation of eIF2alpha. In embodiments, the viral capsid enhancer comprises a Downstream Loop (DLP) from a viral capsid protein, or a variant of the DLP. In some embodiments, the viral capsid enhancer is from a virus belonging to the Togaviridae family, e.g., the Alphavirus genus of the Togaviridae family. In some embodiments, the viral capsid enhancer has a sequence of SEQ ID NO: 1 of WO2018/106615 (which sequence is herein incorporated by reference in its entirety), or a sequence having at least 70%, 80%, 85%, 90%, 95%, or 99% identity thereto. In some embodiments, the sequence has the same secondary structure shown in FIG. 1 of WO2018/106615.

In some embodiments, the self-replicating RNA comprises one or more arterivirus sequences, e.g., as described in International Application WO2017/180770, which is hereby incorporated by reference in its entirety. In some embodiments, the self-replicating RNA comprises ORF7 (or a functional fragment or variant thereof) and/or the self-replicating RNA lacks a functional ORF2a (e.g., fully lacks ORF2a, or comprises a non-functional mutant of ORF2a) of an arterivirus. In some embodiments, the self-replicating RNA lacks a functional ORF2b, ORF3, ORF4, ORF5a, ORF5, or ORF6 or any combination thereof (e.g., fully lacks the sequence(s) or comprises a non-functional mutant of the sequence(s)). In some embodiments, the self-replicating RNA lacks a portion of one or more of ORF2a, ORF2b, ORF3, ORF4, ORF5a, ORF5, or ORF6. In some embodiments, the self-replicating RNA comprises one or more subgenomic (sg) promoters, e.g., situated at a non-native site. In some embodiments, the promoter comprises sg promoter 1, sg promoter 2, sg promoter 3, sg promoter 4, sg promoter 5, sg promoter 6, sg promoter 7, or a functional fragment or variant thereof. In some embodiments, the self-replicating RNA comprises one or more transcriptional termination signals, e.g., T7 transcriptional termination signals, e.g., a mutant T7 transcription termination signal, e.g., a mutant T7 transcription termination signal comprising one or more of (e.g., any two of, or all of) T9001G, T3185A, or G3188A.

In some embodiments, the self-replicating RNA comprises a 5′ UTR, e.g., a mutant alphavirus 5′ UTR, e.g., as described in International Application WO2018/075235, which is hereby incorporated by reference in its entirety. In some embodiments, the mutant alphavirus 5′ UTR comprises one or more nucleotide substitutions at position 1, 2, 4, or a combination thereof. In some embodiments, the mutant alphavirus 5′ UTR comprises a U->G substitution at position 2.

In some embodiments, the cargo molecule includes a protein, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, a homeodomain protein, a scavenger receptor, a scavenger receptor ligand, a Ran GTPase, RanQ69L, and any combination thereof. In some embodiments the protein targets a protein in the acceptor cell for degradation. In some embodiments the protein targets a protein in the acceptor cell for degradation by localizing the protein to the proteasome. In some embodiments, the protein is a wild-type protein. In some embodiments, the protein is a mutant protein. In some embodiments the protein is a fusion or chimeric protein.

In some embodiments, the cargo molecule is or comprises a receptor, a ligand, or a functional portion of either thereof. In some embodiments, the receptor or ligand is chosen from Tables 1-6. In some embodiments, the receptor is a receptor described herein, e.g., an RTK or scavenger receptor.

In some embodiments, the cargo molecule is or comprises a cancer driver, e.g., a protein or gene product encoded by a cancer driver gene as described in Bailey et al. Cell. 2018 Apr. 5;173(2):371-385, the list of which is hereby incorporated by reference.

In some embodiments, the cargo molecule is or comprises a Cluster of Differentiation protein or a functional portion or variant thereof.

In some embodiments a cargo molecule is a protein (or a nucleic acid that encodes it) that is naturally found on a membrane surface of a cell (e.g., on a surface of a plasma membrane).

Exemplary membrane proteins (and/or nucleic acids encoding them) include any described herein, and can be found, for example, in U.S. Patent Publication No. 2016/0289674, the contents of which are hereby incorporated by reference. In some embodiments, a cargo molecule (and/or a nucleic acid that encodes it) has a sequence as set forth in any one of SEQ ID NOs: 8144-16131 of U.S. Patent Publication No. 2016/0289674, or in a functional fragment thereof. In some embodiments, a cargo molecule is a plasma membrane protein (nucleic acid encoding it) as set forth in any one of SEQ ID NOs: 8144-16131 of U.S. Patent Publication No. 2016/0289674, or a fragment, variant, or homolog thereof (or nucleic acid that encodes it) of a plasma membrane protein of.

In some embodiments, a membrane protein relevant to the present disclosure is a therapeutic membrane protein. In some embodiments, a membrane protein relevant to the present disclosure is or comprises a cell surface receptor, a membrane transport protein (e.g., an active or passive transport protein such as, for example, an ion channel protein, a pore-forming protein [e.g., a toxin protein], etc.), a membrane enzyme, and/or a cell adhesion protein).

In some embodiments a membrane protein is a single spanning membrane protein. In some embodiments a single-spanning membrane protein may assume a final topology with a cytoplasmic N- and an exoplasmic C-terminus (N_{cyt}/Cₑₓₒ) or with the opposite orientation (Nₑₓₒ/C_{cyt}).

In some embodiments a membrane protein is a Type I membrane protein comprising an N-terminal cleavable signal sequence and stop-transfer sequence (Nₑₓₒ/C_{cyt}). In some embodiments a signal is at the C terminus. In some embodiments the N-terminal cleavable signal sequence targets nascent peptide to the ER. In some embodiments an N-terminal cleavable signal sequence comprises a hydrophobic stretch of typically 7-15 predominantly apolar residues. In some embodiments a Type I membrane protein comprises a stop-transfer sequence which halts the further translocation of the polypeptide and acts as a transmembrane anchor. In some embodiments a stop transfer sequence comprises an amino acid sequence of about 20 hydrophobic residues. In some embodiments the N-terminus of the Type I membrane protein is extracellular and the C-terminus is cytoplasmic. In some embodiments a Type I membrane protein may be a glycophorin or an LDL receptor.

In some embodiments a membrane protein is a Type II membrane protein comprising a signal-anchor sequence (N_{cyt}/Cₑₓₒ). In some embodiments a signal is at the C terminus. In some embodiments a signal-anchor sequence is responsible for both insertion and anchoring of a Type II membrane protein. In some embodiments a signal-anchor sequence comprises about 18-25 predominantly apolar residues. In some embodiments a signal-anchor sequence lacks a signal peptidase cleavage site. In some embodiments a signal-anchor sequence may be positioned internally within a polypeptide chain. In some embodiments a signal-anchor sequence induces translocation of the C-terminal end of a protein across a cell membrane. In some embodiments the C-terminus of the Type II membrane protein is extracellular and the N-terminus is cytoplasmic. In some embodiments a Type II membrane protein may be a transferrin receptor or a galactosyl transferase receptor.

In some embodiments a membrane protein is a Type III membrane protein comprising a reverse signal-anchor sequence (Nₑₓₒ/C_{cyt}). In some embodiments a signal is at the N terminus. In some embodiments a reverse signal-anchor sequence is responsible for both insertion and anchoring of a Type III membrane protein. In some embodiments a reverse signal-anchor sequence comprises about 18-25 predominantly apolar residues. In some embodiments a signal-anchor sequence lacks a signal peptidase cleavage site. In some embodiments a signal-anchor sequence may be positioned internally within a polypeptide chain. In some embodiments a signal-anchor sequence induces translocation of the N-terminal end of a protein across a cell membrane. In some embodiments the N-terminus of the Type III membrane protein is extracellular and the C-terminus is cytoplasmic. In some embodiments a Type I membrane protein may be a synaptogamin, neuregulin, or cytochrome P-450.

In some embodiments, Type I, Type II, or Type III membrane proteins are inserted into a cell membrane via a cellular pathway comprising SRP, SRP receptor and Sec61 translocon.

In some embodiments a membrane protein is predominantly exposed to cytosol and anchored to a membrane by a C-terminal signal sequence, but which does not interact with an SRP. In some embodiments a protein is cytochrome b₅, or a SNARE protein (e.g., synaptobrevin).

In some embodiments a cargo molecule comprises a signal sequence which localizes the cargo molecule to the cell membrane. In some embodiments a cargo molecule is a nucleic acid wherein the nucleic acid encodes a signal sequence which localizes a protein encoded by the nucleic acid to the cell membrane.

In some embodiments, the cargo molecule includes a small molecule, e.g., ions (e.g. Ca²⁺, Cl⁻, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the cargo molecule includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the cargo molecule includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some embodiments, the cargo molecule is enriched at the donor cell, acceptor cell, or membrane-enclosed body cell membrane. In some embodiments, the cargo molecule is enriched by targeting to the membrane via a peptide signal sequence. In some embodiments, the cargo molecule is enriched by binding with a membrane associated protein, lipid, or small molecule. In some embodiments, the cargo molecule is enriched by dimerizing with a membrane associated protein, lipid, or small molecule. In some embodiments the cargo molecule is chimeric (e.g. a chimeric protein, or nucleic acid) and comprises a domain that mediates binding or dimerization with a membrane associated protein, lipid, or small molecule. Membrane-associated proteins of interest include, but are not limited to, membrane proteins described herein or any protein having a domain that stably associates, e.g., binds to, integrates into, etc., a cell membrane (i.e., a membrane-association domain), where such domains may include myristoylated domains, farnesylated domains, transmembrane domains, and the like. Specific membrane-associated proteins of interest include, but are not limited to: myristoylated proteins, e.g., p 60 v-src and the like; farnesylated proteins, e.g., Ras, Rheb, and CENP-E or CENP-F proteins binding specific lipid bilayer components e.g. AnnexinV, by binding to phosphatidyl-serine, a lipid component of the cell membrane bilayer and the like; membrane anchor proteins; transmembrane proteins, e.g., transferrin receptors and portions thereof; and membrane fusion proteins.

In some embodiments, the cargo molecule is present in a donor cell, membrane-enclosed body, or acceptor cell at a higher level (e.g., a higher copy number) than a membrane-associated agent. In some embodiments, the cargo molecule is present in an acceptor cell at a higher level than a membrane-associated agent or one or more components of thereof (e.g., one, two, or all of membrane-associated moiety, extracellular moiety, or intracellular moiety). In some embodiments, the acceptor cell comprises the cargo molecule but does not comprise the membrane-associated agent or one, two, or all of the membrane-associated moiety, intracellular moiety, or extracellular moiety. In some embodiments, the acceptor cell comprises the cargo molecule and comprises only residual levels of the membrane-associated agent or one, two, or all of the membrane-associated, intracellular, or extracellular moiety (e.g., less than 30, 25, 20, 18, 16, 14, 12, 10, 9, 8, 7, 6, 5, 4, 3, 2, or 1% of cells in a sample of acceptor cells comprise exogenous membrane-associated agent or one, two, or all of the membrane-associated moiety, extracellular moiety, or intracellular moiety).

In some embodiments, the cargo molecule is operably associated or linked (e.g., tethered) to the membrane-associated agent (e.g., the membrane-associated moiety, extracellular moiety, or intracellular moiety) when in the donor cell or membrane-enclosed body, but may separate from the membrane-associated agent when in the target cell, e.g., acceptor cell. In some embodiments, a cargo molecule can be non-covalently associated with a membrane-associated agent in the donor cell or membrane-enclosed body and can dissociate from the membrane-associated agent in the target cell (e.g., acceptor cell). In some embodiments, a cargo molecule can be covalently associated with a membrane-associated agent in the donor cell or membrane-enclosed body and the covalent association can be broken in the target cell (e.g., acceptor cell). Without wishing to be bound by theory, a difference in one or more conditions between the donor cell or membrane-enclosed body and the target cell (e.g., acceptor cell) may promote separation of the cargo molecule from the membrane-associated agent. Such differences in condition include but are not limited to differences in: pH, level of a signaling molecule, level or activity of an enzyme, expression of a gene, or the presence or level of an exogenous agent (e.g., a chemical or synthetic agent).

In some embodiments, the cargo molecule is operably associated or linked (e.g., tethered) to a membrane (e.g., the cell membrane) when in the donor cell or membrane-enclosed body, but may separate from the membrane when in the target cell, e.g., acceptor cell. In some embodiments, a cargo molecule can be non-covalently associated with a membrane (e.g., the cell membrane) in the donor cell or membrane-enclosed body and can dissociate from the membrane-associated agent in the target cell (e.g., acceptor cell). In some embodiments, a cargo molecule can be covalently associated with a membrane (e.g., the cell membrane) in the donor cell or membrane-enclosed body and the covalent association can be broken in the target cell (e.g., acceptor cell). Without wishing to be bound by theory, a difference in one or more conditions between the donor cell or membrane-enclosed body and the target cell (e.g., acceptor cell) may promote separation of the cargo molecule from the membrane (e.g., the cell membrane). Such differences in condition include but are not limited to differences in: pH, level of a signaling molecule, level or activity of an enzyme, expression of a gene, or the presence or level of an exogenous agent (e.g., a chemical or synthetic agent). In some embodiments, the difference in condition comprises a difference in the presence, level, or activity of a nuclease or protease (e.g., which recognizes a cleavable sequence in a cargo molecule). In some embodiments, a target cell (e.g., acceptor cell) comprises a nuclease or protease that recognizes a cleavable sequence in the cargo molecule, e.g., wherein cleavage frees the cargo molecule from its association with the membrane. In some embodiments, a donor cell, membrane-enclosed body, or source cell from which the aforementioned are derived does not comprise a nuclease or protease that recognizes a cleavable sequence in the cargo molecule. Suitable proteases and protease cleavable tags include, but are not limited to, any described herein.

Signal Sequences

In some embodiments, a cargo molecule is a protein (or nucleic acid encoding it) that includes or included a signal sequence directing the protein to a particular site or location (e.g., to the cell surface). Those skilled in the art will appreciate that, in certain instances, a cell uses “sorting signals” which are amino acid motifs that are at least temporarily part of a protein (e.g., when initially produced), to target the protein to particular subcellular location (e.g., to a particular organelle or surface membrane of a target cell). In some embodiments a sorting signal is a signal sequence, a signal peptide, or a leader sequence, which directs a protein to an organelle called the endoplasmic reticulum (ER); in some such embodiments, the protein is then delivered to the plasma membrane. See US20160289674A1. In some such embodiments, the protein is then secreted. In some such embodiments, the protein is then trafficked to the lysosome. In some such embodiments, the protein is then trafficked to the Golgi apparatus. In some such embodiments, the protein is then trafficked to a secretory vesicle, and may then be secreted from the cell. In some such embodiments, the protein is then trafficked to an endosome.

In some embodiments, protein targeting to the ER is cotranslational. In some embodiments protein translocation and membrane insertion are coupled to protein synthesis. In some embodiments a signal sequence may be hydrophobic. In some embodiments a signal sequence may be partially hydrophobic. In some embodiments a signal sequence is recognized by a signal recognition particle (SRP). In some embodiments the SRP recognizing a signal sequence as it emerges from a ribosome. In some embodiments, a nascent peptide chain-ribosome complex is targeted to the ER by binding to an SRP receptor. In some embodiments a signal sequence interacts with an Sec61α subunit of a translocon and initiates translocation of a membrane protein or partial chain of said membrane protein.

In some embodiments, a cargo molecule comprises an in-frame fusion of a protein of interest to the coding sequence of a transmembrane protein, or an in-frame fusion of a protein of interest to the transmembrane domain or membrane-anchoring domain of a protein (e.g. fusion to the transferrin receptor membrane anchor domain). See, e.g., Winndard, P, et al. Development of novel chimeric transmembrane proteins for multimodality imaging of cancer cells, Cancer Biology & Therapy. 12:1889-1899 (2007).

In some embodiments a sorting signal or signal peptide is appended to the N or C terminus of a protein (e.g., membrane protein or secreted protein). See Goder, V. & Spiess, M., Topogenesis of membrane proteins: determinants and dynamics. FEBS Letters. 504(3): 87-93 (2001). In some embodiments the protein is a natural protein. In some embodiments the membrane protein is a synthetic protein.

In some embodiments, a signal emerges from a ribosome only after translation of a transcript has reached a stop codon. In some embodiments insertion of a membrane protein is post-translational.

In some embodiments a signal sequence is selected from Table 7. In some embodiments a signal sequence comprises a sequence selected from Table 7. In some embodiments a signal sequence of Table 7 may be appended to the N-terminus of a protein, e.g., a membrane protein or secreted protein. In some embodiments a signal sequence of Table 7 may be appended to the C-terminus of a protein, e.g., a membrane protein or secreted protein. A person of ordinary skill will appreciate that the signal sequences below are not limited for use with their respective naturally associated proteins. In some embodiments, the nucleic acid includes one or more regulatory elements that direct expression of sequences encoding the membrane protein by the target cell.

TABLE 7
Exemplary signal sequences.

| | | Naturally | Location |
|---|---|---|---|
| SEQ ID | | Associated | Associated Protein |
| NO: | Signal Sequence | Protein | is Directed |
| 2 | MRVKEKYQHL WRWGWKWGTM | HIV-1 gp41 | Plasma membrane |
| | LLGILMICSA TE | | |
| 3 | CAAL | p21ras | Plasma membrane |
| 4 | KKKKKK | p21ras | Plasma membrane |
| 5 | RRRRR | p21ras | Plasma membrane |
| 6 | MRLLLALLGV LLSVPGPPVL S | FGFR4 | Plasma membrane |
| 7 | CSIMNLMCGS TC | ROP7 GTPase | Plasma membrane |
| 8 | GHKSEEKREK MKRTLLKDWK | RGS2 | Plasma membrane |
| | TRLSYFLQNS STPGKPKTGK KSKQQ | | |
| 9 | RSTLKLTTLQ CQYSTVMD | LHR | Plasma membrane\ |
| | | | basolateral cell |
| | | | surface |
| 10 | RQGLHNMEDV YELIENSH | TSHR | Plasma membrane\ |
| | | | basolateral cell |
| | | | surface |
| 11 | MDCRKMARFS YSVIWIMAIS | TDGF1 | Plasma membrane |
| | KVFELGLVAG | | |
| 12 | MPAWGALFLL WATAEA | (GP)IX | Plasma membrane |
| 13 | RDYR | VPAC2 | Plasma membrane |
| 14 | KMALRVALNN KQSGQITVKT | Toe159 | Plasma membrane |
| | SSSDHLSLAI AGLVPIALSI | | |
| | YQKFKPGVSP SYSIY | | |
| 15 | MGSKIVQVFL MLALFATSAL A | Classical | Plasma membrane |
| | | arabinogalactan | |
| | | protein 4 | |
| 16 | MNSKAMQALI FLGFLATSCL A | Classical | Plasma membrane |
| | | arabinogalactan | |
| | | protein 2 | |
| 17 | MGAAASIQTT VN | L1R | Plasma membrane |
| | SVM | GTP-binding protein | Plasma membrane |
| | | Rheb | |
| 18 | FALLGTHGAS G | CD147 | Plasma membrane |
| 19 | RRRTFLK | PlcH | Plasma membrane |
| 20 | MGGKWSKSSV | Nef | Plasma membrane |
| 21 | DDPERE | Nef | Plasma membrane |
| 22 | EEANTGENNS LLHPMS | HIV-1 NA7 | Plasma membrane |
| 23 | SRRGLV | DmsA | Plasma membrane |
| 24 | SRRRFL | TorA/TorA-MalE | Plasma membrane |
| 25 | SRRQFI | SufI | Plasma membrane\ |
| | | | periplasm |
| 26 | QRRDFL | YacK | Plasma membrane\ |
| | | | periplasm |
| 27 | MNKIYSIKYS AATGGLIAVS | Pet (Serine protease | Plasma membrane |
| | ELAKKVICKT NRKISAALLS | pet autotransporter) | |
| | LAVISYTNII YA | | |
| 28 | MNPNQKIITI GSICMVIGIV | Influenza A | Plasma Membrane |
| | SLMLQIGNII SIWVSHSIQT | Neuraminidase | |
| 29 | LRCLACSCFR TPVWPR | prRDH | Plasma Membrane |
| 30 | MGCGCSSHPE | Lck | Plasma Membrane |
| 31 | MPFVNKQFN | BoNT/A-LC | Plasma Membrane |
| 32 | DEQNAKNAAQ DRNSNKSSKG | Yck2p | Plasma Membrane |
| | FFSKLGCC | | |
| 33 | MLCCMRRTKQ | GAP-43 | Plasma Membrane |
| 34 | VTNGSTYILV PLSH | FSHR | Plasma Membrane |
| 35 | AETENFV | M3 mAChR | Plasma Membrane |
| 36 | RARHRRNVDR VSIGSYRT | pIgR | Plasma Membrane |
| 37 | YEDQ | RhBG | Plasma Membrane |
| 38 | LLVTSLLLCELPHPAFL IP | GM-CSF Receptor | Plasma membrane |
| | | (GMCSFR) | |

Chimeric Antigen Receptors

The present disclosure provides, in some embodiments, donor cells, membrane-enclosed bodies, and/or acceptor cells comprising a chimeric antigen receptor (CAR), or a functional fragment thereof. In some embodiments, a cargo molecule as described herein comprises a CAR, or a functional fragment thereof. In some embodiments, a membrane-associated agent is or comprises a CAR. In some embodiments, a membrane-associated agent comprises a CAR, or a functional fragment thereof, in its extracellular moiety, intracellular moiety, or cargo molecule. In some embodiments, a membrane-associated agent is bound to a CAR (e.g., by its extracellular moiety or intracellular moiety). In some embodiments, a membrane-associated agent is operably associated or linked (e.g., tethered) to a CAR (e.g., by its extracellular moiety or intracellular moiety).

In some embodiments, a donor cell, acceptor cell, or membrane-enclosed body comprises a CAR, e.g., a first generation CAR or a nucleic acid encoding a first generation CAR. In some embodiments, a membrane-associated agent or a cargo molecule comprises a CAR, e.g., a first generation CAR or a nucleic acid encoding a first generation CAR. In some embodiments, a first generation CAR comprises an antigen binding domain, a transmembrane domain, and signaling domain. In some embodiments a signaling domain mediates downstream signaling during T-cell activation.

In some embodiments, a donor cell, acceptor cell, or membrane-enclosed body comprises a second generation CAR or a nucleic acid encoding a second generation CAR. In some embodiments, a membrane-associated agent or a cargo molecule comprises a second generation CAR or a nucleic acid encoding a second generation CAR. In some embodiments a second generation CAR comprises an antigen binding domain, a transmembrane domain, and two signaling domains. In some embodiments a signaling domain mediates downstream signaling during T-cell activation. In some embodiments a signaling domain is a costimulatory domain. In some embodiments, a costimulatory domain enhances cytokine production, CAR T-cell proliferation, and or CAR T-cell persistence during T cell activation.

In some embodiments, a donor cell, acceptor cell, or membrane-enclosed body comprises a third generation CAR or a nucleic acid encoding a third generation CAR. In some embodiments, a membrane-associated agent or a cargo molecule comprises a third generation CAR or a nucleic acid encoding a third generation CAR. In some embodiments, a third generation CAR comprises an antigen binding domain, a transmembrane domain, and at least three signaling domains. In some embodiments a signaling domain mediates downstream signaling during T-cell activation. In some embodiments a signaling domain is a costimulatory domain. In some embodiments, a costimulatory domain enhances cytokine production, CAR T-cell proliferation, and or CAR T-cell persistence during T cell activation. In some embodiments, a third generation CAR comprises at least two costimulatory domains. In some embodiments, the at least two costimulatory domains are not the same.

In some embodiments, a donor cell, acceptor cell, or membrane-enclosed body comprises a fourth generation CAR or a nucleic acid encoding a fourth generation CAR. In some embodiments, a membrane-associated agent or a cargo molecule comprises a fourth generation CAR or a nucleic acid encoding a fourth generation CAR. In some embodiments a fourth generation CAR comprises an antigen binding domain, a transmembrane domain, and at least two, three, or four signaling domains. In some embodiments a signaling domain mediates downstream signaling during T-cell activation. In some embodiments a signaling domain is a costimulatory domain. In some embodiments, a costimulatory domain enhances cytokine production, CAR T-cell proliferation, and or CAR T-cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, or IFN-gamma, or functional fragment thereof. In some embodiments a domain which upon successful signaling of the CAR induces expression of a cytokine gene is or comprises a transcription factor or functional domain or fragment thereof. In some embodiments a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

### CAR Antigen Binding Domains

In some embodiments, a CAR (e.g., comprised in a donor cell, acceptor cell, membrane-enclosed body, exogenous membrane-associated agent, or cargo molecule) comprises an antigen binding domain (e.g., a CAR antigen binding domain). In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; or transferrin receptor antibody.

In some embodiments, an antigen binding domain binds to a cell surface antigen of a cell. In some embodiments, a cell surface antigen is characteristic of one type of cell. In some embodiments, a cell surface antigen is characteristic of more than one type of cell.

In some embodiments a CAR antigen binding domain binds a cell surface antigen characteristic of a T-cell. In some embodiments, an antigen characteristic of a T-cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T-cell. In some embodiments, an antigen characteristic of a T-cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen characteristic of a T-cell may be a T-cell receptor. In some embodiments, a T-cell receptor may be AKT1; AKT2; AKT3; ATF2; BCL10; CALM1; CD3D (CD3δ); CD3E (CDR); CD3G (CD3γ); CD4; CD8; CD28; CD45; CD80 (B7-1); CD86 (B7-2); CD247 (CD3); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments a CAR antigen binding domain binds an antigen characteristic of a neoplastic cell, e.g., cancer. In some embodiments an antigen characteristic of a cancer is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Ra1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gp1OO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, 0101E2, TARP, WT1, NY-ESO-1, LAGE-la, MAGE-A1, legumain, HPV E6, E7, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TESL LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A, B, C) CD49f, CD151 CD340, CD200, tkrA, trkB, or trkC, or an antigenic fragment or antigenic portion thereof.

In some embodiments a CAR antigen binding domain binds an antigen characteristic of an infectious disease (e.g. a viral infection or a bacterial infection). In some embodiments an antigen is characteristic of an infectious disease selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Eptstein-Barr virus, CMV, human papillomavirus. In some embodiments an antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds an antigen characteristic of an infectious disease, wherein the antigen is selected from HIV Env, gp120, or CD4-induced epitope on HIV-1 Env. See, e.g., WO2015/077789, the contents of which are herein incorporated by reference. In some embodiments, a CAR antigen binding domain comprises CD4 or an HIV binding fragment thereof.

In some embodiments a CAR antigen binding domain binds an antigen characteristic of an autoimmune or inflammatory disorder. In some embodiments the antigen is characteristic of an autoimmune or inflammatory disorder selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purpura, neuromyelitis optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments an antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds to a ligand expressed on B cells, plasma cells, or plasmablasts. In some embodiments, a CAR antigen binding domain binds an antigen characteristic of an autoimmune or inflammatory disorder, wherein the antigen is selected from CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference.

CAR Transmembrane Domains

In some embodiments, a CAR (e.g., comprised in a donor cell, acceptor cell, membrane-enclosed body, exogenous membrane-associated agent, or cargo molecule) comprises a CAR transmembrane domain. In some embodiments, a membrane-associated moiety of a membrane-associated agent comprises a CAR transmembrane domain. In some embodiments a CAR comprises at least a transmembrane region of the alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments a CAR comprises at least a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

CAR Signaling Domains

In some embodiments, a CAR (e.g., comprised in a donor cell, acceptor cell, membrane-enclosed body, exogenous membrane-associated agent, or cargo molecule) comprises a signaling domain (e.g., a CAR signaling domain). In some embodiments a CAR comprises a signaling domain of one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNF SF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), or functional variant thereof.

In some embodiments a CAR comprises a signaling domain which is a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83.

In some embodiments, a CAR comprises a CD3 zeta domain or an immunoreceptor tyrosine-based activation motif (ITAM), or functional variant thereof. In some embodiments, a CAR comprises a (i) a CD3 zeta domain, or an immunoreceptor tyrosine-based activation motif (ITAM), or functional variant thereof; and (ii) a CD28 domain, or a 4-1BB domain, or functional variant thereof. In some embodiments, a CAR comprises a (i) a CD3 zeta domain, or an immunoreceptor tyrosine-based activation motif (ITAM), or functional variant thereof (ii) a CD28 domain or functional variant thereof; and (iii) a 4-1BB domain, or a CD134 domain, or functional variant thereof. In some embodiments, a CAR comprises a (i) a CD3 zeta domain, or an immunoreceptor tyrosine-based activation motif (ITAM), or functional variant thereof (ii) a CD28 domain or functional variant thereof (iii) a 4-1BB domain, or a CD134 domain, or functional variant thereof; and (iv) a cytokine or costimulatory ligand transgene.

CAR Spacers

In some embodiments a CAR (e.g., comprised in a donor cell, acceptor cell, membrane-enclosed body, exogenous membrane-associated agent, or cargo molecule) comprises one or more spacers. In some embodiments a CAR comprises a spacer between the antigen binding domain and the transmembrane domain. In some embodiments a CAR comprises a spacer between a transmembrane domain and an intracellular signaling domain.

CAR Membrane Protein Payload Agents

In addition to the CARs described herein, various chimeric antigen receptors and nucleotide sequences encoding the same are known in the art and would be suitable for transfer from a donor cell to an acceptor cell, e.g., as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments, a membrane-associated agent and/or a cargo molecule comprising a CAR (e.g., a DNA, a gDNA, a cDNA, an RNA, a pre-MRNA, an mRNA, an miRNA, an siRNA, etc.), e.g., comprised in a donor cell, is transferred to an acceptor cell. In some embodiments the acceptor cell is an effector cell, e.g., a cell of the immune system that expresses one or more Fc receptors and mediates one or more effector functions. In some embodiments, an acceptor cell may include, but may not be limited to, one or more of a monocyte, macrophage, neutrophil, dendritic cell, eosinophil, mast cell, platelet, large granular lymphocyte, Langerhans' cell, natural killer (NK) cell, T-lymphocyte (e.g., T-cell), a Gamma delta T cell, B-lymphocyte (e.g., B-cell) and may be from any organism including but not limited to humans, mice, rats, rabbits, and monkeys.

T Cell Receptor Payloads

In some embodiments, a donor cell, acceptor cell, or membrane-enclosed body comprises a polypeptide comprising a T cell receptor, e.g., a T-cell receptor fusion protein (TFP). In some embodiments, a membrane-associated agent or a cargo molecule comprises a T cell receptor, e.g., a TFP. In some embodiments, the TFP comprises a recombinant polypeptide derived from the various polypeptides comprising the TCR that is generally capable of i) binding to a surface antigen on target cells and ii) interacting with other polypeptide components of the intact TCR complex, typically when co-located in or on the surface of a T-cell. In some embodiments, the TFP incorporates into a TCR when expressed in a T-cell. In some embodiments, the membrane-associated agent or cargo molecule comprises (i) an antigen binding domain operatively linked to (ii) a TCR domain.

The antigen-binding domain may comprise, e.g., an scFv, e.g., an scFv that binds an antigen comprised by a cancer cell, e.g., an antigen at the surface of a cancer cell. The antigen-binding domain may be human or humanized. In some embodiments, the antigen-binding domain is an antigen-binding domain described herein, e.g., in the section entitled “CAR Antigen Binding Domains”.

In some embodiments, the antigen-binding domain binds an Fc domain of an antibody. In some embodiments, the antigen-binding domain selectively binds to an IgG1 antibody. In some embodiments, the antigen-binding domain binds to a cell surface antigen, e.g., a cell surface antigen on the surface of a tumor cell. In some embodiments, the antigen-binding domain comprises a monomer, a dimer, a trimer, a tetramer, a pentamer, a hexamer, a heptamer, an octamer, a nonamer, or a decamer. In some embodiments, the antigen-binding domain does not comprise an antibody or fragment thereof. In some embodiments, the antigen-binding domain comprises a CD16 polypeptide or fragment thereof. In some embodiments, the antigen-binding domain comprises a CD16-binding polypeptide.

In some embodiments, the TFP includes an extracellular domain of a TCR subunit that comprises an extracellular domain or portion thereof of a protein selected from the group consisting of the alpha or beta chain of the T cell receptor, CD3 delta, CD3 epsilon, or CD3 gamma, or a functional fragment or variant thereof. In some embodiments, the TCR domain includes a transmembrane domain, e.g., at least a transmembrane region of a transmembrane domain of a TCR alpha chain, a TCR beta chain, a CD3 epsilon TCR subunit, a CD3 gamma TCR subunit, a CD3 delta TCR subunit, or a CD3 zeta TCR subunit, or a functional fragment or variant thereof.

In further embodiments, the TCR domain comprises a TCR intracellular domain comprising a stimulatory domain selected from an intracellular signaling domain of CD3 epsilon, CD3 gamma, or CD3 delta, or a variant thereof.

In further embodiments, the TCR domain comprises (i) a TCR extracellular domain, (ii) a TCR transmembrane domain, and (iii) a TCR intracellular domain, wherein at least two or all three of (i), (ii), and (iii) are from the same TCR subunit.

In some embodiments, the TCR domain comprises CD3ε or a functional fragment or variant thereof. In some embodiments, the TCR domain (e.g., CD3ε-based TCR domain) binds endogenous CD3ζ. In some embodiments, the TCR domain (e.g., CD3ε-based TCR domain) binds endogenous CD3γ and/or endogenous CD3δ. In some embodiments, the TCR domain comprises CD3α or a functional fragment or variant thereof. In some embodiments, the TCR domain comprises CD3β or a functional fragment or variant thereof. In some embodiments, the TCR domain (e.g., CD3α-based or CD3β-based TCR domain) binds endogenous CD3ζ. In some embodiments, the TCR domain (e.g., CD3α-based TCR domain) binds endogenous CD3β. In some embodiments, the TCR domain (e.g., CD3β-based TCR domain) binds endogenous CD3α. In some embodiments, the TCR domain (e.g., CD3α-based or CD3β-based TCR domain) binds endogenous CD3δ.

In some embodiments, a TFP comprises a TCR subunit comprising at least a portion of a TCR extracellular domain, and a TCR intracellular domain comprising a stimulatory domain from an intracellular signaling domain of CD3 (e.g., CD3 epsilon, CD3 gamma, CD3 delta, TCR alpha, or TCR beta); and a human or humanized antigen binding domain, wherein the TCR subunit and the antigen binding domain are operatively linked, and wherein the TFP incorporates into a TCR when expressed in a T-cell. In some embodiments, a TFP comprises a TCR subunit and a human or humanized antibody domain comprising an antigen binding domain that is an anti-CD19 binding domain or an anti-B-cell maturation antigen (BCMA) binding domain.

Exemplary TFPs are described, e.g., in WO2016187349, WO2018026953, WO2018067993, WO2018098365, WO2018119298, and WO2018232020, each of which is incorporated herein by reference in its entirety.

Pharmaceutical Compositions

In one aspect, the present disclosure is directed in part to a pharmaceutical composition comprising a donor cell or membrane-enclosed body described herein. In some embodiments, a pharmaceutical composition comprises a plurality of donor cells or plurality of membrane-enclosed bodies. In some embodiments, the donor cell or membrane-enclosed body comprises a membrane-associated agent (e.g., comprising one, two, or all three of an intracellular moiety, a membrane-associated moiety, and/or an extracellular moiety) as described herein. In some embodiments, the donor cell or membrane-enclosed body comprises a cargo molecule as described herein. In some embodiments, the membrane-associated agent and the cargo molecule are operably associated or linked. In some embodiments, a pharmaceutical composition described herein comprises one or more pharmaceutically acceptable excipients. In some embodiments, a pharmaceutical composition comprises an acceptor cell or a plurality of acceptor cells. In some embodiments, a pharmaceutical composition comprises a donor cell (e.g., a plurality of donor cells) and an acceptor cell (e.g., a plurality of acceptor cells).

In embodiments, a pharmaceutical composition described herein comprises a molecule that specifically binds to the donor cell (e.g., specifically binds to the membrane-associated agent) and specifically binds to the acceptor cell. In embodiments, the molecule that specifically binds to the donor cell and specifically binds to the acceptor cell is a multispecific molecule, e.g., an antibody molecule (e.g., Fab, F(ab′)2, Fab′, scFv, or di-scFv), e.g., a bispecific antibody molecule.

In embodiments, a pharmaceutical composition described herein has one or more of the following characteristics:

the pharmaceutical composition meets a pharmaceutical or good manufacturing practices (GMP) standard;

the pharmaceutical composition was made according to good manufacturing practices (GMP);

the pharmaceutical composition has a pathogen level below a predetermined reference value, e.g., is substantially free of pathogens;

the pharmaceutical composition has a contaminant level (e.g., nuclear DNA) below a predetermined reference value, e.g., is substantially free of contaminants; or

the pharmaceutical composition has low immunogenicity, e.g., as described herein.

Membrane Transfer Processes

As used herein, a “membrane transfer process” is any process capable of moving a portion of a membrane (e.g., one or more components of said membrane, e.g., a membrane-associated agent) from a first cell (e.g., donor cell) or membrane-enclosed body to a second cell (e.g., target cell, e.g., acceptor cell) or membrane-enclosed body when the first cell or membrane-enclosed body is in contact or close proximity with the second cell or membrane-enclosed body. Exemplary membrane transfer processes include, but are not limited to, a membrane fusion event, a receptor-ligand interaction, a cell bridging event (e.g., an antibody molecule (e.g., a bispecific antibody), or cell to cell contact event. Membrane transfer processes may adapt or use in part components or mechanisms of naturally occurring membrane transfer processes, e.g., trogocytosis or endocytosis.

In some embodiments, a cargo molecule and/or a membrane-associated agent is transferred from a donor cell or membrane-enclosed body to an acceptor cell by a membrane transfer process. In some embodiments, a membrane transfer process comprises contact between a donor cell or membrane-enclosed body (comprising a membrane-associated agent and optionally a cargo molecule) and an acceptor cell. In some embodiments, a membrane transfer process comprises close proximity between a donor cell or membrane-enclosed body (comprising a membrane-associated agent and optionally a cargo molecule) and an acceptor cell. In some embodiments, close proximity comprises a distance of no more than 1, 2, 3, 4, 5, 6, 7, 8, 9, 10, 15, 20, 25, 30, 35, 40, 45, or 50 nm, or no more than 1, 2, 3, 4, 5, 6, 7, 8, 9, or 10 μm. In some embodiments, close proximity comprises a distance of no more than 0.1× (i.e., 0.1 times), 0.2×, 0.3×, 0.4×, 0.5×, 0.6×, 0.7×, 0.8×, 0.9×, 1×, 1.1×, 1.2×, 1.3×, 1.4×, 1.5×, 1.75×, 2×, 2.25×, 2.5×, 2.75×, 3×, 3.25×, 3.5×, 3.75×, or 4× the width (e.g., the average diameter) of the donor cell, acceptor cell, or membrane-enclosed body. In some embodiments, a cargo molecule and/or a membrane-associated agent is transferred from a donor cell or membrane-enclosed body to an acceptor cell by a membrane transfer process, e.g., wherein the level of agent delivered via a membrane transfer process is 0.01-0.6, 0.01-0.1, 0.1-0.3, or 0.3-0.6, or at least 1%, 2%, 3%, 4%, 5%, 10%, 20%, 30%, 40%, 50%, 60%, 70%, 80%, 90% or greater than a reference acceptor cell contacted with a similar donor cell not configured to transfer the cargo molecule and/or a membrane-associated agent. In some embodiments, at least 1%, 2%, 3%, 4%, 5%, 10%, 20%, 30%, 40%, 50%, 60%, 70%, 80%, 90% of cargo molecules and/or exogenous membrane-associated agents in a donor cell composition or preparation that enter an acceptor cell enter via a membrane transfer process. In some embodiments, the membrane transfer process comprises a membrane fusion event, a receptor-ligand interaction, a cell bridging event (e.g., an antibody molecule (e.g., a bispecific antibody), a nanotube transfer, or cell to cell contact event.

Methods of Use

This disclosure provides, in certain aspects, methods of transferring a cargo molecule and/or exogenous membrane-associated agent from a donor cell or membrane-enclosed body to an acceptor cell.

In an aspect, the disclosure provides a method of modifying a cell, comprising contacting the acceptor cell with a donor cell, membrane-enclosed body, or system described herein, under conditions suitable for transfer of the membrane-associated agent to the acceptor cell, wherein the acceptor cell does not comprise a nucleic acid encoding the membrane-associated agent. In some embodiments, modifying the acceptor cell comprises transferring the membrane-associated agent from the donor cell or membrane-enclosed body (e.g., the first donor cell, the second donor cell, or both) to the acceptor cell.

In an aspect, the disclosure provides a method of making a modified cell, comprising: providing an unmodified cell, contacting the unmodified cell with a donor cell, membrane-enclosed body, or system described herein, under conditions suitable for transfer of the membrane-associated agent to the unmodified cell, wherein neither the unmodified cell or modified cell comprise a nucleic acid encoding the membrane-associated agent. In some embodiments, the modified cell comprises membrane-associated agent, and wherein the membrane-associated agent was not produced in the acceptor cell. In some embodiments, the method further comprises providing a donor cell (e.g., a first donor cell, second donor cell, both, a third donor cell, or all three) described herein. In some embodiments, the providing comprises contacting a cell with a nucleic acid encoding the membrane-associated agent, thereby providing a donor cell or membrane-enclosed body comprising a nucleic acid encoding the membrane-associated agent.

In an aspect, the disclosure provides a method of delivering a cargo molecule to a cell, comprising: providing a donor cell, membrane-enclosed body, or system described herein, wherein the donor cell or membrane-enclosed body comprises a membrane-associated agent comprising the cargo molecule; providing an acceptor cell that does not comprise a nucleic acid encoding the membrane-associated agent; and contacting the acceptor cell with the donor cell, membrane-enclosed body, or system under conditions suitable for transfer of the membrane-associated agent to the acceptor cell. In some embodiments, the method further comprises contacting the donor cell (or membrane-enclosed body) and the acceptor cell with a multispecific molecule, e.g., antibody molecule ((e.g., Fab, F(ab′)2, Fab′, scFv, or di-scFv), e.g., a bispecific antibody molecule), that specifically binds to the donor cell or membrane-enclosed body (e.g., specifically binds to the membrane-associated agent) and specifically binds to the acceptor cell, under conditions sufficient to allow the acceptor cell to acquire the membrane-associated agent or cargo molecule.

In some embodiments, acceptor cell acquires a sufficient quantity of the membrane-associated agent and/or cargo molecule to provide a desired function (e.g., relative to a reference cell not receiving the membrane-associated agent and/or cargo molecule). A person of skill in the art will recognize that a sufficient quantity will depend upon the membrane-associated agent and/or cargo molecule in question and the function to be achieved. In some embodiments, a sufficient quantity is a single copy of the membrane-associated agent and/or cargo molecule (e.g., wherein the cargo molecule comprises a virus or nucleic acid that integrates into an acceptor cell genome). In some embodiments, a sufficient quantity comprises at least 1, 2, 3, 4, 5, 6, 7, 8, 9, 10, 20, 30, 40, 50, 60, 70, 80, 90, 95, 96, 97, 98, or 99% resurfacing of the acceptor cell with donor cell/membrane-enclosed body/membrane-containing substrate derived membrane (e.g., comprising membrane-associated agent and/or cargo molecule).

In some embodiments of any of the above methods, the method further comprises one, two, three, four or all of:
i) expanding the acceptor cell or population comprising acceptor cells;
ii) selecting the acceptor cell or population comprising acceptor cells;
iii) enriching for the acceptor cell or population comprising acceptor cells;
iv) purifying the acceptor cell or population comprising acceptor cells; or
v) formulating the acceptor cell or population comprising acceptor cells.

In some embodiments of any of the above methods, the method further comprises one, two, three, four or all of:
i) expanding the donor cell or population comprising donor cells;
ii) selecting the donor cell or population comprising donor cells;
iii) enriching for the donor cell or population comprising donor cells;
iv) purifying the donor cell or population comprising donor cells; or
v) formulating the donor cell or population comprising donor cells.

In some embodiments, the contacting occurs in vitro or ex-vivo. In some embodiments, the contacting occurs in vivo.

In an aspect, the disclosure provides method of modulating, e.g., enhancing or decreasing, a biological function in a subject, a target tissue, or a cell, comprising administering to the subject, or contacting the target tissue or the cell with, a donor cell or membrane-enclosed body described herein, an acceptor cell described herein, a system described herein, or a pharmaceutical composition described herein.

In some embodiments, a method of modulating, e.g., increasing or decreasing, a biological function comprises modulating an interaction between a receptor and a ligand or an interleukin and a receptor, e.g., a receptor, ligand, interleukin, or pair thereof described in Tables 1-6, by administering to a subject, or contacting a target tissue or cell with, a donor cell or membrane-enclosed body described herein, an acceptor cell described herein, a system described herein, or a pharmaceutical composition described herein. In some embodiments, the method comprises modulating (e.g., increasing or decreasing) the signaling activity of a receptor, ligand, interleukin, or pair thereof described in Tables 1-6, or described herein (e.g., an RTK, scavenger receptor, or Cluster of Differentiation protein). In some embodiments, the donor cell, membrane-enclosed body, acceptor cell, system, or pharmaceutical composition comprises a membrane-associated agent or cargo molecule comprising the receptor, ligand, or interleukin.

In an aspect, the disclosure provides a method of delivering or targeting a function to a subject, comprising administering to the subject a donor cell or membrane-enclosed body described herein, an acceptor cell described herein, a system described herein, or a pharmaceutical composition described herein, wherein the donor cell, membrane-enclosed body, the acceptor cell or the pharmaceutical composition is administered in an amount and/or time such that the function in the subject is delivered or targeted.

In some embodiments, the subject has a cancer, an inflammatory disorder, autoimmune disease, a chronic disease, inflammation, damaged organ function, an infectious disease, a degenerative disorder, a genetic disease, or an injury.

This disclosure provides, in certain aspects, a method of delivering a donor cell or membrane-enclosed body comprising a membrane-associated agent and optionally a cargo molecule as described herein to a human subject, a target tissue, or an acceptor cell, comprising administering to the human subject, or contacting the target tissue or the acceptor cell with, a donor cell or membrane-enclosed body described herein, a composition comprising one or more donor cells described herein, or a pharmaceutical composition described herein, thereby administering the donor cell to the subject.

This disclosure provides, in certain aspects, a method of delivering a membrane-associated agent (e.g., comprising or operably associated or linked to a cargo molecule) to a subject, a target tissue, or an acceptor cell, comprising administering to the subject, or contacting the target tissue or the acceptor cell with, a donor cell or membrane-enclosed body described herein or a composition or preparation described herein (e.g., a pharmaceutical composition described herein), wherein the donor cell, membrane-enclosed body, composition, or preparation is administered in an amount and/or time such that the membrane-associated agent and/or cargo molecule are delivered.

This disclosure provides, in certain aspects, a method of delivering a cargo molecule to a subject, a target tissue, or an acceptor cell, comprising administering to the subject, or contacting the target tissue or the acceptor cell with, a donor cell or membrane-enclosed body described herein or a composition or preparation described herein (e.g., a pharmaceutical composition described herein), wherein the donor cell, membrane-enclosed body, composition, or preparation is administered in an amount and/or time such that the cargo molecule is delivered.

This disclosure provides, in certain aspects, a method of modulating, e.g., enhancing, a biological function in a subject, a target tissue, or a cell (e.g., an acceptor cell), comprising administering to the subject, or contacting the target tissue or the cell with, a donor cell, membrane-enclosed body, composition, or preparation comprising a membrane-associated agent (e.g., comprising or operably associated or linked to a cargo molecule) described herein, e.g., a pharmaceutical composition described herein, thereby modulating the biological function in the subject.

This disclosure provides, in certain aspects, a method of delivering or targeting a membrane protein function to a subject, comprising administering to the subject a donor cell, membrane-enclosed body, composition, or preparation described herein that comprises a membrane-associated agent (e.g., comprising or operably associated or linked to a cargo molecule), wherein the donor cell, membrane-enclosed body, composition, or preparation is administered in an amount and/or time such that the membrane protein function is delivered or targeted in the subject. In some embodiments, the membrane-associated agent has the membrane protein function to be delivered or targeted. In some embodiments, the cargo molecule has the membrane protein function to be delivered or targeted. In embodiments, the subject has a cancer, an inflammatory disorder, autoimmune disease, a chronic disease, inflammation, damaged organ function, an infectious disease, a degenerative disorder, a genetic disease, or an injury.

The administration of a pharmaceutical composition described herein may be delivered, for example, by way of oral, inhaled, transdermal or parenteral (including intravenous, intratumoral, intraperitoneal, intramuscular, intracavity, and subcutaneous) administration. The donor cells or membrane-enclosed bodies may be administered alone or formulated as a pharmaceutical composition.

The donor cell, membrane-enclosed body, composition, or preparation may be administered, in some embodiments, in the form of a unit-dose composition, such as a unit dose oral, parenteral, transdermal or inhaled composition. Such compositions are generally prepared by admixture and are suitably adapted for oral, inhaled, transdermal or parenteral administration, and as such may be in the form of tablets, capsules, oral liquid preparations, powders, granules, lozenges, reconstitutable powders, injectable and infusible solutions or suspensions or suppositories or aerosols.

In some embodiments, delivery of a membrane-associated agent and/or cargo molecule via a donor cell or membrane-enclosed body, as described herein, may induce or block cellular differentiation, de-differentiation, or trans-differentiation. The acceptor cell may be a precursor cell. Alternatively, the acceptor cell may be a differentiated cell, and the cell fate alteration includes driving de-differentiation into a pluripotent precursor cell, or blocking such de-differentiation. In situations where a change in cell fate is desired, effective amounts of a donor cell or membrane-enclosed body described herein comprising (e.g., as a cargo molecule) a cell fate inductive molecule or signal is introduced into a target cell under conditions such that an alteration in cell fate is induced. In some embodiments, a donor cell or membrane-enclosed body described herein is useful to reprogram a subpopulation of cells from a first phenotype to a second phenotype. Such a reprogramming may be temporary or permanent. Optionally, the reprogramming induces a target cell to adopt an intermediate phenotype.

Also provided are methods of reducing cellular differentiation in a target cell population. For example, a target cell population containing one or more precursor cell types is contacted with a donor cell or membrane-enclosed body or composition described herein, under conditions such that the composition reduces the differentiation of the precursor cell. In certain embodiments, the target cell population (e.g., a population comprising a plurality of acceptor cells as described herein) contains injured tissue in a mammalian subject or tissue affected by a surgical procedure. The precursor cell is, e.g., a stromal precursor cell, a neural precursor cell, or a mesenchymal precursor cell.

A donor cell, membrane-enclosed body, or composition thereof described herein, comprising a membrane-associated agent may be used to deliver a cargo molecule (e.g., comprised in, operably associated to or linked to, or not attached to, the membrane-associated agent) to a cell tissue or subject. Delivery of a membrane-associated agent and/or cargo molecule by administration of a donor cell, membrane-enclosed body or composition described herein may modify cellular protein expression levels. In certain embodiments, the delivered agent or cargo molecule directs upregulation of (via expression in the cell, delivery in the cell, or induction within the cell) of one or more polypeptides or nucleic acids that provides a functional activity which is substantially absent or reduced in the cell into which the agent or cargo molecule is delivered. For example, the missing functional activity may be enzymatic, structural, signaling or regulatory in nature. In related embodiments, the administered composition directs up-regulation of one or more polypeptides or nucleic acids that increases (e.g., synergistically) a functional activity which is present but substantially deficient in the cell in which the membrane protein payload agent is upregulated. In related embodiments, the administered composition directs down-regulation of one or more polypeptides or nucleic acids that decreases (e.g., synergistically) a functional activity which is present or upregulated in the cell in which the polypeptide or nucleic acid is downregulated. In certain embodiments, the administered agent or cargo molecule directs upregulation of certain functional activities and downregulation of other functional activities.

In embodiments, the donor cell, membrane-enclosed body, or composition, or the membrane-associated agent and/or cargo molecule transferred from same, mediates an effect on an acceptor cell, and the effect lasts for at least 1, 2, 3, 4, 5, 6, or 7 days, 2, 3, or 4 weeks, or 1, 2, 3, 6, or 12 months. In some embodiments, the effect lasts for less than 1, 2, 3, 4, 5, 6, or 7 days, 2, 3, or 4 weeks, or 1, 2, 3, 6, or 12 months.

### Ex-Vivo Applications

In embodiments, the donor cell, membrane-enclosed body, or composition described herein is delivered ex-vivo to a cell or tissue, e.g., a human cell or tissue. In embodiments, the donor cell or membrane-enclosed body transfers a membrane-associated agent and/or a cargo molecule to an acceptor cell in the tissue. In some embodiments, the membrane-associated agent and/or cargo molecule improves function of a cell or tissue ex-vivo, e.g., improves cell viability, signaling, respiration, or other function (e.g., another function described herein). In some embodiments, the donor cell and/or acceptor cell is an ex vivo cell.

In some embodiments, the donor cell, membrane-enclosed body, or composition is delivered to an ex vivo tissue that is in an injured state (e.g., from trauma, disease, hypoxia, ischemia or other damage).

In some embodiments, the donor cell, membrane-enclosed body, or composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). The composition can be delivered to the tissue or organ before, during and/or after transplantation.

In some embodiments, the donor cell, membrane-enclosed body, or composition is delivered, administered or contacted with a cell (e.g., an acceptor cell), e.g., in a cell preparation. The cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

### In Vivo Uses

The donor cells, membrane-enclosed bodies, and compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein). In one embodiment, the subject has cancer. In one embodiment, the subject has an infectious disease.

In some embodiments, the source of the donor cells or membrane-enclosed bodies (e.g., as described herein) is from the same subject that is administered the donor cells or a composition comprising donor cells. In other embodiments, they are different, e.g., are allogeneic. For example, the source of the donor cells and recipient tissue may be autologous (from the same subject) or heterologous (from different subjects). In either case, the donor tissue for donor cells described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

A donor cell, membrane-enclosed body, or composition described herein may be administered to a subject having a cancer, an autoimmune disease, an infectious disease, a metabolic disease, a neurodegenerative disease, or a genetic disease (e.g., enzyme deficiency). In some embodiments, a tissue of the subject is in need of regeneration.

In some embodiments, a therapeutically effective amount of donor cells, membrane-enclosed bodies, or a composition described herein is administered to a subject. In some embodiments, a therapeutically effective amount of a substance (e.g., a donor cell, membrane-enclosed body, exogenous membrane-associated agent, and/or cargo molecule) is an amount that is sufficient, when administered to a subject who has or is susceptible to a disease, disorder, and/or condition, to treat, and/or delay the onset of the disease, disorder, and/or condition. For example, in embodiments the effective amount of donor cells, membrane-enclosed bodies, exogenous membrane-associated agents, and/or cargo molecules in a formulation to treat a disease, disorder, and/or condition is the amount that alleviates, ameliorates, relieves, inhibits, delays onset of, reduces severity of and/or reduces incidence of one or more symptoms or features of the disease, disorder, and/or condition.

In some embodiments, a subject is treated with a donor cell, membrane-enclosed body, or a composition as described herein. In some embodiments, the treatment partially or completely alleviates, ameliorates, relieves, inhibits, delays onset of, reduces severity of, and/or reduces incidence of one or more symptoms, features, and/or causes of a particular disease, disorder, and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. In some embodiments, treatment partially or completely ameliorates the root cause of the relevant disease, disorder, and/or condition. In some embodiments, the disease, disorder, or condition is selected from cancer, an inflammatory disorder, autoimmune disease, a chronic disease, inflammation, damaged organ function, an infectious disease, a degenerative disorder, a genetic disease, and/or an injury.

In some embodiments, the donor cells, membrane-enclosed body, or composition are effective to treat the disease, e.g., cancer. In some embodiments, the donor cells or composition are effective to reduce the number of cancer cells in the subject compared to the number of cancer cells in the subject before administration. In some embodiments, the donor cells or composition are effective to reduce the number of cancer cells in the subject compared to the expected course of disease without treatment. In some embodiments, the subject experiences a complete response or partial response after administration of the donor cells or composition.

In some embodiments, the donor cell or membrane-enclosed body is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 DOI: 10.1038/srep01462). Thus, in some embodiments, the donor cell is co-administered with an inhibitor of supressyn, e.g., a siRNA or inhibitory antibody.

### Non-Human Applications

Compositions described herein (e.g., comprising donor cells or membrane-enclosed bodies comprising exogenous membrane-associated agents and/or cargo molecules) may also be used to similarly modulate the cell or tissue function or physiology of a variety of other organisms including but not limited to: farm or working animals (horses, cows, pigs, chickens etc.), pet or zoo animals (cats, dogs, lizards, birds, lions, tigers and bears etc.), aquaculture animals (fish, crabs, shrimp, oysters etc.), plants species (trees, crops, ornamentals flowers etc.), fermentation species (saccharomyces etc.). Donor cells, membrane-enclosed bodies, and compositions described herein can be made from such non-human sources and administered to a non-human target cell or tissue or subject.

Donor cells, acceptor cells, membrane-enclosed bodies, and/or cargo molecules can be autologous, allogeneic or xenogeneic to the subject. Donor cells, membrane-enclosed bodies, and/or cargo molecules can be autologous, allogeneic or xenogeneic to the target cell, e.g., acceptor cell.

Compositions comprising the donor cells, membrane-enclosed bodies, exogenous membrane-associated agents, and/or cargo molecules described herein may be administered or targeted to the circulatory system, hepatic system, renal system, cardio-pulmonary system, central nervous system, peripheral nervous system, musculoskeletal system, lymphatic system, immune system, sensory nervous systems (sight, hearing, smell, touch, taste), digestive system, endocrine systems (including adipose tissue metabolic regulation), and reproductive system.

In embodiments, a donor cell, membrane-enclosed body, or composition described herein is delivered ex-vivo to a cell or tissue, e.g., a human cell or tissue. In some embodiments, the donor cell, membrane-enclosed body, or composition is delivered to an ex vivo tissue that is in an injured state (e.g., from trauma, disease, hypoxia, ischemia or other damage).

In some embodiments, the donor cell, membrane-enclosed body, or composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the donor cell, membrane-enclosed body, or composition improves viability, respiration, or other function of the transplant. The donor cell, membrane-enclosed body, or composition can be delivered to the tissue or organ before, during and/or after transplantation.

In some embodiments, a donor cell, membrane-enclosed body, or composition described herein is delivered ex-vivo to an acceptor cell or tissue derived from a subject. In some embodiments, the acceptor cell or tissue is readministered to the subject (i.e., the cell or tissue is autologous), e.g., as a donor cell for an acceptor cell within the subject.

The donor cells or membrane-enclosed bodies may transfer a membrane-associated agent (e.g., comprising or operably associated or linked to a cargo molecule) and/or a cargo molecule to an acceptor cell from any mammalian (e.g., human) tissue, e.g., from epithelial, connective, muscular, or nervous tissue or cells, and combinations thereof. The membrane-associated agent and/or cargo molecule can be delivered to any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)′; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

In embodiments, the donor cell or membrane-enclosed body targets an acceptor cell in a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, adipose tissue (e.g., brown adipose tissue or white adipose tissue) or eye, when administered to a subject, e.g., wherein at least 0.1%, 0.5%, 1%, 1.5%, 2%, 2.5%, 3%, 4%, 5%, 10%, 20%, 30%, 40%, 50%, 60%, 70%, 80%, or 90% of the membrane-associated agents and/or cargo molecules in a population of administered donor cells or membrane-enclosed bodies, remain present in the target tissue after 24, 48, or 72 hours. In some embodiments, the donor cell or membrane-enclosed body targets the acceptor cell via an extracellular moiety described herein (e.g., comprising a target domain described herein), e.g., comprised in a membrane-associated agent described herein.

In embodiments, the donor cell or membrane-enclosed body may transfer a membrane-associated agent and/or cargo molecule to an acceptor cell from a source of stem cells or progenitor cells, e.g., bone marrow stromal cells, marrow-derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts.

### Specific Delivery to Target Cells

In some embodiments, a donor cell, membrane-enclosed body, or composition described herein delivers a cargo molecule (e.g., operably associated with or linked to a membrane-associated agent) preferentially to a target acceptor cell compared to a non-target cell. Accordingly, in certain embodiments, a donor cell or membrane-enclosed body described herein has one or both of the following properties: (i) when the plurality of donor cells or membrane-enclosed bodies are contacted with a cell population comprising target acceptor cells and non-target cells, under conditions suitable for transfer of the cargo molecule from a donor cell or membrane-enclosed body to an acceptor cell, the cargo molecule is present in at least 2-fold, 5-fold, 10-fold, 20-fold, 50-fold, or 100-fold more in target acceptor cells than in non-target cells, or (ii) the donor cells or membrane-enclosed bodies of the plurality transfer the cargo molecule at a higher rate to a target acceptor cell than with a non-target cell by at least at least 50%.

In some embodiments, the donor cells or membrane-enclosed bodies transfer the cargo molecules to target cells at a rate such that the cargo molecule is delivered to at least 10%, 20%, 30%, 40%, 50%, 60%, 70%, 80%, or 90%, of target cells after 24, 48, or 72 hours. In embodiments, the amount of targeted transfer is about 30%-70%, 35%-65%, 40%-60%, 45%-55%, or 45%-50%. In embodiments, the amount of transfer is about 20%-40%, 25%-35%, or 30%-35.

In some embodiments, the donor cells, membrane-enclosed body, or composition delivers at least 40%, 45%, 50%, 55%, 60%, 65%, 70%, 75%, 80%, 85%, 90%, 95%, 96%, 97%, 98%, or 99% of the cargo molecule to the target acceptor cell population compared to a reference cell population or to a non-target cell population. In some embodiments, the donor cells, membrane-enclosed bodies, or composition transfer at least 40%, 45%, 50%, 55%, 60%, 65%, 70%, 75%, 80%, 85%, 90%, 95%, 96%, 97%, 98%, or 99% more of the cargo molecule to the target acceptor cell population compared to the reference cell population or to the non-target cell population.

### Methods of Treatment

The donor cells, membrane-enclosed bodies, acceptor cells, cargo molecules, exogenous membrane-associated agents, and/or compositions described herein may be used to modulate one or more biological function in a subject, e.g., a patient, e.g., a human patient.

In some embodiments, the biological function is selected from:

modulating, e.g., increasing or decreasing, an interaction between two cells;

modulating, e.g. increasing or decreasing, an immune response;

modulating, e.g. increasing or decreasing, recruitment of cells to a target tissue;

decreasing the growth rate of a cancer; or

reducing the number of cancerous cells in the subject.

In some aspects, provided herein is a method of administering a donor cell or membrane-enclosed body to a subject, e.g., a human subject, comprising administering to the subject a donor cell or membrane-enclosed body, or a composition comprising a plurality of donor cells or membrane-enclosed bodies, a donor cell composition, a membrane-enclosed body composition, or a pharmaceutical composition as described herein, thereby administering the donor cell or membrane-enclosed body to the subject.

In some aspects, provided herein a method of delivering a cargo molecule (e.g., operably associated with or linked to a membrane-associated agent as described herein) to a subject, comprising administering to the subject a donor cell, membrane-enclosed body, or a composition comprising a plurality of donor cells or membrane-enclosed bodies, a donor cell or membrane-enclosed body composition, or a pharmaceutical composition as described herein, wherein the donor cells or membrane-enclosed bodies are administered in an amount and/or time such that the cargo molecule is delivered

In some aspects, provided herein is a method of modulating, e.g., enhancing, a biological function in a subject, comprising administering to the subject a donor cell, membrane-enclosed body, or a composition comprising a plurality of donor cells or membrane-enclosed bodies, a donor cell or membrane-enclosed body composition, or a pharmaceutical composition as described herein, thereby modulating the biological function in the subject.

In some aspects, provided herein is a method of delivering or targeting a function to a subject, comprising administering to the subject a donor cell, membrane-enclosed body, or a composition comprising a plurality of donor cells or membrane-enclosed bodies, a donor cell or membrane-enclosed body composition, or a pharmaceutical composition as described herein, wherein the donor cells or membrane-enclosed bodies are administered in an amount and/or time such that the function in the subject is delivered or targeted.

In some aspects, provided herein is a method of treating a disease or disorder in a subject or patient comprising administering to the subject a donor cell, membrane-enclosed body, or a composition comprising a plurality of donor cells or membrane-enclosed bodies, a donor cell or membrane-enclosed body composition, or a pharmaceutical composition as described herein, wherein the donor cells or membrane-enclosed bodies are administered in an amount and/or time such that the disease or disorder is treated.

In some embodiments, the subject has a cancer, an inflammatory disorder, autoimmune disease, a chronic disease, inflammation, damaged organ function, an infectious disease, metabolic disease, degenerative disorder, genetic disease (e.g., a genetic deficiency or a dominant genetic disorder), or an injury. In some embodiments, the subject has an infectious disease and the cargo molecule comprises an antigen for the infectious disease. In some embodiments, the subject has a genetic deficiency and the cargo molecule comprises a protein for which the subject is deficient, or a nucleic acid (e.g., a DNA, a gDNA, a cDNA, an RNA, a pre-mRNA, an mRNA, etc.) encoding the protein, or a DNA encoding the protein, or a chromosome encoding the protein, or a nucleus comprising a nucleic acid encoding the protein. In some embodiments, the subject has a dominant genetic disorder, and the cargo molecule comprises or is associated with a nucleic acid inhibitor (e.g., siRNA or miRNA) of the dominant mutant allele. In some embodiments, the subject has a dominant genetic disorder, and the cargo molecule comprises or is associated with a nucleic acid inhibitor (e.g., siRNA or miRNA) of the dominant mutant allele, and the cargo molecule comprises or is associated with an mRNA encoding a non-mutated allele of the mutated gene that is not targeted by the nucleic acid inhibitor. In some embodiments, the subject is in need of vaccination. In some embodiments, the subject is in need of regeneration, e.g., of an injured site.

In some embodiments, the donor cell, membrane-enclosed body, composition, or preparation is administered to the subject at least 1, 2, 3, 4, or 5 times.

In some embodiments, the donor cell, membrane-enclosed body, composition, or preparation is administered to the subject systemically (e.g., orally, parenterally, subcutaneously, intravenously, intramuscularly, intraperitoneally) or locally. In some embodiments, the donor cell or membrane-enclosed body composition or preparation is administered to the subject such that the donor cell or membrane-enclosed body composition or preparation reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments (e.g., wherein the subject has an autoimmune disease), the donor cell or membrane-enclosed body composition or preparation is co-administered with an immunosuppressive agent, e.g., a glucocorticoid, cytostatic, antibody, or immunophilin modulator. In some embodiments (e.g., wherein the subject has a cancer or an infectious disease), the donor cell or membrane-enclosed body composition or preparation is co-administered with an immunostimulatory agent, e.g., an adjuvant, interleukin, cytokine, or chemokine. In some embodiments, administration of the donor cell or membrane-enclosed body composition or preparation results in upregulation or downregulation of a gene in a target cell in the subject, e.g., wherein the donor cell or membrane-enclosed body comprises a transcriptional activator or repressor, a translational activator or repressor, or an epigenetic activator or repressor.

In some embodiments, when the plurality of donor cells or membrane-enclosed bodies are contacted with a cell population comprising target acceptor cells and non-target cells, the cargo molecules are present in substantial amounts in at least 2-fold, 5-fold, 10-fold, 20-fold, 50-fold, or 100-fold more target acceptor cells than non-target cells. In some embodiments, the donor cells of the plurality transfer cargo molecules at a higher rate with a target acceptor cell than with a non-target cell by at least at least 50%.

In some embodiments, the disease or disorder is selected from cancer, autoimmune disorder, or infectious disease. In some embodiments, the subject has a cancer. In some embodiments, cargo molecule comprises a neoantigen. In some embodiments, the donor cell or membrane-enclosed body or composition thereof is administered to the subject at least 1, 2, 3, 4, or 5 times. In some embodiments, the donor cell or membrane-enclosed body or composition thereof is administered to the subject systemically (e.g., orally, parenterally, subcutaneously, intravenously, intramuscularly, intraperitoneally) or locally. In some embodiments, the donor cell or membrane-enclosed body or composition thereof is administered to the subject such that the donor cell or membrane-enclosed body or composition thereof reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments, the donor cell or membrane-enclosed body or composition thereof is co-administered with an immunosuppressive agent, e.g., a glucocorticoid, cytostatic, antibody, or immunophilin modulator. In some embodiments, the donor cell or membrane-enclosed body or composition thereof is co-administered with an immunostimulatory agent, e.g., an adjuvant, interleukin, cytokine, or chemokine.

The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a tumor may be a disperse tumor or a liquid tumor. In some embodiments, a relevant cancer may be characterized by a hematologic tumor. In general, examples of different types of cancers known in the art include, for example, leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

In some embodiments, the plurality of donor cells or membrane-enclosed bodies has a local, distal, or systemic effect (e.g., via the transfer of cargo molecules to acceptor cells).

In some embodiments, any of the methods disclosed herein, further comprises a step of monitoring one or more of cancer progression, tumor recession, tumor volume, decrease in neoplastic cell number, quantity of fused cells, quantity of fused cells comprising a membrane protein payload agent, quantity of fused cells expressing a nucleic acid protein payload, and quantity of membrane protein disposed in membrane of a fused cell.

In some embodiments, any of the methods disclosed herein, further comprises a step of monitoring adverse events in the organism. In some embodiments, the adverse event includes one or more of cytokine release syndrome, fever, tachycardia, chills, anorexia, nausea, vomiting, myalgia, headaches, capillary leak syndrome, hypotension, pulmonary edema, coagulopathy, renal dysfunction, kidney injury, macrophage-activation syndrome, hemophagocytic lymphohistiocytosis, organ failure, cerebral edema, bystander inflammation from T cell activation, neurologic symptoms, encephalopathy, confusion, hallucination, delirium, obtundation, aphasia, seizures, B-cell aplasia, tumor lysis syndrome, and graft versus host disease.

In some embodiments, the organism is a human. In some embodiments, the human has a disease, disorder, or condition. In some embodiments, presence of the membrane protein payload agent in the cell membrane lipid bilayer of the target cell improves one or more symptoms of the disease, disorder, or condition.

Additional Therapeutic Agents

In some embodiments, the donor cell or membrane-enclosed body or composition thereof is co-administered with an additional agent, e.g., a therapeutic agent, to a subject, e.g., a recipient, e.g., a recipient described herein. In some embodiments, the co-administered therapeutic agent is an immunosuppressive agent, e.g., a glucocorticoid (e.g., dexamethasone), cytostatic (e.g., methotrexate), antibody (e.g., Muromonab-CD3), or immunophilin modulator (e.g., Ciclosporin or rapamycin). In embodiments, the immunosuppressive agent decreases immune mediated clearance of donor cells, or membrane-enclosed bodies, exogenous membrane-associated agents, and/or cargo molecules. In some embodiments the donor cell or membrane-enclosed body or composition thereof is co-administered with an immunostimulatory agent, e.g., an adjuvant, an interleukin, a cytokine, or a chemokine.

In some embodiments, the donor cell or membrane-enclosed body or composition thereof and the immunosuppressive agent are administered at the same time, e.g., contemporaneously administered. In some embodiments, the donor cell or membrane-enclosed body or composition thereof is administered before administration of the immunosuppressive agent. In some embodiments, the donor cell or membrane-enclosed body or composition thereof is administered after administration of the immunosuppressive agent.

In some embodiments, the immunosuppressive agent is a small molecule such as ibuprofen, acetaminophen, cyclosporine, tacrolimus, rapamycin, mycophenolate, cyclophosphamide, glucocorticoids, sirolimus, azathioprine, or methotrexate.

In some embodiments, the immunosuppressive agent is an antibody molecule, including but not limited to: muromonab (anti-CD3), Daclizumab (anti-IL12), Basiliximab, Infliximab (Anti-TNFα), or rituximab (Anti-CD20).

In some embodiments, co-administration of the donor cell or membrane-enclosed body or composition thereof with the immunosuppressive agent results in enhanced persistence of the donor cell or membrane-enclosed body or composition thereof, exogenous membrane-associated agent, and/or cargo molecule in the subject compared to administration of the donor cell or membrane-enclosed body or composition thereof alone. In some embodiments, the enhanced persistence of the donor cell or membrane-enclosed body or composition thereof, exogenous membrane-associated agent, and/or cargo molecule in the co-administration is at least 10%, 20%, 30%, 40%, 50%, 60%, 70%, 80%, 90% or longer, compared to persistence of the donor cell or membrane-enclosed body or composition thereof, exogenous membrane-associated agent, and/or cargo molecule when administered alone. In some embodiments, the enhanced persistence of the donor cell or membrane-enclosed body or composition thereof, exogenous membrane-associated agent, and/or cargo molecule in the co-administration is at least 1, 2, 3, 4, 5, 6, 7, 10, 15, 20, 25, or 30 days or longer, compared to survival of the donor cell or membrane-enclosed body or composition thereof, exogenous membrane-associated agent, and/or cargo molecule when administered alone.

In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys or apes), ungulate (e.g., cattle, buffalo, sheep, goat, pig, camel, llama, alpaca, deer, horses, or donkeys), carnivore (e.g., dogs or cats), rodent (e.g., rats or mice), or lagomorph (e.g., rabbits). In some embodiments, the subject is a bird, such as a member of the avian taxa Galliformes (e.g., chickens, turkeys, pheasants, or quail), Anseriformes (e.g., ducks or geese), Paleaognathae (e.g., ostriches or emus), Columbiformes (e.g., pigeons or doves), or Psittaciformes (e.g., parrots). In some embodiments, the subject is an invertebrate such as an arthropod (e.g, insects, arachnids, or crustaceans), a nematode, an annelid, a helminth, or a mollusc. In some embodiments, the subject is an invertebrate agricultural pest or an invertebrate that is parasitic on an invertebrate or vertebrate host. In some embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In some embodiments, the subject is a eukaryotic alga (unicellular or multicellular). In some embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

Methods of Manufacturing

The disclosure provides, in some aspects, a method of manufacturing a donor cell, membrane-enclosed body, or a composition, comprising:
a) providing a source cell comprising, e.g., expressing, a membrane-associated agent;
b) producing a donor cell from the source cell, wherein the donor cell comprises a lipid bilayer, a lumen, a membrane-associated agent, and a cargo molecule (e.g., operably associated with or linked to the membrane-associated agent), thereby making a donor cell; and
c) formulating the donor cell, e.g., as a pharmaceutical composition suitable for administration to a subject.

In some aspects, the present disclosure provides a method of manufacturing a donor cell composition, comprising:
a) providing a plurality of donor cells described herein or a donor cell composition described herein; and
b) formulating the donor cells, e.g., as a pharmaceutical composition suitable for administration to a subject.

In some aspects, the present disclosure provides a method of manufacturing a donor cell composition, comprising:
a) providing, e.g., producing, a plurality of donor cells or a donor cell preparation described herein; and
b) assaying a sample of the plurality (e.g., of the preparation) to determine whether one or more (e.g., 2, 3, or more) standards are met. In embodiments, the standard(s) are chosen from:

donor cells in the sample transfer a membrane-associated agent at a higher rate with a target acceptor cell than with a non-target cell, e.g., by at least at least 1%, 2%, 3%, 4%, 5%, 10%, 20%, 30%, 40%, 50%, 60%, 70%, 80%, 90%;

donor cells in the sample transfer a membrane-associated agent at a higher rate with a target acceptor cell than other cells, e.g., by at least 10%, 20%, 30%, 40%, 50%, 60%, 70%, 80%, or 90%;

donor cells in the sample transfer a membrane-associated agent to target acceptor cells at a rate such that a cargo molecule in the donor cell (e.g., operably associated with or linked to the membrane-associated agent) is delivered to at least 10%, 20%, 30%, 40%, 50%, 60%, 70%, 80%, or 90%, of target acceptor cells after 24, 48, or 72 hours;

the membrane-associated agent is present at a copy number, per donor cell (e.g., on average in the sample), of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies;

the cargo molecule is present at a copy number, per donor cell (e.g., on average in the sample), of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies;

the cargo molecule is detectable in donor cells and/or acceptor cells of the sample (e.g., on average in the sample) at a copy number of at least, or no more than, 10, 50, 100, 500, 1,000, 2,000, 5,000, 10,000, 20,000, 50,000, 100,000, 200,000, 500,000 1,000,000, 5,000,000, 10,000,000, 50,000,000, 100,000,000, 500,000,000, or 1,000,000,000 copies;

the ratio of the copy number of the membrane-associated agent to the copy number of the cargo molecule is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000;

donor cells of the sample are characterized by a lipid composition substantially similar to that of the source cell or wherein one or more of CL, Cer, DAG, HexCer, LPA, LPC, LPE, LPG, LPI, LPS, PA, PC, PE, PG, PI, PS, CE, SM and TAG is within 10%, 15%, 20%, 25%, 30%, 35%, 40%, 45%, 50%, or 75% of the corresponding lipid level in the source cell;

donor cells of the sample are characterized by a proteomic composition similar to that of the source cell;

donor cells of the sample are characterized by a ratio of lipids to proteins that is within 10%, 20%, 30%, 40%, or 50% of the corresponding ratio in the source cell;

donor cells of the sample are characterized by a ratio of proteins to nucleic acids (e.g., DNA) that is within 10%, 20%, 30%, 40%, or 50% of the corresponding ratio in the source cell;

donor cells of the sample are characterized by a ratio of lipids to nucleic acids (e.g., DNA) that is within 10%, 20%, 30%, 40%, or 50% of the corresponding ratio in the source cell;

donor cells of the sample are characterized by a half-life in a subject, e.g., in an experimental animal such as a mouse, that is within 1%, 2%, 3%, 4%, 5%, 10%, 20%, 30%, 40%, 50%, 60%, 70%, 80%, 90%, 100% of the half-life of a reference cell;

donor cells of the sample are characterized by a metabolic activity level that is within 1%, 2%, 3%, 4%, 5%, 10%, 20%, 30%, 40%, 50%, 60%, 70%, 80%, 90%, or 100% of the metabolic activity in a reference cell, e.g., the source cell;

donor cells of the sample are characterized by a miRNA content level of at least at least 1%, 2%, 3%, 4%, 5%, 10%, 20%, 30%, 40%, 50%, 60%, 70%, 80%, 90%, or greater than that of the source cell;

the donor cell has a soluble:non-soluble protein ratio is within 1%, 2%, 3%, 4%, 5%, 10%, 20%, 30%, 40%, 50%, 60%, 70%, 80%, 90%, or greater than that of the source cell, e.g., within 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of that of the source cell;

donor cells of the sample are characterized by an LPS level less than 5%, 1%, 0.5%, 0.01%, 0.005%, 0.0001%, 0.00001% or less of the LPS content of the source cell or a reference cell;

donor cells of the sample are capable of signal transduction, e.g., transmitting an extracellular signal, e.g., by at least 1%, 2%, 3%, 4%, 5%, 10%, 20%, 30%, 40%, 50%, 60%, 70%, 80%, 90%, 100% more than a negative control;

donor cells of the sample are capable of secreting a protein, e.g., at a rate at least 1%, 2%, 3%, 4%, 5%, 10%, 20%, 30%, 40%, 50%, 60%, 70%, 80%, 90%, or 100% greater than the reference cell; or

donor cells of the sample are characterized by low immunogenicity, e.g., as described herein; and

c) (optionally) approving the plurality of donor cells or the donor cell composition for release if one or more of the standards is met or (optionally) formulating the plurality of donor cells or the donor cell composition as a drug product if the one or more standards is met.

The present disclosure also provides, in some aspects, a method of manufacturing a donor cell composition, comprising:
a) providing, e.g., producing, a plurality of donor cells described herein or a donor cell composition or preparation described herein; and
b) assaying a sample of the plurality or preparation to determine the presence or level of one or more of the following factors:

an immunogenic molecule, e.g., an immunogenic protein, e.g., as described herein;

a pathogen, e.g., a bacterium or virus; or

a contaminant (e.g., a nuclear structure or component such as nuclear DNA); and
i) c) (optionally) approving the plurality of donor cells or donor cell composition for release if one or more of the factors is deviates significantly (e.g., by more than a specified amount) from a reference value or (optionally) formulating the plurality of donor cells or the donor cell composition as a drug product if the one or more factors does not significantly deviate (e.g., does not deviate by more than the specified about) from the reference value.

In some aspects, provided herein is a method of manufacturing a donor cell composition, comprising:
i) providing a plurality of donor cell, a donor cell composition, or a pharmaceutical composition as described herein; and
b) assaying one or more donor cells from the plurality to determine whether one or more (e.g., 2, 3, or all) of the following standards are met:

the donor cell transfers membrane-associated agents and/or cargo molecules (e.g., operably associated with or linked to the membrane-associated agents) at a higher rate with a target acceptor cell than with a non-target cell, e.g., by at least at least 10%;

the donor cell transfers membrane-associated agents and/or cargo molecules (e.g., operably associated with or linked to the membrane-associated agents) at a higher rate with a target acceptor cell than with other cells, e.g., by at least 50%;

the donor cell transfers membrane-associated agents and/or cargo molecules (e.g., operably associated with or linked to the membrane-associated agents) to target acceptor cells at a rate such that an agent in the donor cell is delivered to at least 10% of target acceptor cells after 24 hours;

the membrane-associated agent is present in an acceptor cell at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);

the cargo molecule is present in an acceptor cell at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);

the donor cell comprises a membrane-associated agent at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);

the donor cell comprises a cargo molecule at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);

the ratio of the copy number of the membrane-associated agent to the copy number of the cargo molecule is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000;

the donor cell comprises a ratio of lipids to proteins that is within 10%, 20%, 30%, 40%, or 50% of the corresponding ratio in the source cell;

the donor cell comprises a ratio of proteins to nucleic acids (e.g., DNA) that is within 10%, 20%, 30%, 40%, or 50% of the corresponding ratio in the source cell;

the donor cell comprises a ratio of lipids to nucleic acids (e.g., DNA) that is within 10%, 20%, 30%, 40%, or 50% of the corresponding ratio in the source cell;

the donor cell comprises a metabolic activity level that is within 90% of the metabolic activity in a reference cell, e.g., the source cell;

the donor cell has a miRNA content level of at least 1% than that of the source cell;

the donor cell has a soluble:non-soluble protein ratio is within 90% of that of the source cell;

the donor cell has an LPS level less than 5% of the lipid content of donor cells;

the donor cell and/or compositions or preparations thereof, are capable of signal transduction, e.g., an otherwise similar donor cell in the absence of insulin;

the donor cell and/or compositions or preparations thereof, are capable of secreting a protein, e.g., at a rate at least 5% greater than a reference cell or

the donor cell has low immunogenicity, e.g., as described herein; and
i) c) (optionally) approving the plurality of donor cells or donor cell composition for release if one or more of the standards is met;

thereby manufacturing a donor cell drug product composition

In some aspects, provided herein is a method of manufacturing a donor cell composition, comprising:
a) providing a plurality of donor cells, a donor cell composition, or a pharmaceutical composition as described herein; and
b) assaying one or more donor cell from the plurality to determine the presence or level of one or more of the following factors:

an immunogenic molecule, e.g., an immunogenic protein, e.g., as described herein;

a pathogen, e.g., a bacterium or virus; or

a contaminant;
i) c) (optionally) approving the plurality of donor cells or donor cell composition for release if one or more of the factors is below a reference value;

thereby manufacturing a donor cell drug product composition.

In some embodiments of the methods of making herein, providing a source cell expressing a membrane-associated agent comprises expressing a membrane-associated agent in the source cell or upregulating expression of an endogenous membrane-associated agent in the source cell. In some embodiments, the method comprises inactivating the nucleus of the source cell.

In some embodiments, at least one donor cell of the plurality of donor cells is derived from a source cell.

In embodiments, the donor cell is from a mammalian cell having a modified genome, e.g., to reduce immunogenicity (e.g., by genome editing, e.g., to remove an MHC protein). In embodiments, the method further comprises contacting the source cell of step a) with an immunosuppressive agent, e.g., before or after inactivating the nucleus, e.g., enucleating the cell.

In some embodiments, the donor cell does not comprise Cre or GFP, e.g., EGFP.

In some embodiments of any of the compositions described herein, the composition (e.g., donor cell composition) comprises a donor cell, acceptor cell, and/or membrane-enclosed body, e.g., as described herein.

Source Cells

In some embodiments, a donor cell, acceptor cell, or membrane-enclosed body is derived from a source cell.

In some embodiments, the source cell or target cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial glial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell. In some embodiments, the source cell or target cell is a white blood cell or a stem cell. In some embodiments, the source cell or target cell is selected from a neutrophil, a lymphocyte (e.g., a T cell, a B cell, a natural killer cell), a macrophage, a granulocyte, a mesenchymal stem cell, a bone marrow stem cell, an induced pluripotent stem cell, an embryonic stem cell, or a myeloblast.

In some embodiments, the source cell is a cell grown under adherent or suspension conditions. In some embodiments, the source cell is a primary cell, a cultured cell, an immortalized cell, or a cell line. In some embodiments, the source cell is allogeneic, e.g., obtained from a different organism of the same species as the target cell. In some embodiments, the source cell is autologous, e.g., obtained from the same organism as the target cell. In some embodiments, the source cell is heterologous, e.g., obtained from an organism of a different species from the target cell.

In some embodiments, the source cell comprises or further comprises a second agent that is exogenous to the source cell, e.g., a therapeutic agent, e.g., a protein or a nucleic acid (e.g., an RNA, e.g., an mRNA or miRNA). In some embodiments, the second agent is present at least, or no more than, 10, 20, 50, 100, 200, 500, 1,000, 2,000, 5,000, 10,000, 20,000, 50,000, 100,000, 200,000, 500,000 or 1,000,000 copies comprised by the cell (e.g., donor cell or acceptor cell) or membrane-enclosed body, or is present at an average level of at least, or no more than, 10, 20, 50, 100, 200, 500, 1,000, 2,000, 5,000, 10,000, 20,000, 50,000, 100,000, 200,000, 500,000 or 1,000,000 copies per cell (e.g., donor cell or acceptor cell) or membrane-enclosed body.

In some embodiments, the cell (e.g., donor cell or acceptor cell) or membrane-enclosed body has an altered, e.g., increased or decreased level of one or more endogenous molecules as compared to the source cell, e.g., protein or nucleic acid, e.g., due to treatment of the source cell, e.g., mammalian source cell with a siRNA or gene editing enzyme. In some embodiments, the cell (e.g., donor cell or acceptor cell) or membrane-enclosed body comprises at least, or no more than, 10, 20, 50, 100, 200, 500, 1,000, 2,000, 5,000, 10,000, 20,000, 50,000, 100,000, 200,000, 500,000 or 1,000,000 copies of the endogenous molecule, or is present at an average level of at least, or no more than, 10, 20, 50, 100, 200, 500, 1,000, 2,000, 5,000, 10,000, 20,000, 50,000, 100,000, 200,000, 500,000 or 1,000,000 copies of the endogenous molecule per cell (e.g., donor cell or acceptor cell) or membrane-enclosed body. In some embodiments, the endogenous molecule (e.g., an RNA or protein) is present in the cell (e.g., donor cell or acceptor cell) or membrane-enclosed body at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸ greater than its concentration in the source cell. In some embodiments, the endogenous molecule (e.g., an RNA or protein) is present in the cell (e.g., donor cell or acceptor cell) or membrane-enclosed body at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸ less than its concentration in the source cell.

In some embodiments, provided donor cells, acceptor cells, membrane-enclosed bodies, and/or compositions or preparations thereof, comprise less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells have a functional nucleus. In some embodiments, at least 10%, 20%, 30%, 40%, 50%, 60%, 70%, 80%, 90%, 95%, or 99% of donor cells, acceptor cells, membrane-enclosed bodies in a composition or preparation described herein comprise an organelle, e.g., a mitochondrion.

In some embodiments, provided donor cells, acceptor cells, membrane-enclosed bodies, and/or compositions or preparations thereof, comprise at least 0.01%-0.05%, 0.05%-0.1%, 0.1%-0.5%, 0.5%-1%, 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% donor cells, acceptor cells, membrane-enclosed bodies wherein: i) the membrane-associated agent is present at a copy number of at least 1,000 copies per donor cell, acceptor cell, or membrane-enclosed body, ii) the ratio of the copy number of the membrane-associated agent to the copy number of the cargo molecule per donor cell, acceptor cell, membrane-enclosed body is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000, or iii) the membrane protein payload agent is present at a copy number of at least 1,000 copies per donor cell, acceptor cell, membrane-enclosed body.

In embodiments, the source cell is a primary cell, immortalized cell or a cell line. In embodiments, the donor cell, acceptor cell, membrane-enclosed body is from a source cell having a modified genome, e.g., having reduced immunogenicity (e.g., by genome editing, e.g., to remove an WIC protein, e.g., WIC complex). In embodiments, the source cell is from a cell culture treated with an immunosuppressive agent. In embodiments, the source cell is substantially non-immunogenic, e.g., using an assay described herein. In embodiments, the source cell comprises an exogenous agent, e.g., a therapeutic agent. In embodiments, the source cell is a recombinant cell.

In some embodiments, the source cell is from a cell culture treated with an anti-inflammatory signal. In some embodiments, a method of making described herein further comprises contacting the source cell with an anti-inflammatory signal, e.g., before or after inactivating the nucleus, e.g., enucleating the cell.

### ENUMERATED EMBODIMENTS

1. A donor cell comprising

a membrane-associated agent, the agent comprising:
a membrane-associated moiety, and
one or both of an extracellular moiety or an intracellular moiety,

wherein the membrane-associated agent is configured to be transferred to an acceptor cell;

optionally wherein the donor cell comprises a cargo molecule configured to be transferred to an acceptor cell; and

wherein at least one of the membrane-associated moiety, extracellular moiety, intracellular moiety, or the cargo molecule is exogenous to the donor cell.

2. A donor cell comprising

a membrane-associated agent, the agent comprising:
a membrane-associated moiety, and one or both of an extracellular moiety or an intracellular moiety, optionally wherein the donor cell comprises a cargo molecule; and

wherein at least one of the membrane-associated moiety, extracellular moiety, intracellular moiety, or the cargo molecule is exogenous to the donor cell.

wherein the membrane-associated agent and cargo molecule, if present, are transferred to an acceptor cell.

3. A donor cell comprising:

a membrane-associated agent, the agent comprising:
a membrane-associated moiety, and
one or both of an extracellular moiety or an intracellular moiety

wherein the membrane-associated agent is configured to be transferred to an acceptor cell;

optionally wherein the donor cell comprises a cargo molecule configured to be transferred to the acceptor cell; and

wherein at least one of the membrane-associated moiety, extracellular moiety, intracellular moiety, or cargo molecule is present at a different level in the donor cell than a source cell from which the donor cell is derived, e.g., is differentially expressed.

4. A donor cell comprising:

a membrane-associated agent, the agent comprising:
a membrane-associated moiety, and
one or both of an extracellular moiety or an intracellular moiety optionally wherein the donor cell comprises a cargo molecule;

wherein at least one of the membrane-associated moiety, extracellular moiety, intracellular moiety, or cargo molecule is present at a different level in the donor cell than a source cell from which the donor cell is derived, e.g., is differentially expressed, and

wherein the membrane-associated agent and cargo molecule, if present, are transferred to an acceptor cell.

5. An acceptor cell comprising:

a membrane-associated agent, the agent comprising:
a membrane-associated moiety, and
one or both of an extracellular moiety or an intracellular moiety,

wherein the acceptor cell does not comprise a nucleic acid encoding the membrane-associated agent (e.g., wherein the acceptor cells is not genetically modified to express the membrane-associated agent),

optionally wherein the acceptor cell comprises a cargo molecule, e.g., received from a donor cell,

wherein at least one of the membrane-associated moiety, extracellular moiety, intracellular moiety, or cargo molecule is exogenous to the acceptor cell, and

optionally wherein the acceptor cell comprises, e.g., received, the membrane-associated agent from a donor cell.

6. An acceptor cell comprising:

a membrane-associated agent, the agent comprising:
a membrane-associated moiety,
and one or both of an extracellular moiety or an intracellular moiety,

wherein the acceptor cell does not substantially express, e.g., does not express, a nucleic acid encoding the membrane-associated agent,

optionally wherein the acceptor cell comprises a cargo molecule, e.g., received from a donor cell, wherein the acceptor cell does not substantially express (e.g., does not express) a nucleic acid encoding the cargo molecule, and

optionally wherein the acceptor cell received the membrane-associated agent from the donor cell.

7. The donor cell or acceptor cell of any preceding embodiment, wherein the membrane-associated moiety, the extracellular moiety, the intracellular moiety, the cargo molecule, or a combination thereof is exogenous to the donor cell.
8. The donor cell or acceptor cell of any preceding embodiment, wherein the membrane-associated moiety, the extracellular moiety, the intracellular moiety, the cargo molecule, or a combination thereof is differentially expressed by the donor cell, e.g., expressed at a different level (e.g., an increased level) in the donor cell than the membrane-associated moiety, the extracellular moiety, the intracellular moiety, the cargo molecule, or a combination thereof are endogenously expressed in the donor cell or source cell from which the donor cell was derived.
9. The donor cell or acceptor cell of any preceding embodiment, wherein the membrane-associated moiety, the extracellular moiety, the intracellular moiety, the cargo molecule, or a combination thereof is exogenous to the acceptor cell.
10. The donor cell or acceptor cell of any preceding embodiment, wherein the membrane-associated moiety, the extracellular moiety, the intracellular moiety, the cargo molecule, or a combination thereof is differentially expressed by the acceptor cell, e.g., expressed at a different level (e.g., an increased level) in the acceptor cell than the membrane-associated moiety, the extracellular moiety, the intracellular moiety, the cargo molecule, or a combination thereof are endogenously expressed in the acceptor cell or source cell from which the acceptor cell was derived.
11. The donor cell or acceptor cell of any preceding embodiment, wherein the membrane-associated moiety is a transmembrane moiety.
12. The donor cell or acceptor cell of any preceding embodiment, wherein the donor cell and/or acceptor cell is purified, e.g., isolated, from its natural state, e.g., wherein the donor cell or acceptor cell is an in vitro or ex vivo cell.
13. The donor cell of any preceding embodiment, wherein the membrane-associated agent is transferred (e.g., delivered) from the donor cell to the acceptor cell via a membrane transfer process, e.g., chosen from one or more of: a membrane fusion event, a receptor-ligand interaction, a cell bridging event (e.g., an antibody molecule (e.g., a bispecific antibody), or cell to cell contact event.
14. The acceptor cell of any preceding embodiment, which comprises a level of the membrane-associated agent or cargo molecule from the donor cell of at least 1, 2, 3, 4, 5, 6, 7, 8, 9, 10, 20, 50, 100, or 500 nM or μM, or at least 1, 2, 3, 4, or 5 mM, or at least 10, 50, 100, 200, 300, 400, 500, or 1000 membrane-associated agents per μm² of acceptor cell membrane.
15. The acceptor cell of any preceding embodiment, which comprises a level of membrane-associated agent or cargo molecule that is at least 0.01, 0.1, 1, 2, 3, 4, 5, 6, 7, 8, 9, 10, 20, 30, 40, 50, 60, 70, 80, 90, 95, or 99% of the level of membrane-associated agent or cargo molecule present in the donor cell.
16. The acceptor cell of any preceding embodiment, wherein at least about 0.01%, 0.1%, 1%, 5%, 10%, 20% of the lipid content in the acceptor cell is exogenous to the acceptor cell, e.g., is derived from the donor cell.
17. The acceptor cell of any preceding embodiment, wherein at least one biological function of the acceptor cell, or the plurality of acceptor cells is modulated (e.g., increased or decreased) by the transfer of the membrane-associated agent or cargo molecule, e.g., wherein the biological function is chosen from:
(i) modulating, e.g., increasing or decreasing a level or activity of a molecule (e.g., a protein, nucleic acid, or metabolite, drug, or toxin) in the acceptor cell or the plurality of acceptor cells;
(ii) modulating, e.g., increasing or decreasing, enzyme activity in the acceptor cell or the plurality of acceptor cells;
(iii) modulating, e.g., increasing or decreasing, a genetic or an epigenetic event in the acceptor cell or the plurality of acceptor cells;
(iv) modulating, e.g., promoting or inhibiting, acceptor cell differentiation; (v) modulating acceptor cell reprogramming;
(vi) activating a signaling pathway in an acceptor cell;
(vii) delivering a cargo, e.g., a gene editing complex; or
(viii) modifying cell adhesion and trafficking.
   18. The donor cell or acceptor cell of any preceding embodiment, wherein the donor cell, the acceptor cell, or both are not immune cells.
   19. The donor cell or acceptor cell of any of embodiments 1-17, wherein the donor cell, acceptor cell, or both are immune cells (e.g., immune cells (e.g., immune effector cells), e.g., neutrophils, lymphocytes (e.g., T cells, B cells, or NK cells), PMNs (e.g., granulocytes), monocytes, dendritic cells, or macrophages, granulocytes, mesenchymal stem cells, bone marrow stem cells, induced pluripotent stem cells, embryonic stem cells, or myeloblasts).
   20. The donor cell or acceptor cell of any preceding embodiment, wherein the donor cell is not a naturally antigen-presenting cell (e.g., a cell comprising an MEW and antigen peptide on its cell membrane).
   21. The donor cell or acceptor cell of any preceding embodiment, wherein the donor cell is not a TCR-bearing cell (e.g., a cell comprising a TCR on its cell membrane).
   22. The donor cell or acceptor cell of any preceding embodiment, wherein the acceptor cell is not a naturally occurring an antigen-presenting cell (e.g., a cell comprising an WIC and antigen peptide on its cell membrane).
   23. The donor cell or acceptor cell of any preceding embodiment, wherein the acceptor cell is not a TCR-bearing cell (e.g., a cell comprising a TCR on its cell membrane).
   24. The donor cell or acceptor cell of any preceding embodiment, wherein the acceptor cell is not an erythroid or platelet cell.
   25. The donor cell or acceptor cell of any preceding embodiment, wherein the membrane-associated agent is not delivered to the acceptor cell via a physical disruption method, e.g., hypotonic loading.
   26. The donor cell or acceptor cell of any preceding embodiment, wherein the membrane-associated agent is not delivered to the acceptor cell via a synthetic lipid carrier, e.g., a liposome.
   27. The donor cell or acceptor cell of any preceding embodiment, wherein the membrane-associated agent is not transferred to the acceptor cell by genetic engineering.
   28. The donor cell or acceptor cell of any preceding embodiment, wherein the membrane-associated agent does not comprise a TCR polypeptide or a domain thereof.
   29. The donor cell or acceptor cell of any preceding embodiment, wherein the membrane-associated agent does not comprise an WIC polypeptide or a domain thereof.
   30. The donor cell or acceptor cell of any preceding embodiment, wherein the membrane-associated moiety is operatively associated or linked (e.g., tethered to) to the intracellular moiety.
   31. The donor cell or acceptor cell of any preceding embodiment, wherein the membrane-associated moiety is operatively associated or linked (e.g., tethered to) to the cargo molecule.
   32. The donor cell or acceptor cell of any preceding embodiment, wherein the membrane-associated moiety is operatively associated or linked to (e.g., tethered to) the extracellular moiety.
   33. The donor cell or acceptor cell of any preceding embodiment, wherein the membrane-associated moiety is operatively associated or linked to (e.g., tethered to) the extracellular moiety and the intracellular moiety.
   34. The donor cell or acceptor cell of any preceding embodiment, wherein the membrane-associated moiety is operatively associated or linked to (e.g., tethered to) the extracellular moiety and the cargo molecule.
   35. The donor cell or acceptor cell of any preceding embodiment, wherein the membrane-associated moiety is operatively associated or linked to (e.g., tethered to) the intracellular moiety and the cargo molecule.
   36. The donor cell or acceptor cell of any preceding embodiment, wherein the cargo molecule is operatively associated or linked to (e.g., tethered to) the extracellular moiety or the intracellular moiety.
   37. The donor cell or acceptor cell of any preceding embodiment, wherein the membrane-associated moiety is a transmembrane moiety that comprises a transmembrane domain from a receptor.
   38. The donor cell or acceptor cell of any preceding embodiment, wherein the transmembrane moiety comprises a transmembrane domain from a receptor chosen from Notch receptor or thrombomodulin.
   39. The donor cell or acceptor cell of any of embodiments 1-37, wherein the membrane-associated moiety comprises an amino acid sequence that interacts with a membrane component, e.g., a membrane component in the inner leaflet or outer leaflet of the donor cell or acceptor cell (e.g., a protein or fatty acid component of the donor cell or acceptor cell membrane).
   40. The donor cell or acceptor cell of embodiment 39, wherein the amino acid sequence that interacts with the membrane component comprises a lipidation modification sequence (e.g., that is recognized and/or modified by a fatty acid transferase enzyme present in the donor cell), e.g., a myristoylation or palmitoylation (MYR/PA) sequence (e.g., a MYR/PA sequence from an LCK tyrosine kinase).
   41. The donor cell or acceptor cell of either of embodiments 39 or 40, wherein the amino acid sequence that interacts with the membrane component comprises a lipidation modification chosen from one or more of a N-myristoylation, N-palmitoylation, S-palmitoylation, or GPI anchored.
   42. The donor cell or acceptor cell of any of embodiments 39-41, wherein the membrane-associated moiety comprises a myristolyation or palmitoylation (MYR/PA)-binding sequence (e.g., a MYR/PA sequence from an LCK tyrosine kinase).
   43. The donor cell or acceptor cell of any preceding embodiment, wherein the membrane associated moiety comprises a cleavage site recognized by a protease, e.g., TEV protease or RHBDL2.
   44. The donor cell or acceptor cell of any preceding embodiment, wherein the intracellular moiety comprises a cleavage site recognized by a protease, e.g., TEV protease or RHBDL2.
   45. The donor cell or acceptor cell of any preceding embodiment, wherein the protease is not expressed in the donor cell (e.g., is exogenous to the donor cell).
   46. The donor cell or acceptor cell of any preceding embodiment, wherein the protease is present (e.g., expressed) in the acceptor cell, e.g., the protease is exogenous or endogenous to the acceptor cell.
   47. The donor cell or acceptor cell of any preceding embodiment, wherein the extracellular moiety comprises a specificity portion and optionally an accessory portion, e.g., for detection or association with a cargo molecule.
   48. The donor cell or acceptor cell of any preceding embodiment, wherein the extracellular moiety, e.g., the specificity portion, comprises a targeting domain, a transfer promoting moiety, or both.
   49. The donor cell or acceptor cell of any preceding embodiment, wherein the extracellular moiety, e.g., the specificity portion (e.g., the targeting domain), comprises one or more of an antibody or functional fragment thereof (e.g., a Fab, F(ab′)2, Fab′, scFv, or di-scFv), a streptavidin domain (e.g., associated with a biotinylated agent, e.g., a biotinylated antibody), a receptor (e.g., a surface receptor) (e.g., that specifically binds a ligand on the acceptor cell), a ligand (e.g., a ligand that binds a receptor on an acceptor cell), a cell surface protein, a sugar, or a lipid.
   50. The donor cell or acceptor cell of any preceding embodiment, wherein the extracellular moiety, e.g., the specificity portion (e.g., the transfer promoting moiety), comprises a selectin, e.g., E-selectin, P-selectin, L-selectin, or a portion of any thereof (e.g., the extracellular and/or transmembrane domains of the selectin); a claudin, a gap junction protein, an annexin, an integrin, a lectin, a tight junction protein, a desmosomal protein, a member of the immunoglobulin superfamily of molecules (e.g., an antibody or functional fragment thereof), e.g., an HLA-G domain or portion thereof; or a cell adhesion molecule involved in the leukocyte adhesion cascade.
   51. The donor cell or acceptor cell of any preceding embodiment, wherein the extracellular moiety, e.g., the accessory portion, comprises one or more of a tag (e.g., a label (e.g., a fluorescent or radio label) or a cleavage site), a reporter agent, or a marker.
   52. The donor cell or acceptor cell of any preceding embodiment, wherein the extracellular moiety, e.g., the accessory portion, comprises one or more of a Lumio tag or a TEV protease tag.
   53. The donor cell or acceptor cell of any preceding embodiment, wherein the extracellular moiety (and optionally the membrane-associated moiety) comprises a trafficking receptor, e.g., a chemokine receptor, e.g., a CCR protein, a CXCR protein, or a formyl peptide receptor (FPR) protein. In some embodiments, an extracellular moiety comprises an activation or inhibition receptor, e.g., a Notch receptor, interleukin (IL) receptor, or a cluster of differentiation (CD) molecule.
   54. The donor cell or acceptor cell of any preceding embodiment, wherein the extracellular moiety (and optionally the membrane-associated moiety) comprises an activation or inhibition receptor (e.g., a checkpoint receptor, interleukin (IL) receptor, cluster of differentiation (CD) molecule, or Notch receptor).
   55. The donor cell or acceptor cell of any preceding embodiment, wherein the extracellular moiety (and optionally the membrane-associated moiety) comprises a reprogramming receptor.
   56. The donor cell or acceptor cell of any preceding embodiment, wherein the extracellular moiety (and optionally the membrane-associated moiety) comprises a therapeutic protein.
   57. The donor cell or acceptor cell of any preceding embodiment, wherein the extracellular moiety (and optionally the membrane-associated moiety) comprises a synthetic moiety.
   58. The donor cell of any preceding embodiment, wherein the extracellular moiety binds to a target marker, e.g., a receptor or antigen, on the acceptor cell membrane.
   59. The donor cell or acceptor cell of any preceding embodiment, wherein the extracellular moiety is connected to the membrane-associated moiety by a linker, e.g., a flexible linker.
   60. The donor cell or acceptor cell of any preceding embodiment, wherein the specificity portion and the accessory portion are connected to each other by a linker, e.g., flexible linker.
   61. The donor cell or acceptor cell of any preceding embodiment, wherein the intracellular moiety comprises a functional portion and optionally an accessory portion.
   62. The donor cell or acceptor cell of any preceding embodiment, wherein the intracellular moiety, e.g., functional portion, comprises one or more of an antibody or functional fragment thereof (e.g., a Fab, F(ab′)2, Fab′, scFv, or di-scFv), a reporter agent (e.g., a fluorescent tag), a signaling protein, an enzyme (or functional portion thereof), a transcription factor, an epigenetic remodeling agent, a protein binding domain, an RNA-binding protein or domain, a hydrophobic domain, a lipid raft targeting domain, or drug-binding domain.
   63. The donor cell or acceptor cell of any preceding embodiment, wherein the intracellular moiety, e.g., the functional portion, comprises EGFP, β-lactamase, Cre recombinase, a CRISPR/Cas protein (e.g., Cas9) and optionally a guide RNA, or a functional portion or variant of any thereof.
   64. The donor cell or acceptor cell of any preceding embodiment, wherein the intracellular moiety, e.g., accessory portion, comprises one or more of a tag (e.g., a label (e.g., a fluorescent or radio label) or a cleavage site), a reporter agent, a marker, a stability-enhancing domain, a domain that inhibits recycling, or a drug-binding domain for inducibility.
   65. The donor cell or acceptor cell of any preceding embodiment, wherein the intracellular moiety, e.g., accessory portion, comprises a Lumio tag, a TEV protease cleavage site, or a rhomboid protease cleavage site, e.g., RHBDL2.
   66. The donor cell or acceptor cell of any preceding embodiment, wherein the intracellular moiety is connected to the membrane-associated moiety by a linker.
   67. The donor cell or acceptor cell of any preceding embodiment, wherein the functional portion is connected to the accessory portion by a linker, e.g., a linker, e.g., a flexible linker.
   68. The donor cell or acceptor cell of any preceding embodiment, wherein the cargo molecule is operably associated or linked (e.g., tethered) to the membrane-associated agent.
   69. The donor cell or acceptor cell of any preceding embodiment, wherein the cargo molecule is operably associated or linked (e.g., tethered) to the membrane-associated moiety, extracellular moiety, or intracellular moiety.
   70. The donor cell or acceptor cell of any preceding embodiment, wherein the cargo molecule is covalently linked to the membrane-associated moiety, extracellular moiety, or intracellular moiety.
   71. The donor cell or acceptor cell of any preceding embodiment, wherein the cargo molecule is non-covalently associated with the extracellular moiety or intracellular moiety.
   72. The donor cell or acceptor cell of any of embodiments 1-67, wherein the cargo molecule is not associated or linked with the membrane-associated agent.
   73. The donor cell or acceptor cell of any preceding embodiment, wherein the cargo molecule is associated or linked (e.g., tethered) to the membrane.
   74. The donor cell or acceptor cell of any preceding embodiment, wherein the cargo molecule is chosen from a small molecule (e.g., a drug molecule), a nucleic acid (e.g., an RNA (e.g., an mRNA, siRNA, piRNA, lncRNA, miRNA, or viral RNA) or a DNA (e.g., a vector or viral DNA)), protein (e.g., a cleavable polypeptide (e.g., a zymogen)), a protein-bound molecule, a protein-bound antibody molecule (e.g., bispecific), a lipid (e.g., a lipid containing signaling molecule), or an organelle.
   74a. The donor cell or acceptor cell of any preceding embodiment, wherein the donor or acceptor cell does not comprise a nucleic acid encoding the cargo molecule.
   75. The donor cell or acceptor cell of embodiment 71, wherein the intracellular moiety comprises an antibody molecule (e.g., an antibody or functional fragment thereof (e.g., a Fab, F(ab′)2, Fab′, scFv, or di-scFv)) that binds the cargo molecule.
   76. The acceptor cell of any preceding embodiment, wherein the cargo molecule is released from the membrane-associated moiety or the intracellular moiety, e.g., is cleaved free of the membrane-associated agent by a protease.
   77. The acceptor cell of embodiment 76, wherein the acceptor cell comprises a higher level of cargo molecule than one, two, or all of membrane-associated moiety, extracellular moiety, or intracellular moiety.
   78. The acceptor cell of either embodiment 76 or 77, wherein the acceptor cell does not comprise one, two, or all of membrane-associated moiety, intracellular moiety, and extracellular moiety, or comprises only residual levels of membrane-associated, intracellular, or extracellular moiety (e.g., less than 30, 25, 20, 18, 16, 14, 12, 10, 9, 8, 7, 6, 5, 4, 3, 2, or 1% of cells in a sample of acceptor cells comprise membrane-associated moiety and optionally extracellular moiety).
   79. The donor cell or acceptor cell of any of embodiments 1-71 or 73-78, wherein the cargo molecule is connected to the membrane-associated moiety or intracellular moiety by a linker, e.g., a flexible linker.
   80. The donor cell or acceptor cell of any preceding embodiment, wherein the membrane-associated agent is an exogenous membrane-associated agent, e.g., wherein at least one of the membrane-associated moiety, extracellular moiety, intracellular moiety, or an operably associated cargo molecule is exogenous to the donor cell or acceptor cell.
   81. The donor cell or acceptor cell of embodiment 80, wherein the exogenous membrane-associated agent comprises a fusion protein.
   82. The donor cell or acceptor cell of either of embodiments 80 or 81, wherein the exogenous membrane-associated agent comprises an extracellular moiety comprising an HLA-G domain, an intracellular moiety comprising a Lumio tag an MS2 Coat Protein (MCP), and a cargo molecule comprising an mRNA encoding a polypeptide.
   83. The donor cell or acceptor cell of either of embodiments 80 or 81, wherein the exogenous membrane-associated agent comprises a membrane-associated moiety comprising a palmitoyl moiety or myristoyl moiety (e.g., connected to a lipidation modification sequence (e.g., from a Lck tyrosine kinase) or to the terminus of a peptide chain) and the intracellular moiety comprises EGFP.
   84. The donor cell or acceptor cell of either of embodiments 80 or 81, wherein the exogenous membrane-associated agent comprises a membrane-associated moiety comprising a palmitoyl moiety or myristoyl moiety (e.g., connected to a lipidation modification sequence (e.g., from a Lck tyrosine kinase) or to the terminus of a peptide chain) and the intracellular moiety comprises a Lumio tag and an enzyme, e.g., a reporter enzyme, e.g., β-lactamase.
   85. The donor cell or acceptor cell of either of embodiments 80 or 81, wherein the exogenous membrane-associated agent comprises an extracellular moiety comprising the extracellular domain of E-selectin, a membrane-associated moiety comprising the transmembrane domain of E-selectin, and an intracellular moiety comprising a Lumio tag and an enzyme, e.g., a reporter enzyme, e.g., β-lactamase.
   86. The donor cell or acceptor cell of either of embodiments 80 or 81, wherein the exogenous membrane-associated agent comprises an extracellular moiety comprising the extracellular domain of Notch receptor, a membrane-associated moiety comprising the transmembrane domain of Notch receptor, and an intracellular moiety comprising Cre recombinase, wherein the Notch receptor domains include the T-secretase cleavage site.
   87. The donor cell or acceptor cell of either of embodiments 80 or 81, wherein the exogenous membrane-associated agent comprises an extracellular moiety comprising an HLA-G domain, a membrane-associated moiety comprising a thrombomodulin transmembrane domain, and an intracellular moiety comprising a Lumio tag and Cre recombinase.
   88. The donor cell or acceptor cell of either of embodiments 80 or 81, wherein the exogenous membrane-associated agent comprises a membrane-associated moiety comprising a palmitoyl moiety or myristoyl moiety (e.g., connected to a lipidation modification sequence (e.g., from a Lck tyrosine kinase) or to the terminus of a peptide chain), and an intracellular moiety comprising a Lumio tag, a TEV protease tag, and Cre recombinase.
   89. The donor cell or acceptor cell of either of embodiments 80 or 81, wherein the exogenous membrane-associated agent comprises an extracellular moiety comprising a streptavidin domain, a membrane-associated moiety comprising a thrombomodulin transmembrane domain, and an intracellular moiety comprising a Lumio tag and an enzyme, e.g., a reporter enzyme, e.g., β-lactamase.
   90. The donor cell or acceptor cell of any preceding embodiment, which is a naturally occurring cell, e.g., a mammalian cell, e.g., a human cell.
   91. The donor cell or acceptor cell of any preceding embodiment, which is a primary cell or an immortalized cell, e.g., a cell line (e.g., a human cell line).
   92. The donor or acceptor cell of any preceding embodiment, which is a synthetic cell.
   93. The donor cell of any preceding embodiment, wherein the donor cell is capable of transferring the membrane-associated agent to at least two, at least three, at least four, at least five, or more (e.g., any or all) types of acceptor cells.
   94. The donor cell of any preceding embodiment, which is capable of transferring the membrane-associated agent to a specific acceptor cell type (e.g., and not to other acceptor cell types).
   95. The donor cell of any of any preceding embodiment, wherein the donor cell is a hematopoietic cell, an embryonic cell, a somatic stem cell, an endothelial cell, a fibroblast, an epithelial cell, a cancer cell, or a diseased cell.
   96. The donor cell of any of any preceding embodiment, wherein the donor cell is a cancer cell, a T cell, a B cell, or a cell derived from any thereof.
   97. The donor cell of any of any preceding embodiment, wherein the donor cell is not a 293 cell, HEK cell, human endothelial cell, or a human epithelial cell, monocyte, macrophage, dendritic cell, or stem cell.

98. The donor cell of any of any preceding embodiment, wherein the acceptor cell is an immune cell (e.g., an immune effector cell), e.g., a neutrophil, a lymphocyte (e.g., a T cell, B cell, or NK cell), a PMN (e.g., a granulocyte), a monocyte, a dendritic cell, or a macrophage, a granulocyte, a mesenchymal stem cell, a bone marrow stem cell, an induced pluripotent stem cell, an embryonic stem cell, or a myeloblast.

99. The donor cell of any of any of embodiments 1-97, wherein the acceptor cell is a somatic stem cell, a hematopoietic cell, a nerve cell, a neuroglial cell, a muscle cell, a cartilage cell, a bone cell, an endothelial cell, an epithelial cell, a fibroblast, an adipocyte, a gamete, a cancer cell, or a diseased cell.
100. The acceptor cell of any of any preceding embodiment, wherein the acceptor cell is a somatic stem cell, a hematopoietic cell, a nerve cell, a neuroglial cell, a muscle cell, a cartilage cell, a bone cell, an endothelial cell, an epithelial cell, a fibroblast, an adipocyte, a gamete, a cancer cell, or a diseased cell.
101. The acceptor cell of any of 5-92, wherein the acceptor cell is an immune cell (e.g., an immune effector cell), e.g., a neutrophil, a lymphocyte (e.g., a T cell, B cell, or NK cell), a PMN (e.g., a granulocyte), a monocyte, a dendritic cell, or a macrophage, a granulocyte, a mesenchymal stem cell, a bone marrow stem cell, an induced pluripotent stem cell, an embryonic stem cell, or a myeloblast.
102. The acceptor cell of any of any preceding embodiment, wherein the acceptor cell is not a 293 cell, HEK cell, human endothelial cell, or a human epithelial cell, monocyte, macrophage, dendritic cell, or stem cell.
103. The donor cell of any of embodiments 1-96, wherein the donor cell is a B cell.
104. The acceptor cell of any of embodiments 5-92, wherein the acceptor cell is a T cell.
105. The donor cell of any of embodiments 1-96, wherein the donor cell is a B cell and the acceptor cell is a T cell.
106. The donor cell of any of embodiments 1-96, wherein the donor cell is a T cell.
107. The acceptor cell of any of embodiments 5-92, wherein the acceptor cell is a B cell.
108. The donor cell of any of embodiments 1-96, wherein the donor cell is a T cell and the acceptor cell is a B cell.
109. The donor cell of any preceding embodiment, wherein the donor cell comprises a decreased level of a non-essential component, e.g., a component not essential for cell function.
110. The donor cell of any preceding embodiment, wherein expression of at least one other membrane-associated protein in the donor cell is modulated, e.g., decreased (e.g., knocked down or sequestered (e.g., bound to non-transferrable cell component)).
111. The donor cell of any preceding embodiment, wherein at least one other membrane-associated protein in the donor cell is operatively associated or linked to (e.g., tethered to) a cytoskeletal component of the donor cell, e.g., and is not transferred to the acceptor cell.
112. The donor cell or acceptor cell of any preceding embodiment, wherein the donor cell is autologous to the acceptor cell.
113. The donor cell or acceptor cell of any of embodiments 1-111, wherein the donor cell is allogeneic to the acceptor cell.
114. The donor cell or acceptor cell of any preceding embodiment, wherein the donor cell and acceptor cell are homotypic.
115. The donor cell or acceptor cell of any preceding embodiment, which is obtained from an apheresis sample, a blood draw, a cell line, or a tissue biopsy.
116. The donor cell of any preceding embodiment,

wherein the membrane-associated agent comprises an intracellular moiety,

wherein the membrane-associated moiety or intracellular moiety comprises a cleavage site recognized by a protease, e.g., TEV protease or RHBDL2, and

wherein the donor cell does not comprise appreciable levels of functional protease (e.g., does not comprise the protease), e.g., does not comprise RHBDL2.

117. The donor cell of embodiment 116, wherein the donor cell comprises a mutation, e.g., a deletion, insertion, or substitution, in the gene encoding the protease, e.g., that abrogates expression or function of the protease.
118. The donor cell of embodiment 116, wherein the donor cell comprises or has been contacted with an agent that modulates, e.g., decreases, the expression of the protease, e.g., a miRNA, siRNA, RNAi, or morpholino.
119. The donor cell of embodiment 116, wherein the donor cell does not comprise a nucleic acid encoding the protease.
120. The acceptor cell of any preceding embodiment,

wherein the membrane-associated agent comprises an intracellular moiety,

wherein the membrane-associated moiety or intracellular moiety comprises a cleavage site recognized by a protease, e.g., a rhomboid protease, e.g., RHBDL2,

wherein the acceptor cell does not comprise a nucleic acid encoding the membrane-associated agent, and

wherein the acceptor cell comprises the protease, e.g., comprises RHBDL2.

121. The acceptor cell of embodiment 120, wherein the intracellular moiety is no longer covalently associated with the membrane-associated moiety, e.g., has been cleaved free of the membrane-associated agent by the protease.
122. The acceptor cell of embodiment 121, wherein the acceptor cell comprises a higher level of intracellular moiety than membrane-associated moiety and optionally extracellular moiety.
123. The acceptor cell of either embodiment 121 or 122, wherein the acceptor cell does not comprise membrane-associated moiety and optionally extracellular moiety, or comprises only residual levels of membrane-associated moiety and optionally extracellular moiety (e.g., less than 30, 25, 20, 18, 16, 14, 12, 10, 9, 8, 7, 6, 5, 4, 3, 2, or 1% of cells in a sample of acceptor cells comprise membrane-associated moiety and optionally extracellular moiety).
124. The acceptor cell of any of embodiments 120-123,

wherein the protease is exogenous to the acceptor cell.

125. The acceptor cell of embodiment 124,

wherein the acceptor cell comprises an exogenous nucleic acid encoding the protease.

126. The acceptor cell of any of embodiments 120-123,

wherein the protease is endogenous to the acceptor cell.

127. The acceptor cell of embodiment 126,

wherein the nucleic acid sequence encoding the endogenous protease is operably linked to an exogenous transcriptional regulatory element, e.g., an exogenous promoter, enhancer or both.

128. The donor cell of any preceding embodiment, wherein the donor cell is a progenitor cell, e.g., is a type of cell that is a progenitor cell to the acceptor cell.
129. The acceptor cell of any preceding embodiment, wherein the acceptor cell is a differentiated cell, e.g., is a type of cell that differentiates from the donor cell.
130. The donor cell or acceptor cell of embodiment 128 or 129, wherein the membrane-associated agent comprises a potency factor (e.g., the extracellular moiety or intracellular moiety comprises the potency factor), e.g., that promotes deprogramming or reprogramming of a differentiated cell to a more pluripotent state.
131. The donor or acceptor cell of any preceding embodiment, wherein the membrane-associated agent comprises a polypeptide or domain that is expressed endogenously in the donor or acceptor cell.
132. The donor or acceptor cell of embodiment 131, wherein the membrane-associated agent is expressed (e.g., present) at a level of at least 1.1× (1.1 times), 1.5×, 2×, 3×, 4×, 5×, 10×, 50×, or 100× the level of the endogenously expressed polypeptide or domain (and optionally no higher than 200×, 100×, 50×, 10×, 5×, 4×, 3×, 2×, or 1.5× the level of the endogenously expressed polypeptide or domain).
133. The donor cell of any preceding embodiment, wherein the membrane-associated agent is expressed transiently, e.g., from an extra-chromosomal nucleic acid.
134. The donor cell of any of embodiments 1-132, wherein the membrane-associated agent is stably expressed from a genome-integrated nucleic acid.
135. The donor cell or acceptor cell of any preceding embodiment, wherein the extracellular moiety is exogenous to the donor or acceptor cell (e.g., the donor cell, the acceptor cell, or both).
136. The donor cell or acceptor cell of any preceding embodiment, wherein the intracellular moiety is exogenous to the donor or acceptor cell (e.g., the donor cell, the acceptor cell, or both).
137. The donor cell or acceptor cell of any preceding embodiment, wherein the membrane-associated moiety is exogenous to the donor or acceptor cell (e.g., the donor cell, the acceptor cell, or both).
138. The donor or acceptor cell of any preceding embodiment, wherein the membrane-associated agent comprises a cellular migration factor (e.g., the extracellular moiety, intracellular moiety, or cargo moiety comprises the cellular migration factor).
139. The donor or acceptor cell of embodiment 138, wherein the cellular migration factor comprises CCR7 and promotes migration of an acceptor cell toward CCL19 and CCL21 ligands (e.g., toward a cell expressing CCL19 and CCL21 ligands).
140. The donor or acceptor cell of any preceding embodiment, wherein the membrane-associated agent comprises IL2RA or a functional fragment or variant thereof (e.g., the extracellular moiety or intracellular moiety comprises the IL2RA or a functional fragment or variant thereof).
141. The donor or acceptor cell of any preceding embodiment, wherein the membrane-associated agent comprises Basp1 or a functional fragment or variant thereof (e.g., the extracellular moiety or intracellular moiety comprises the Basp1 or a functional fragment or variant thereof).
142. The donor or acceptor cell of any preceding embodiment, wherein the membrane-associated agent comprises a therapeutic polypeptide or a functional fragment or variant thereof (e.g., the extracellular moiety or intracellular moiety comprises therapeutic polypeptide or a functional fragment or variant thereof).
143. The donor or acceptor cell of any preceding embodiment, wherein the membrane-associated agent is a fusion polypeptide, e.g., a synthetic fusion polypeptide.
144. The donor or acceptor cell of any preceding embodiment, wherein the extracellular domain comprises a ligand that is bound by a receptor on the acceptor cell.
145. The donor or acceptor cell of any preceding embodiment, wherein the extracellular domain comprises a chemokine group receptor, the acceptor cell comprises a D6 scavenger receptor, or both.
146. The donor or acceptor cell of any preceding embodiment, wherein the extracellular moiety comprises a targeting domain that binds to a first target cell moiety and a second target cell moiety.
147. The donor or acceptor cell of embodiment 146, wherein the acceptor cell comprises the first target cell moiety.
148. The donor or acceptor cell of embodiment 147, wherein the acceptor cell does not comprise the second target cell moiety.
149. The donor or acceptor cell of any of embodiments 146-148, wherein the extracellular moiety promotes the interaction of the donor cell, the acceptor cell, and a third cell comprising the second target cell moiety.
150. A composition, e.g., a preparation, comprising a plurality (e.g., population) of donor cells of any preceding embodiment.
151. A composition, e.g., a preparation, comprising a plurality (e.g., population) of acceptor cells of any preceding embodiment.
152. The composition of embodiment 150, which transfers (e.g., delivers) a detectable amount and/or a biologically effective amount of the membrane-associated agent or the cargo molecule to at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of a plurality of target cells, e.g., acceptor cells.
153. The composition of embodiment 151, wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of the cells in the plurality comprise a detectable amount and/or a biologically effective amount of the membrane-associated agent or the cargo molecule.
154. A system, e.g., a reaction mixture, comprising:

the donor cell of any preceding embodiment, and

an acceptor cell,

wherein the donor cell and acceptor cell are provided under conditions suitable for transfer of the membrane-associated agent and/or cargo molecule from the donor cell to the acceptor cell,

wherein the acceptor cell does not comprise a nucleic acid encoding the membrane-associated agent and/or the cargo molecule, or differentially expresses the membrane-associated agent and/or cargo molecule, e.g., relative to an endogenously-expressed membrane-associated agent and/or cargo molecule, if any.

155. The system of embodiment 154, further comprising an antibody a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell.
156. A system, e.g., a reaction mixture, comprising:

the donor cell of any preceding embodiment, and

a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell.

157. A system, e.g., a reaction mixture, comprising:

an acceptor cell of any preceding embodiment, and

a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell.

158. A system, e.g., a reaction mixture, comprising:

a donor cell of any preceding embodiment,

an acceptor cell of any preceding embodiment, and

a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell.

159. A pharmaceutical composition comprising the donor cell or composition of any of the preceding embodiments.
160. The pharmaceutical composition of embodiment 159, further comprising a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell.
161. A pharmaceutical composition comprising the acceptor cell or composition of any of the preceding embodiments.
162. The pharmaceutical composition of any of embodiments 159-161, wherein:
i) the pharmaceutical composition meets a pharmaceutical or good manufacturing practices (GMP) standard;
ii) the donor cell or acceptor cell was made according to good manufacturing practices (GMP);
iii) the donor cell or acceptor cell has a pathogen level below a predetermined reference value, e.g., is substantially free of pathogens; or
iv) the donor cell or acceptor cell has a contaminant level below a predetermined reference value, e.g., is substantially free of contaminants.
   163. A method of modifying an acceptor cell, comprising:

contacting the acceptor cell with a donor cell or composition comprising a plurality of donor cells of any preceding embodiment, under conditions suitable for transfer of the membrane-associated agent and/or cargo molecule to the acceptor cell,

wherein the acceptor cell does not comprise a nucleic acid encoding the membrane-associated agent and/or cargo molecule,

thereby modifying the acceptor cell.

164. A method of modifying an acceptor cell, comprising:

contacting the acceptor cell with a donor cell or composition comprising a plurality of donor cells of any preceding embodiment, under conditions suitable for transfer of the membrane-associated agent and/or cargo molecule to the acceptor cell,

wherein after the transfer the acceptor cell comprises an increased amount of the membrane-associated agent and/or cargo molecule,

thereby modifying the acceptor cell.

165. The method of embodiment 163, wherein after the transfer the acceptor cell comprises an increased amount of the membrane-associated agent and/or cargo molecule.
166. The method of embodiment 164, wherein the acceptor cell does not comprise a nucleic acid encoding the membrane-associated agent and/or cargo molecule.
167. The method of any of embodiments 163-166, wherein modifying the acceptor cell comprises transferring the membrane-associated agent from the donor cell (e.g., the first donor cell, the second donor cell, or both) to the acceptor cell.
168. A method of making a modified cell, comprising:

providing an unmodified cell,

contacting the unmodified cell with a donor cell or composition comprising a plurality of donor cells of any preceding embodiment, under conditions suitable for transfer of the membrane-associated agent and/or cargo molecule to the unmodified cell,

thereby making a modified cell,

wherein:

(i) neither the unmodified cell or modified cell comprise a nucleic acid encoding the membrane-associated agent,

(ii) after the transfer the modified cell comprises an increased amount of the membrane-associated agent and/or cargo molecule than the unmodified cell, or

both (i) and (ii).

169. The method of embodiment 168, wherein the modified cell comprises membrane-associated agent, and wherein the membrane-associated agent was not produced in the acceptor cell.
170. The method of either of embodiments 168 or 169, further comprising:

providing a donor cell or composition comprising a plurality of donor cells of any preceding embodiment.

171. The method of embodiment 170, wherein providing comprises contacting a cell with a nucleic acid encoding the membrane-associated agent, thereby providing a donor cell comprising the membrane-associated agent.
172. A method of delivering a cargo molecule to a cell, comprising:

providing the donor cell or the composition comprising a plurality of donor cells of any preceding embodiment, wherein the donor cell or plurality of donor cells comprise the cargo molecule;

providing an acceptor cell that does not comprise a nucleic acid encoding the membrane-associated agent and/or cargo molecule; and

contacting the acceptor cell with the donor cell or composition under conditions suitable for transfer of the membrane-associated agent to the acceptor cell,

thereby delivering the cargo molecule to the cell.

173. A method of delivering a cargo molecule to a cell, comprising:

providing the donor cell or the composition comprising a plurality of donor cells of any preceding embodiment, wherein the donor cell or plurality of donor cells comprise the cargo molecule;

providing an acceptor cell; and

contacting the acceptor cell with the donor cell or composition under conditions suitable for transfer of the cargo molecule to the acceptor cell,

wherein after the transfer the acceptor cell comprises an increased amount of the cargo molecule,

thereby delivering the cargo molecule to the cell.

174. The method of any of embodiments 163-173, further comprising contacting the donor cell and the acceptor cell with a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell.
175. The method of any of embodiments 163-174, further comprising one, two, three, four or all of:
i) expanding the acceptor cell or population comprising acceptor cells;
ii) selecting the acceptor cell or population comprising acceptor cells;
iii) enriching for the acceptor cell or population comprising acceptor cells;
iv) purifying the acceptor cell or population comprising acceptor cells; or
v) formulating the acceptor cell or population comprising acceptor cells.
   176. The method of any of embodiments 163-175, further comprising:

providing a donor cell or composition comprising a plurality of donor cells of any preceding embodiment.

177. The method of embodiment 176, wherein providing comprises contacting a cell with a nucleic acid encoding the membrane-associated agent, thereby providing a donor cell comprising the membrane-associated agent.
178. The method of any of any of embodiments 163-177, wherein the contacting occurs in vitro or ex-vivo.
179. The method of any of embodiments any of embodiments 163-177, wherein the contacting occurs in vivo.
180. The method of any of embodiments, 163-179, wherein the cell, unmodified cell, or acceptor cell is from a subject or in a subject.
181. A method of modulating, e.g., enhancing or decreasing, a biological function in a subject, a target tissue, or a cell, comprising administering to the subject, or contacting the target tissue or the cell with:

a donor cell or composition comprising a plurality of donor cells described herein,

an acceptor cell or composition comprising a plurality of acceptor cells described herein,

a system described herein, or

a pharmaceutical composition described herein,

thereby modulating the biological function in the subject.
182. A method of delivering or targeting a function to a subject, comprising administering to the subject:

a donor cell or composition comprising a plurality of donor cells described herein,

an acceptor cell or composition comprising a plurality of acceptor cells described herein, a system described herein, or

a pharmaceutical composition described herein,

wherein the donor cell, the acceptor cell, compositions comprising pluralities of the same, or the pharmaceutical composition is administered in an amount and/or time such that the function in the subject is delivered or targeted.
183. The method of any of embodiments 180-182, wherein the subject has a cancer, an inflammatory disorder, autoimmune disease, a chronic disease, inflammation, damaged organ function, an infectious disease, a degenerative disorder, a genetic disease, or an injury.
184. A composition comprising a plurality of donor cells, the plurality of donor cells comprising:
   (a) a membrane-associated agent, the agent comprising:
      a membrane-associated moiety, and
      one or both of an extracellular moiety or an intracellular moiety, and
   (b) optionally a cargo molecule,
   wherein the membrane-associated agent and optionally the cargo molecule are configured to be transferred to an acceptor cell;
   wherein at least one of the membrane-associated moiety, extracellular moiety, intracellular moiety, or the cargo molecule is exogenous to the plurality of donor cells,
   optionally wherein the plurality of donor cells transfers a detectable amount or a biologically effective amount of the membrane-associated agent and/or the cargo molecule to at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of a plurality of acceptor cells, and
   wherein the plurality of donor cells and acceptor cells are not immune cells.
185. A composition comprising a plurality of donor cells, the plurality of donor cells comprising:
   (a) a membrane-associated agent, the agent comprising:
      a membrane-associated moiety, and
      one or both of an extracellular moiety or an intracellular moiety, and
   (b) optionally a cargo molecule,
   wherein the membrane-associated agent and optionally the cargo molecule are configured to be transferred to an acceptor cell;
   wherein at least one of the membrane-associated moiety, extracellular moiety, intracellular moiety, or cargo molecule is present at a different level in the donor cell than a source cell from which the donor cell is derived,
   optionally wherein the plurality of donor cells transfers a detectable amount or a biologically effective amount of the membrane-associated agent and/or the cargo molecule to at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of a plurality of acceptor cells, and
   wherein the plurality of donor cells and acceptor cells are not immune cells.
186. A composition comprising a plurality of donor cells, the plurality of donor cells comprising:
   (a) a membrane-associated agent, the agent comprising:
      a membrane-associated moiety, and
      one or both of an extracellular moiety or an intracellular moiety, and
   (b) optionally a cargo molecule,
   wherein the membrane-associated agent and optionally the cargo molecule are configured to be transferred to an acceptor cell;
   wherein at least one of the membrane-associated moiety, extracellular moiety, intracellular moiety, or the cargo molecule is exogenous to the plurality of donor cells, and
   wherein the membrane-associated moiety or intracellular moiety comprises a protease cleavage site recognized by a protease that is not expressed in the plurality of donor cells.
187. A composition comprising a plurality of donor cells, the plurality of donor cells comprising:
   (a) a membrane-associated agent, the agent comprising:
      a membrane-associated moiety, and
      one or both of an extracellular moiety or an intracellular moiety, and
   (b) optionally a cargo molecule,
   wherein the membrane-associated agent and optionally the cargo molecule are configured to be transferred to an acceptor cell,
   wherein at least one of the membrane-associated moiety, extracellular moiety, intracellular moiety, or cargo molecule is present at a different level in the plurality of donor cells than a source cell from which the plurality of donor cells are derived, and
   wherein the membrane-associated moiety or intracellular moiety comprises a protease cleavage site recognized by a protease that is not expressed in the plurality of donor cells.
188. A composition comprising a plurality of donor cells, the plurality of donor cells comprising:
   (a) a membrane-associated agent, the agent comprising:
      a membrane-associated moiety, and
      one or both of an extracellular moiety or an intracellular moiety, and
   (b) a cargo molecule,
   wherein the membrane-associated agent is configured to be transferred to an acceptor cell,
   wherein at least one of the membrane-associated moiety, extracellular moiety, intracellular moiety, or the cargo molecule is exogenous to the plurality of donor cells,
   wherein the extracellular moiety comprises a targeting domain that binds to a first target cell moiety on the acceptor cell, and
   wherein the intracellular moiety is non-covalently associated with, e.g., binds to, the cargo molecule.
189. A composition comprising a plurality of donor cells, the plurality of donor cells comprising:
   (a) a membrane-associated agent, the agent comprising:
      a membrane-associated moiety, and
      one or both of an extracellular moiety, an intracellular moiety, and
   (b) a cargo molecule,
   wherein the membrane-associated agent is configured to be transferred to an acceptor cell;
   wherein at least one of the membrane-associated moiety, extracellular moiety, intracellular moiety, or cargo molecule is present at a different level in the donor cell than a source cell from which the donor cell is derived,
   wherein the extracellular moiety comprises a targeting domain that binds to a first target cell moiety on the acceptor cell, and
   wherein the intracellular moiety is non-covalently associated with, e.g., binds to, the cargo molecule.
      190. The composition of either of embodiments 189, wherein the targeting domain also binds to a second target cell moiety.
      191. The composition of embodiment 190, wherein the acceptor cell does not comprise the second target cell moiety.
192. A composition comprising a plurality of acceptor cells, the plurality of acceptor cells comprising:
   (a) a membrane-associated agent, the membrane-associated agent comprising:
      a membrane-associated moiety, and
      one or both of an extracellular moiety or an intracellular moiety, and
   (b) optionally a cargo molecule,
   wherein the acceptor cells do not comprise a nucleic acid encoding the membrane-associated agent,
   wherein at least one of the membrane-associated moiety, extracellular moiety, intracellular moiety, or cargo molecule is exogenous to the acceptor cell,
   optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell, and
   wherein the plurality acceptor cells are not immune cells.
193. A composition comprising a plurality of acceptor cells, the plurality of acceptor cells comprising:
   (a) a membrane-associated agent, the agent comprising:
      a membrane-associated moiety;
      one or both of an extracellular moiety or an intracellular moiety; and
   (b) optionally a cargo molecule;
   wherein the acceptor cells do not substantially express a nucleic acid encoding the membrane-associated agent or, if a cargo molecule is present, do not substantially express a nucleic acid encoding the cargo molecule,
   optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell, and
   wherein the plurality acceptor cells are not immune cells.
194. A composition comprising a plurality of acceptor cells, the plurality of acceptor cells comprising:
   (a) a membrane-associated agent, the membrane-associated agent comprising:
      a membrane-associated moiety, and
      one or both of an extracellular moiety or an intracellular moiety, and
   (b) optionally a cargo molecule,
   wherein the acceptor cells do not comprise a nucleic acid encoding the membrane-associated agent and, if a cargo molecule is present, do not comprise a nucleic acid encoding the cargo molecule,
   wherein at least one of the membrane-associated moiety, extracellular moiety, intracellular moiety, or cargo molecule is exogenous to the acceptor cell,
   optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell, and
   wherein:
      (1) the membrane-associated moiety or intracellular moiety comprises a protease cleavage site recognized by a protease present in the plurality of acceptor cells, or
      (2) the plurality of acceptor cells comprises a membrane-associated agent comprising a cleaved fragment corresponding to cleavage by said protease at said protease cleavage site.
195. A composition comprising a plurality of acceptor cells, the plurality of acceptor cells comprising:
   (a) a membrane-associated agent, the agent comprising:
      a membrane-associated moiety;
      one or both of an extracellular moiety or an intracellular moiety; and
   (b) optionally a cargo molecule;
   wherein the acceptor cells do not substantially express a nucleic acid encoding the membrane-associated agent or, if a cargo molecule is present, do not substantially express a nucleic acid encoding the cargo molecule,
   optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell, and
   wherein:
      (1) the membrane-associated moiety or intracellular moiety comprises a protease cleavage site recognized by a protease present in the plurality of acceptor cells, or
      (2) an acceptor cell of the plurality of acceptor cells comprises a membrane-associated agent comprising a cleaved fragment corresponding to cleavage by said protease at said protease cleavage site.
196. A composition comprising a plurality of acceptor cells, the plurality of acceptor cells comprising:
   (a) a membrane-associated agent, the agent comprising:
      a membrane-associated moiety, and
      one or both of an extracellular moiety or an intracellular moiety, and
   (b) a cargo molecule,
   wherein the acceptor cells do not comprise a nucleic acid encoding the membrane-associated agent and, do not comprise a nucleic acid encoding the cargo molecule,
   wherein at least one of the membrane-associated moiety, extracellular moiety, intracellular moiety, or cargo molecule is exogenous to the acceptor cell,
   optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell,
   wherein the extracellular moiety comprises a targeting domain that binds to a first target cell moiety and a second target cell moiety and wherein the acceptor cell comprises the first target cell moiety or the second target cell moiety but not both, and
   wherein the intracellular moiety is associated, e.g., non-covalently, with the cargo molecule.
197. A composition comprising a plurality of acceptor cells, the plurality of acceptor cells comprising:
   (a) a membrane-associated agent, the agent comprising:
      a membrane-associated moiety;
      one or both of an extracellular moiety or an intracellular moiety; and
   (b) a cargo molecule;
   wherein the acceptor cells do not substantially express a nucleic acid encoding the membrane-associated agent and do not substantially express a nucleic acid encoding the cargo molecule,
   optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell,
   wherein the extracellular moiety comprises a targeting domain that binds to a first target cell moiety and a second target cell moiety and wherein the acceptor cell comprises the first target cell moiety or the second target cell moiety but not both, and
   wherein the intracellular moiety is associated, e.g., non-covalently with the cargo molecule.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Example 1. Membrane Transfer from a Donor Cell (Donor) to an Acceptor Cell (Acceptor

This example demonstrates the transfer of cell surface material (membrane, including lipids and proteins) from an immortalized donor cell line (K562) to an acceptor cell (THP-1). Cell-cell contact is accomplished by co-culturing the cells

Donor Cell Membrane Labeling

3×10⁶ K562 donor cells are cultured in their preferred growth media at 37 C in the presence of 5% CO₂. The cells are washed in PBS and stained with PKH26, a lipophilic fluorescent dye, according to manufacturer protocols. Following 3-5 washes and quenching with FBS, the cells are returned to suspension medium.

Cell Identity Labeling

To distinguish donor cells from acceptor cells, one or both of the cell types are labeled with Cell unique, non-transferable intracellular dyes, intracellular fluorescent proteins, or antibodies against non-transferable surface proteins. Minimally transferred dyes that covalently crosslink to cytoplasmic structures (Thermo CellTracker Green or Thermo CellTracker Orange, RedDot1, etc.) are used in cases where non-transferable surface markers are lacking.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Measurement

Transfer of membrane from donor to acceptor is measured by flow cytometry or fluorescence microscopy to identify acceptor cells that have acquired membrane from donors. These positive acceptors co-stain for their unique identity marker and the lipophilic dye that is applied only to the donor cells. Importantly, identity dyes may sometimes transfer at low levels indicating transfer of labeled membrane proteins from the donor cells to the acceptor cells in addition to lipid transfer. The two cell types can still be distinguished since the amount of identity dye transferred will be orders of magnitude less than the total amount of cytoplasmic material stained by the identity tracker dye before washing and co-culture.

Co-Culture of K562 (CMFDA/PKH26) with THP-1 (DeepRed)

### SUMMARY

The following protocol is to co-culture CMFDA+PK26+K562 cells with DeepRed+ THP-1 cells at Donor:Acceptor ratios of 1:1 and 2:1 with either 25, 50, or 100K total cells per well in 96-well plates.

Reagents
PKH26 (1 mM, Sigma MIDI26), stored at 4° C. which includes the dye solution and the Diluent C
CellTracker Green CMFDA (ThermoFisher C2925) reconstituted stored at −20° C., 3 μL
CellTracker DeepRed (ThermoFisher C34565) reconstituted stored at −20° C., 2 μL
15 mL polypropylene conical tubes
Centrifuge
FBS
Vi-cell
Fix buffer (2% formaldehyde in 1×PBS), prepared on the day of, stored at 4° C.
96-well flat bottom black-walled plates
96-well flat bottom clear plates
96-well V-bottom clear plates
Anhydrous DMSO (Sigma 276855)
1×PBS (Ca²⁺, Mg²⁺ free)
RPMI (no serum)
R10+
15 mL polypropylene conical tubes
5 mL FACS tubes
Violet Amine live/dead (thermoFisher L34955)

PKH26 Preparation

Bring to room temperature the dye solution and the Diluent C, keeping dye protected from light and cap tight to prevent evaporation.

Inspect dye for crystals and dissolve if needed.

Prepare the 2× Dye Dilutions:
a) PKH-A: Add 2 μL of the dye concentrate to 0.498 mL of Diluent C (4 μM=2× for 2 μM)
b) PKH-B: Add 2 μL of EtOH to 0.498 mL Diluent C.

CellTracker Green Preparation

Bring the dye to room temperature.

Prepare the 2× Dye Dilutions:
a) Green-A: Add 2 μL of the dye stock to 2 mL serum-free RPMI (now 1004). Add 100 μL of Solution A to 900 μL of serum-free RPMI (now 10 μM=2× for 0.5 μM)
b) Green-B: Add 2 μL of the DMSO to 2 mL 1×PBS. Add 100 μL to 900 μL.

Warm to 37° C.

CellTracker DeepRed

Bring the dye to room temperature.

Prepare the 2× Dye Dilutions:
a) Red-A: Add 1 μL of the dye stock to 1 mL serum-free RPMI (now 1 μM=2× for 0.5 μM).
b) Red-B: Add 1 μL of the DMSO to 1 mL 1×PBS.

Warm to 37° C.

Live/Dead Violet Prep/Usage

To the 2 μL aliquot, add 198 μL 1×PBS

Add 5 μL dye to 100 μL of cells in 1×PBS

Staining Conditions

| | | | | | Cell | |
|---|---|---|---|---|---|---|
| | Cells | PKH | CMFDA | DeepRed | input | Use |
| 1 | K562 | − | − | − | 1M | co-culture and |
| | | | | | | unstained ref |
| 2 | K562 | + | + | − | 1M | co-culture and |
| | | | | | | CMFDA+ ref |
| 3 | K562 | + | − | − | 1M | PKH ref |
| 4 | THP-1 | − | − | − | 1M | co-culture and |
| | | | | | | unstained ref |
| 5 | THP-1 | − | − | + | 1M | Co-culture and |
| | | | | | | DeepRed ref |

Co-Culture Preparations (Everything at 0.5×10\hat{ }6)

| | | | | Volume (μL) of cells at | |
|---|---|---|---|---|---|
| | | | | 0.5 × 10\hat{ }6/mL | |
| | K562 stain | THP-1 stain | E:T | K562 | THP-1 |
| 1 | Unstained | — | NA | — | — |
| 2 | — | Unstained | NA | — | — |
| 3 | Unstained | Unstained | 1 | 200 | 200 |
| 4 | Stained | Stained | 1 | 200 | 200 |
| 5 | Unstained | Stained | 1 | 200 | 200 |
| 6 | Stained | Unstained | 1 | 200 | 200 |
| 7 | Unstained | Unstained | 2 | 250 | 125 |
| 8 | Stained | Stained | 2 | 250 | 125 |
| 9 | Unstained | Stained | 2 | 250 | 125 |
| 10 | Stained | Unstained | 2 | 250 | 125 |

Co-Culture Conditions (n=36)

Everything listed in the “Co-culture prep” section at 3 different inputs/well: 25, 50, and 100K/well.

TABLE 8

| | | | 1:1 | | | 2:1 | | | Donor:Acceptor (K562:THP) | |
|---|---|---|---|---|---|---|---|---|---|---|
| | | | 25 | 50 | 100 | 25 | 50 | 100 | Total cells/well (×10\hat{ }3) | |
| | K562 | THP-1 | (12.5:12.5) | (25:25) | (50:50) | (16.7:8.3) | (33.3:16.7) | (66.7:33.3) | Cell input | Additional Flow control |
| 1 | Unst | — | | | | | | | | 25K K562 unstained |
| 2 | — | Unst | | | | | | | | 25K THP unstained |
| 3 | Unst | Unst | | | | | | | | 25K K562 PKH only |
| 4 | Stain | Stain | | | | | | | | 25K THP DeepRed |
| 5 | Unst | Stain | | | | | | | | 25K K562 CMFDA |
| 6 | Stain | Unst | | | | | | | | |
| | | uL/wel | 50 | 100 | 200 | 50 | 100 | 200 | | |

All these get L/D

Flow and Imaging Samples

The flow samples are those outlined in the co-culture plate above.

The imaging samples are those outlined in the co-culture plate above, minus those in green
a) Image at 30 min and 60 min post-co-culture

Procedure

Count K562 and THP-1 cells and measure viability using Vi-cell.

For K562 Cells
a) Remove 3×10⁶ cells into a new 15 mL conical tube.
b) Wash with PBS.
c) Aspirate, wash again with 10 mL PBS.
d) During the wash, prepare dye dilution as outlined above (this should be performed immediately before adding to cells).
e) Resuspend cells in 150 μL Diluent C (cells should not be sitting in Diluent C for extended periods). Distribute 50 μL (1×10⁶ cells) to each of 3 15 mL tubes.
f) Add:
   i) 50 μL (1×10⁶ cells) PKH-A to dual-stained samples
   ii) 50 μL (1×10⁶ cells) PKH-B to unstained samples
   iii) 50 μL (1×10⁶ cells) PKH-B to CMFDA+ reference sample
g) This should be performed rapidly and mixed immediately by pipetting.
h) Incubate for 5 min at RT.
i) Add 500 μL 100% FBS to quench the staining. Incubate for 1 min.
j) Pellet cells by centrifugation for 10 min at 300 g at RT.
k) Resuspend in 5 mL R10+ and transfer to new 15 mL conical tube. Rinse original tube with 5 mL R10+ and transfer to the new tube.
l) Pellet 5 min at 300 g at RT.
m) Wash again with 5 mL RPMI **no serum**.
n) Resuspend in 5 mL RPMI
   i) From the “dual-stained sample”, remove 125 μL (25K) to a 5 mL FACS tube with 1×PBS—for PKH+ ref control
      (1) Pellet. Resuspend in 200 μL 2% fix. Set aside.
o) For the remaining dual-stained, unstained, and CMFDA+ samples, pellet.
p) Resuspend each in 50 μL RPMI.
q) Add:
   i) 50 μL Green-A to dual-stained tube (0.975×10⁶ cells)
   ii) 50 μL Green-B to unstained tube (1×10⁶ cells)
   iii) 50 μL Green-A to CMFDA+ ref. (1×10⁶ cells)
r) Incubate 15 min at 37° C., 5% CO₂.
s) Wash with 5 mL R10+
t) Wash one more:
   i) Add 5 mL R10+ to dual-stained.
      (1) Pellet.
   ii) Add 5 mL R10+ to unstained, remove 125 μL (25K) to a 5 mL FACS tube with 1×PBS—for unstained ref. control
      (1) Pellet the 25K cells, resuspend in 200 μL 2% fix. Set aside.
      (2) Pellet the remaining unstained.
   iii) Add 5 mL PBS to the CMFDA+ ref:
      (1) Pellet the entire sample. Resuspend in 200 μL 2% fix, transfer to 5 mL FACS tube, set aside.
u) Resuspend in R10+ at 0.5×10⁶/mL:
   i) 1.950 mL to the dual stained sample (0.975×10⁶ cells)
   ii) 1.950 mL to the unstained sample (0.975×10⁶ cells)

For THP-1 Cells
a) Remove 2×10⁶ live cells into a new 15 mL conical tube.
b) Wash cells with RPMI (no serum, Ca²⁺, or Mg²⁺) by pelleting at 300 g for 5 min at RT.
c) Resuspend in 100 μL RPMI.
d) Distribute into each 15 mL conical tube:
   i) 50 μL (1×10⁶ cells) for stained THP and DeepRed ref. control
   ii) 50 μL (1×10⁶ cells) for unstained THP
e) Add:
   i) 50 μL Red-A to stained tube
   ii) 50 μL Red-B to unstained tube
f) Incubate 15 min at 37° C., 5% CO₂.
g) Add up to 5 mL R10+
   i) From stained tube, remove 125 μL (25K) to a 5 mL FACS tube with 1×PBS—for DeepRed+ ref control
      (1) spin down, resuspend in 200 μL 2% fix. Set aside.
   ii) From unstained, remove 125 μL (25K) to a 5 mL FACS tube with 1×PBS—for unstained THP sample
      (1) spin down, resuspend in 200 μL 2% fix. Set aside.
h) To the remaining stained and unstained, spin down.
i) Wash once more with 5 mL R10+
j) Resuspend in R10+ at 0.5×10⁶/mL:
   i) 1.950 mL to the stained sample (0.975×10⁶ cells)
   ii) 1.950 mL to the unstained sample (0.975×10⁶ cells)

Co-Culture
a) Combine the stained and unstained K562 and THP-1 cells according to the table above
b) Distribute either 50, 100, or 200 μL of each of the cell mixtures to 96-well clear or black plates (for flow cytometry or imaging), according to the plate layout above.
c) Centrifuge plate for 5 min at 100 g.
d) Culture at 37° C., 5% CO₂ for 1 hr.
e) For flow samples, harvest cells by pipetting and transfer to 96-well v-bottom plates.
   i) Pellet 300× g for 5 min.
   ii) Stain with L/D violet (5 uL).
   iii) Wash with PBS.
   iv) Fix with 100 μL 2% fix buffer with vigorous pipetting.

Run samples on flow cytometer or image via confocal.

Using the protocol and experimental steps described above, the transfer of membrane and membrane-associated agents was assayed between exemplary donor cells, K562 cells, to exemplary acceptor cells (Jurkat or THP-1 cells).

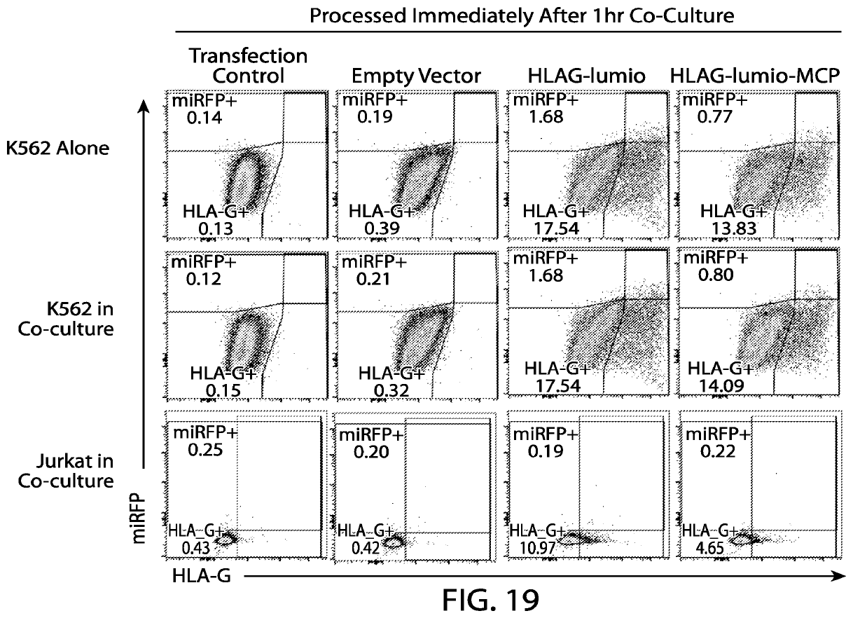

FIG. 19 shows flow cytometry data analyzing the membrane content of the K562 donor cells and Jurkat acceptor cells using the above protocol. The results demonstrate transfer of an exemplary membrane-associated agent, a fusion protein comprising HLAG, a Lumio tag, and MCP, from the K562 cells to the Jurkat cells.

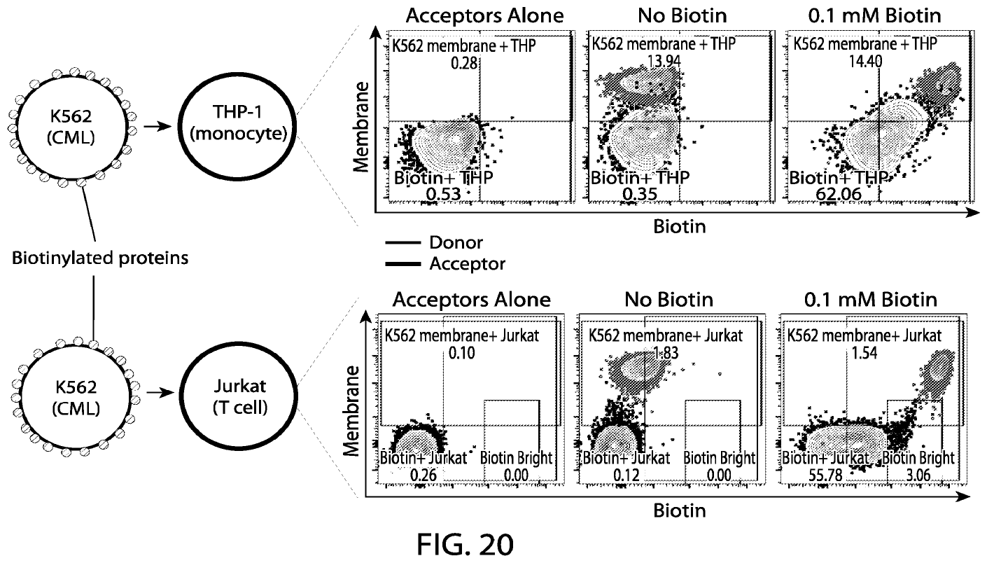

FIG. 20 shows flow cytometry data analyzing the membrane content of the K562 donor cells and THP-1 acceptor cells or Jurkat acceptor cells using the above protocol. The results demonstrate that biotinylated membrane proteins from K562 cells were transferred to THP-1 or Jurkat cells upon co-culturing.

### Example 2. Correct Localization of Transproteins in Acceptors after Transfer

This example demonstrates correct localization of transproteins in acceptor cells after transfer from donor cells.

Transfer of proteins to acceptors is intended to provide the acceptor with functions that are similar to the function of the protein in the donor cells. This requires that the transproteins localize to the membrane of the acceptor cells with similar orientation and access to the cytoplasmic compartment.

Expressing a Cytoplasm-Facing Reporter Protein in Donors and Acceptors

Baculoviral constructs that encode membrane-tethered fluorescent proteins are used to label K562 donor cells. Specifically, we use the CellLights plasma membrane-GFP reagent (Thermo), which encodes EGFP fused to a myristoylation/palmitoylation signal from Lck, which localizes it to the inner leaflet of the plasma membrane. The reagent is also available as a fusion to RFP and CFP, allowing similar labeling of THP-1 acceptor cells.

Cell-Cell Contact

Donor and acceptor cells are monocultured separately in suspension media after labeling. To co-culture, the acceptor cells are added to the donor cell cultures at a ratio of 1:2. Overall cell concentration is varied to meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Assaying Cargo Localization

Acceptor cells are isolated after contact-mediated transfer procedures and fixed with 10% PFA for 15 minutes. Suspension cells can be cytospun prior to fixation. After brief washes, HRP substrates are applied to the fixed cells allowing HRP to cleave the substrate and generate either colorimetric or fluorescent signal. Localization of the signal is determined using microscopic imaging.

### Example 3. Functionality of Proteins after Transfer to Acceptors

This example demonstrates that membrane material transferred from a donor to an acceptor becomes properly oriented in the acceptor membrane and is able to perform its function. Chimeric antigen receptors, when expressed on T cell plasma membranes, allow the T cells to kill target cells that express surface proteins recognized by the CAR's extracellular antigen-binding scFv domain. CARs are often installed in T cells by genetic engineering. Here, we show installation of CARs in T cells by transferring them as transproteins from a B cell donor that has been genetically engineered to express the CAR. This enables the creation of a CAR T cell without genetic modification of the T cell itself. The transferred CAR transprotein can be tested for function by exposing the acceptor cell to target cells expressing the target of the CAR and measurement of killing.

Cell Membrane Labeling

Donors and acceptors are cultured in their preferred growth media at 37° C. in the presence of 5% CO₂. Prior to membrane transfer assays, the donor cells are stained with a lipophilic, fluorescent dye according to manufacturer protocols (dyes for membrane staining include PKH26, PKH67, DiR, DiO, DiL). Alternatively, donor cells may also be labeled by biotinylation using Sulfo-NHS-SS-Biotin, which covalently modifies the amine groups of surface proteins with biotin (use of biotin is demonstrated in FIG. 20). Extensive washing, including changing of tubes or wells, is crucial following dye exposure to remove all dye from the media. Following 3-5 washes, the cells are returned to suspension medium.

Expressing Cargo in Donors

A lentiviral construct that encodes a CD19-targeting CAR is used to transfect donor cells, generating a CD19 CAR-expressing donor cell line.

Cell Identity Labeling

To distinguish donor cells from acceptor cells, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Measurement

Transfer of membrane from donor to acceptor is measured by flow cytometry or fluorescence microscopy to identify acceptor cells that have acquired membrane from donors. These positive acceptors co-stain for their unique identity marker and the lipophilic dye that is applied only to the donor cells.

Measurement of Cargo Function in Acceptors

Detection of the CAR on the acceptor cells (positive for the appropriate CellTracker dye) suggests proper orientation of the CAR on the T cell membrane. As a definitive functional test, CAR function is measured in the acceptor cells using cytotoxicity assays. Specifically, positive acceptor cells are exposed to CD19-expressing target cells. Target cell cytotoxicity is assayed by loss of fluorescence or other cell viability assays.

### Example 4. Delivery to the Cytoplasm

This example demonstrates the use of engineered fusion proteins designed to deliver specific proteins to the cytoplasm of an acceptor after cleavage by acceptor-specific protease activity.

Knock-Out of Intramembrane Protease in Donor Cells

Targeting constructs are introduced into K562 donor cells that are designed to knock out RHBDL2, a rhomboid protease that is known to conduct intramembrane cleavage of thrombomodulin. This prevents cleavage of the transmembrane domain of thrombomodulin in the K562 donor cells.

Expression of an Engineered Fusion Receptor Protein in Donor Cells

A protein is engineered to contain a rhomboid protease-sensitive transmembrane domain (derived from thrombomodulin), an intracellular cargo for delivery to the cytoplasm (GFP is the prototype fusion cargo in this example), and an extracellular domain consisting of a tethered CCL chemokine for mediating transfer. A plasmid expressing the engineered protein is transfected into donor K562 cells, after which successfully transformed cells can be identified by membrane GFP fluorescence. The shuttle protein is also identified by cell surface staining of the CCL domain with an antibody.

Cell-Cell Contact

Donor and acceptor cells (Lymphocytes expressing the D6 scavenger receptor) are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes.

Assay for Transfer of the Engineered Receptor

Cells are subjected to flow cytometry and acceptor cells (identified by cell identity labeling) are analyzed for surface expression of engineered receptor using an antibody against the extracellular CCL domain.

Assay for Release of Passenger Cargo

EGFP localization in acceptors is expected to be cytoplasmic after cleavage of the transmembrane domain by RHBDL2 in the acceptor. This is confirmed and quantified via fluorescent microscopy. EGFP signal is compared with control acceptors that had been exposed to donors expressing only EGFP (to measure increase over baseline based on any background cytoplasmic transfer).

### Example 5. Use of Progenitors as Donors for Cellular Reprogramming

For cell reprogramming, differentiated cells are treated to cause them to revert to a more pluripotent state, resembling a progenitor cell. This is most often accomplished with transcription factors, but can also be accomplished by stimulating combinations of surface signaling receptors. To further enable this sort of cell reprogramming, progenitor cells can be used as donors in order to install reprogramming receptors on differentiated cells that would otherwise lack the receptors. Exemplary experimental conditions for testing cell reprogramming are provided in this example.

Cell Isolation

Differentiated cells and their related progenitors are isolated from blood or tissue using negative selection.

Cell Membrane Labeling

Donors and are cultured in their preferred growth media at 37° C. in the presence of 5% CO₂. Prior to membrane transfer process assays, the donor cells are stained with a lipophilic, fluorescent dye according to manufacturer protocols (dyes for membrane staining include PKH26, PKH67, DiR, DiO, DiL). Extensive washing, including changing of tubes or wells, is crucial following dye exposure to remove all dye from the media. Following 3-5 washes, the cells are returned to suspension medium.

Cell Identity Labeling

To distinguish donor progenitor cells from acceptor differentiated cells, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes.

Cell-Cell Contact

Donor progenitor cells and differentiated acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Measurement

Transfer of membrane from donor to acceptor is measured by flow cytometry or fluorescence microscopy to identify acceptor cells that have acquired membrane from donors. These positive acceptors co-stain for their unique identity marker and the lipophilic dye that is applied only to the donor cells.

Receptor Stimulation

To induce reprogramming, ligands are added to the acceptor cells that bind to transproteins obtained from the progenitor donors.

Confirming Reprogramming

Stimulated acceptors are assayed for differentiation markers that indicate they have been reprogrammed and re-differentiated.

### Example 6. Autologous Transfer

Cell Isolation from Whole Blood

A sufficient quantity of patient blood is collected based on the required number of cells. Required cell numbers are determined by the concentration of the cells of interest in blood (or apheresis pack), the efficiency of transfer for a given cell pair (based on previous measurements), the ratio of donor to acceptor (based ratio optimization screens), the cell loss due to isolation (based on our pairwise measurements), and the number of acceptor cells desired as outputs. The desired donor and/or acceptor cells are each isolated by depletion of all other cell types. This can be done from a single blood draw or separate draws from a single individual for donor and/or acceptor, which produces greater yields (less target cell loss). Negative selection is done by staining with magnetically functionalized antibodies or fluorescent antibodies and subsequent exposure to a magnetic field or FACS. Staining buffers include Fc receptor blocking reagents to prevent FcR-antibody-mediated transfer, cell-cell adhesion, or antibody sequestration.

Cell Membrane Labeling

Donors and are cultured in their preferred growth media at 37° C. in the presence of 5% CO₂. Prior to assays, the donor cells are stained with a lipophilic, fluorescent dye according to manufacturer protocols (dyes for membrane staining include PKH26, PKH67, DiR, DiO, DiL). Extensive washing, including changing of tubes or wells, is crucial following dye exposure to remove all dye from the media. Following 3-5 washes, the cells are returned to suspension medium.

Cell Identity Labeling

To distinguish donor cells from acceptor cells, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Membrane Transfer Measurement

Transfer of membrane from donor to acceptor is measured by flow cytometry or fluorescence microscopy to identify acceptor cells that have acquired membrane from donors. These transfer-positive acceptors co-stain for their unique identity marker and the lipophilic dye that is applied only to the donor cells.

### Example 7. Allogenic Transfer

Cell Isolation from Whole Blood

Sufficient quantities of donor and acceptor blood are collected based on the required number of cells. Required cell numbers are determined by the concentration of the cells of interest in blood (or apheresis pack), the efficiency of transfer for a given cell pair (based on previous measurements), the ratio of donor to acceptor (based ratio optimization screens), the cell loss due to isolation (based on our pairwise measurements), and the number of acceptor cells desired as outputs. The desired donor and/or acceptor cells are each isolated by depletion of all other cell types. Negative selection is done by staining with magnetically functionalized antibodies or fluorescent antibodies and subsequent exposure to a magnetic field or FACS. Staining buffers include Fc receptor blocking reagents to prevent FcR-antibody-mediated transfer, cell-cell adhesion, or antibody sequestration.

Cell Membrane Labeling

Donors and are cultured in their preferred growth media at 37° C. in the presence of 5% CO₂. Prior to assays, the donor cells are stained with a lipophilic, fluorescent dye according to manufacturer protocols (dyes for membrane staining include PKH26, PKH67, DiR, DiO, DiL). Extensive washing, including changing of tubes or wells, is crucial following dye exposure to remove all dye from the media. Following 3-5 washes, the cells are returned to suspension medium.

Cell Identity Labeling

To distinguish donor cells from acceptor cells, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes. Alternatively, since the cells are allogenic, they can also be identified by antibody staining of patient specific surface markers, if available.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Measurement

Transfer of membrane from donor to acceptor is measured by flow cytometry or fluorescence microscopy to identify acceptor cells that have acquired membrane from donors. These positive acceptors co-stain for their unique identity marker and the lipophilic dye that is applied only to the donor cells.

### Example 8. Homotypic Transfer

The transfer of proteins among cells of the same type could be a powerful way to spread therapeutic effects in tissues.

Cell Isolation from Whole Blood

Cells of a single type are collected from patient blood via negative magnetic selection.

Cell Identity Labeling

To distinguish donor cells from acceptor cells, 10-90% of the cells are designated as donors and are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes. The remaining cells can be labeled with a dye that emits at a different wavelength or left unlabeled.

Cell Membrane Labeling

Prior to assays, 10-90% of the cells are designated as donor cells, and are stained with a lipophilic, fluorescent dye according to manufacturer protocols (dyes for membrane staining include PKH26, PKH67, DiR, DiO, DiL). Extensive washing, including changing of tubes or wells, is crucial following dye exposure to remove all dye from the media. Following 3-5 washes, the cells are returned to suspension medium.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes. Contact between the donor cells and acceptor cells is sufficient to drive transfer for some cell pairs.

Measurement

Transfer of membrane from donor to acceptor is measured by flow cytometry or fluorescence microscopy to identify acceptor cells that have acquired membrane from donors. These positive acceptors co-stain for their unique identity marker and the lipophilic dye that is applied only to the donor cells.

### Example 9. Donation of Overexpressed Cargo Molecule

Donors are modified to express high levels of proteins to be transferred to acceptors. Donors can be modified to express a cargo molecule comprising a protein either transiently or via stable genome integration.

Donor Cargo Molecule Overexpression

A mammalian expression construct is designed to express the desired cargo molecule from a strong promoter. The expression construct is transfected into donor cells. Successfully transformed cells are monocultured to allow for protein expression before co-culture with acceptors. Control donors are transfected with an empty plasmid.

Cell Identity Labeling

To distinguish donor cells from acceptor cells, 10-90% of the cells are designated as donors and are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes. The remaining cells can be labeled with a dye that emits at a different wavelength or left unlabeled.

Cell Membrane Labeling

Prior to assays, 10-90% of the cells are designated as donor cells, and are stained with a lipophilic, fluorescent dye according to manufacturer protocols (dyes for membrane staining include PKH26, PKH67, DiR, DiO, DiL). Extensive washing, including changing of tubes or wells, is crucial following dye exposure to remove all dye from the media. Following 3-5 washes, the cells are returned to suspension medium.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Measurement of Cargo Molecule on Acceptors

Transfer of membrane from donor to acceptor is measured by flow cytometry or fluorescence microscopy to identify acceptor cells that have acquired membrane from donors. These positive cells are also assayed for the overexpressed protein, e.g., cargo molecule, using antibodies. Antibody staining is then measured by flow cytometry or microscopy comparing acceptors that were exposed to cargo molecule-expressing donors versus donors with empty plasmids.

### Example 10. Donation of Exogenous Cargo Molecule

This example shows transfer of an exogenous cargo molecule from a donor cell to an acceptor cell.

Donors are modified to express engineered proteins for donation to acceptors.

Exogenous Cargo Molecule Expression

A mammalian expression construct is designed to express the desired cargo molecule from a strong promoter. The expression construct is transfected into donor cells. Successfully transformed cells are monocultured to allow for protein expression before co-culture with acceptors.

Cell Identity Labeling

To distinguish donor cells from acceptor cells, 10-90% of the cells are designated as donors and are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers or membrane dyes. The remaining cells can be labeled with a dye that emits at a different wavelength or left unlabeled.

Cell Membrane Labeling

Prior to assays, 10-90% of the cells are designated as donor cells, and are stained with a lipophilic, fluorescent dye according to manufacturer protocols (dyes for membrane staining include PKH26, PKH67, DiR, DiO, DiL). Extensive washing, including changing of tubes or wells, is crucial following dye exposure to remove all dye from the media. Following 3-5 washes, the cells are returned to suspension medium.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Contact between the donor cells and acceptor cells is sufficient to drive membrane transfer for some cell pairs.

Measurement of Cargo Molecule on Acceptors

Transfer of membrane from donor to acceptor is measured by flow cytometry or fluorescence microscopy to identify acceptor cells that have acquired membrane from donors. These positive cells are also assayed for the exogenous protein, e.g., cargo molecule, using antibodies. Antibody staining is then measured by flow cytometry or microscopy.

### Example 11. Driving Transfer of a Specific Cargo Molecule from a Donor to an Acceptor Via Bispecifics

This example demonstrates that soluble bispecific linkers can be used to designate a receptor on a donor cell for transfer by an acceptor cell. By tethering the two receptors together, internalization of the acceptor-side receptor drives transfer of the donor-side receptor into the acceptor. This strategy could be used in vitro (this example) or in vivo (example 5) to promote transfer among receptor pairs that do not transfer spontaneously between the two cell types.

Donor Cell Membrane Labeling

Donors are cultured in suspension media at 37° C. in the presence of 5% CO₂. Prior to transfer assays, the cells are transferred to staining buffer and stained with a lipophilic, fluorescent dye according to manufacturer protocols (dyes could include PKH26, PKH67, DiR, DiO, DiL, etc.) to track the lipid components of donor-derived membranes and whether there is transfer to acceptor cells. Extensive washing, including changing of tubes or wells, is crucial following dye exposure to remove all dye from the media. Following 3-5 washes, the cells are returned to suspension medium.

Cell Identity Labeling

To distinguish donor cells from acceptor cells, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Bispecific Design

The basic design of bispecifics requires 2 affinity domains connected by a linker. The affinity domains are selected based on binding to the receptors of interest on the donor cell and acceptor cell. They can be assembled from proteins or protein domains known to bind the receptor of interest or antibody-derived variable domains. Target receptors on acceptors are chosen based on frequency and strength of uptake and recycling activity in the acceptor cells. Target receptors on donors are the desired cargo molecule or receptors that have been observed to passively shuttle the desired cargo molecule, usually through binding interactions with the target receptor or lipid raft co-occupancy.

Co-Culture in the Presence of Bispecific Linker

To co-culture, the donors and acceptors are admixed at ratios ranging from 1:1 to 10:1 in the presence of the soluble bispecific linker. The linker can be added to donor cells just prior to mixture with acceptor cells, but should not be applied to acceptor cells in the absence of donors. This could cause internalization of the acceptor target receptor before linker binding to the donor target receptor. Bispecific linker should be dosed to saturate the donor target receptor using an excess of linker that is at least double the number of donor target receptor molecules in the sample. Overall cell concentration must meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. Bispecific-mediated conjugation between the donor and acceptor cells promotes transfer of the donor target receptor by the acceptor.

Measurement

Transfer of membrane from donors to the acceptors is measured by flow cytometry to identify acceptor cells that have acquired lipophilic dye-stained membrane from donors. These positive acceptors co-stain for their unique identity marker and the lipophilic dye that is applied only to the donors.

### Example 12. Use of a Donor-Expressed Membrane-Associated Agent to Drive Transfer by an Acceptor

This example demonstrates the use of a donor cell that is engineered to express a membrane-associated agent, e.g., a chimeric surface receptor, that is designed to be transferred to a specific acceptor. The chimeric receptor itself, a cargo molecule that transfers along with the chimeric receptor, or both may be intended for transfer.

Chimeric Receptor Design

The basic design of chimeric receptor requires an affinity domain (or domains), e.g., corresponding to an extracellular moiety, that is connected to a membrane anchoring domain (such as a transmembrane domain), e.g., corresponding to a membrane-associated moiety. An intracellular domain, e.g., corresponding to an intracellular moiety, may also be included to mediate intracellular signaling or binding to a cargo molecule. The affinity domain(s) is selected based on binding to receptors on the acceptor cell that are known to mediate transfer. Affinity domains can be assembled from proteins or protein domains known to bind the receptor of interest or antibody-derived variable domains. Target receptors on acceptors are chosen based on frequency and strength of uptake and recycling activity of those receptors in the acceptor cells. Linker domains or epitope-containing domains (for tracking chimeric receptors with corresponding antibodies) may be included between these basic components to provide spacing, flexibility, and/or chimeric receptor detection. The complete chimeric receptor protein is introduced into donor cells via lentiviral transfection to generate a stably-expressing donor cell line.

Co-Culture of Engineered Donors with Acceptors

To co-culture, the chimeric receptor-edited donors are mixed with acceptors at ratios ranging from 1:1 to 10:1. Overall cell concentration must meet or exceed the numbers required for all cells to settle to the bottom of the plate or well in a monolayer. This ensures that donor cells come in contact with acceptor cells after settling to the culture surface. The settling process can be accelerated via a 5-minute centrifugation with a swinging bucket rotor at 100 g. Settled cells are left undisturbed for 90 minutes. Chimeric receptor-mediated conjugation between the donor and acceptor cells promotes transfer of the donor target receptor by the acceptor.

Cell Membrane Labeling (Optional)

The engineered donor cells and/or acceptors can be subjected to labeling prior to co-culture. Isolated donor cells are cultured in suspension media at 37° C. in the presence of 5% CO₂. The cells are transferred to staining buffer and stained with a lipophilic, fluorescent dye according to manufacturer protocols (dyes could include PKH26, PKH67, DiR, DiO, DiL, etc.) to track the lipid components of donor-derived membranes and whether there is transfer to acceptor cells. Extensive washing, including changing of tubes or wells, is crucial following dye exposure to remove all dye from the media. Following 3-5 washes, the cells are returned to sterile saline for adoptive transfer back to experimental mice.

Donor Cell Identity Labeling (Optional)

To distinguish donor cells from acceptor cells, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cell type-specific markers.

Acceptor Cell Isolation and Measurement

After co-culture, the acceptor cells are isolated for analysis. Isolation can be conducted using FACS or MACS for surface markers on the acceptor cell or optional markers. Lipophilic dye positivity is quantified via FACS as a measure of the acceptor activity by the target cell type. The chimeric receptor can also be directly measured if an epitope module was included in its extracellular structure. Alternatively, if the chimeric receptor is designed to bind and co-transfer other proteins as cargo molecules, those proteins can be assayed directly with antibodies for surface-exposed proteins or downstream western blotting of lysates of putative acceptor cells.

### Example 13. Demonstration of Trafficking Receptor Transfer

This example demonstrates the use of trafficking receptors as cargo molecules to redirect acceptor cells to specific tissues or sub-tissue sites within the body. The trafficking receptor only needs to persist on the acceptor cell for long enough to mediate the migration. Arrival at the target site allows the cell to perform an innate function or the cell can be modified with additional signals to alter behavior.

Donor Cell Membrane Labeling

Donors are cultured in suspension media at 37° C. in the presence of 5% CO₂. Prior to assays, the cells are transferred to staining buffer and stained with a lipophilic, fluorescent dye according to manufacturer protocols (dyes could include PKH26, PKH67, DiR, DiO, DiL, etc.) to track the lipid components of donor-derived membranes and whether there is transfer to acceptor cells. Extensive washing, including changing of tubes or wells, is crucial following dye exposure to remove all dye from the media. Following 3-5 washes, the cells are returned to suspension medium.

Cell Identity Labeling

To distinguish the donor cells from the acceptor cell, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Expression of the Chemokine Receptor, CCR7, in Donor Cells

A plasmid expressing CCR7 and EGFP is transfected into K562 cells, after which successfully transformed cells can be identified by EGFP expression. CCR7 expression is confirmed on the exterior of the EGFP-positive cells via flow cytometry using an antibody against the extracellular portion of CCR7. For in vivo experiments, the plasmid is modified to include luciferase expression.

Cell-Cell Contact

The CCR7-expressing K562 donors are initially monocultured. To the monocultures, NHYG-1 cells, an NK cell line, are added as acceptor cells. Co-culture ratios range from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes. The NK cells target K562 cells for killing, but transfer CCR7 in the process.

Cell Trafficking In Vitro

To test function of CCR7 on the acceptor NHYG-1 cells, the cells are added to the upper portion of Transwell plates and assayed for migration to the lower chamber, which is supplemented with CCL19 and CCL21 ligands.

Cell Trafficking In Vivo

To further test the function of CCR7 transprotein in NHYG-1 cells, the cells are injected into the tail veins of mice. One day after injection, the mice are sacrificed, and lymph nodes are resected for ex vivo luciferase imaging. Control mice are injected with NHYG-1 cells that were previously co-cultured with unmodified K562 cells.

### Example 14. Demonstration of Activation Receptor Transfer

This example demonstrates the delivery of activation/inhibition receptors as cargo molecules to modify the behavior of acceptor cells.

Donor Cell Membrane Labeling

Donors are cultured in suspension media at 37 C in the presence of 5% CO₂. Prior to assays, the cells are transferred to staining buffer and stained with a lipophilic, fluorescent dye according to manufacturer protocols (dyes could include PKH26, PKH67, DiR, DiO, DiL, etc.) to track the lipid components of donor-derived membranes and whether there is transfer to acceptor cells. Extensive washing, including changing of tubes or wells, is crucial following dye exposure to remove all dye from the media. Following 3-5 washes, the cells are returned to suspension medium.

Cell Identity Labeling

To distinguish the donor cells from the acceptor cell, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Expression of the Activation Receptor, IL2RA, in Donor Cells

A plasmid expressing IL2RA and EGFP is transfected into B cells, after which successfully transformed cells can be identified by EGFP expression. IL2RA expression is confirmed on the exterior of the EGFP-positive cells via flow cytometry using an antibody against the extracellular portion.

Cell-Cell Contact

The IL2RA-expressing B cell donors are initially monocultured. To the monocultures, Jurkat cells, a T cell line, are added as acceptor cells. Co-culture ratios range from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes for transfer to occur.

Cell Activity

Jurkat cell growth is assessed in the presence of IL-2 comparing Jurkats exposed to IL2RA B cells versus WT B cells. Proliferation is measured at several time points.

### Example 15. Demonstration of Reprogramming Receptor Transfer

This example demonstrates donation of a membrane receptor, Basp1, that signals upstream of the reprogramming factor, Sox2. Basp1 stimulation has been shown to functionally mimic Sox2 in reprogramming experiments (Blanchard et al. Nature Biotech 2017)

Donor Cell Membrane Labeling

Donors are cultured in suspension media at 37° C. in the presence of 5% CO₂. Prior to assays, the cells are transferred to staining buffer and stained with a lipophilic, fluorescent dye according to manufacturer protocols (dyes could include PKH26, PKH67, DiR, DiO, DiL, etc.) to track the lipid components of donor-derived membranes and whether there is transfer to acceptor cells. Extensive washing, including changing of tubes or wells, is crucial following dye exposure to remove all dye from the media. Following 3-5 washes, the cells are returned to suspension medium.

Cell Identity Labeling

To distinguish the donor cells from the acceptor cell, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Expression of the reprogramming receptor, Basp1, in donor cells

A plasmid co-expressing Basp1 and EGFP is transfected into donor cells, after which successfully transformed cells can be identified by EGFP expression. Basp1 expression is confirmed on the exterior of the EGFP-positive cells via flow cytometry using an antibody against the extracellular portion.

Expression of other reprogramming factors in acceptor cells

A second plasmid is designed to deliver c-Myc, Oct4, and Klf4, but lacks Sox2 (the experiment is intended to show reprogramming by replacing Sox2 with Basp1). This plasmid is transfected into mouse embryonic fibroblasts (MEFs), which are the acceptors and cells to be reprogrammed.

Cell-Cell Contact

The Basp-1-expressing donors are initially monocultured. To the monocultures, transformed MEFs are added as acceptor cells. Co-culture ratios range from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes for transfer to occur.

Reprogramming Assays

Ability of the MEFs to be reprogrammed to a pluripotent state is measured by expression of Oct4 and Nanog, measured by QPCR and/or immunofluorescence. Cells exposed to Basp-1 agonists are compared to cells treated with a vehicle alone to determine the functional contribution of Basp-1 signaling to reprogramming.

### Example 16. Therapeutic Protein Transfer

This example demonstrates the transfer of a therapeutic protein to replace a loss of function in acceptor cells.

Donor Cell Membrane Labeling

Donors are cultured in suspension media at 37° C. in the presence of 5% CO₂. Prior to assays, the cells are transferred to staining buffer and stained with a lipophilic, fluorescent dye according to manufacturer protocols (dyes could include PKH26, PKH67, DiR, DiO, DiL, etc.) to track the lipid components of donor-derived membranes and whether there is transfer to acceptor cells. Extensive washing, including changing of tubes or wells, is crucial following dye exposure to remove all dye from the media. Following 3-5 washes, the cells are returned to suspension medium.

Cell Identity Labeling

To distinguish the donor cells from the acceptor cell, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Expression of the Therapeutic Protein, CFTR, in Donor Cells

A plasmid co-expressing CFTR and EGFP is transfected into donor pulmonary dendritic cells (pDCs), after which successfully transformed cells can be identified by EGFP expression. CFTR expression is confirmed on the exterior of the EGFP-positive cells via flow cytometry using an antibody against the extracellular portion.

Cell-Cell Contact

The CFTR-expressing pDC donors are initially monocultured. To the monocultures, CFTR−/− airway epithelial cells (AECs) are added as acceptor cells. Co-culture ratios range from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5 minute centrifugation with a swinging bucket rotor at 100 g. Settled cells are left undisturbed for 90 minutes for transfer to occur.

Assay for Rescued CFTR

The AECs are stained for CFTR protein and analyzed via flow cytometry.

### Example 17. Transfer of a Cargo Molecule Comprising a Synthetic Moiety

This example demonstrates installation of a synthetic protein containing a traceable fluorescent domain.

Cell Identity Labeling

To distinguish the donor cells from the acceptor cell, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Expression of Cell Surface-Localized GFP

A plasmid expressing a transferrin-GFP fusion protein is transfected into donor cells, after which successfully transformed cells can be identified by surface GFP expression.

Cell-Cell Contact

Donor and acceptor cells are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes. Contact between the donor cells and acceptor cells is sufficient to drive transfer for some cell pairs.

Assay for Transferred Surface GFP

Cells are subjected to flow cytometry and acceptor cells (identified by cell identity labeling) are analyzed for surface GFP expression. Anti-GFP antibodies are also applied to confirm that the GFP is surface-exposed, not just membrane-associated.

### Example 18. Transfer of a Membrane-Associated Agent Comprising a Synthetic Moiety

This example demonstrates the transfer of a synthetic protein containing an extracellular domain (e.g., corresponding to an extracellular moiety) that binds to a protein expressed on the surface of a desired acceptor (e.g., corresponding to a target cell moiety). The binding drives transfer from the engineered cell to the acceptor. In this specific example, a membrane-tethered chemokine is engineered into a donor cell line (K562) to target a scavenger receptor expressed on vascular endothelial cells (intended acceptor), which express the D6 scavenger receptor (that binds broadly to CC-group receptors).

Cell Identity Labeling

To distinguish the donor cells from the acceptor cell, one or both of the cell types are labeled with unique non-transferrable or minimally transferred dyes that covalently crosslink to cytoplasmic structures (Thermo CellTracker Green or Thermo CellTracker Orange, RedDot1, etc.). Fluorescence emission wavelengths are chosen to avoid overlap with any other identity trackers in use and the membrane dye from the previous step. As an alternative identity labeling strategy, fluorescent antibodies are used to label cells.

Expression of an Engineered Donor-Driving Receptor

A protein is engineered to contain a transmembrane domain connected via a short linker peptide to a CCL-group chemokine. A plasmid expressing the engineered protein is transfected into donor K562 cells, after which successfully transformed cells can be identified by GFP co-expressed from the same plasmid.

Cell-Cell Contact

Donor and acceptor cells (vascular endothelial cells) are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes.

Assay for Transfer of the Engineered Receptor

Cells are subjected to flow cytometry and acceptor cells (identified by cell identity labeling) are analyzed for surface expression of engineered receptor using an antibody against the extracellular CCL domain.

### Example 19. Transfer of a Cargo Molecule Using a Membrane-Associated Agent as a Shuttle

This example demonstrates the use of engineered proteins as trafficking adaptors between cells. The membrane-associated agents are designed to be modular with 3 major domains: an intracellular moiety that binds a cargo molecule, a membrane-associated moiety comprising a transmembrane domain that localizes the protein to the plasma membrane, and an extracellular moiety comprising a targeting domain that gives the membrane-associated agent specificity for donation to specific acceptor cells. The cargo molecule could be any agent in the donor cell that can be bound by the intracellular moiety, including proteins, RNAs, and organelles. In some cases, the cargo molecule can be expressed via an introduced plasmid.

Expression of a Membrane-Associated Agent and GFP in Donor Cells

A protein is engineered to contain a transmembrane domain, an intracellular binding domain containing scFv against GFP, and an extracellular domain consisting of a tethered CCL chemokine. A plasmid expressing the membrane-associated agent is transfected into donor K562 cells, after which successfully transformed cells can be identified with an antibody against the chemokine domain or expression of co-expressed EGFP, which should localize to the inner leaflet of the cell membrane through binding to the membrane-associated agent's intracellular moiety-scFv. A control donor is also generated which lacks the membrane-associated agent, but still expresses EGFP.

Cell-Cell Contact

Donor and acceptor cells (Lymphocytes expressing the D6 scavenger receptor) are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes.

Assay for Transfer of the Membrane-Associated Agent and EGFP Passenger Cargo

Cells are subjected to flow cytometry and acceptor cells (identified by cell identity labeling) are analyzed for surface expression of EGFP using an antibody against the extracellular CCL domain. EGFP is expected in only the acceptors which contacted shuttle-containing donors.

### Example 20. Transfer of Cargo Molecule to Acceptor Cytoplasm Using a Cleavable Membrane-Associated Agent

This example demonstrates the use of engineered proteins as trafficking adaptors between cells. In this variation, the membrane-associated agent contains a domain that can be cleaved by endogenous enzymes in the acceptor, allowing the cargo molecule to be released into the cytoplasm of the acceptor. The enzymes are knocked out in the donor cells to prevent cleavage prior to transfer.

Knock-Out of Intramembrane Protease in Donor Cells

Targeting constructs are introduced into K562 donor cells that are designed to knock out RHBDL2, a rhomboid protease that is known to conduct intramembrane cleavage of thrombomodulin. This prevents cleavage of the transmembrane domain of thrombomodulin in the K562 donor cells.

Expression of a Membrane-Associated Agent in Donor Cells

A protein is engineered to contain a rhomboid protease-sensitive membrane-associated moiety comprising a transmembrane domain (derived from thrombomodulin), an intracellular moiety containing scFv against GFP, and an extracellular moiety comprising a tethered CCL chemokine. A plasmid expressing the membrane-associated agent is transfected into donor K562 cells, after which successfully transformed cells can be identified by GFP co-expressed from the same plasmid (should localize to the membrane through binding the shuttle protein). The membrane-associated agent is also identified by cell surface staining of the CCL domain with an antibody.

Cell-Cell Contact

Donor and acceptor cells (Lymphocytes expressing the D6 scavenger receptor) are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes.

Assay for Transfer of the Membrane-Associated Agent

Cells are subjected to flow cytometry and acceptor cells (identified by cell identity labeling) are analyzed for surface expression of engineered receptor using an antibody against the extracellular CCL domain.

Assay for Release of Cargo Molecule

EGFP localization in acceptors is expected to be cytoplasmic after cleavage of the transmembrane domain by RHBDL2 in the acceptor. This is confirmed and quantified via fluorescent microscopy.

### Example 21. Transfer of mRNA Cargo Molecule Using an Acceptor-Cleavable Membrane-Associated Agent

This example demonstrates the use of engineered proteins as trafficking adaptors between cells. In this variation, the membrane-associated agent contains a domain that can be cleaved by endogenous enzymes in the acceptor, allowing the cargo molecule to be released into the cytoplasm of the acceptor. The enzymes are knocked out in the donor cells to prevent cleavage prior to transfer.

Knock-Out of Intramembrane Protease in Donor Cells

Targeting constructs are introduced into K562 donor cells that are designed to knock out RHBDL2, a rhomboid protease that is known to conduct intramembrane cleavage of thrombomodulin. This prevents cleavage of the transmembrane domain of thrombomodulin in the K562 donor cells.

Expression of a Membrane-Associated Agent in Donor Cells

A membrane-associated agent is engineered to contain a rhomboid protease-sensitive membrane-associated moiety comprising a transmembrane domain (derived from thrombomodulin), an intracellular moiety containing an MS2 bacteriophage coat protein, and an extracellular moiety consisting of a tethered CCL chemokine. A plasmid expressing the membrane-associated agent is transfected into donor K562 cells, after which successfully transformed cells can be identified by GFP co-expressed from the same plasmid. The protein orientation can be confirmed using an antibody against the chemokine domain.

Expression of mRNA Cargo Molecule

The same plasmid that expresses the membrane-associated agent is designed to also express EGFP with an MS2 binding sequence in the 3′ UTR. The mRNA transcribed from this ORF is capable of binding to the intracellular MS2 domain of the membrane-associated agent. This membrane-associated agent-mRNA complex can be transferred to acceptor cells via binding of the extracellular moiety to proteins on the surface of an acceptor.

Cell-Cell Contact

Donor and acceptor cells (Lymphocytes expressing the D6 scavenger receptor) are monocultured separately in suspension media after labeling. To co-culture, the acceptor cells are added to the donor cell cultures at ratios ranging from 1:1 to 10:1. Overall cell concentration should meet or exceed the numbers required for all cells to settle to the bottom of the plate or well in a monolayer. This ensures that all cells are in contact with other cells after settling to the culture surface. The settling process can be accelerated via a 5-minute centrifugation with a swinging bucket rotor at 100 g. Settled cells are left undisturbed for 90 minutes.

Assay for Transfer of the Engineered Receptor

Cells are subjected to flow cytometry and acceptor cells (identified by cell identity labeling) are analyzed for surface expression of membrane-associated agent using an antibody against the extracellular CCL domain.

Assay for mRNA Function in Acceptors

Translation of the EGFP mRNA is measured in acceptors following cell-cell contact relative to cells that were contacted with donors that do not express the membrane-associated agent (to account for the possibility of transfer of donor-translated EGFP protein). EGFP intensity is measured via microscopy.

### Example 22. Simple Membrane Transfer from a Donor to an Acceptor

This example demonstrates transfer of cell surface material (membrane, including lipids and proteins) from an immortalized donor cell line (K562) to an acceptor cell (THP-1). Cell-cell contact is accomplished by co-culturing the cells.

Donor Cell Membrane Labeling

K562 donor cells are cultured in their preferred growth media at 37° C. in the presence of 5% CO₂. The cells are washed in PBS and stained with PKH26, a lipophilic fluorescent dye, according to manufacturer protocols. Following 3-5 washes and quenching with FBS, the cells are returned to suspension medium.

Cell Identity Labeling

To distinguish donor cells from acceptor cells, one or both of the cell types are labeled with unique, non-transferable intracellular dyes, intracellular fluorescent proteins, or antibodies against non-transferable surface proteins. In this case, we label K562 donor cells with CellTracker Green and THP-1 cells with CellTracker Deep Red (ThermoFisher). These markers are more reliable than surface protein markers since they cannot be significantly transferred.

Assay

Donor and acceptor cells are monocultured separately in suspension media after labeling. To co-culture, 1.5×10⁵ acceptor cells are added to 1.5×10⁵ donor cells in a 96-well plate for a co-culture ratio of 1:2. At this plate surface area and cell density, all cells are in contact with other cells after settling to the culture surface. The settling process is accelerated via a 5-minute centrifugation with a swinging bucket rotor at 100 g and no brake. Settled cells are left undisturbed for 60 minutes in the incubator (37 C, 5% CO₂, humidified). Contact between the donor cells and acceptor cells is sufficient to drive transfer for this cell pairing.

Measurement

Transfer of membrane from donor (K562) to acceptor (THP-1) is measured by flow cytometry and fluorescence microscopy to identify acceptor cells that have acquired PKH26-labelled membrane from donors. These positive acceptors co-stain for their unique identity marker (Deep Red CellTracker dye) and the lipophilic dye that is applied only to the donor cells.

### Example 23. Assay for Cytoplasmic Access and Function of Transproteins in Acceptors

Transfer of proteins to acceptors is intended to provide the acceptor with functions that are similar to the function of the protein in the donor cells. This requires that the transproteins localize to the membrane of the acceptor cells with similar orientation and access to the cytoplasmic compartment, for proper signaling.

Donor Cell Membrane Labeling

K562 donor cells are cultured in preferred growth media at 37° C. in the presence of 5% CO₂. The cells are washed in PBS and stained with PKH26, a lipophilic fluorescent dye, according to manufacturer protocols. Following 3-5 washes and quenching with FBS, the cells are returned to suspension medium.

Donor Identity Labeling

Donor K562 cells are labeled with CellTracker Deep Red (ThermoFisher) according to manufacturer protocols.

Acceptor Cell Labeling and Substrate Loading

Acceptor THP-1 cells are loaded with CCF2-AM (ThermoFisher), which is a cell-permeable substrate that fluoresces green after cell entry and cleavage by endogenous esterases. This green fluorescence acts to identify THP-1 cells as acceptors. The CCF2-AM can be cleaved a second time, by Beta-Lactamase, which converts the fluorescent signal to a blue emission. This allows Beta-Lactamase activity to be measured as cytoplasmic blue fluorescence in acceptors.

Expressing a Cytoplasm-Facing Reporter Protein in Donors and Acceptors

Plasmid constructs that encode membrane-tethered enzyme reporters are used to label K562 donor cells. Specifically, we use a chimeric receptor which encodes an enzyme reporter, Beta-Lactamase, fused to a myristoylation/palmitoylation signal from Lck, which localizes the receptor to the inner leaflet of the donor plasma membrane. A 6-amino acid lumio tag is included in the linker to identify the protein itself after transfer and can be visualized regardless of orientation in the membrane via addition of a simple reagent that fluoresces upon binding the lumio tag. Plasmid encoding the chimeric receptor, termed MP-Lumio-Blac, is transiently transfected into the Kj562 donor cells using Lipofectamine 3000, according to manufacturer protocols. After expression of the receptor is confirmed by a secondary, plasmid-encoded fluorescent marker, donors are ready for co-culture.

Assay

Donor and acceptor cells are monocultured separately in suspension media after labeling. To co-culture, 1.5×10⁵ acceptor cells are added to 1.5×10⁵ of the receptor-expressing donor cells in a 96-well plate for a co-culture ratio of 1:2. At this plate surface area and cell density, all cells are in contact with other cells after settling to the culture surface. The settling process is accelerated via a 5-minute centrifugation with a swinging bucket rotor at 100 g and no brake. Settled cells are left undisturbed for 60 minutes in the incubator (37° C., 5% CO₂, humidified). Contact between the donor cells and acceptor cells is sufficient to drive transfer.

Assaying Membrane-Associated Agent Localization and Orientation

Transfer of membrane from donor (K562) to acceptor (THP-1) is measured by flow cytometry and fluorescence microscopy to identify acceptor cells that have acquired PKH26-labelled membrane from donors. Successful transfer of the MP-Lumio-Blac receptor is confirmed by applying a lumio ligand that emits red fluorescence (Thermo) to the cells and checking for co-expression with the acceptor cytoplasm marker, CCF2-AM (green). The lumio tag should be membrane associated if the receptor has transferred to the acceptor plasma membrane. To assay cytoplasmic access and proper orientation of MP-Lumio-Blac, blue fluorescence is also measured (resulting from cleavage of CCF2-AM by Beta-Lactamase). If the receptor is properly oriented, the blue fluorescence should accumulate throughout the cytoplasm of the acceptor cells. The ratio of blue to green fluorescence in the acceptors provides quantification of relative Beta-Lactamase activity across cells.

### Example 24. Engineering and Use of Membrane-Associated Agents with Acceptor-Binding Activity

This shows the use of a membrane-associated agent, e.g., a chimeric receptor, that is engineered to bind to an acceptor (e.g., a target cell moiety) and be transferred to the acceptor. The example also includes testing for properly-localized function of the acceptor.

Donor Identity Labeling

Donor Huvec cells are labeled with CellTracker Deep Red (ThermoFisher) according to manufacturer protocols.

Acceptor Cell Labeling and Substrate Loading

Acceptor Jurkat cells are loaded with CCF2-AM (ThermoFisher), which is a cell-permeable substrate that fluoresces green after cell entry and cleavage by endogenous esterases. This green fluorescence acts to identify THP-1 cells as acceptors. The CCF2-AM can be cleaved a second time, by Beta-Lactamase, which converts the fluorescent signal to a blue emission. This allows Beta-Lactamase activity to be measured as cytoplasmic blue fluorescence in acceptors.

Expressing a Chimeric Receptor with Acceptor-Binding Activity

Plasmid constructs that encode membrane-tethered enzyme reporters are used to label Huvec donor cells. Specifically, a membrane-associated agent, e.g., chimeric receptor, is used comprising an enzyme reporter, Beta-Lactamase, fused to the transmembrane and extracellular domains of E-selectin, which is known to bind adhesion receptors on activated T cells. A 6-amino acid lumio tag is included in the linker to identify the protein itself after transfer and can be visualized regardless of orientation in the membrane via addition of a simple reagent that fluoresces upon binding the lumio tag. Plasmid encoding the chimeric receptor, termed Selectin-Lumio-Blac, is transiently transfected into the Huvec donor cells using Lipofectamine 3000, according to manufacturer protocols. After expression of the receptor confirmed by a secondary, plasmid-encoded fluorescent marker, donors are ready for co-culture.

Assay

Donor and acceptor cells are monocultured separately in suspension media after labeling. To co-culture, 1.5×10⁵ acceptor cells are added to 1.5×10⁵ of the receptor-expressing donor cells in a 96-well plate for a co-culture ratio of 1:2. At this plate surface area and cell density, the donor huvecs form a complete monolayer at the bottom of the well. Thus, donor and acceptor cells are in contact with other after the jurkats settle to the huvec monolayer. The settling process is accelerated via a 5-minute centrifugation with a swinging bucket rotor at 100 g and no brake. Settled cells are left undisturbed for 60 minutes in the incubator (37° C., 5% CO₂, humidified). The assay is performed in parallel wells using both activated and non-activated Jurkat cells as acceptors.

Assaying Transfer, Localization, and Orientation

Successful transfer of the Selectin-Lumio-Blac receptor is confirmed by applying a lumio ligand that emits red fluorescence (Thermo) to the cells and checking for co-expression with the acceptor cytoplasm marker, CCF2-AM (green). The lumio tag should be membrane associated if the receptor has transferred to the acceptor plasma membrane. To assay cytoplasmic access and proper orientation of Selectin-Lumio-Blac, blue fluorescence is also measured (resulting from cleavage of CCF2-AM by Beta-Lactamase). If the receptor is properly oriented, the blue fluorescence should accumulate throughout the cytoplasm of the acceptor cells. The ratio of blue to green fluorescence in the acceptors provides quantification of relative Beta-Lactamase activity across cells.

### Example 25. Transfer of Activation/Inhibition Receptors to Cells

This example shows the use of donors to modify the state of a cell, in this case its activation state

Donor Cell Membrane Labeling

Ramos donor cells are cultured in their preferred growth media at 37° C. in the presence of 5% CO₂. The cells are washed in PBS and stained with PKH26, a lipophilic fluorescent dye, according to manufacturer protocols. Following 3-5 washes and quenching with FBS, the cells are returned to suspension medium.

Cell Identity Labeling

To distinguish donor cells from acceptor cells, one or both of the cell types are labeled with unique, non-transferable intracellular dyes, intracellular fluorescent proteins, or antibodies against non-transferable surface proteins. In this case, we label Ramos donor cells with CellTracker Green and Jurkat cells with CellTracker Deep Red (ThermoFisher). These markers are more reliable than surface protein markers since they cannot be significantly transferred.

Overexpression of Membrane-Associated Agent Comprising Activation Receptor

IL-1R type I is involved in T cell activation through IL-1. Since the type II receptor (which is inhibitory rather than activating) competes with the type I receptor for IL-1, increasing the relative amount of the type I variant should result in greater ability of T cells to be activated by IL-1. To overexpress IL-1R on Ramos donors, we transiently transfect them with a plasmid expressing IL-1R type I from a strong CMV promoter. Plasmid-expressing Ramos cells are selected based on a plasmid-expressed reporter and used for assays.

Assay

Donor and acceptor cells are monocultured separately in suspension media after labeling. To co-culture, 1.5×10⁵ acceptor cells are added to 1.5×10⁵ donor cells in a 96-well plate for a co-culture ratio of 1:2. At this plate surface area and cell density, all cells are in contact with other cells after settling to the culture surface. The settling process is accelerated via a 5-minute centrifugation with a swinging bucket rotor at 100 g and no brake. Settled cells are left undisturbed for 60 minutes in the incubator (37° C., 5% CO₂, humidified). Contact between the donor cells and acceptor cells is sufficient to drive transfer for this cell pairing.

Measurement

Transfer of membrane from donor (Ramos) to acceptor (Jurkat) is measured by flow cytometry and fluorescence microscopy to identify acceptor cells that have acquired PKH26-labelled membrane from donors. These positive acceptors co-stain for their unique identity marker (Deep Red CellTracker dye) and the lipophilic dye that is applied only to the donor cells.

Functional Assays—Measuring Change in Cell Behavior

Jurkat activation in the presence of IL-1 is measured using T cell activation assays, including IFN-γ release.

### Example 26. Transfer of a Cargo Molecule Comprising Therapeutic Moiety as a Bystander

Chimeric antigen receptors (CARs), when expressed on T or NK cell plasma membranes, allow the cells to kill target cells that express surface proteins recognized by the CAR's extracellular antigen-binding scFv domain. CARs are currently installed in T cells or NK cells by genetic engineering. Here, we demonstrate transfer of a therapeutic chimeric antigen receptor (CAR) as a cargo molecule to Jurkat cells (T cell line) using a donor that over-expresses both the CAR and a membrane-associated agent, e.g., a transfer-inducing receptor. The CAR is intended to transfer as a bystander rather than by direct binding of the CAR itself to a target cell moiety on the acceptor.

Expressing Cargo Molecule in Donors

A plasmid construct that encodes a CD19-targeting CAR is used to transiently transfect K562 cells, generating a CD19 CAR-overexpressing donor cell line. The plasmid also overexpresses HLA-G.

Cell Membrane Labeling

The K562 cell donors are cultured in preferred growth media at 37 C in the presence of 5% CO₂. Prior to assays, the K562 cells are stained with PKH26, to track transfer of membrane to acceptor cells. Extensive washing, including changing of tubes or wells, is crucial following dye exposure to remove all dye from the media. Following 3-5 washes, the cells are returned to suspension medium.

Assay

Donor and acceptor cells are monocultured separately in suspension media after labeling. To co-culture, 1.5×10⁵ acceptor cells are added to 1.5×10⁵ donor cells in a 96-well plate for a co-culture ratio of 1:2. At this plate surface area and cell density, all cells are in contact with other cells after settling to the culture surface. The settling process is accelerated via a 5-minute centrifugation with a swinging bucket rotor at 100 g and no brake. Settled cells are left undisturbed for 60 minutes in the incubator (37° C., 5% CO₂, humidified).

Measurement

Transfer of membrane from K562 to Jurkat cells is measured by flow cytometry and fluorescence microscopy to identify cells that have acquired PKH26.

Measurement of Cargo Function in Acceptors

Detection of the CAR on the acceptor cells (positive for the appropriate CellTracker dye) suggests proper orientation of the CAR on the T cell membrane. As a definitive functional test, CAR function is measured in the acceptor cells using cytotoxicity assays. Specifically, positive acceptor cells are exposed to CD19-expressing target cells. Target cell cytotoxicity is assayed by loss of fluorescence or other cell viability assays.

### Example 27. Comprehensive Assays for Functional Changes in Acceptors

To characterize changes in acceptor cell states, gene expression analysis is used.

Donor Cell Membrane Labeling

K562 donor cells are cultured in their preferred growth media at 37° C. in the presence of 5% CO₂. The cells are washed in PBS and stained with PKH26, a lipophilic fluorescent dye, according to manufacturer protocols. Following 3-5 washes and quenching with FBS, the cells are returned to suspension medium.

Cell Identity Labeling

To distinguish donor cells from acceptor cells, one or both of the cell types are labeled with unique, non-transferable intracellular dyes, intracellular fluorescent proteins, or antibodies against non-transferable surface proteins. In this case, we label K562 donor cells with CellTracker Green and THP-1 cells with CellTracker Deep Red (ThermoFisher). These markers are more reliable than surface protein markers since they cannot be significantly transferred.

Assay

Donor and acceptor cells are monocultured separately in suspension media after labeling. To co-culture, 1.5×10⁵ acceptor cells are added to 1.5×10⁵ donor cells in a 96-well plate for a co-culture ratio of 1:2. At this plate surface area and cell density, all cells are in contact with other cells after settling to the culture surface. The settling process is accelerated via a 5-minute centrifugation with a swinging bucket rotor at 100 g and no brake. Settled cells are left undisturbed for 60 minutes in the incubator (37° C., 5% CO₂, humidified). Contact between the donor cells and acceptor cells is sufficient to drive transfer for this cell pairing.

Measurement

Transfer of membrane from donor (K562) to acceptor (THP-1) is measured by flow cytometry and fluorescence microscopy to identify acceptor cells that have acquired PKH26-labelled membrane from donors. These positive acceptors co-stain for their unique identity marker (Deep Red CellTracker dye) and the lipophilic dye that is applied only to the donor cells.

Acceptor Cell State Profiling

To confirm transfer, RNA is collected from acceptor cells and processed according to existing protocols for RNA Seq. Gene expression profiles will be compared between acceptors that have been co-cultured with donor cells versus monocultured (the pre-transfer condition). Comparative gene expression analysis provides a profile of functional changes driven by the co-culture. The cells are also co-cultured in a Transwell system to determine effects driven by non-contact forms of communication such as cytokine signaling and exosome release.

### Example 28. Density-Dependent Membrane Transfer from Ramos Cells to Jurkat Cells

This example demonstrates contact-dependent transfer of membrane from Ramos cells (a B cell line) to Jurkat cells (a T cell line), with increasing membrane transfer with increasing co-culture density.

Ramos cells were co-cultured with Jurkat cells at a Donor:Acceptor ratio of 1:1 at 300,000 total cells per well in 96-well plates for 1 hr. Ramos cells were stained with CMFDA (for tracking cell identity) and PKH26 for specifically tracking the cell membrane. Putative Jurkat acceptor cells were stained with DeepRed CellTracker dye. In these experiments, the CMFDA and DeepRed identity dyes do not transfer between cells and can be used to reliably identify donors and acceptors. The PKH26 dye, being a lipophilic fluorescent dye, intercalates with the donor cell membrane and can transfer to Jurkat acceptors, to identify instances of membrane transfer from donor to acceptor. Increasing membrane transfer was observed from Ramos to Jurkat cells with increasing co-culture density. Less than 5% of Jurkat cells accepted membrane from the Ramos donors across all conditions. This level of transfer provides a baseline for transfer using unstimulated cells.

### Staining Conditions

Cells were stained according to the below table:

TABLE 9

| | | | | | Cell | |
|---|---|---|---|---|---|---|
| | Cells | PKH | CMFDA | DeepRed | input | Use |
| 1 | Ramos | − | − | − | 1.5 × 10\hat{ }6 | co-culture and |
| | | | | | | unstained ref |
| 2 | Ramos | + | + | − | 1.5 × 10\hat{ }6 | co-culture and |
| | | | | | | PKH+ ref |
| 3 | Ramos | − | + | − | 1.0 × 10\hat{ }6 | CMFDA+ ref |
| 4 | Jurkat | − | − | − | 1.5 × 10\hat{ }6 | co-culture and |
| | | | | | | unstained ref |
| 5 | Jurkat | − | − | + | 1.5 × 10\hat{ }6 | Co-culture and |
| | | | | | | DeepRed ref |

### Procedure

Vi-cell was used to obtain cell counts and measure viability of Ramos and Jurkat cells.

For the Ramos cells, the following protocol was followed. 4×10⁶ cells were removed into a new 15 mL conical tube, washed with PBS, aspirated, and washed again with 10 mL PBS. During the wash a dye dilution was prepared per the staining table above. The cells were resuspended in 200 μL of diluent and distributed to 15 mL tubes: 75 μL (1.5×10⁶ cells) to dual-stained samples, 75 μL (1.5×10⁶ cells) to unstained samples, and 50 μL (1×10⁶ cells) to CMFDA+ reference sample. PKH dye preparations were added to the samples: 75 μL (1.5×10⁶ cells) of PKH-A to dual-stained samples, 75 μL (1.5×10⁶ cells) of PKH-B to unstained samples, and 50 μL (1×10⁶ cells) of PKH-B to CMFDA+ reference sample. The cells were mixed immediately by pipetting and incubated for 5 min. at room temperature (RT). 500 μL of 100% FBS was added to quench the staining and the samples were incubated an addition 1 min.

The cells were pelleted by centrifugation for 10 min at 300 g at RT, then resuspended in 5 mL R10+ and transferred to a new 15 mL conical tube. Cells were pelleted for 5 min at 300 g at RT, washed again with 5 mL RPMI without serum, and resuspended in 5 mL RPMI. 167 μL (50K cells) was removed from the “dual-stained sample” to a 5 mL FACS tube with 1×PBS—for PKH+ ref. control; this was pelleted then resuspended in 150 μL of 2% fix solution and set aside. The remaining dual-stained, unstained, and CMFDA+ samples were pelleted, then resuspended each in RPMI (75 μL to dual-stained samples, 75 μL to unstained samples, and 50 μL to CMFDA+ reference sample). 75 μL of Green A was added to the dual-stained tube (1.45×10⁶ cells), 75 μL of Green B to unstained tube (1.5×10⁶ cells), and 50 μL of Green A to CMFDA+ reference tube (1×10⁶ cells). Samples were incubated for 15 min at 37 C, 5% CO₂ and washed with 5 mL R10+. Samples were washed once more by: adding 5 mL R10+ to dual-stained followed by pelleting; adding 5 mL serum-supplemented R10+ to unstained, removing 167 μL (50K) to a 5 mL FACS tube with 1×PBS—for unstained ref. control (pelleting the 50K cells, resuspending in 150 μL 2% fix solution and setting aside), and pelleting the remaining unstained; and adding 5 mL PBS to the CMFDA+ ref, pelleting the entire sample, resuspending in 300 μL 2% fix solution, transferring to 5 mL FACS tube, and setting it aside. 0.75 mL R10+ was added to the pelleted dual-stained tube (containing ˜2×10⁶/mL) and 0.75 mL of R10+ was added to the pelleted unstained tube (containing ˜2×10⁶/mL). Cells were counted by vi-cell by making a 1:10 dilution (50 μL sample+450 μL R10+) and volumes were adjusted 1.5×10⁶/mL.

For the Jurkat cells, the following protocol was followed. 3×10⁶ live cells were removed into a new 15 mL conical tube. Cells were washed with RPMI (no serum, Ca²⁺, or Mg²⁺) by pelleting at 300 g for 5 min at RT and resuspending in 150 μL RPMI, then distributed into 15 mL conical tubes as follows: 75 μL (1.5×10⁶ cells) for stained Jurkats and DeepRed ref. control, 75 μL (1.5×10⁶ cells) for unstained Jurkats. 75 μL Red-A was added to the stained tube and 75 μL Red-B was added to the unstained tube. Tubes were incubated 15 min at 37° C., 5% CO₂, and R10+ was added up to 5 mL in each tube. 167 μL (50K cells) was removed from the stained tube to a 5 mL FACS tube with 1×PBS—for DeepRed+ ref. control; cells were spun down, then resuspended in 150 μL 2% fix solution and set aside. 167 μL (50K cells) was removed from the unstained tube to a 5 mL FACS tube with 1×PBS—for unstained Jurkats sample; cells were spun down, then resuspended in 150 μL 2% fix solution and set aside. Remaining stained and unstained sample were spun down, washed with 5 ml R10+, and resuspended in 0.75 mL R10+ each (each tube at ˜2×10⁶/mL). Cells were counted by vi-cell by making a 1:10 dilution (50 μL sample+450 μL R10+), with volume adjusted to make 1.5×10⁶/mL.

Co-Culture

Ramos and Jurkat cells were combined and stained according to the stain table above. 200 μL of each of the cell mixtures was distributed to 96-well clear or black plates (for flow cytometry or imaging), according to the plate layout above. The plates were centrifuged for 1 min. at 100 g with the brake off, then cultured at 37° C., 5% CO₂ for 1 hr. Cells were harvested for flow samples by pipetting and transferring to 96-well 5 mL FACS tubes. The cells were pelleted at 300 g for 5 min., stained with L/D violet (5 uL), washed with PBS, and fixed with 150 μL 2% fix buffer with vigorous pipetting. Samples were then run on a flow cytometer.

### Results

The percentage of Jurkat cells positive for Ramos membrane transfer (transfer positive) is plotted in FIG. X1. As the co-culture cell density increased, so did the percentage of transfer positive Jurkat cells.

### Example 29. Transfer from K562 Cells to THP-1 Cells with Stimulation

This example demonstrates increased transfer of membrane from a donor cell to an acceptor cell upon cell stimulation (compared to unstimulated conditions) and further demonstrates the direct correlation between co-culture cell density and transfer rates. K562 donor cells were co-cultured with THP-1 acceptor cells at a 1:1 ratio and 300,000 cells per well in 96-well plates. Co-cultures were incubated for 1 hour before the cells were fixed and analyzed by flow cytometry.

### Procedure

Approx. 5 million THP-1 cells were treated with DMSO and ˜100 nM phorbol 12-myristate-13-acetate (PMA) 48 hours prior to co-culture. 2.5 million cells were then transferred into two T-25 flasks for DMSO and PMA respectively.

K562 and THP-1 cells were counted and viability measured using Vi-cell by the following protocol.

For K562 cells, 7×10⁶ cells were removed into a new 15 mL conical tube, washed with PBS, aspirated, then washed again with 10 mL PBS. During the wash, dye dilution was prepared as outlined in Example 28. Cells were resuspended in 350 μL diluent and distributed to 15 mL tubes as follows: 150 μL (3×10⁶ cells) to dual-stained samples, 150 μL (3×10⁶ cells) to unstained samples, and 50 μL (1×10⁶ cells) to CMFDA+ reference sample. PKH dye was added as follows: 150 μL (3×10⁶ cells) PKH-A to dual-stained samples, 150 μL (3×10⁶ cells) PKH-B to unstained samples, and 50 μL (1×10⁶ cells) PKH-B to CMFDA+ reference sample. Cells were mixed with dye solution rapidly and immediately by pipetting then incubated for 5 min. at RT. 500 μL 100% FBS was added to quench the staining, and cells were incubated for 1 min. Cells were pelleted by centrifugation for 10 min. at 300 g at RT, then resuspended in 5 mL R10+ and transferred to new 15 mL conical tubes (the original tubes were rinsed with 5 mL R10+ and transferred to the new tube too). Cells were pelleted 5 min. at 300 g at RT, washed again with 5 mL RPMI (without serum), and resuspended in 5 ml RPMI. From the “dual-stained sample”, 84 μL (0.05 m) was removed to a 5 mL FACS tube with 1×PBS—for PKH+ ref. control; these were pelleted, resuspended in 150 μL 2% fix solution and set aside. The remaining dual-stained, unstained, and CMFDA+ samples were pelleted, then resuspended in RPMI as follows: 150 μL to dual-stained samples, 150 μL to unstained samples, and 50 μL to CMFDA+ reference sample. Dye was added as follows: 150 μL Green-A to dual-stained tube (2.95×10⁶ cells), 150 μL Green-B to unstained tube (3×10⁶ cells), and 50 μL Green-A to CMFDA+ ref. (1×10⁶ cells). Cells were incubated 15 min. at 37° C., 5% CO₂ and washed with 5 mL R10+. Cells were washed once more as follows: 5 mL of R10+ was added to dual-stained, then cells were pelleted; 5 mL R10+ was added to unstained, then 84 μL (0.05 m) was removed to a 5 mL FACS tube with 1×PBS—for unstained ref. control, the 50K cells were pelleted, resuspended in 150 μL 2% fix solution and set aside, while the remaining unstained cells were pelleted; 5 mL PBS was added to the CMFDA+ ref, the entire sample was pelleted, resuspended in 300 μL 2% fix solution, transferred to a 5 mL FACS tube, and set aside. Pelleted cells were resuspended in 1 mL R10+(bringing both dual-stained and unstained tubes to −3×10⁶/mL). The cells were counted by vi-cell by making a 1:10 dilution (50 μL sample+450 μL R10+), and volume was adjusted to make 1.5×10⁶/mL.

For THP-1 cells (previously stimulated by DMSO and PMA), T-25 flasks were washed with PBS, then ice-cold 5 mM EDTA was added and incubated for 40 min. on ice. Cells were detached by tapping the side of the flask and pipetting. 1 mL of trypsin was added and incubated for 5 min. at 37° C. Cells were gently resuspended and removed to a flask of R10+. Cells were spun down, resuspended in 10 mL of RPMI and counted. 6×10⁶ live cells were removed into a new 15 mL conical tube, washed with RPMI (no serum, Ca²⁺, or Mg²⁺) by pelleting at 300 g for 5 min at RT, then resuspended in 300 μL RPMI. Sample was distributed into 15 mL conical tubes as follows: 150 μL (3×10⁶ cells) for stained THP-1 and DeepRed ref. control (dmso), 150 μL (3×10⁶ cells) for unstained THP-1 (dmso), 150 μL (3×10⁶ cells) for stained THP-1 and DeepRed ref control (dmso), and 150 μL (3×10⁶ cells) for unstained THP-1 (dmso). Dye was added to samples as follows: 150 μL Red-A to stained tubes, 150 μL Red-B to unstained tubes. Tubes were incubated 15 min at 37° C., 5% CO₂, then R10+ was added up to 5 mL. From the first stained tube, 84 μL (0.05 m) were removed to a 5 mL FACS tube with 1×PBS—for DeepRed+ ref. control; the cells were spun down, resuspended in 150 μL 2% fix solution, and set aside. From the first unstained tube, 167 μL (0.05 m) were removed to a 5 mL FACS tube with 1×PBS—for unstained THP-1 sample; the cells were spun down, resuspended in 150 μL 2% fix solution, and set aside. The second stained and unstained tubes were spun down, washed once more with 5 mL R10+, then resuspended in 1 mL R10+ each (to −3×10⁶/mL). Cells were counted by vi-cell making a 1:10 dilution (50 μL sample+450 μL R10+). Volume was adjusted to make 1.5×10⁶/mL.

TABLE 10

| | | | | Needed | | | | |
|---|---|---|---|---|---|---|---|---|
| | K562 | THP1 | E:T | (uL) | K562 | THP1 | Volume | Comments |
| K562 | Unstained | — | NA | 0.4 | 0.2 | | | |
| THP-1 (dmso) | — | Unstained | NA | 0.4 | | 0.2 | | |
| K562 | Stained | — | NA | 0.4 | 0.2 | | | |
| THP-1 (dmso) | — | stained | NA | 0.4 | | 0.2 | | |
| THP-1 (dmso) | Unstained | Unstained | 1 | 0.4 | 0.3 | 0.3 | 0.6 | not useful |
| THP-1 (dmso) | Stained | Stained | 1 | 0.4 | 0.3 | 0.3 | 0.6 | 200, 100, |
| | | | | | | | | 50 uL, |
| | | | | | | | | 150 + 50 |
| THP-1 (dmso) | Unstained | Stained | 1 | 0.4 | 0.3 | 0.3 | 0.6 | not useful |
| THP-1 (dmso) | Stained | Unstained | 1 | 0.4 | 0.3 | 0.3 | 0.6 | 200, 100, |
| | | | | | | | | 50 uL, |
| | | | | | | | | 150 + 50 |
| THP-1 (PMA) | — | Unstained | | 0.4 | | 0.2 | | |
| THP-1 (PMA) | — | stained | | 0.4 | | 0.2 | | |
| THP-1 (PMA) | Unstained | Unstained | 1 | 0.4 | 0.3 | 0.3 | 0.6 | |
| THP-1 (PMA) | Stained | Stained | 1 | 0.4 | 0.3 | 0.3 | 0.6 | |
| THP-1 (PMA) | Unstained | Stained | 1 | 0.4 | 0.3 | 0.3 | 0.6 | |
| THP-1 (PMA) | Stained | Unstained | 1 | 0.4 | 0.3 | 0.3 | 0.6 | |

### Co-Culture

Stained and unstained K562 and THP-1 were combined according to the table above. 200 μL (0.3 m cells) of each of the cell mixtures were distributed to 96-well clear or black plates for flow cytometry or imaging. The plate was centrifuged for 1 min at 100 g with the brake off. Cells were cultured at 37° C., 5% CO₂ for 1 hr. For flow samples, cells were harvested by pipetting and transferring to 5 mL FACS tubes, pelleted at 300 g for 5 min, stained with L/D violet (5 μL), then washed with PBS. Cells were fixed with 200 μL 2% fix solution with vigorous pipetting and pulse vortexing, then run on the flow cytometer.

### Results

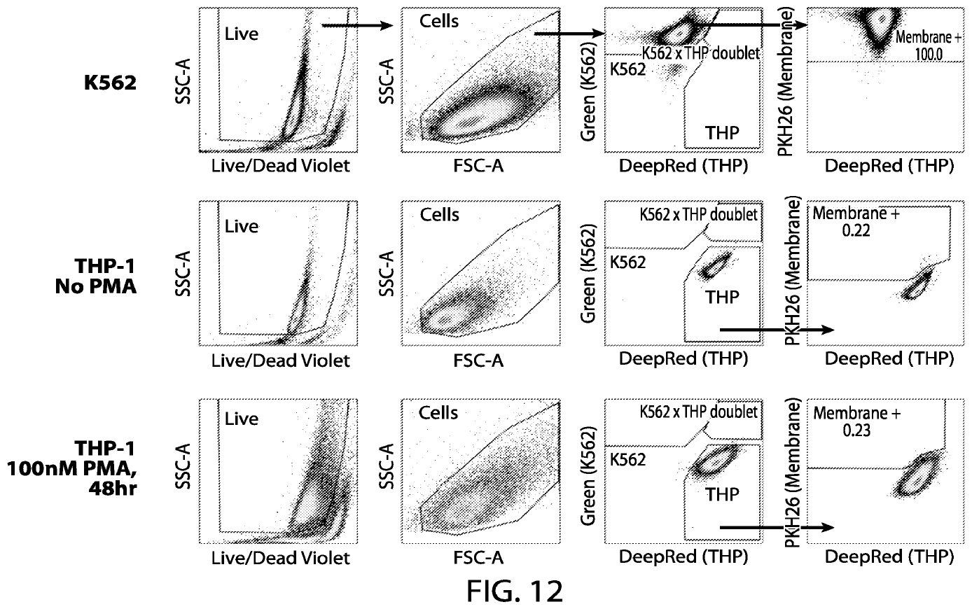

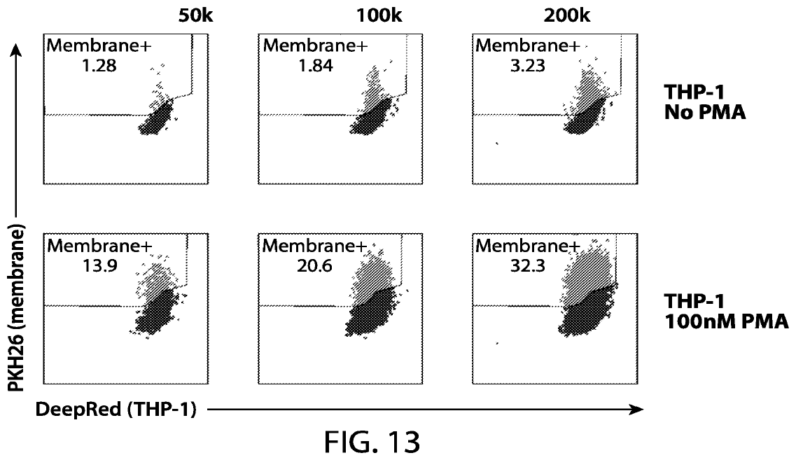

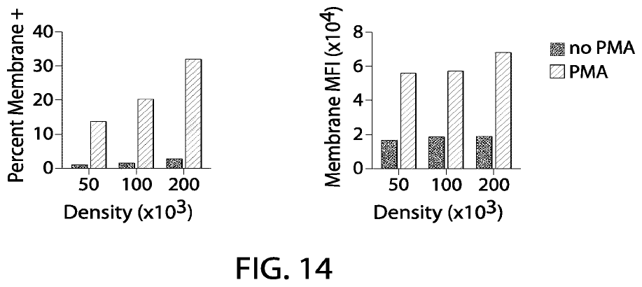

Cells were gated by live cells, side scatter-by-forward scatter, and cell tracker dyes to segregate donors (CMFDA/Green-stained K562) from acceptors (DeepRed-stained THP-1). Finally, membrane positive/negative gates were set based on PKH26 membrane dye on K562 donor cells prior to any co-culture (FIG. 12). Cells gated for DeepRed dye positivity (shown by red dots), indicating the THP-1 acceptors, are seen in FIG. 13. The Y-axis indicates PKH26 membrane dye. Gates were duplicated directly from the donor-only staining in FIG. 12. The data is quantified and compared in FIG. 14. These results clearly show transfer from donor K562 cells to activated acceptor THP-1 cells, and that transfer rates depend on cell density as well as activation. From these experiments, THP-1 activation with PMA drives approximately a 10-fold increase in transfer.

### Example 30. Transfer from K562 or JEG-3 Cells to Mixed Primary Peripheral Blood Mononuclear Cells (PBMCs) and THP-1 Cells

This experiment demonstrates: 1) a system for screening a donor cell for ability to transfer to multiple acceptor cells at once, 2) that most PBMC subsets can act as acceptors, 3) that individual acceptors can repeatedly receive a transfer from distinct donors, and 4) that increasing donor:acceptor ratio increases incidence of transfer. Here, K562 or JEG-3 donor cells, THP-1 acceptor cells, and PBMC donor/acceptor cells were membrane-labeled with cell tracker dyes to distinguish the 3 input populations after co-culture. The K562/JEG-3 and PBMC groups were also stained with membrane dyes to track membrane transfer among the cells. THP-1 cells were included to have an internal control, since transfer had already been observed from K562 cells to THP-1 cells (Example 29) and from JEG-3 cells to THP-1 cells (data not shown here). The cells were co-cultured at a ratio of 50:5:1 (K562 or JEG-3:PBMCs:THP-1) for 1 hour. Both stimulated (PBMCs with 10 ug/mL PMA & 500 μg/mL ionomycin for 7 hours, and THP-1s with 100 nM PMA for 2 days) and unstimulated conditions were tested. Widespread transfer from K562 and JEG-3 cells to all PBMC subsets was observed at varying incidences. The presence of PBMCs did not prevent transfer to THP-1 cells in the mixed assay. Finally, individual THP-1 cells were capable of accepting from both PBMCs and cell line donors in the same assay.

### PBMC Preparation Procedure

PBMCs were thawed as follows. The vial of frozen cells was submerged in 37° C. water bath until only about 1 pea-sized amount of ice remains, ˜1 min. 30 mL of warm R10+ with 50 U/mL benzonase was prepared, along with a 15 mL conical tube with 5 mL warm R10+ with benzonase. The outside of the vial was sprayed with EtOH and wiped down. Warm R10+ was added dropwise into the vial, until the vial is full. The contents of the vial were gently mixed with a transfer pipette and transferred to the 15 mL tube. The vial was rinsed with 1 mL warm R10+, transferred to tube, and rinsed once more. The tube was topped off to 10 mL, then pelleted at 200 g for 5 min. Pellet was aspirated and resuspended in 5 mL warm R10+/benzonase (to be 3×10⁶/mL). Cells were counted and viability measured by vi-cell.

PBMCs were stimulated as follows. 4×10⁶ PBMCs were removed and set aside at 37° C. Two samples of 3×10⁶ cells each were removed to use as unstimulated and stimulated PBMCs. Cells were spun down and resuspended in 2 mL R10+ and transferred to a 6-well plate. The tubes were rinsed twice and transferred to their corresponding places on the 6-well plate, which were then mixed. The cells were incubated 37° C. for 4 hr.

### Cell Labeling Procedure

K562 and JEG-3 cells were counted by vi-cell and viability was assessed. 13×10⁶ JEG-3 and 15×10⁶ K562 cells were removed into new 15 mL conical tubes. Cells were washed with PBS, aspirated, then washed again with 10 mL PBS. Dye dilution was prepared as outlined in Example 28. Cells were resuspended in 650 μL diluent for JEG-3 and 750 μL diluent for K562. K562 cells were distributed to 15 mL tubes as follows: 600 μL (12×10⁶ cells) to dual-stained samples and 150 μL (3×10⁶ cells) to unstained samples. JEG-3 cells were distributed to 15 mL tubes as follows: 500 μL (10×10⁶ cells) to dual-stained samples and 150 μL (3×10⁶ cells) to unstained samples. PKH dye (2 μM PKH67) was added to K562 cells as follows: 600 μL PKH-A to dual-stained samples and 150 μL PKH-B to unstained samples. PKH dye (8 μM PKH67) was added to JEG-3 cells as follows: 500 μL PKH-A to dual-stained samples and 150 μL PKH-B to unstained samples. Samples were mixed immediately by pipetting and incubated for 5 min. at RT. 1.2 ml 100% FBS was added to quench the staining and samples were incubated for 1 min. Cells were pelleted by centrifugation for 10 min at 300 g at RT, resuspended in 5 mL R10+ and transferred to new 15 mL conical tubes. The original tubes were rinsed with 5 mL R10+ and this too was transferred to the corresponding new tube. Cells were pelleted 5 min at 300 g at RT, and washed with 5 mL RPMI (without serum). JEG-3 cells were resuspended in 5 mL RPMI. 25 μL (0.05 m) were removed from the “dual-stained sample” to a 5 mL FACS tube with 1×PBS—for PKH67+ ref. control, while the rest of the cells were pelleted 5 min at 300× g at RT. K562 cells were resuspended in RPMI as follows: 600 μL to dual-stained samples and 150 μL to unstained samples. JEG-3 cells were resuspended in RPMI as follows: 500 μL to dual-stained samples and 150 μL to unstained samples. Dye (20 μM Violet) was added to K562 cells as follows: 600 μL Violet-A to dual-stained tube and 150 μL Violet-B to unstained tube. Dye (10 μM Violet) was added to JEG-3 cells as follows: 500 μL Violet-A to dual-stained tube and 150 μL Violet-B to unstained tube. Cells were incubated 15 min at 37° C., 5% CO₂, washed 2 times with 5 mL R10+, and resuspended in 1 ml of R10+. Cells were counted by vi-cell by making a 1:10 dilution (50 μL sample+450 μL R10+). Sample volume was adjusted to make: 10×10⁶ in 2 mL for dual-stained and 2.5×10⁶ in 0.5 mL for unstained for K562 (5×10⁶/mL), and 7.5×10⁶ in 1.5 mL for JEG-3 dual-stained and 2.5×10⁶ in 0.5 mL for unstained (5×10⁶/mL).

PMA-activated THP-1 cells were labeled and counted as follows. Media was removed from cells and cells were washed with PBS. Pre-warmed trypsin-EDTA (1 mL) was added and the cells were incubated for 3-5 min. Cells were gently dislodged by pipetting media, then spun down, resuspended in media, and counted. 2×10⁶ live cells were removed into a new 15 mL conical tube. Cells were washed with RPMI (no serum, Ca²⁺, or Mg²⁺) by pelleting at 300 g for 5 min at RT. Cells were resuspended in 100 μL RPMI, then distributed into 15 mL conical tubes as follows: 50 μL (1×10⁶ cells) for stained THP-1 (activated) and 50 μL (1×10⁶ cells) for unstained THP-1 (activated). Dye was added to the THP-1 cells as follows: 50 μL Red-A to stained tubes and 50 μL Red-B to unstained tubes. Cells were incubated 15 min at 37° C., 5% CO₂, then 5 mL R10+ was added, the cells were spun down, and washed with 5 mL R10+. Cells were resuspended in 1 mL R10+ in both the dual-stained tube and unstained tube (to −1×10⁶/mL). Cells were counted by vi-cell by making a 1:10 dilution (50 μL sample+450 μL R10+), and viability was assessed. Volume was adjusted to 0.1×10⁶/mL.

PBMC cells were labeled and counted as follows. 3×10⁶ cells were removed into a new 15 mL conical tube for both unstimulated and stimulated. Cells were washed with PBS, aspirated, and washed again with 10 mL PBS. Dye dilutions were prepared as described in Example 28. Cells were resuspended in 150 μL diluent, then distributed in 15 mL tubes as follows: 100 μL (2.0×10⁶ cells) to dual-stained samples and 50 μL (1×10⁶ cells) to unstained samples. PKH dye was added to cells as follows: 100 μL PKH-C to dual-stained samples and 50 μL PKH-D to unstained samples. Cells were mixed immediately and rapidly by pipetting, then incubated for 1 min. at RT. 500 μL 100% FBS were added to quench the staining and samples were incubated for 1 min. Cells were pelleted by centrifugation for 10 min at 300 g at RT, resuspended in 5 mL R10+, transferred to new 15 mL conical tubes, and the original tubes were rinsed with 5 mL R10+ and this was transferred to the corresponding new tube. Cells were pelleted for 5 min at 300 g at RT, washed with 5 mL RPMI (without serum) and pelleted again. Cells were resuspended in RPMI as follows: 100 μL to dual-stained samples and 50 μL to unstained samples. Dye was added to the cells as follows: 100 μL DeepRed-A to dual-stained tube and 50 μL DeepRed-B to unstained tube. Cells with dye were incubated 15 min at 37° C., 5% CO₂, then washed twice with 5 mL R10+ and then both dual-stained tube and unstained tube cells were resuspended in 1 mL each of R10+. Cells were counted by vi-cell by making a 1:10 dilution (50 μL sample+450 μL R10+). Volume was adjusted to make 1.7×10⁶ cells in 1.7 mL R10+ for dual-stained tube and 0.5×10⁶ cells in 0.5 mL R10+ for unstained (1×10⁶/mL).

### Co-Culture Procedure

Cells were combined according to the table below. Cell mixtures were distributed to 24-well clear plates. The plate was centrifuged for 1 min at 100 g with brake off, then cells were cultured at 37° C., 5% CO₂ for 1 hr.

TABLE 11

| | | | | | | | Total | | | | |
|---|---|---|---|---|---|---|---|---|---|---|---|
| | | | | | | Donor:PBMC: | volume | | | | |
| | | | Donor | PBMC | THP-1 | (THP-1) | (mL) | Donor | PBMC | THP-1 | |
| 1 | Donor | K562 | Unstained | — | — | NA | | 0.3 | | | |
| 2 | Donor | K562 | Stained | — | — | NA | | 0.3 | | | |
| 3 | | JEG-3 | Unstained | — | — | NA | | 0.3 | | | |
| 4 | | JEG-3 | Stained | — | — | NA | | 0.3 | | | |
| 5 | | PBMC (−) | — | unstained | — | NA | | | 0.3 | | |
| 6 | | PBMC (−) | — | stained | — | NA | | | 0.3 | | |
| 7 | K562xPBMC | PBMC (−) | Stained | Stained | — | 5 | 0.8 | 0.4 | 0.4 | | pipette 0.6 ml |
| | | | | | | | | | | | and 120 uL |
| 8 | JEG-3xPBMC | PBMC (−) | Stained | Stained | — | 5 | 0.8 | 0.4 | 0.4 | | pipette 0. [symbol] |
| | | | | | | | | | | | 12 [symbol] |
| 9 | | PBMC (+) | — | Unstained | — | | | | 0.3 | | |
| 10 | | PBMC (+) | — | stained | — | | | | 0.3 | | |
| 11 | K562xPBMC | PBMC (+) | Stained | Stained | — | 5 | 0.8 | 0.4 | 0.4 | | pip [symbol] 0. [symbol] |
| | | | | | | | | | | | 12 [symbol] |
| 12 | JEG-3xPBMC | PBMC (+) | Stained | Stained | — | 5 | 0.8 | 0.4 | 0.4 | | pipette 0.6 ml |
| | | | | | | | | | | | and 120 uL |
| 13 | | THP-1 | — | — | unstained | NA | | | | 0.5 | |
| 14 | | THP-1 | — | — | stained | NA | | | | 0.5 | |
| 15 | K562xPBMCxTHP1 | THP-1 | Stained | Stained | Stained | 50:10:1 | 1.2 | 0.4 | 0.4 | 0.4 | pipette 0.9 ml |
| | | | | (PBMC:U) | | | | | | | and 180 uL |
| 16 | K562xPBMCxTHP1 | THP-1 | Stained | Stained | Stained | 50:10:1 | 1.2 | 0.4 | 0.4 | 0.4 | pipette 0.9 ml |
| | | | | (PBMC:S) | | | | | | | and 180 uL |

[symbol]

indicates data missing or illegible when filed

TABLE 12

| | | | | | | | Total | | | |
|---|---|---|---|---|---|---|---|---|---|---|
| | | | | | | | volume | | | |
| | | | Donor | PBMC | THP-1 | Donor:PBMC | (mL) | Donor | PBMC | |
| 1 | K562xPBMC | PBMC (−) | Stained | Stained | — | 1 | 0.8 | 0.4 | 0.4 | pipette 0.6 ml |
| | | | | | | | | | | and 120 uL |
| 2 | JEG-3xPBMC | PBMC (−) | Stained | Stained | — | 1 | 0.8 | 0.4 | 0.4 | pipette 0.6 ml |
| | | | | | | | | | | and 120 uL |
| 3 | K562xPBMC | PBMC (+) | Stained | Stained | — | 1 | 0.8 | 0.4 | 0.4 | pipette 0.6 ml |
| | | | | | | | | | | and 120 uL |
| 4 | JEG-3xPBMC | PBMC (+) | Stained | Stained | — | 1 | 0.8 | 0.4 | 0.4 | pipette 0.6 ml |
| | | | | | | | | | | and 120 uL |

TABLE 13

| | 5:1 | | | | | | | |
|---|---|---|---|---|---|---|---|---|
| A and B | C and D | A | B | C | D | E | F | |
| K562 | K562xPBMC (−) | unstained | stained | 600 | 120 | unstained | stained | JEG3 |
| THP-1 | K562xTHP- | unstained | stained | 900 | 180 | 600 | 120 | JEGxPBMC |
| | 1xPBMC (−) | | | | | | | (−) |
| PBMC (−) | K562xPBMC (+) | unstained | stained | 600 | 120 | 600 | 120 | JEGxPBMC |
| | | | | | | | | (+) |
| PBMC (+) | K562xTHP- | unstained | stained | 900 | 180 | | | |
| | 1xPBMC (+) | | | | | | | |

TABLE 14

| | | | | | | |
|---|---|---|---|---|---|---|
| 1:1 | A | B | C | D | E | F |

| | | |
|---|---|---|
| K562xPBMC (−) | 600 | 120 |
| K562xPBMC (+) | 600 | 120 |
| JEGxPBMC (−) | 600 | 120 |
| JEGxPBMC (+) | 600 | 120 |

Cells were stained for live/dead analysis. Samples were transferred from 24-well to 5 mL FACS tubes at 4° C., rinsing with ice cold PBS. Cells were pelleted, resuspended in 100 μL 1×L/D violet, and stained for 30 min at 4° C. in the dark. Samples were transferred to a 96-well V bottom plate and washed with ice cold PBS.

Antibody staining of cells was performed as follows. Cells were resuspended in 40 μL ice cold PBS, 5 μL FcBlock was added, and cells were incubated for 20 min 4° C. in the dark. 55 μL cold antibody cocktail mix was added, then cells were incubated 30 min 4° C. in the dark. Cells were washed with 200 μL ice cold WB, resuspended in 1× cold SA-PECy7, then mixed and incubated 20 min 4° C. in the dark. Cells were washed with 200 μL ice cold WB, fixed in 250 μL cold fix solution, then samples were run on a flow cytometer.

### Results

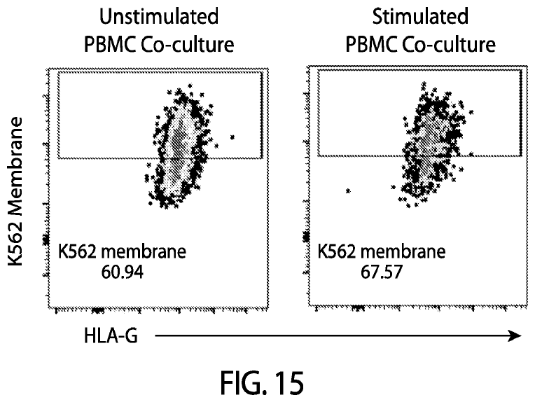

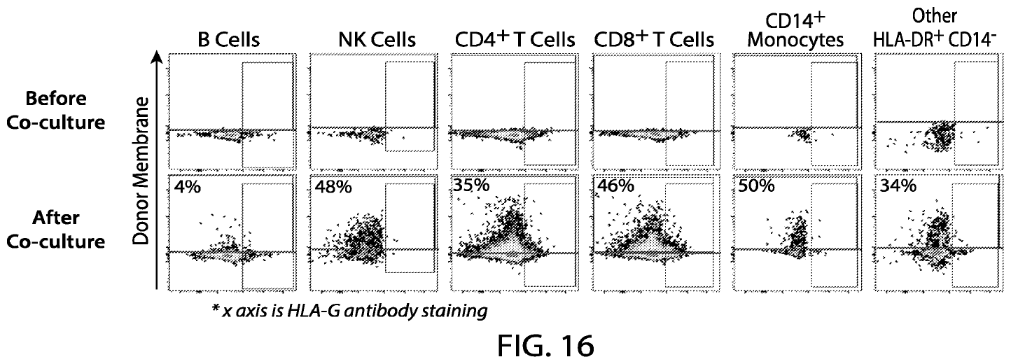

Flow cytometry confirms that K562 transfer to THP-1 cells is maintained in the presence of PBMCs (FIG. 15). FIG. 16 shows transfer incidence before and after co-culture from K562 to PBMC subsets, with transfer incidence calculated in green on each chart: 4% for B cells, 48% for NK cells, 35% for CD4+ T cells, 46% for CD8+ T cells, 50% for CD14+ monocytes, and 34% for Other HLA-DR+CD14− cells. Transfer is only observed after co-culture of K562 cells with PBMCs.

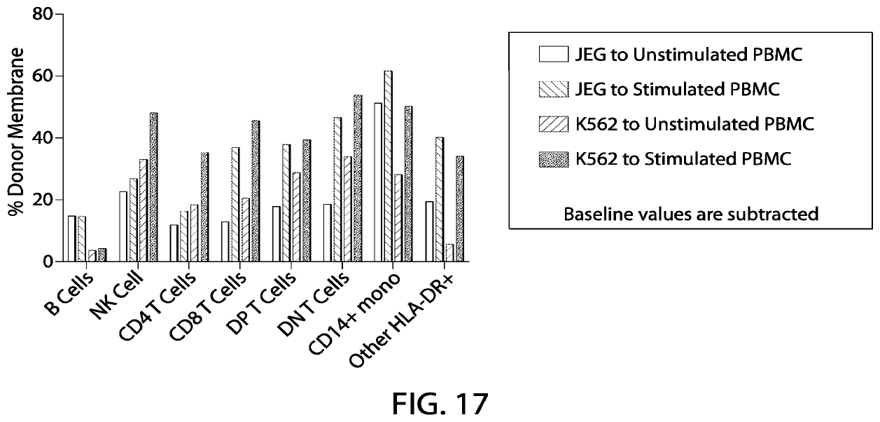

Transfer of membrane from JEG-3 donor cells and K562 donor cells to PBMC subsets, stimulated and unstimulated, was quantified and is shown in FIG. 17. Transfer incidence varied based on PBMC subset and which donor cell was donating membrane, though both JEG-3 and K562 were observed to transfer membrane to acceptor THP-1 cells. Generally, stimulated THP-1 cells received more membrane than unstimulated, though both were competent to receive membrane from donor cells.

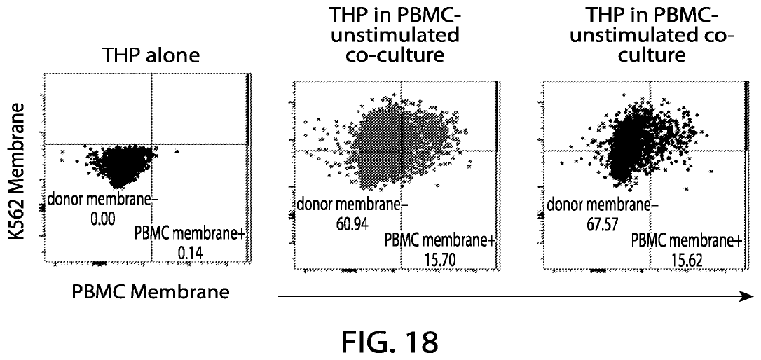

Acceptor activity of THP-1 cells was assessed to determine the extent of membrane transfer from either or both donor cell type (FIG. 18). When THP-1 cells alone (not co-cultured) were analyzed, no donor membrane was observed. Unstimulated and stimulated THP-1 cells were observed to have donor membrane from K562 donor cells only, PBMC donor cells only, and both K562 and PBMC donor cells. This shows that an acceptor cell can receive membrane from two different donor cells present in the same co-culture mixture.

### Example 31. Therapeutic Ex Vivo Cell Modification

This example demonstrates transfer of a membrane-associated agent or cargo molecule, e.g., a chimeric antigen receptor (CAR), to patient immune cells. The donor cell is engineered in advance to express the CAR. To provide the therapy, the engineered CAR-expressing cells are placed in contact with patient cells either ex vivo or extracorporeal. The CAR is transferred to the patient cells as protein. Genetic material from the donor cell, including the engineered gene encoding the CAR, does not enter the acceptor cell. Patient acceptor cells are returned to the patient. The donors may be engineered to transfer multiple protein cargos to the acceptor to enhance efficacy of the cell therapy. This could include chemokines for tissue homing and/or activation receptors.

Cell modification—CAR: First, donor cells (Ramos cell line) are engineered to express CARs that target cell surface antigens expressed by cancer cells from the patient. This requires knowledge of patient tumor surface antigens in some cases. In other cases, surface antigens may be common to the cancer cells and their normal cells of origin. In this example the CAR targets Mucin 1 (MUC1), which is often expressed on the surface of several epithelial cancers.

Cell modification—CAR Shuttle: In this example, the desired acceptor cells (patient T and NK cells in this example) do not express the target antigen of the CAR. As such, the CAR can be transferred to the acceptors as a cargo molecule (wherein the CAR receptor on the donor does not bind to a target cell moiety, e.g., surface receptor, on the acceptor). To accomplish this, the donors are engineered to express a membrane-associated agent that: (1) binds the acceptor cell to drive transfer and (2) associates with the CAR to include it in the transfer. To accomplish this, the extracellular moiety of the membrane-associated agent binds to target cell moieties, e.g., surface antigens, on the T and NK cells. In this example, we will target IL-2Rβ (CD122), which is expressed on both T and NK cells, via the extracellular moiety of the membrane-associated agent. The intracellular moiety is designed to bind the CAR protein with low affinity. The membrane associated agent is expressed in the donor Ramos cells.

Contact-mediated Transfer: Ramos cells (donors) expressing both the CAR and the membrane-associated agent will be placed in culture with patient T and NK cells (acceptors). Culture conditions are designed to promote direct cell-cell contact between the donors and acceptors. The cells are kept under contact conditions for at least 1 minute and up to 4 hours, e.g., 1 hour.

Acceptor purification: After contact-mediated transfer, the T and NK cells are re-isolated and donor Ramos cells are stringently excluded using cell sorting for cell-specific markers.

Transfer validation: Transfer is measured by flow cytometry. A sample of the acceptor cells is stained for the MUC1-targeting CAR protein. The staining and analysis are conducted without cell permeabilization to confirm proper out-facing orientation of the CAR specificity domain on the T and NK cells. For functional validations, the post-transfer T and NK cells are cultured with target cells expressing MUC1 and assessed for killing activity.

Administration to patient: After being separated from Ramos donor cells, Acceptor cells are returned intravenously to the patient.

### Example 32. Cell Therapy Through Endothelial Transfer

This example demonstrates transfer to or from extravasating cells at sites of endothelial transmigration. In this case, proteins of interest are installed in endothelial cells through protein-level transfer, mRNA delivery, or genetic engineering. The endothelial cells express cargo molecules for donation to extravasating cells. This system benefits from natural transfer selectivity since only cells that migrate through the endothelial wall receive the cargo molecule.

Donor endothelium engineering—2-step cargo installation: Donor T cells are engineered to express the cargo molecule of interest. In this example, T cells are given a chimeric antigen receptor via the same process outlined in Example 31: Ramos cells transfer the cancer-specific CAR cargo molecule to T cells carried by a membrane-associated agent, e.g., an engineered CAR shuttle. First, donor cells (Ramos cell line) are engineered to express CARs that target cell surface antigens expressed by cancer cells from the patient. This requires knowledge of patient tumor surface antigens in some cases. In other cases, surface antigens may be common to the cancer cells and their normal cells of origin. In this example the CAR targets Mucin 1 (MUC1), which is often expressed on the surface of several epithelial cancers. In this example, the desired acceptor cells (patient T cells in this example) do not express the target antigen of the CAR. As such, the CAR can be transferred to the acceptors as a cargo molecule (wherein the CAR receptor on the donor does not bind to a target cell moiety, e.g., surface receptor, on the acceptor). To accomplish this, the donors are engineered to express a membrane-associated agent that: (1) binds the acceptor cell to drive transfer and (2) associates with the CAR to include it in the transfer. To accomplish this, the extracellular moiety of the membrane-associated agent binds to target cell moieties, e.g., surface antigens, on the T cells. In this example, we will target CD8 (CD122), which is expressed on cytotoxic T cells, via the extracellular moiety of the membrane-associated agent.

For transfer to T cells and subsequent re-transfer to endothelial cells in vivo, the extracellular moiety of the membrane-associated agent includes 2 affinities: one that binds CD8 (noted above) and a second that binds to e-selectin, expressed by inflamed endothelial cells. As before the intracellular moiety is designed to bind the CAR protein with low affinity. The membrane associated agent is co-expressed in the donor Ramos cells.

Contact-mediated Transfer: Ramos cells (donors) expressing both the cargo molecule CAR and the bi-specific membrane-associated agent will be placed in culture with patient T cells (acceptor #1). Culture conditions are designed to promote direct cell-cell contact between the donors and acceptors. The cells are kept under contact conditions for at least 1 minute and up to 4 hours, e.g., 1 hour.

Acceptor purification: After contact-mediated transfer, the T cells are re-isolated and donor Ramos cells are stringently excluded using cell sorting for cell-specific markers.

Administration to patient: After being separated from Ramos donor cells, acceptor T cells are returned intravenously to the patient.

Extravasation-mediated Transfer: In the bloodstream, T cells are capable of recognizing sites of inflammation through adhesion receptors displayed by inflamed endothelial cells (including E-selectin). The T cells adhere to these receptors and undergo a process of rolling prior to extravasation through the endothelial wall. In the process, T cells donate to endothelial cells. Since E-selectin is one of the 2 specificities included in the membrane-associated agent, the T cells donate to the endothelium as they transmigrate. As with the first contact-mediated transfer from Ramos cells, the CAR co-transfers with the membrane-associated agent.

Final Extravasation-mediated Transfer: As further endogenous patient T cells continue to extravasate at the site of inflammation, the further endogenous patient T cells may acquire the membrane-associated agent and CAR. In this way, subsequent T cells that respond to the inflammation may be able to receive the CAR.

### Example 33. Cell Therapy Through Endothelial Transfer Using mRNA

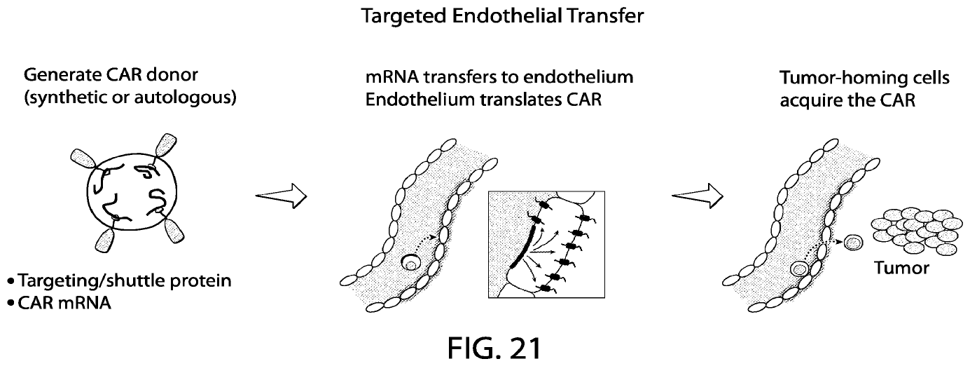

This example follows the same sequence of transfers as Example 32, but uses mRNA to amplify the amount of protein that can ultimately be transferred in the final exchange (see FIG. 21).

Cargo molecule and membrane-associated agent engineering: Donor Ramos cells are engineered to express an mRNA cargo molecule encoding a protein of interest. In this example, the mRNA encodes the same chimeric antigen receptor (CAR) as in Example 32. Here, the mRNA is capable of binding intracellularly to the bi-specific membrane-associated agent through the agent's intracellular moiety which includes an mRNA-binding domain. As in Example 32, the membrane-associated agent has a bi-specific extracellular moiety targeting T cells (via anti-CD3) and endothelial cells (via anti-E-selectin).

Contact-mediated Transfer: Ramos cells (donors) expressing both the CAR mRNA and the bi-specific membrane-associated agent will be placed in culture with patient T cells (acceptor #1). Culture conditions are designed to promote direct cell-cell contact between the donors and acceptors. The cells are kept under contact conditions for at least 1 minute and up to 4 hours, e.g., 1 hour.

Acceptor purification: After contact-mediated transfer, the T cells are re-isolated and donor Ramos cells are stringently excluded using cell sorting for cell-specific markers.

Administration to patient: After being separated from Ramos donor cells, acceptor T cells are returned intravenously to the patient.

Extravasation-mediated Transfer: In the bloodstream, T cells are capable of recognizing sites of inflammation through adhesion receptors displayed by inflamed endothelial cells (including E-selectin). The T cells adhere to these receptors and undergo a process of rolling prior to extravasation through the endothelial wall. In the process, T cells donate to endothelial cells. Since E-selectin is one of the 2 target specificities included in the membrane-associated agent, the T cells donate to the endothelium as they transmigrate. As with the first contact-mediated transfer from Ramos cells, the CAR mRNA co-transfers with the membrane-associated agent. Final Extravasation-mediated Transfer: Once in the endothelial cells, the CAR mRNA is translated, generated CAR protein. As further endogenous patient T cells continue to extravasate at the site of inflammation, the further endogenous patient T cells may acquire the membrane-associated agent and CAR. In this way, subsequent T cells that respond to the inflammation may be able to receive the CAR.

### Example 34. Transfer of siRNA-Cy3 from J76 Cells to Ramos Cells

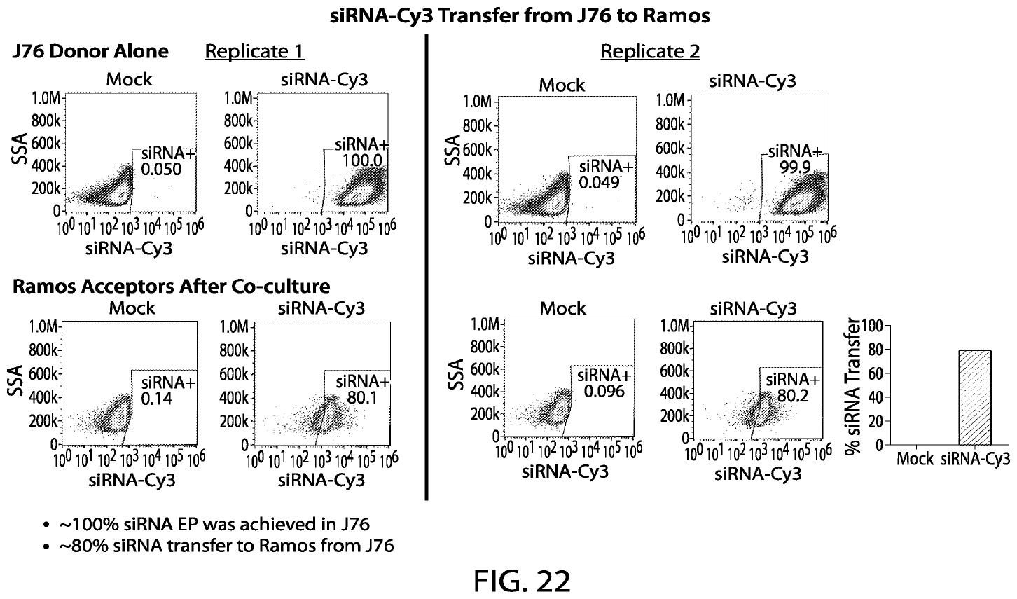

In this example, labeled non-coding cytoplasmic RNA was transferred from J76 cells to Ramos cells. Briefly, J76 cells were transfected with mock and 0.5 uM siRNA-Cy3 for 30 min with Neon electroporation device. Mock and siRNA transfected J76 cells were washed twice and labeled with cell tracker DeepRed dye. Ramos cells were washed and labeled with cell tracker green dye. Cells were then co-cultured in a V-bottom plate at 5:1 ratio of J76:Ramos for a total of 0.36 million cells per well. Plate was spun at 50 g for 10 s and incubated at 37° C. for 1 h. After 1 h, cells were washed with cold PBS, stained with live dead NIR fixable dye and fixed for FACS analyses. As shown in FIG. 22, a substantial amount of siRNA-Cy3 was successfully transferred from J76 cells to Ramos cells.

### Example 35: Co-Culture of K562-HLA-G Donor Cells with THP and Jurkat Acceptor Cells

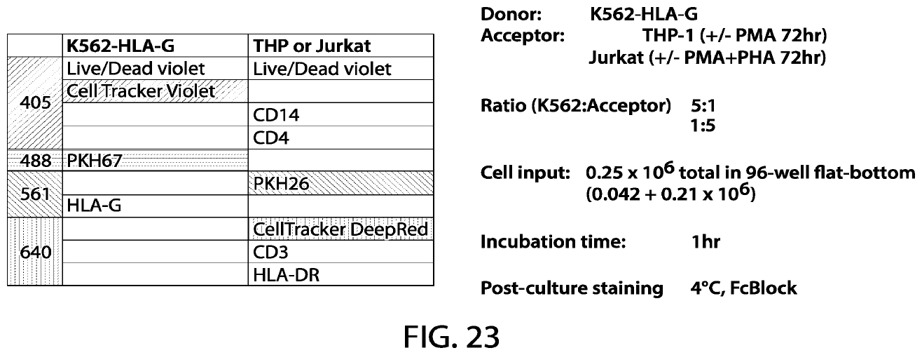

In this example, the transfer of the surface protein HLA-G from donor K562-HLA-G cells (a myeloid cell line stably transfected to express the HLA-G protein on its surface) to acceptor cells is demonstrated. Acceptor cells tested include Jurkat cells (a T cell line) and THP-1 cells (a monocytic cell line). Also shown is that the extent of transfer can be modulated not only by changing the ratio of donors to acceptors but also by changing the activation state of the acceptors. In brief, K562 donor cells were co-cultured with THP-1 and Jurkat acceptor cells at 1:5 and 5:1 ratios with 250,000 cells per well in 96-well plates. Co-cultures were incubated for 1 hour before the cells were stained and fixed and analyzed by flow cytometry (FIG. 23).

Procedure

Approximately 2.5 million Jurkat cells were transferred to each of two T-25 flasks. One flask was treated with 50 ng/mL PMA and 1 μg/mL leucoagglutinin (PHA-L). The other flask was treated with DMSO. Treatment occurred ˜72 hour prior to co-culture.

Approximately 2 million THP-1 cells were transferred to each of two T-25 flasks. One flask was treated with ˜100 nM phorbol 12-myristate-13-acetate (PMA). The other flask was treated with DMSO. Treatment occurred ˜48 hours prior to co-culture.

On the day of co-culture, 7×10⁶ K562-HLA-G cells were moved into a 15 mL conical tube and washed twice with PBS. During the wash, PKH67 dye dilution, was prepared as outlined in Example 28. Cells were resuspended in 350 μL Diluent C, and distributed to 15 mL tubes as follows: 150 μL to dual-stained samples, 150 μL to unstained samples, and 50 μL for Cell tracker Violet BMQC+ reference sample. An equal volume of 4 μM PKH67 dye (or Diluent C-only) was added as follows: 150 μL PKH26 to dual-stained samples, 150 μL Diluent C to unstained samples, and 50 μL Diluent C to Violet+ reference sample. Cells were mixed with dye solution rapidly and immediately by pipetting then incubated for 5 minutes at room temperature. 500 μL 100% FBS was added to quench the staining, and cells were incubated for 1 minute. Cells were pelleted by centrifugation for 10 minutes at 300 g at room temperature, then resuspended in 5 mL R10+ and transferred to new 15 mL conical tubes (the original tubes were rinsed with 5 mL R10+ and transferred to the new tube too). Cells were pelleted 5 minutes at 300 g at room temperature, washed again with 5 mL RPMI (without serum), and resuspended in 5 ml RPMI. From the “dual-stained sample”, 84 μL (0.05×10⁶) was removed to a 5 mL FACS tube with 1×PBS—for PKH26+ ref control; these were pelleted, resuspended in 150 μL 2% fix solution and set aside. The remaining dual-stained, unstained, and Violet+ samples were pelleted, then resuspended in RPMI as follows: 150 μL to dual-stained samples, 150 μL to unstained samples, and 50 μL to Violet+ reference sample. Dye was added as follows: 150 μL Violet dye to dual-stained tube, 150 μL DMSO-only (no dye) to unstained tube, and 50 μL Violet dye to Violet+ ref. Cells were incubated 15 min. at 37° C., 5% CO₂ and washed with 5 mL R10+. Cells were washed once more as follows: 5 mL of R10+ was added to dual-stained, then cells were pelleted; 5 mL R10+ was added to unstained, then 84 μL (0.05×10⁶) was removed to a 5 mL FACS tube with 1×PBS—for unstained ref. control, the 50K cells were pelleted, resuspended in 150 μL 2% fix solution and set aside, while the remaining unstained cells were pelleted; 5 mL PBS was added to the Violet+ ref, the entire sample was pelleted, resuspended in 300 μL 2% fix solution, transferred to a 5 mL FACS tube, and set aside. Dual-stained and remaining unstained cells were resuspended in 1 mL R10+. The cells were counted by vi-cell by making a 1:10 dilution (50 μL sample+450 μL R10+), and volume was adjusted to make 1.2×10⁶/mL.

For THP-1 cells (both activated and inactivated), T-25 flasks were washed with PBS, then ice-cold 5 mM EDTA was added and incubated for 40 min. on ice. Cells were detached by tapping the side of the flask and pipetting. 1 mL of trypsin was added and incubated for 5 min. at 37 C. Cells were gently resuspended and removed to a 15 mL tube of R10+. For Jurkat cells (both activated and inactivated), cells were harvested from their flasks by pipetting and transferred to a 15 mL tube. All Jurkat and THP-1 cells were washed at 300× g for 5 min, resuspended in 2 mL of RPMI and counted by Vi-cell. Each cell condition was split into 2 tubes (with 0.2-2×10⁶ live cells per tube) and then washed with RPMI by pelleting at 300 g for 5 min at RT. Cells were resuspended in a volume of Diluent C to make a 20×10⁶ cell/mL solution and an equal volume of either 4 μM PKH26 or Diluent C was added to each “dual-stained” and “unstained” conditions of each cell type. Cells were mixed with dye solution rapidly and immediately by pipetting then incubated for 5 minutes at room temperature. 500 μL 100% FBS was added to quench the staining, and cells were incubated for 1 minute. Cells were pelleted by was added to quench the staining, and cells were incubated for 1 minute. Cells were pelleted by centrifugation for 10 min. at 300 g at room temperature, then resuspended in 5 mL R10+ and transferred to new 15 mL conical tubes (the original tubes were rinsed with 5 mL R10+ and transferred to the new tube too). Cells were pelleted 5 minutes at 300 g at room temperature, washed again with 5 mL RPMI (without serum), and resuspended in 5 ml RPMI. From the “dual-stained sample”, 0.05×10⁶ cells were removed to a 5 mL FACS tube with 1×PBS—for PKH26+ ref. control; these were pelleted, resuspended in 150 μL 2% fix solution and set aside. The remaining dual-stained and unstained samples were pelleted, then resuspended in a volume of RPMI to make 20×10⁶ cell/mL. An equal volume of Cell Tracker Deep Red dye or DMSO control was added to the “dual-stained” and “unstained” samples, respectively. Cells were incubated 15 min. at 37° C., 5% CO₂ and washed with 5 mL R10+. Cells were washed once more as follows: 5 mL of R10+ was added to dual-stained, then cells were pelleted; 5 mL R10+ was added to unstained, then 84 μL (0.05×10⁶) was removed to a 5 mL FACS tube with 1×PBS—for unstained ref. control, the 50K cells were pelleted, resuspended in 1500, 2% fix solution and set aside, while the remaining unstained cells were pelleted. Dual-stained and remaining unstained cells were resuspended in 0.25 mL R10+. The cells were counted by vi-cell by making a 1:20 dilution (25 μL sample+475 μL R10+), and volume was adjusted to make 1.2×10⁶/mL.

Co-Culture

Stained K562 cells, THP-1 cells (unstimulated and stimulated), and Jurkat cells (unstimulated and stimulated) were combined according to the table below, giving ratios of donors to acceptors of either 1:5 or 5:1, always totaling 250,000 cells per well in a 96-well flat-bottom plate. The plate was centrifuged for 1 min at 100 g with the brake off. Cells were cultured at 37° C., 5% CO₂ for 1 hr.

| | | Donor | | | | Acceptor | | | | |
|---|---|---|---|---|---|---|---|---|---|---|
| | | | Cell | Membrane | Cells/well | | Cell | Membrane | Cells/well | Donor:Acceptor |
| | Sample Type | Cell | Tracker | Label | (×10\hat{ }6) | Cell | Tracker | Label | (×10\hat{ }6) | ratio |
| 1 | Co-Culture | K562 | Violet | PKH67 | 0.042 | THP (−) | Deep Red | PKH26 | 0.21 | 1:5 |
| 2 | Co-Culture | K562 | Violet | PKH67 | 0.042 | THP (+) | Deep Red | PKH26 | 0.21 | 1:5 |
| 3 | Co-Culture | K562 | Violet | PKH67 | 0.21 | THP (−) | Deep Red | PKH26 | 0.042 | 5:1 |
| 4 | Co-Culture | K562 | Violet | PKH67 | 0.21 | THP (+) | Deep Red | PKH26 | 0.042 | 5:1 |
| 5 | Co-Culture | K562 | Violet | PKH67 | 0.042 | Jurkat (−) | Deep Red | PKH26 | 0.21 | 1:5 |
| 6 | Co-Culture | K562 | Violet | PKH67 | 0.042 | Jurkat (+) | Deep Red | PKH26 | 0.21 | 1:5 |
| 7 | Co-Culture | K562 | Violet | PKH67 | 0.21 | Jurkat (−) | Deep Red | PKH26 | 0.042 | 5:1 |
| 8 | Co-Culture | K562 | Violet | PKH67 | 0.21 | Jurkat (+) | Deep Red | PKH26 | 0.042 | 5:1 |
| 9 | Single cell type | K562 | — | — | 0.21 | | | | | |
| 10 | Single cell type | K562 | Violet | PKH67 | 0.21 | | | | | |
| 11 | Single cell type | | | | | THP (−) | — | | 0.21 | |
| 12 | Single cell type | | | | | THP (+) | — | | 0.21 | |
| 13 | Single cell type | | | | | THP (−) | Deep Red | | 0.21 | |
| 14 | Single cell type | | | | | THP (+) | Deep Red | | 0.21 | |
| 15 | Single cell type | | | | | Jurkat (−) | — | | 0.21 | |
| 16 | Single cell type | | | | | Jurkat (+) | — | | 0.21 | |
| 17 | Single cell type | | | | | Jurkat (−) | Deep Red | | 0.21 | |
| 18 | Single cell type | | | | | Jurkat (+) | Deep Red | | 0.21 | |

Cell Staining

Cells were harvested by pipetting and transferring to a 96-well V-bottom plate. Cells were washed with cold PBS by centrifuging at 300 g for 5 minutes, and then stained with L/D violet (5 μL) for 30 minutes at 4° C. Cells were then washed with WB, resuspended in WB and treated with an Fc Receptor blocking reagent for 20 minutes at 4° C. Then a cocktail of biotin- or fluorescently-conjugated antibodies specific for HLA-G, CD3, CD4, CD14, and HLA-DR were added to the cells and incubated for 30 min at 4° C. Cells were washed with WB by centrifuging at 300 g for 5 min. Cell were stained with a fluorescently-labeled streptavidin for 20 min at 4° C., washed with PBS at 300 g for 5 min, and then fixed with 250 μL 2% fix solution with vigorous pipetting, and then run on the flow cytometer.

Results

Flow cytometry data of cultured cells were analyzed by gating on side scatter-by-forward scatter, then live cells, and then cell tracker dyes to segregate donors (BMQC/Violet-stained K562) from acceptors (DeepRed-stained THP-1 or Jurkat cells). HLA-G positive/negative gates were set based on acceptor cells in the absence of donors. These gates were duplicated on the donors from co-culture samples to determine the percent HLA-G acquisition. Additionally, the median fluorescence intensity (MFI) of the HLA-G channel was quantified on the total donor population as a measure of per-cell change in HLA-G molecules.

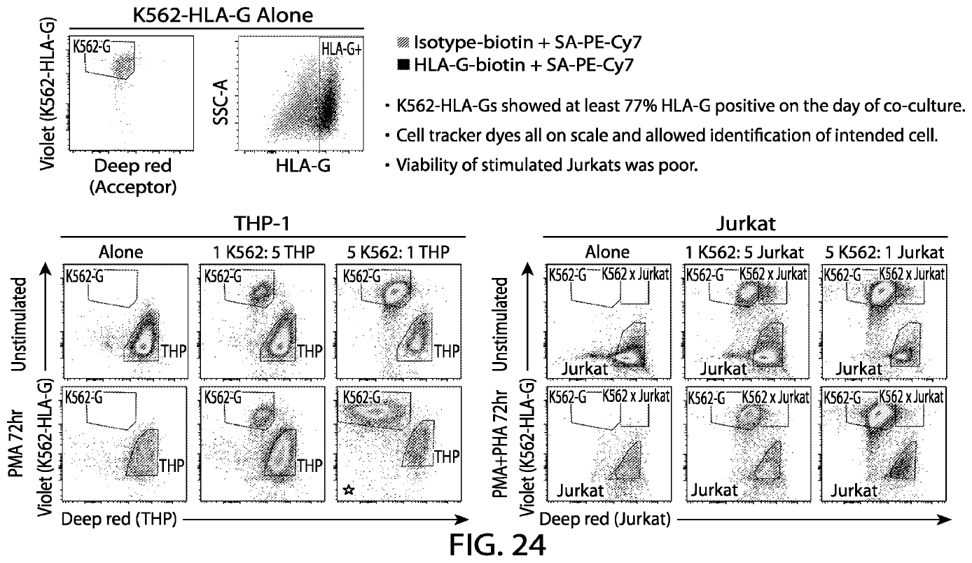

Exemplary flow sorting data for each co-culture setting are shown in FIG. 24. As shown in the top panel, K562-HLA-G cells showed at least 77% HLA-G positivity on the day of co-culture, demonstrating that the donor cells expressed HLA-G as expected. As shown in the bottom left panel, transfer of HLA-G from K562 cells to THP-1 cells increased in a dose-dependent fashion for both stimulated and unstimulated cells, with greater transfer observed in the 5:1 donor:acceptor conditions compared to the respective 1:5 donor:acceptor conditions. As shown in the bottom right panel, transfer of HLA-G from K562 cells to Jurkat cells also increased in a dose-dependent fashion for stimulated and unstimulated cells, with greater transfer observed in the 5:1 donor:acceptor condition compared to the respective 1:5 donor:acceptor condition.

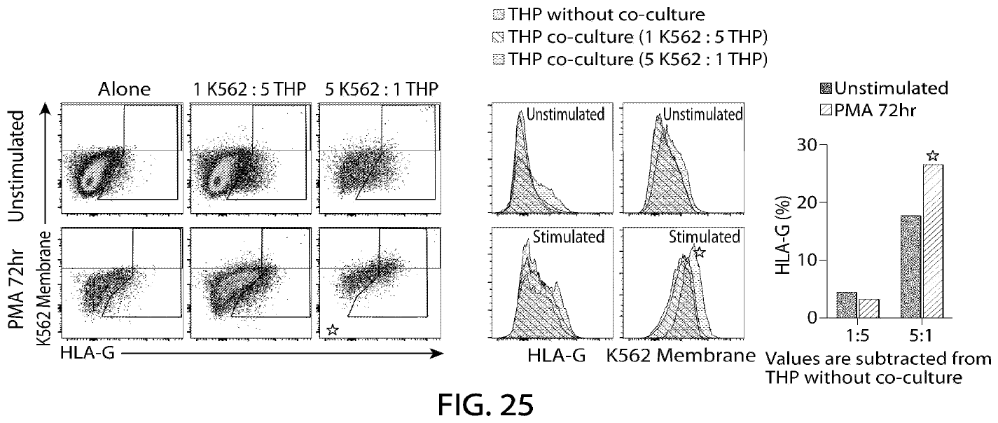

For THP-1 acceptor cells, a dose-dependent transfer of HLA-G from K562 cells was observed (FIG. 25). As noted above, stimulation-dependent increase in HLA-G was observed in the 5:1 donor:acceptor condition. Also observed was an increase in K562 membrane transferred to the THP-1 cells.

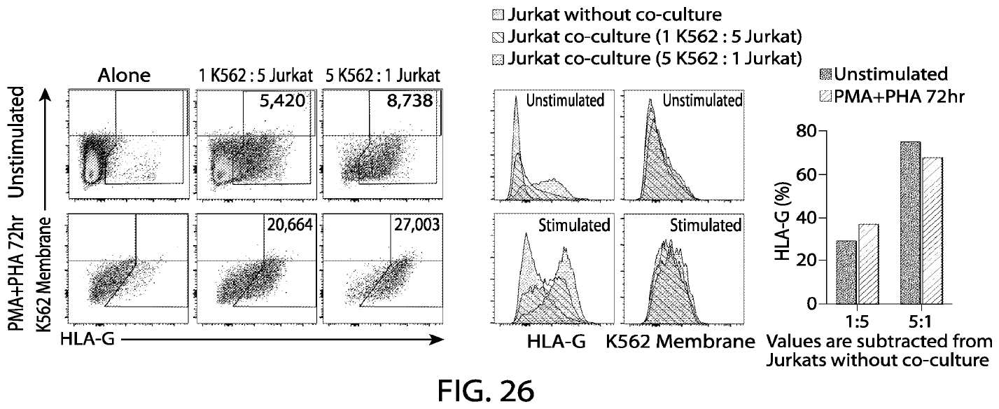

For Jurkat acceptor cells, a dose-dependent transfer of HLA-G from K562 donor cells was also observed (FIG. 26). A similar percentage of Jurkat cells that received HLA-G was observed between the unstimulated and stimulated conditions, although relative per cell levels of HLA-G transferred was higher on stimulated Jurkat cells compared to unstimulated Jurkat cells (as shown in scatter plots in FIG. 26). Also observed was an increase in K562 membrane transferred to the Jurkat cells.

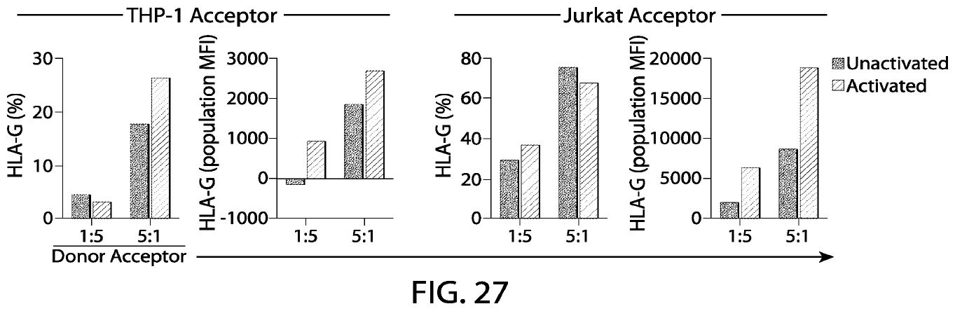

FIG. 27 shows bar graphs of the percent HLA-G-positive and the population MFI of HLA-G of the THP-1 and Jurkat accepter cells (both inactivated and activated) following co-culture. The percent and MFI values shown are background subtracted from the single donor controls. These graphs demonstrate that HLA-G was acquired by both THP-1 and Jurkat cells in a dose dependent manner (percent and MFI of HLA-G increased from 1:5 to 5:1) and in an activation state-dependent manner (percent (for THP-1 cells) and MFI (for THP-1 and Jurkat cells) of HLA-G increased between the inactivated and activated state).

### Example 36: Non-Targeted, Bi-Directional Transfer of Plasma Membrane

In this example, non-targeted, bi-directional transfer of plasma membrane was demonstrated between K562 cells (a myeloid cell line) and human peripheral blood mononuclear cells (PBMCs).

Cell Preparation

On the day of co-culture, a frozen sample of human PBMC were thawed and rested in media at 37° C. and 5% CO₂ for 2 hr. These cells were then labeled with the lipophilic dye PKH26 (2 μM) and then washed and labeled with the cytoplasmic dye Cell Tracker Deep Red (0.75 μM).

On the day of co-culture, K562 cells were harvested from their flasks, labeled with the lipophilic dye PKH67 (2 μM) and then washed and labeled with the cytoplasmic dye Cell Tracker Violet/BMQC (20 μM).

Co-Culture

Labeled donor K562 cells were combined with labeled PBMC acceptor cells at a 10:1 ratio (1.5×10⁶ K562+0.15×10⁶ PBMC) in a 24-well plate. Labeled K562 cells and PBMCs were also added to individual wells as controls. The plate was centrifuged for 1 min at 100 g with the brake off. Cells were incubated at 37° C., 5% CO₂ for 1 hour.

Cell Staining

Cells were harvested by pipetting and transferred to a 96-well V-bottom plate. Cells were then washed and stained with a viability dye for 30 min at 4° C. Cells were then washed and treated with a Fc receptor blocking reagent for 20 min at 4° C. A cocktail of biotin- or fluorescently-conjugated antibodies specific for HLA-G, CD3, CD4, CD14, HLA-DR, CD8, CD19, CD16, and CD56 were directly added to the cells and incubated for 30 min at 4° C. Cells were then washed and stained with a fluorescently-labeled streptavidin for 20 min at 4° C., washed again, and then fixed. Fixed cells were then analyzed by flow cytometry.

Analysis and Results

Flow cytometry data were analyzed by gating on side scatter (SSC)-by-forward scatter (FSC), then live cells, and then cell tracker dyes to segregate donors (BMQC/Violet-labeled K562 cells) from acceptors (DeepRed-labeled PBMCs). Among PBMC, the following cell subsets were further identified by the following antibody staining patterns: CD4+ T cells (CD3+CD4+), CD8+ T cells, (CD3+CD8+), B cells (CD3-CD19+), NK cells (CD3−CD19−CD56+), CD14+ monocytes (SSC-high, HLA-DR+CD14+), and other antigen presenting cells (SSC-high, HLA-DR+CD14−).

To analyze transfer of membrane between cells, gates were set based on control cells that had been incubated in in the absence of the other cell type: A PKH67-positive gate was made on the PKH26-labeled PBMC control cells (separately on each cell subset); A PKH26-positive gate was made on the PKH67-positive K562 control cells. These gates then were applied to the same cell types in the samples that had been incubated as a co-culture. This allowed identification of PKH26-positive PBMC subsets that had become positive for PKH67 (from the K562 cells) and PKH67-positive K562 cells that had become positive for PKH26 (from the PBMCs).

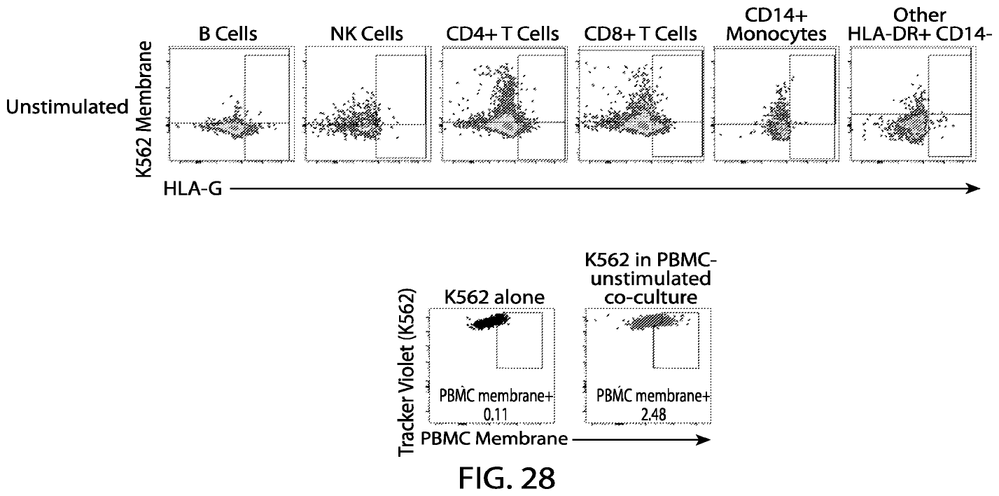

As shown in FIG. 28, this analysis demonstrated that multiple PBMC subsets (including leukocytes) acquired membrane, to varying degrees, from K562 cells (4 to 28% of PBMC subsets were positive for K562 membrane). FIG. 28 also shows that K562 cells acquired membrane from PBMC, as demonstrated by 2.5% of K562 cell being positive for PBMC membrane following incubation.

### Example 37: Transfer of GFP Protein from Donor J76 Cells to Acceptor Ramos Cells

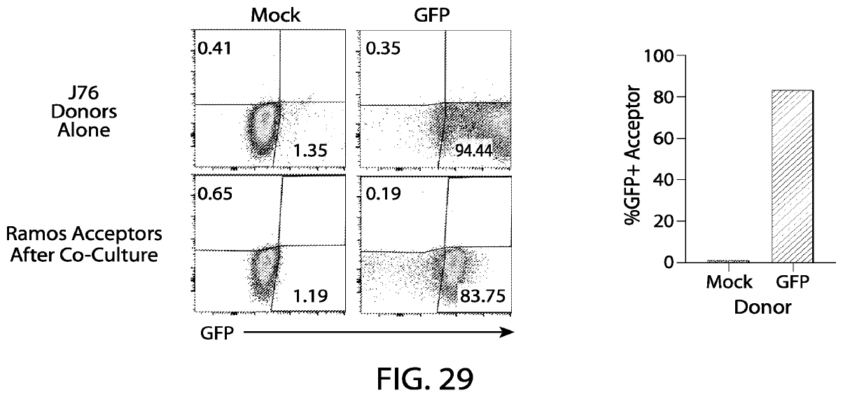

In this example, an exogenous protein, GFP, was expressed in donor cells and then transferred to acceptor cells. Briefly, J76 cells were transfected with either a negative control mock construct or a construct expressing GFP-MS2. The transfected J76 cells were then co-cultured for one hour with Ramos acceptor cells at a ratio of 5 J76 cells: 1 Ramos cell. The Ramos acceptor cells were then assessed for the presence of GFP by flow cytometry. As shown in FIG. 29, Ramos cells co-cultured with J76 cells transfected with the GFP construct showed substantial GFP signal—i.e., 83.75% of such acceptor cells were GFP-positive. In contrast, Ramos cells co-cultured with J76 cells transfected with the mock construct showed minimal GFP signal—only 1.19% of such acceptor cells showed fluorescent signal. These data demonstrate successful transfer of GFP protein from J76 donor cells to Ramos acceptor cells.

What is claimed is:

1. A composition comprising a plurality of donor cells, the plurality of donor cells comprising:
(a) a membrane-associated agent, the agent comprising:
a membrane-associated moiety, and
one or both of an extracellular moiety or an intracellular moiety, and
(b) optionally a cargo molecule,
wherein the membrane-associated agent and optionally the cargo molecule are configured to be transferred to an acceptor cell;
wherein at least one of the membrane-associated moiety, extracellular moiety, intracellular moiety, or the cargo molecule is exogenous to the plurality of donor cells, and
wherein the plurality of donor cells transfers a detectable amount or a biologically effective amount of the membrane-associated agent and/or the cargo molecule to a plurality of acceptor cells.

2. A composition comprising a plurality of donor cells, the plurality of donor cells comprising:
(a) a membrane-associated agent, the agent comprising:
a membrane-associated moiety, and
one or both of an extracellular moiety or an intracellular moiety, and
(b) optionally a cargo molecule,
wherein the membrane-associated agent and optionally the cargo molecule are configured to be transferred to an acceptor cell;
wherein at least one of the membrane-associated moiety, extracellular moiety, intracellular moiety, or cargo molecule is present at a different level in the donor cell than a source cell from which the donor cell is derived, and
wherein the plurality of donor cells transfers a detectable amount or a biologically effective amount of the membrane-associated agent and/or the cargo molecule to a plurality of acceptor cells.

3. A composition comprising a plurality of donor cells, the plurality of donor cells comprising:
(a) a membrane-associated agent, the agent comprising:
a membrane-associated moiety, and
one or both of an extracellular moiety or an intracellular moiety, and
(b) optionally a cargo molecule,
wherein the membrane-associated agent and optionally the cargo molecule are configured to be transferred to an acceptor cell;
wherein at least one of the membrane-associated moiety, extracellular moiety, intracellular moiety, or the cargo molecule is exogenous to the plurality of donor cells, and
wherein the membrane-associated moiety or intracellular moiety comprises a protease cleavage site recognized by a protease that is not expressed in the plurality of donor cells.

4. A composition comprising a plurality of donor cells, the plurality of donor cells comprising:
(a) a membrane-associated agent, the agent comprising:
a membrane-associated moiety, and
one or both of an extracellular moiety or an intracellular moiety, and
(b) optionally a cargo molecule,
wherein the membrane-associated agent and optionally the cargo molecule are configured to be transferred to an acceptor cell,
wherein at least one of the membrane-associated moiety, extracellular moiety, intracellular moiety, or cargo molecule is present at a different level in the plurality of donor cells than a source cell from which the plurality of donor cells is derived, and
wherein the membrane-associated moiety or intracellular moiety comprises a protease cleavage site recognized by a protease that is not expressed in the plurality of donor cells.

5. The composition of either of claim 3 or 4, wherein the protease is present in the acceptor cell.

6. The composition of claim 5, wherein the protease is exogenously expressed in the acceptor cell.

7. The composition of claim 5, wherein the protease is endogenously expressed in the acceptor cell.

8. A composition comprising a plurality of donor cells, the plurality of donor cells comprising:
(a) a membrane-associated agent, the agent comprising:
a membrane-associated moiety, and
one or both of an extracellular moiety or an intracellular moiety, and
(b) a cargo molecule,
wherein the membrane-associated agent is configured to be transferred to an acceptor cell,
wherein at least one of the membrane-associated moiety, extracellular moiety, intracellular moiety, or the cargo molecule is exogenous to the plurality of donor cells,
wherein the extracellular moiety comprises a targeting domain that binds to a first target cell moiety and a second target cell moiety and wherein the acceptor cell comprises the first target cell moiety or the second target cell moiety but not both, and
wherein the intracellular moiety is associated, e.g., non-covalently, with the cargo molecule.

9. A composition comprising a plurality of donor cells, the plurality of donor cells comprising:
(a) a membrane-associated agent, the agent comprising:
a membrane-associated moiety, and
one or both of an extracellular moiety, an intracellular moiety, and
(b) a cargo molecule,
wherein the membrane-associated agent is configured to be transferred to an acceptor cell;
wherein at least one of the membrane-associated moiety, extracellular moiety, intracellular moiety, or cargo molecule is present at a different level in the donor cell than a source cell from which the donor cell is derived,
wherein the extracellular moiety comprises a targeting domain that binds to a first target cell moiety and a second target cell moiety and wherein the acceptor cell comprises the first target cell moiety or the second target cell moiety but not both, and
wherein the intracellular moiety is non-covalently associated with, e.g., binds to, the cargo molecule.

10. The composition of any of claims 1-9, wherein the donor cell comprises a cargo molecule configured to be transferred to an acceptor cell.

11. The composition of any of claims 1-10, wherein at least one of the membrane-associated moiety, extracellular moiety, intracellular moiety, or cargo molecule is differentially expressed.

12. A composition comprising a plurality of acceptor cells, the plurality of acceptor cells comprising:
(a) a membrane-associated agent, the membrane-associated agent comprising:
a membrane-associated moiety, and
one or both of an extracellular moiety or an intracellular moiety, and
(b) optionally a cargo molecule,
wherein the acceptor cells do not comprise a nucleic acid encoding the membrane-associated agent,
wherein at least one of the membrane-associated moiety, extracellular moiety, intracellular moiety, or cargo molecule is exogenous to the acceptor cell,
optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell.

13. A composition comprising a plurality of acceptor cells, the plurality of acceptor cells comprising:
(a) a membrane-associated agent, the agent comprising:
a membrane-associated moiety;
one or both of an extracellular moiety or an intracellular moiety; and
(b) optionally a cargo molecule;
wherein the acceptor cells do not substantially express a nucleic acid encoding the membrane-associated agent or, if a cargo molecule is present, do not substantially express a nucleic acid encoding the cargo molecule,
optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell.

14. A composition comprising a plurality of acceptor cells, the plurality of acceptor cells comprising:
(a) a membrane-associated agent, the membrane-associated agent comprising:
a membrane-associated moiety, and
one or both of an extracellular moiety or an intracellular moiety, and
(b) optionally a cargo molecule,
wherein the acceptor cells do not comprise a nucleic acid encoding the membrane-associated agent and, if a cargo molecule is present, do not comprise a nucleic acid encoding the cargo molecule,
wherein at least one of the membrane-associated moiety, extracellular moiety, intracellular moiety, or cargo molecule is exogenous to the acceptor cell,
optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell, and
wherein:
(1) the membrane-associated moiety or intracellular moiety comprises a protease cleavage site recognized by a protease present in the plurality of acceptor cells, or
(2) the plurality of acceptor cells comprises a membrane-associated agent comprising a cleaved fragment corresponding to cleavage by said protease at said protease cleavage site.

15. A composition comprising a plurality of acceptor cells, the plurality of acceptor cells comprising:
(a) a membrane-associated agent, the agent comprising:
a membrane-associated moiety;
one or both of an extracellular moiety or an intracellular moiety; and
(b) optionally a cargo molecule;
wherein the acceptor cells do not substantially express a nucleic acid encoding the membrane-associated agent or, if a cargo molecule is present, do not substantially express a nucleic acid encoding the cargo molecule,
optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell, and
wherein:
(1) the membrane-associated moiety or intracellular moiety comprises a protease cleavage site recognized by a protease present in the plurality of acceptor cells, or
(2) an acceptor cell of the plurality of acceptor cells comprises a membrane-associated agent comprising a cleaved fragment corresponding to cleavage by said protease at said protease cleavage site.

16. A composition comprising a plurality of acceptor cells, the plurality of acceptor cells comprising:
(a) a membrane-associated agent, the agent comprising:
a membrane-associated moiety, and
one or both of an extracellular moiety or an intracellular moiety, and
(b) a cargo molecule,
wherein the acceptor cells do not comprise a nucleic acid encoding the membrane-associated agent and, do not comprise a nucleic acid encoding the cargo molecule,
wherein at least one of the membrane-associated moiety, extracellular moiety, intracellular moiety, or cargo molecule is exogenous to the acceptor cell,
optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell,
wherein the extracellular moiety comprises a targeting domain that binds to a first target cell moiety and a second target cell moiety and wherein the acceptor cell comprises the first target cell moiety or the second target cell moiety but not both, and
wherein the intracellular moiety is associated, e.g., non-covalently, with the cargo molecule.

17. A composition comprising a plurality of acceptor cells, the plurality of acceptor cells comprising:
(a) a membrane-associated agent, the agent comprising:
a membrane-associated moiety;
one or both of an extracellular moiety or an intracellular moiety; and
(b) a cargo molecule;
wherein the acceptor cells do not substantially express a nucleic acid encoding the membrane-associated agent and do not substantially express a nucleic acid encoding the cargo molecule,
optionally wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality comprise a detectable amount and/or a biologically effective amount of the membrane-associated agent and/or the cargo molecule from a donor cell,
wherein the extracellular moiety comprises a targeting domain that binds to a first target cell moiety and a second target cell moiety and wherein the acceptor cell comprises the first target cell moiety or the second target cell moiety but not both, and
wherein the intracellular moiety is associated, e.g., non-covalently with the cargo molecule.

18. The composition of any of claim 12, 14, or 16, wherein the plurality of acceptor cells received the membrane-associated agent from one or more donor cells.

19. The composition of any of claim 12, 14, 16, or 18, wherein the plurality of acceptor cells received the cargo molecule from one or more donor cells.

20. The composition of any preceding claim, wherein the plurality of donor cells or the plurality of acceptor cells are purified.

21. The composition of any preceding claim, wherein the membrane-associated agent is transferred from one or more donor cells to one or more acceptor cells through a membrane transfer process.

22. The composition of claim 21, wherein the membrane transfer process is a membrane fusion event, a receptor-ligand interaction, a cell bridging event, or cell to cell contact event.

23. The composition of any of claim 1-11 or 20-22, wherein the plurality of donor cells transfers a detectable amount or a biologically effective amount of the membrane-associated agent or the cargo molecule to at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of the cells in a plurality of acceptor cells.

24. The composition of any of claims 12-22, wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality of acceptor cells comprise a detectable amount and/or a biologically effective amount of the membrane-associated agent or the cargo molecule.

25. The composition of any of claim 12-22 or 24, wherein the acceptor cells comprise a level of membrane-associated agent or cargo molecule that is at least 0.01, 0.1, 1, 2, 3, 4, 5, 6, 7, 8, 9, 10, 20, 30, 40, 50, 60, 70, 80, 90, 95, or 99% of the level of membrane-associated agent or cargo molecule present in the donor cells.

26. The composition of any of claim 12-22, 24, or 25, wherein at least one biological function of the plurality of acceptor cells is modulated by the transfer of the membrane-associated agent or cargo molecule, wherein the biological function is selected from the group consisting of:
(ix) modulating, e.g., increasing or decreasing a level or activity of a molecule (e.g., a protein, nucleic acid, or metabolite, drug, or toxin) in the acceptor cell or the plurality of acceptor cells;
(x) modulating, e.g., increasing or decreasing, enzyme activity in the acceptor cell or the plurality of acceptor cells;
(xi) modulating, e.g., increasing or decreasing, a genetic or an epigenetic event in the acceptor cell or the plurality of acceptor cells;
(xii) modulating, e.g., promoting or inhibiting, acceptor cell differentiation;
(xiii) modulating acceptor cell reprogramming;
(xiv) activating a signaling pathway in an acceptor cell;
(xv) delivering a cargo, e.g., a gene editing complex; and
(xvi) modifying cell adhesion and trafficking.

27. The composition of any preceding claim, wherein the membrane-associated moiety is operatively associated or linked to the intracellular moiety.

28. The composition of any preceding claim, wherein the membrane-associated moiety is operatively associated or linked to the cargo molecule.

29. The composition of any preceding claim, wherein the membrane-associated moiety is operatively associated or linked to the extracellular moiety.

30. The composition of any preceding claim, wherein the membrane-associated moiety is a transmembrane moiety that comprises a transmembrane domain from a receptor.

31. The composition of any preceding claim, wherein the membrane associated moiety comprises a cleavage site recognized by a protease.

32. The composition of claim 31, wherein the protease is TEV protease or RHBDL2.

33. The composition of any preceding claim, wherein the extracellular moiety comprises a specificity portion, an accessory portion, or both.

34. The composition of any preceding claim, wherein the extracellular moiety comprises a targeting domain, a transfer promoting moiety, or both.

35. The composition of any preceding claim, wherein the extracellular moiety comprises one or more of an antibody or functional fragment or derivative thereof (e.g., an scFv), a streptavidin domain, a receptor, a ligand, a cell surface protein, a sugar, or a lipid.

36. The composition of any preceding claim, wherein the extracellular moiety comprises a selectin, a claudin, a gap junction protein, an annexin, an integrin, a lectin, a tight junction protein, a desmosomal protein, or a cell adhesion molecule involved in the leukocyte adhesion cascade.

37. The composition of any preceding claim, wherein the extracellular moiety comprises a trafficking receptor.

38. The composition of claim 37, wherein the trafficking receptor is a chemokine receptor.

39. The composition of any preceding claim, wherein the extracellular moiety or the membrane-associated moiety comprises an activation or inhibition receptor.

40. The composition of any preceding claim, wherein the intracellular moiety comprises a functional portion, an accessory portion, or both.

41. The composition of any preceding claim, wherein the intracellular moiety comprises one or more of an antibody or functional fragment thereof, a reporter agent, a signaling protein, an enzyme (or functional portion thereof), a transcription factor, an epigenetic remodeling agent, a protein binding domain, a nucleic acid-binding protein or domain (e.g., an RNA-binding protein or domain or an DNA-binding protein or domain), a hydrophobic domain, a lipid raft targeting domain, or drug-binding domain.

42. The composition of any preceding claim, wherein the intracellular moiety comprises EGFP, β-lactamase, Cre recombinase, a CRISPR/Cas protein and a guide RNA, or a functional portion or variant of any thereof.

43. The composition of any preceding claim, wherein the cargo molecule is operably associated or linked to the membrane-associated agent.

44. The composition of any preceding claim, wherein the cargo molecule is operably associated, linked, covalently linked, or non-covalently linked to the membrane-associated moiety, extracellular moiety, or intracellular moiety.

45. The composition of any preceding claim, wherein the cargo molecule is not associated or linked with the membrane-associated agent.

46. The composition of any preceding claim, wherein the cargo molecule is associated or linked to the membrane.

47. The composition of any preceding claim, wherein the cargo molecule is chosen from a small molecule, a nucleic acid, a peptide, a protein, a protein-bound molecule, a protein-bound antibody molecule, a lipid, a carbohydrate, or an organelle.

48. The composition of any of preceding claim, wherein the cargo molecule is released from the membrane-associated moiety or the intracellular moiety.

49. The composition of any preceding claim, wherein the membrane-associated agent is an exogenous membrane-associated agent.

50. The composition of claim 49, wherein the exogenous membrane-associated agent comprises a fusion protein.

51. The composition of any preceding claim, wherein the donor cells or acceptor cells are primary cells or immortalized cells.

52. The composition of any preceding claim, wherein the donor cells or acceptor cells are synthetic cells.

53. The composition of any preceding claim, wherein the donor cells are hematopoietic cells, immune cells, embryonic cells, somatic stem cells, endothelial cells, fibroblasts, epithelial cells, cancer cells, or diseased cells.

54. The composition of any preceding claim, wherein the acceptor cells are neutrophils, lymphocytes, PMNs, monocytes, dendritic cells, macrophages, granulocytes, mesenchymal stem cells, bone marrow stem cells, induced pluripotent stem cells, embryonic stem cells, or myeloblasts.

55. The composition of any preceding claim, wherein the acceptor cells are somatic stem cells, hematopoietic cells, nerve cells, neuroglial cells, muscle cells, cartilage cells, bone cells, endothelial cells, epithelial cells, fibroblasts, adipocytes, gametes, cancer cells, or diseased cells.

56. The composition of any preceding claim, wherein the acceptor cells are somatic stem cells, hematopoietic cells, nerve cells, neuroglial cells, muscle cells, cartilage cells, bone cells, endothelial cells, epithelial cells, fibroblasts, adipocytes, gametes, cancer cells, or diseased cells.

57. The composition of any preceding claim, wherein the acceptor cells are neutrophils, lymphocytes, PMNs, monocytes, dendritic cells, macrophages, granulocytes, mesenchymal stem cells, bone marrow stem cells, induced pluripotent stem cells, embryonic stem cells, or myeloblasts.

58. The composition of any preceding claim, wherein the donor cells comprise a decreased level of a non-essential component.

59. The composition of any preceding claim, wherein expression of at least one other membrane-associated protein in the donor cells is modulated.

60. The composition of any preceding claim, wherein at least one other membrane-associated protein in the donor cells is operatively associated or linked to a cytoskeletal component of the donor cell.

61. The composition of any preceding claim, wherein the donor cells are autologous to the acceptor cells.

62. The composition of any of claims 1-60, wherein the donor cells are allogeneic to the acceptor cells.

63. The composition of any preceding claim, wherein the donor cells and acceptor cells are homotypic.

64. The composition of any preceding claim, wherein the donor cells or acceptor cells are obtained from an apheresis sample, a blood draw, a cell line, or a tissue biopsy.

65. The composition of any preceding claim, wherein the membrane-associated agent comprises a polypeptide or domain that is expressed endogenously in the donor or acceptor cells.

66. The composition of claim 65, wherein the membrane-associated agent is expressed at a level of at least 1.1×, 1.5×, 2×, 3×, 4×, 5×, 10×, 50×, or 100× the level of the endogenously expressed polypeptide or domain.

67. The composition of any preceding claim, wherein the extracellular moiety is exogenous to the donor or acceptor cells.

68. The composition of any preceding claim, wherein the intracellular moiety is exogenous to the donor or acceptor cells.

69. The composition of any preceding claim, wherein the membrane-associated moiety is exogenous to the donor or acceptor cells.

70. The composition of any preceding claim, wherein the plurality of acceptor cells are immune cells.

71. The composition of any preceding claim, wherein the plurality of acceptor cells are not immune cells.

72. The composition of any preceding claim, wherein the plurality of donor cells are immune cells.

73. The composition of any preceding claim, wherein the plurality of donor cells are not immune cells.

74. The composition of any preceding claim, wherein the plurality of donor cells transfers a detectable amount or a biologically effective amount of the membrane-associated agent and/or the cargo molecule to at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of the plurality of acceptor cells.

75. The composition of any preceding claim, wherein at least 0.1%, 0.2%, 0.3%, 0.4%, 0.5%, 1%, 2%, 3%, 4%, 5%, 6%, 7%, 8%, 9%, 10%, 15%, 20%, 25%, 30%, 40%, 50%, 60%, 70%, 75%, 80%, 85%, 90%, 95%, 96%, 97%, 98%, or 99% of cells in the plurality of acceptor cells comprise a detectable amount and/or a biologically effective amount of the membrane-associated agent and/or the cargo molecule from a donor cell.

76. A system comprising:
the composition comprising a plurality of donor cells of the composition of any of claim 1-11, 20-23, 27-47, 49-56, or 58-75, and
an acceptor cell,
wherein the donor cells and acceptor cell are provided under conditions suitable for transfer of the membrane-associated agent, cargo molecule, or both from the donor cell to the acceptor cell,
wherein the acceptor cell differentially expresses the membrane-associated agent and/or cargo molecule.

77. A pharmaceutical composition comprising the plurality of donor cells of the composition of any of claim 1-11, 20-23, 27-47, 49-56, or 58-75.

78. A pharmaceutical composition comprising the plurality of acceptor cells of the composition of any of claim 12-22, 24-52, 54-57, or 64-75.

79. The pharmaceutical composition of claim 77 or 78, wherein:
i) the pharmaceutical composition meets a pharmaceutical or good manufacturing practices (GMP) standard;
ii) the donor cell or acceptor cell was made according to good manufacturing practices (GMP);
iii) the donor cell or acceptor cell has a pathogen level below a predetermined reference value; or
iv) the donor cell or acceptor cell has a contaminant level below a predetermined reference value.

80. A method of modifying an acceptor cell, comprising:
contacting the acceptor cell with a composition comprising a plurality of donor cells of the composition of any of claim 1-11, 20-23, 27-47, 49-56, or 58-75, under conditions suitable for transfer of the membrane-associated agent and/or cargo molecule to the acceptor cell,
wherein after the transfer the acceptor cell comprises an increased amount of the membrane-associated agent and/or cargo molecule,
thereby modifying the acceptor cell.

81. A method of making a modified cell, comprising:
providing an unmodified cell,
contacting the unmodified cell with a plurality of donor cells of the composition of any of claim 1-11, 20-23, 27-47, 49-56, or 58-75, under conditions suitable for transfer of the membrane-associated agent and/or cargo molecule to the unmodified cell,
thereby making a modified cell,
wherein after the transfer the modified cell comprises an increased amount of the membrane-associated agent and/or cargo molecule than the unmodified cell.

82. A method of delivering a cargo molecule to a cell, comprising:
providing the composition comprising a plurality of donor cells of the composition of any of claim 1-11, 20-23, 27-47, 49-56, or 58-75, wherein the donor cells comprise the cargo molecule; and
contacting the cell with the plurality of donor cells under conditions suitable for transfer of the membrane-associated agent to the acceptor cell,
thereby delivering the cargo molecule to the cell.

83. A method of modulating a biological function, a target tissue, or a cell in a subject comprising administering to the subject, or contacting the target tissue or the cell with, a composition comprising a plurality of donor cells, a composition comprising a plurality of acceptor cells, a system, or a pharmaceutical composition of any of claims 1-79, thereby modulating the biological function in the subject.

84. The method of any of claims 80-83, wherein the plurality of donor cells is stimulated prior to the administering or contacting step to improve transfer of the membrane-associated agent and/or cargo molecule to the cell or acceptor cell.

85. The method of claim 84, wherein stimulation comprises treating the plurality of donor cells with an activating agent.

86. The method of claim 85, wherein the activating agent comprises PMA or PHA-L, or both.
